SEQUENCE LISTING (186 sequences)

SEQ ID NO: 1 - protein, 8 aa, Artificial Sequence
  CDR1, NRCoV2-1a and NRCoV2-1d
GSTLDYYA

SEQ ID NO: 2 - protein, 8 aa, Artificial Sequence
  CDR1, NRCoV2-1c
GSILDYYA

SEQ ID NO: 3 - protein, 8 aa, Artificial Sequence
  CDR1, NRCoV2-02
GFTFSNYA

SEQ ID NO: 4 - protein, 8 aa, Artificial Sequence
  CDR1, NRCoV2-03
GITFSYYA

SEQ ID NO: 5 - protein, 8 aa, Artificial Sequence
  CDR1, NRCoV2-04
GSPFSNVV

SEQ ID NO: 6 - protein, 8 aa, Artificial Sequence
  CDR1, NRCoV2-05
GFIFSNYA

SEQ ID NO: 7 - protein, 8 aa, Artificial Sequence
  CDR1, NRCoV2-06
VSTFSSYA

SEQ ID NO: 8 - protein, 8 aa, Artificial Sequence
  CDR1, NRCoV2-07
GVTLDYYA

SEQ ID NO: 9 - protein, 8 aa, Artificial Sequence
  CDR1, NRCoV2-08
GFTLDDYA

SEQ ID NO: 10 - protein, 8 aa, Artificial Sequence
  CDR1, NRCoV2-10
GNTFSRSN

SEQ ID NO: 11 - protein, 8 aa, Artificial Sequence
  CDR1, NRCoV2-11
GSSLDSYS

SEQ ID NO: 12 - protein, 8 aa, Artificial Sequence
  CDR1, NRCoV2-11a
GFTLDSYN

SEQ ID NO: 13 - protein, 8 aa, Artificial Sequence
  CDR1, NRCoV2-12
GRTFRNYV

SEQ ID NO: 14 - protein, 8 aa, Artificial Sequence
  CDR1, NRCoV2-14
GTTFSHYA

SEQ ID NO: 15 - protein, 8 aa, Artificial Sequence
  CDR1, NRCoV2-15
GSTSGRNT

SEQ ID NO: 16 - protein, 8 aa, Artificial Sequence
  CDR1, NRCoV2-17
GSPFSQLA

SEQ ID NO: 17 - protein, 8 aa, Artificial Sequence
  CDR1, NRCoV2-18
GITISGYN

SEQ ID NO: 18 - protein, 8 aa, Artificial Sequence
  CDR1, NRCoV2-19
GLTLNSYA

SEQ ID NO: 19 - protein, 8 aa, Artificial Sequence
  CDR1, NRCoV2-20
GRTFSNYV

SEQ ID NO: 20 - protein, 8 aa, Artificial Sequence
  CDR1, NRCoV2-21, NRCoV2-MRed03, and NRCoV2-MRed05
GFTLDYYA

SEQ ID NO: 21 - protein, 8 aa, Artificial Sequence
  CDR1, NRCoV2-SR01
GFTFDNYA

SEQ ID NO: 22 - protein, 8 aa, Artificial Sequence
  CDR1, NRCoV2-SR02
EFTLNYYS

SEQ ID NO: 23 - protein, 8 aa, Artificial Sequence
  CDR1, NRCoV2-SR03
GSIFSNNH

SEQ ID NO: 24 - protein, 8 aa, Artificial Sequence
  CDR1, NRCoV2-SR04
GRTFSSHT

SEQ ID NO: 25 - protein, 8 aa, Artificial Sequence
  CDR1, NRCoV2-SR13
GSRFGSKH

SEQ ID NO: 26 - protein, 8 aa, Artificial Sequence
  CDR1, NRCoV2-SR16
GTTFSRYH

SEQ ID NO: 27 - protein, 8 aa, Artificial Sequence
  CDR1, NRCoV2-S2A3
GRPYSNYA

SEQ ID NO: 28 - protein, 8 aa, Artificial Sequence
  CDR1, NRCoV2-S2A4
GSPFRSNV

SEQ ID NO: 29 - protein, 8 aa, Artificial Sequence
  CDR1, NRCoV2-S2B3
ASTFGDSA

SEQ ID NO: 30 - protein, 8 aa, Artificial Sequence
  CDR1, NRCoV2-S2H4
GFTFNLYS

SEQ ID NO: 31 - protein, 8 aa, Artificial Sequence
  CDR1, NRCoV2-S2F3
VRILSVPA

SEQ ID NO: 32 - protein, 8 aa, Artificial Sequence
  CDR1, NRCoV2-S2G3
GSTFGIFL

SEQ ID NO: 33 - protein, 8 aa, Artificial Sequence
  CDR1, NRCoV2-S2G4
GSTFSGYA

SEQ ID NO: 34 - protein, 8 aa, Artificial Sequence
  CDR1, NRCoV2-S202
GITVSRIG

SEQ ID NO: 35 - protein, 8 aa, Artificial Sequence
  CDR1, NRCoV2-MRed02
GNIFSINS

SEQ ID NO: 36 - protein, 8 aa, Artificial Sequence
  CDR1, NRCoV2-MRed04
GNSFSINT

SEQ ID NO: 37 - protein, 8 aa, Artificial Sequence
  CDR1, NRCoV2-MRed06
GFTLAYYA

SEQ ID NO: 38 - protein, 8 aa, Artificial Sequence
  CDR1, NRCoV2-MRed07
GSIGPFNT

SEQ ID NO: 39 - protein, 8 aa, Artificial Sequence
  CDR1, NRCoV2-MRed11
GFTFSSYA

SEQ ID NO: 40 - protein, 8 aa, Artificial Sequence
  CDR1, NRCoV2-MRed18
TTVFGRNA

SEQ ID NO: 41 - protein, 8 aa, Artificial Sequence
  CDR1, NRCoV2-MRed19
TIIFKGQT

SEQ ID NO: 42 - protein, 8 aa, Artificial Sequence
  CDR1, NRCoV2-MRed20
GLSFSSYD

SEQ ID NO: 43 - protein, 8 aa, Artificial Sequence
  CDR1, NRCoV2-MRed22
GSVFASNA

SEQ ID NO: 44 - protein, 8 aa, Artificial Sequence
  CDR1, NRCoV2-MRed25
GHTFSRYG

SEQ ID NO: 45 - protein, 8 aa, Artificial Sequence
  CDR2, NRCoV2-1a and  NRCoV2-1c
VSSSDGST

SEQ ID NO: 46 - protein, 8 aa, Artificial Sequence
  CDR2, NRCoV2-1d
VSSSDGNT

SEQ ID NO: 47 - protein, 8 aa, Artificial Sequence
  CDR2, NRCoV2-02
ISGRGDDT

SEQ ID NO: 48 - protein, 7 aa, Artificial Sequence
  CDR2, NRCoV2-03
MSNMDST

SEQ ID NO: 49 - protein, 7 aa, Artificial Sequence
  CDR2, NRCoV2-04
ISGGGIA

SEQ ID NO: 50 - protein, 8 aa, Artificial Sequence
  CDR2, NRCoV2-05
INSGGGDT

SEQ ID NO: 51 - protein, 7 aa, Artificial Sequence
  CDR2, NRCoV2-06
IGFVGAT

SEQ ID NO: 52 - protein, 8 aa, Artificial Sequence
  CDR2, NRCoV2-07
ISSNGRRN

SEQ ID NO: 53 - protein, 8 aa, Artificial Sequence
  CDR2, NRCoV2-08
ISRSGTTT

SEQ ID NO: 54 - protein, 7 aa, Artificial Sequence
  CDR2, NRCoV2-10
ISSRGIS

SEQ ID NO: 55 - protein, 8 aa, Artificial Sequence
  CDR2, NRCoV2-11
ISRYYSST

SEQ ID NO: 56 - protein, 8 aa, Artificial Sequence
  CDR2, NRCoV2-11a
ISRYYEST

SEQ ID NO: 57 - protein, 11 aa, Artificial Sequence
  CDR2, NRCoV2-12
VAAISWGGTEI

SEQ ID NO: 58 - protein, 7 aa, Artificial Sequence
  CDR2, NRCoV2-14
ISVFGST

SEQ ID NO: 59 - protein, 7 aa, Artificial Sequence
  CDR2, NRCoV2-15
VSTSGAT

SEQ ID NO: 60 - protein, 7 aa, Artificial Sequence
  CDR2, NRCoV2-17
ISPTGNR

SEQ ID NO: 61 - protein, 7 aa, Artificial Sequence
  CDR2, NRCoV2-18
INSGGST

SEQ ID NO: 62 - protein, 7 aa, Artificial Sequence
  CDR2, NRCoV2-19
LTSGGTG

SEQ ID NO: 63 - protein, 11 aa, Artificial Sequence
  CDR2, NRCoV2-20
VAVISGSDTET

SEQ ID NO: 64 - protein, 7 aa, Artificial Sequence
  CDR2, NRCoV2-21
ISSGGST

SEQ ID NO: 65 - protein, 8 aa, Artificial Sequence
  CDR2, NRCoV2-SR01
ISGNGGVT

SEQ ID NO: 66 - protein, 8 aa, Artificial Sequence
  CDR2, NRCoV2-SR02
IRYSGGGI

SEQ ID NO: 67 - protein, 7 aa, Artificial Sequence
  CDR2, NRCoV2-SR03
ISSGGKT

SEQ ID NO: 68 - protein, 10 aa, Artificial Sequence
  CDR2, NRCoV2-SR04
ISMGGNTNYA

SEQ ID NO: 69 - protein, 7 aa, Artificial Sequence
  CDR2, NRCoV2-SR13
ISSGGST

SEQ ID NO: 70 - protein, 7 aa, Artificial Sequence
  CDR2, NRCoV2-SR16
ISTSGAV

SEQ ID NO: 71 - protein, 15 aa, Artificial Sequence
  CDR2, NRCoV2-S2A3
KQRELVAAISSGGTT

SEQ ID NO: 72 - protein, 7 aa, Artificial Sequence
  CDR2, NRCoV2-S2A4
ISTGGSR

SEQ ID NO: 73 - protein, 7 aa, Artificial Sequence
  CDR2, NRCoV2-S2B3
ISTGSNT

SEQ ID NO: 74 - protein, 10 aa, Artificial Sequence
  CDR2, NRCoV2-S2H4
INSGDRDSTT

SEQ ID NO: 75 - protein, 7 aa, Artificial Sequence
  CDR2, NRCoV2-S2F3
ITSGGST

SEQ ID NO: 76 - protein, 7 aa, Artificial Sequence
  CDR2, NRCoV2-S2G3
ITSGGAT

SEQ ID NO: 77 - protein, 7 aa, Artificial Sequence
  CDR2, NRCoV2-S2G4
ISSDGDK

SEQ ID NO: 78 - protein, 7 aa, Artificial Sequence
  CDR2, NRCoV2-S202
ISAGGST

SEQ ID NO: 79 - protein, 7 aa, Artificial Sequence
  CDR2, NRCoV2-MRed02
IWSDSRT

SEQ ID NO: 80 - protein, 7 aa, Artificial Sequence
  CDR2, NRCoV2-MRed04
IWSDTTT

SEQ ID NO: 81 - protein, 8 aa, Artificial Sequence
  CDR2, NRCoV2-MRed03 and NRCoV2-MRed05
ISSSDGST

SEQ ID NO: 82 - protein, 8 aa, Artificial Sequence
  CDR2, NRCoV2-MRed06
ISSSDGSA

SEQ ID NO: 83 - protein, 7 aa, Artificial Sequence
  CDR2, NRCoV2-MRed07
ITRGGVT

SEQ ID NO: 84 - protein, 8 aa, Artificial Sequence
  CDR2, NRCoV2-MRed11
INSGGGST

SEQ ID NO: 85 - protein, 7 aa, Artificial Sequence
  CDR2, NRCoV2-MRed18
VSDGGTP

SEQ ID NO: 86 - protein, 7 aa, Artificial Sequence
  CDR2, NRCoV2-MRed19
MTTSGSA

SEQ ID NO: 87 - protein, 8 aa, Artificial Sequence
  CDR2, NRCoV2-MRed20
IRESGSGT

SEQ ID NO: 88 - protein, 7 aa, Artificial Sequence
  CDR2, NRCoV2-MRed22
ISSRGST

SEQ ID NO: 89 - protein, 8 aa, Artificial Sequence
  CDR2, NRCoV2-MRed25
ISWRGDST

SEQ ID NO: 90 - protein, 20 aa, Artificial Sequence
  CDR3, NRCoV2-1a
AADYSMRPLWVSRWHRDYEY

SEQ ID NO: 91 - protein, 20 aa, Artificial Sequence
  CDR3, NRCoV2-1c
AADYSMRRFAVGRWHRDYEY

SEQ ID NO: 92 - protein, 20 aa, Artificial Sequence
  CDR3, NRCoV2-1d
AADYSMRPFAVGRWHRDYEY

SEQ ID NO: 93 - protein, 15 aa, Artificial Sequence
  CDR3, NRCoV2-02
TKGPDLYYFGSGYSD

SEQ ID NO: 94 - protein, 14 aa, Artificial Sequence
  CDR3, NRCoV2-03
NIYGPTYSTRRNEY

SEQ ID NO: 95 - protein, 7 aa, Artificial Sequence
  CDR3, NRCoV2-04
WSSYEST

SEQ ID NO: 96 - protein, 15 aa, Artificial Sequence
  CDR3, NRCoV2-05
SKGPVSSYYGSGYDY

SEQ ID NO: 97 - protein, 10 aa, Artificial Sequence
  CDR3, NRCoV2-06
NARHYGGSEY

SEQ ID NO: 98 - protein, 21 aa, Artificial Sequence
  CDR3, NRCoV2-07
AAVQDVHGDNYYCTSPNEYNV

SEQ ID NO: 99 - protein, 19 aa, Artificial Sequence
  CDR3, NRCoV2-08
AADYQYSTYCLGYDAHYEY

SEQ ID NO: 100 - protein, 9 aa, Artificial Sequence
  CDR3, NRCoV2-10
YAADDLGDY

SEQ ID NO: 101 - protein, 19 aa, Artificial Sequence
  CDR3, NRCoV2-11
AARSRDFSSPFSATDTYTS

SEQ ID NO: 102 - protein, 19 aa, Artificial Sequence
  CDR3, NRCoV2-11a
AARSRDFSSPISATDKYGS

SEQ ID NO: 103 - protein, 18 aa, Artificial Sequence
  CDR3, NRCoV2-12
AADRGLSYYYTRTTEYNY

SEQ ID NO: 104 - protein, 11 aa, Artificial Sequence
  CDR3, NRCoV2-14
HAVNADIGGDY

SEQ ID NO: 105 - protein, 10 aa, Artificial Sequence
  CDR3, NRCoV2-15
YAAYGGGGDY

SEQ ID NO: 106 - protein, 10 aa, Artificial Sequence
  CDR3, NRCoV2-17
QAANVNGGDY

SEQ ID NO: 107 - protein, 8 aa, Artificial Sequence
  CDR3, NRCoV2-18
SLHTSHDY

SEQ ID NO: 108 - protein, 23 aa, Artificial Sequence
  CDR3, NRCoV2-19
AADRARLRFGCSLNFRREVAYDY

SEQ ID NO: 109 - protein, 18 aa, Artificial Sequence
  CDR3, NRCoV2-20
AADRGMSYYYTRATEYYY

SEQ ID NO: 110 - protein, 22 aa, Artificial Sequence
  CDR3, NRCoV2-21
AADHRGRSLRFGCSSSTTDYLY

SEQ ID NO: 111 - protein, 23 aa, Artificial Sequence
  CDR3, NRCoV2-SR01
AATGIRSTWSVYGCSRLAGPYDY

SEQ ID NO: 112 - protein, 18 aa, Artificial Sequence
  CDR3, NRCoV2-SR02
AADRLYSRACPTAGGRNY

SEQ ID NO: 113 - protein, 17 aa, Artificial Sequence
  CDR3, NRCoV2-SR03
NRGGWEYRSSYYIMGPH

SEQ ID NO: 114 - protein, 17 aa, Artificial Sequence
  CDR3, NRCoV2-SR04
NTAALVGNRLLPMATIT

SEQ ID NO: 115 - protein, 20 aa, Artificial Sequence
  CDR3, NRCoV2-SR13
NMGGWDYRSNTYIPGSRSDY

SEQ ID NO: 116 - protein, 17 aa, Artificial Sequence
  CDR3, NRCoV2-SR16
NTGGWDYRSSTFIMGLN

SEQ ID NO: 117 - protein, 18 aa, Artificial Sequence
  CDR3, NRCoV2-S2A3
NTGSLSYGGSVYYPSYDN

SEQ ID NO: 118 - protein, 15 aa, Artificial Sequence
  CDR3, NRCoV2-S2A4
HAAARDSHGIYLLDT

SEQ ID NO: 119 - protein, 11 aa, Artificial Sequence
  CDR3, NRCoV2-S2B3
NYRSIYYGQNF

SEQ ID NO: 120 - protein, 20 aa, Artificial Sequence
  CDR3, NRCoV2-S2H4
ALVFGYTSRDYCLTPKRGNY

SEQ ID NO: 121 - protein, 10 aa, Artificial Sequence
  CDR3, NRCoV2-S2F3
NLRDILSQPF

SEQ ID NO: 122 - protein, 11 aa, Artificial Sequence
  CDR3, NRCoV2-S2G3
YTTKRDDASVY

SEQ ID NO: 123 - protein, 9 aa, Artificial Sequence
  CDR3, NRCoV2-S2G4
NKHWWTGDW

SEQ ID NO: 124 - protein, 10 aa, Artificial Sequence
  CDR3, NRCoV2-S202
NYGPGYRKAA

SEQ ID NO: 125 - protein, 14 aa, Artificial Sequence
  CDR3, NRCoV2-MRed02 and NRCoV2-MRed04
AADRGFVVRGQYDY

SEQ ID NO: 126 - protein, 20 aa, Artificial Sequence
  CDR3, NRCoV2-MRed03
ATDAFATCDSWYAQIAQYDF

SEQ ID NO: 127 - protein, 23 aa, Artificial Sequence
  CDR3, NRCoV2-MRed05
ATGPQAYYSGSYYFQCPQAGMDY

SEQ ID NO: 128 - protein, 17 aa, Artificial Sequence
  CDR3, NRCoV2-MRed06
ATDSFSSCSDYESGMDF

SEQ ID NO: 129 - protein, 10 aa, Artificial Sequence
  CDR3, NRCoV2-MRed07
YANYGWAIPY

SEQ ID NO: 130 - protein, 15 aa, Artificial Sequence
  CDR3, NRCoV2-MRed11
ATTISDGSSWSTKSY

SEQ ID NO: 131 - protein, 11 aa, Artificial Sequence
  CDR3, NRCoV2-MRed18
NYYNYYYGRNF

SEQ ID NO: 132 - protein, 11 aa, Artificial Sequence
  CDR3, NRCoV2-MRed19
YMHSVYYGIDY

SEQ ID NO: 133 - protein, 20 aa, Artificial Sequence
  CDR3, NRCoV2-MRed20
AAKPPFYGSGTYSTPRAYLY

SEQ ID NO: 134 - protein, 10 aa, Artificial Sequence
  CDR3, NRCoV2-MRed22
NAREFTGFDY

SEQ ID NO: 135 - protein, 17 aa, Artificial Sequence
  CDR3, NRCoV2-MRed25
AAEMWGTATIVASRYTY

SEQ ID NO: 136 - protein, 127 aa, Artificial Sequence
  VHH, NRCoV2-1a
EVKLVQSGGGSVQPGGSLRLSCAASGSTLDYYAIGWFRQAPGKEREWVSCVSSSDGSTLYADSVKGRFTISRDNAKNTVYLQMNSLKPEDTAVYVCAADYSMRPLWVSRWHRDYEYWGQGTQVTVSS

SEQ ID NO: 137 - protein, 127 aa, Artificial Sequence
  VHH, NRCoV2-1c
EVQLVESGGGSVQPGGSLRLSCAASGSILDYYAVGWFRQAPGKEREWVSSVSSSDGSTLYADSVKGRFTISRDDAKNTIYLQMDNLEPEDTAVYVCAADYSMRRFAVGRWHRDYEYWGQGTQVTVSS

SEQ ID NO: 138 - protein, 127 aa, Artificial Sequence
  VHH, NRCoV2-1d
EVQLVESGGGSVQPGGSLRLSCAASGSTLDYYAIGWFRQAPGKEREWVSSVSSSDGNTLYADSVKGRFTISRDNAKNTVYLQMNSLKAEDTAVYVCAADYSMRPFAVGRWHRDYEYWGQGTQVTVSS

SEQ ID NO: 139 - protein, 122 aa, Artificial Sequence
  VHH, NRCoV2-02
EVQLVESGGGLVQAGGSLRLSCAASGFTFSNYAMNWVRQAPGKGLEWVSGISGRGDDTRYADSVKGRFTISRDNAKNTLFLQMRSLRPEDTGVYRCTKGPDLYYFGSGYSDRGQGTQVTVSS

SEQ ID NO: 140 - protein, 120 aa, Artificial Sequence
  VHH, NRCoV2-03
EVQLVSSGGGLVQAGGSLRLSCTASGITFSYYAMGWYRQAPGQPRELVASMSNMDSTIYADSVKGRFTISRDNAKTTIYLQMNNLKPEDTAVYFCNIYGPTYSTRRNEYWGQGTQVTVSS

SEQ ID NO: 141 - protein, 113 aa, Artificial Sequence
  VHH, NRCoV2-04
AVQLVDSGGGLVQPGGSLRLSCAASGSPFSNVVMAWYRQAPGKQRERVAFISGGGIADYIMSVKGRFTISRDNAKNTVYLQMNSLKPEDTAVYYCWSSYESTWGQGTQVTVSS

SEQ ID NO: 142 - protein, 122 aa, Artificial Sequence
  VHH, NRCoV2-05
EVKLVQSGGGLVQPGGSLRLSCAASGFIFSNYAMNWVRQAPGKGLEWVSGINSGGGDTRYADSVKGRFTVSRDNAKNTLYLQMNSLKPEDTGVYYCSKGPVSSYYGSGYDYRGQGTQVTVSS

SEQ ID NO: 143 - protein, 116 aa, Artificial Sequence
  VHH, NRCoV2-06
EVQLVQSGGGLVQAGESLRLSCAASVSTFSSYAMGWYRQAPGKQRELVASIGFVGATYYIDSVKGRFTISRDNAKKTAYLQMNDLKPDDTAVYYCNARHYGGSEYWGQGTQVTVSS

SEQ ID NO: 144 - protein, 128 aa, Artificial Sequence
  VHH, NRCoV2-07
QVQLVQSGGGLVQPGGSLRLSCAASGVTLDYYAIGWFRQAPGKEREAVSCISSNGRRNHYVASVRGRFTISRDNAKSTVYLQMNSLKPEDTAVYYCAAVQDVHGDNYYCTSPNEYNVWGQGTQVTVSS

SEQ ID NO: 145 - protein, 126 aa, Artificial Sequence
  VHH, NRCoV2-08
EVQLQQSGGGLVQPGGSLRLSCAASGFTLDDYAIGWFRQSPGKEREWVTCISRSGTTTYYTASVKGRFTFSRDNAKNTAYLQMNSLRPEDTAVYYCAADYQYSTYCLGYDAHYEYWGQGTQVTVSS

SEQ ID NO: 146 - protein, 115 aa, Artificial Sequence
  VHH, NRCoV2-10
QLQLQESGGGLVQPGGSLTLSCAASGNTFSRSNMHWYRQAPGAQREWVAAISSRGISTYAYSAKGRFTISRDNAKNTVSLQMNSLKPEDTAVYYCYAADDLGDYWGQGTQVTVSS

SEQ ID NO: 147 - protein, 126 aa, Artificial Sequence
  VHH, NRCoV2-11
EVQLVSSGGGLVQPGGSLRLSCAASGSSLDSYSVSWFRQAPGKEREWISFISRYYSSTYYTDSVKGRFTTSRDGDQKTVHLQMNSLKPEDTAVYYCAARSRDFSSPFSATDTYTSWGQGTQVTVSS

SEQ ID NO: 148 - protein, 126 aa, Artificial Sequence
  VHH, NRCoV2-11a
EVQLQQSGGGLVQPGGSLRLSCAASGFTLDSYNIAWFRQAPGKEREWISYISRYYESTYYSDSVKGRFTTSRDGDKKTVSLQMNSLKSEDTAVYYCAARSRDFSSPISATDKYGSWGQGTQVTVSS

SEQ ID NO: 149 - protein, 128 aa, Artificial Sequence
  VHH, NRCoV2-12
EVQLQQSGGGLVQAGGSLRLSCAASGRTFRNYVMGWFRQAPQAPGKDHEFVAAISWGGTEIYYADSVKGRFTISRDNAKNTVYLQMNSLKPEDTAVYYCAADRGLSYYYTRTTEYNYWGQGTQVTVSS

SEQ ID NO: 150 - protein, 117 aa, Artificial Sequence
  VHH, NRCoV2-14
EVQLVSSGGGLVQAGGSLRLSCEASGTTFSHYAVGWYRQAPGKQREWVASISVFGSTTYGGSVAGRFTISRDNDKNTVDLQMNSLKPEDTAVYYCHAVNADIGGDYWGQGTQVTVSS

SEQ ID NO: 151 - protein, 116 aa, Artificial Sequence
  VHH, NRCoV2-15
EVQLVSSGGGLVEAGGSLRLSCIASGSTSGRNTMGWFRQAPGKQREWVAIVSTSGATNYAGSVKGRFTLSRDNAKNAVYLQMNNLKPEDTAVYYCYAAYGGGGDYWGQGTQVTVSS

SEQ ID NO: 152 - protein, 116 aa, Artificial Sequence
  VHH, NRCoV2-17
EVQLQQSGGGLVQTGGSLRLSCAAAGSPFSQLAMSWYRQISGKERAWVASISPTGNRSYSKIAKGRFTISRDNAKNTVTLQMTSLKPEDTAAYICQAANVNGGDYWGQGTQVTVSS

SEQ ID NO: 153 - protein, 114 aa, Artificial Sequence
  VHH, NRCoV2-18
EVQLVESGGGLVQAGGSLRLSCVASGITISGYNMAWWRQTRGKQTERVAFINSGGSTTYSDSVKGRFTISRDNGKNTAYLQMNSLNAEDTADYFCSLHTSHDYWGQGTQVTVSS

SEQ ID NO: 154 - protein, 129 aa, Artificial Sequence
  VHH, NRCoV2-19
EVQLLESGGGLVLPGGSLRLSCAVSGLTLNSYAIGWFRQAPGKEREGLSCLTSGGTGVYAESVKGRFTISRDNAENTVYLQMNSLKPEDTAVYYCAADRARLRFGCSLNFRREVAYDYWGQGTQVTVSS

SEQ ID NO: 155 - protein, 128 aa, Artificial Sequence
  VHH, NRCoV2-20
EVQLQQSGGGLVQPGGSLRLSCAASGRTFSNYVVGWFRQAPQAPGKDHEFVAVISGSDTETYYADSVKGRFTISRDNAKNTVYLQMNSLKPEDTAVYYCAADRGMSYYYTRATEYYYWGQGTQVTVSS

SEQ ID NO: 156 - protein, 128 aa, Artificial Sequence
  VHH, NRCoV2-21
EVQLVESGGGLVQPGGSLRLSCATSGFTLDYYAIGWFRQAPGKEREWVSCISSGGSTFYVDSVKGRFTISRDNAKDTVYLQMSSLKPDDTAVYYCAADHRGRSLRFGCSSSTTDYLYWGQGTQVTVSS

SEQ ID NO: 157 - protein, 130 aa, Artificial Sequence
  VHH, NRCoV2-SR01
EVQLVQSGGGSVQAGGSLRLSCVASGFTFDNYAIGWFRQAPGKEREGVSCISGNGGVTIYADSVKGRFTISRDNAKNLVYLQMNSLKPEDTAVYYCAATGIRSTWSVYGCSRLAGPYDYWGQGTQVTVSS

SEQ ID NO: 158 - protein, 125 aa, Artificial Sequence
  VHH, NRCoV2-SR02
EVQLVDSGGGLVQAGGSLRLSCTASEFTLNYYSIGWFRQSPGKEREGVSCIRYSGGGIDYADSVKGRFTISRDNAKNTVYLTMNSLKPEDTAVYYCAADRLYSRACPTAGGRNYWGQGTQVTVSS

SEQ ID NO: 159 - protein, 123 aa, Artificial Sequence
  VHH, NRCoV2-SR03
AVQLVDSGGGLVQAGGSLRLSCAASGSIFSNNHMGWYRQAPGKQRELVAAISSGGKTNYADFAKGRFTISRDNAKNMVYLQMNSLKPEDTAVYYCNRGGWEYRSSYYIMGPHWGQGTQVTVSS

SEQ ID NO: 160 - protein, 126 aa, Artificial Sequence
  VHH, NRCoV2-SR04
QVQLVESGGGLVQAGGSLRLSCAASGRTFSSHTVGWYRQAPGKQRDLVAAISMGGNTNYADYADSVKGRFTISRDNAKNTLYLQMNSLKPEDTAVYYCNTAALVGNRLLPMATITWGQGTQVTVSS

SEQ ID NO: 161 - protein, 126 aa, Artificial Sequence
  VHH, NRCoV2-SR13
EVQLVESGGGLVTTGGSLRLSCATSGSRFGSKHMAWYRQAPGKQRDLVAAISSGGSTHYGSSVKGRFTISRDNAKSTVYLQMNSLNPEDTAVFYCNMGGWDYRSNTYIPGSRSDYWGQGTQVTVSS

SEQ ID NO: 162 - protein, 123 aa, Artificial Sequence
  VHH, NRCoV2-SR16
QVQLVQSAGGLVQAGGSLRLSCVVSGTTFSRYHMGWHRQAPGKQRDFVAGISTSGAVTYADSAKGRFTISRDNAKNTVYLEMNSLKLEDTALYYCNTGGWDYRSSTFIMGLNWGQGTQVTVSS

SEQ ID NO: 163 - protein, 132 aa, Artificial Sequence
  VHH, NRCoV2-S2A3
EVQLQQSGGGLVQAGGSLRLSCAASGRPYSNYAMAWYRQAPGKQHELVAGKQRELVAAISSGGTTKYADSVKARFTISRDNAKNTVYLQMNILRPEDTAVYYCNTGSLSYGGSVYYPSYDNWGQGTQVTVSS

SEQ ID NO: 164 - protein, 121 aa, Artificial Sequence
  VHH, NRCoV2-S2A4
QVQLVQSGGGLVQAGGSLRLSCAVSGSPFRSNVMEWYRQAPGKQRELVASISTGGSRTYTDSVKGRFTISRDNAKNEAFLQMNSLKPEDTAVYYCHAAARDSHGIYLLDTWGQGTQVTVSS

SEQ ID NO: 165 - protein, 117 aa, Artificial Sequence
  VHH, NRCoV2-S2B3
QVQLVDSGGGLVQAGGSLRLSCAASASTFGDSAMGYYRQAPGKQRELVATISTGSNTNYADSVKGRFTISRDDAKNTVYLQMNSLKPEDTAVYYCNYRSIYYGQNFWGQGTQVTVSS

SEQ ID NO: 166 - protein, 129 aa, Artificial Sequence
  VHH, NRCoV2-S2H4
QVQLVQSGGGLVQAGGSLRLSCAASGFTFNLYSIAWFRQAPGKEREGVSCINSGDRDSTTYYADSVKGRFTISRDNAKHTAYLQMDSLKPEDTAVYYCALVFGYTSRDYCLTPKRGNYWGQGTQVTVSS

SEQ ID NO: 167 - protein, 116 aa, Artificial Sequence
  VHH, NRCoV2-S2F3
EVQLVQSGGGLVQAGGSLRLSCATSVRILSVPAMGWYRQAPGKEREMVAVITSGGSTNYADSVKGRFTISRDNAKNTVYLQMNSLKLEDTAVYQCNLRDILSQPFWGQGTQVTVSS

SEQ ID NO: 168 - protein, 117 aa, Artificial Sequence
  VHH, NRCoV2-S2G3
QVQLVQSGGGSVQAGGSLRLSCAASGSTFGIFLMGWRRQAPGKQRELVAHITSGGATNYADSVKGRFTISRDNAKNTVYLQMNSLEPEDTAVYYCYTTKRDDASVYWGQGTQVTVSS

SEQ ID NO: 169 - protein, 115 aa, Artificial Sequence
  VHH, NRCoV2-S2G4
QVQLVQSGGGLVQAGGSLTLSCAPSGSTFSGYATNWYRQAPGKQRELVATISSDGDKNYADSVKGRFTISRDNAKNTVYLQMNSLKPEDTAVYYCNKHWWTGDWWGQGTQVTVSS

SEQ ID NO: 170 - protein, 116 aa, Artificial Sequence
  VHH, NRCoV2-S202
QVQLVQSGGGLVQAGGSLRLSCAASGITVSRIGMGWYRQAPGKQRDMVAVISAGGSTNYADSVKGRFTISRDNAKNTVYLQMDSLKPEDTAVYYCNYGPGYRKAAWGQGTQVTVSS

SEQ ID NO: 171 - protein, 120 aa, Artificial Sequence
  VHH, NRCoV2-MRed02
EVQLVESGGGLVQPGGSLRLSCAASGNIFSINSMGWFRQAPGKERDVVATIWSDSRTSYADSVKGRFTISTDNTRTKVYLQMSSLNPEDTAVYYCAADRGFVVRGQYDYWGQGTQVTVSS

SEQ ID NO: 172 - protein, 127 aa, Artificial Sequence
  VHH, NRCoV2-MRed03
EVQLVESGGGLVQPGGSLRLSCAAIGFTLDYYAIGWFRQAPGKEREGVSCISSSDGSTYYADSVKGRFTISRDNAKNTVYLQMNSLKPEDTAVYYCATDAFATCDSWYAQIAQYDFRGQGTQVTVSS

SEQ ID NO: 173 - protein, 120 aa, Artificial Sequence
  VHH, NRCoV2-MRed04
EVQLVESGGGLVQPGRSLRLSCAASGNSFSISTMGWFRQAPGKERELVASIWSDTTTSYADSVKGRFTISTDNTRTKVYLQMSSLNPEDTAVYYCAADRGFVVRGQYDYWGQGTQVTVSS

SEQ ID NO: 174 - protein, 130 aa, Artificial Sequence
  VHH, NRCoV2-MRed05
EVQLVESGGGLVQPGGSLRLSCAASGFTLDYYAIGWFRQAPGKEREGVSCISSSDGSTLYADSVKGRFTISRDNAKNTVYLQMNSLKPEDTAVYYCATGPQAYYSGSYYFQCPQAGMDYWGKGTQVTVSS

SEQ ID NO: 175 - protein, 124 aa, Artificial Sequence
  VHH, NRCoV2-MRed06
EVQLVESGGGLVQAGGSLRLSCAASGFTLAYYAIGWFRQAPGKEREGVSCISSSDGSAHYADSVKGRFTISRDNAKNTVSLQMNSLKPEDTAVYYCATDSFSSCSDYESGMDFWGKGTQVTVSS

SEQ ID NO: 176 - protein, 116 aa, Artificial Sequence
  VHH, NRCoV2-MRed07
EVQLVESGGGLVQPGGSLTLSCAASGSIGPFNTMGWYRQAPGNQREPAAIITRGGVTNYADSVKGRFTISRDNAKNAVYLQMDSLKPDDTAVYYCYANYGWAIPYWGNGTQVTVSS

SEQ ID NO: 177 - protein, 122 aa, Artificial Sequence
  VHH, NRCoV2-MRed11
EVQLVESGGGLVQAGGSLRLSCAASGFTFSSYAMSWHRQAPGKGLEWVSAINSGGGSTSYADSVKGRFAISRDNAKNTLYLQMNSLKPEDTAVYYCATTISDGSSWSTKSYRGQGTQVTVSS

SEQ ID NO: 178 - protein, 117 aa, Artificial Sequence
  VHH, NRCoV2-MRed18
EVQLVESGGGLVQPGGSLRLSCAASTTVFGRNAMGWYRQAPGKERELVATVSDGGTPNYADSVKGRFTISRDNAKNTIYLQMNSLEPEDTAVYYCNYYNYYYGRNFWGQGTQVTVSS

SEQ ID NO: 179 - protein, 116 aa, Artificial Sequence
  VHH, NRCoV2-MRed19
EVQLVESGGGLVQPGGSLRLSCAASTIIFKGQTMGWFRQAPGNERELVATMTTSGSANYADSVKGRFTISRDNEKTVTLQMNSLKPEDTALYYCYMHSVYYGIDYWGKGTQVTVSS

SEQ ID NO: 180 - protein, 127 aa, Artificial Sequence
  VHH, NRCoV2-MRed20
EVQLVESGGGSVQAGGSLRLSCAASGLSFSSYDMGWFRQAPGKEREFVAAIRESGSGTYYADSVKGRFTISRDNAKNTVYLQVSSLKPEDTAVYTCAAKPPFYGSGTYSTPRAYLYWGQGTQVTVSS

SEQ ID NO: 181 - protein, 116 aa, Artificial Sequence
  VHH, NRCoV2-MRed22
EVQLVESGGGLVQPGGSLRLSCAASGSVFASNAMGWYRQAPGKQRELVATISSRGSTNYADSVKGRFTISRDNAKNTVYLQMNSLGPEDTAVYYCNAREFTGFDYWGQGTQVTVSS

SEQ ID NO: 182 - protein, 124 aa, Artificial Sequence
  VHH, NRCoV2-MRed25
EVQLVESGGGLVQAGGSLRLSCAASGHTFSRYGMGWFRQAPGKEREFVAAISWRGDSTYYRDSVNGRFTISRDNAKNTVYLGMNSLKPEDTAVYYCAAEMWGTATIVASRYTYWGQGTQVTVSS

SEQ ID NO: 183 - protein, 143 aa, Artificial Sequence
  VHH consensus sequence
  SITE (1)..(1): Xaa is Ala, Glu, or Gln
  SITE (2)..(2): Xaa is Leu or Val
  SITE (3)..(3): Xaa is Lys or Gln
  SITE (5)..(5): Xaa is Leu, Gln, or Val
  SITE (6)..(6): Xaa is Asp, Glu, Gln, or Ser
  SITE (8)..(8): Xaa is Ala or Gly
  SITE (11)..(11): Xaa is Leu or Ser
  SITE (13)..(13): Xaa is Glu, Leu, Gln, or Thr
  SITE (14)..(14): Xaa is Ala, Pro, or Thr
  SITE (16)..(16): Xaa is Glu, Gly, or Arg
  SITE (19)..(19): Xaa is Arg or Thr
  SITE (23)..(23): Xaa is Ala, Glu, Ile, Thr, or Val
  SITE (24)..(24): Xaa is Ala, Pro, Thr, or Val
  SITE (25)..(25): Xaa is Ala, Ile, or Ser
  SITE (26)..(26): Xaa is Ala, Glu, Gly, Thr, or Val
  SITE (27)..(27): Xaa is Phe, His, Ile, Leu, Asn, Arg, Ser, Thr, or Val
  SITE (28)..(28): Xaa is Ile, Pro, Arg, Ser, Thr, or Val
  misc_feature (29)..(29): Xaa is Phe, Gly, Ile, Leu, Ser, Val, or Tyr
  misc_feature (30)..(30): Xaa is Ala, Asp. Gly, Lys, Asn, Pro, Arg, or Ser
  misc_feature (31)..(31): Xaa is Asp, Phe, Gly, His, Ile, Leu, Asn, Gln, Arg, Ser, Val, Tyr
  misc_feature (32)..(32): Xaa is Phe, His, Ile, Lys, Leu, Asn, Pro, Gln, Ser, Val, or Tyr
  misc_feature (33)..(33): Xaa is Ala, Asp, Gly, His, Leu, Asn, Ser, Thr, or Val
  misc_feature (34)..(34): Xaa is Ile, Met, Thr, or Val
  misc_feature (35)..(35): Xaa is Ala, Glu, Gly, His, Asn, or Ser
  misc_feature (36)..(36): Xaa is Trp or Tyr
  misc_feature (37)..(37): Xaa is Phe, His, Arg, Val, Trp, or Tyr
  misc_feature (40)..(40): Xaa is Ala, Ile, Ser, or Thr
  misc_feature (41)..(41): Xaa is Pro or absent
  misc_feature (42)..(42): Xaa is Gln or absent
  misc_feature (43)..(43): Xaa is Ala or absent
  misc_feature (44)..(44): Xaa is Pro, Arg, or Ser
  misc_feature (46)..(46): Xaa is Ala, Lys, Asn, or Gln
  misc_feature (47)..(47): Xaa is Asp, Glu, Gly, Pro, or Gln
  misc_feature (48)..(48): Xaa is His or absent
  misc_feature (49)..(49): Xaa is Glu or absent
  misc_feature (50)..(50): Xaa is Leu or absent
  misc_feature (50)..(50): Xaa is Val or absent
  misc_feature (51)..(51): Xaa can be any naturally occurring amino acid
  misc_feature (52)..(52): Xaa is Ala or absent
  misc_feature (53)..(53): Xaa is Gly or absent
  misc_feature (54)..(54): Xaa is Lys or absent
  misc_feature (55)..(55): Xaa is Gln or absent
  misc_feature (56)..(56): Xaa is His, Leu, Arg, or Thr
  misc_feature (57)..(57): Xaa is Ala, Asp, or Glu
  misc_feature (58)..(58): Xaa is Ala, Phe, Gly, Leu, Met, Pro, Arg, Val, or Trp
  misc_feature (58)..(58): Xaa is Lys, Asn, or Gln
  misc_feature (59)..(59): Xaa is Ala, Ile, Leu, or Val
  misc_feature (60)..(60): Xaa is Ala, Ser, or Thr
  misc_feature (61)..(61): Xaa is Ala, Cys, Phe, Gly, His, Ile, Ser, Thr, Val, or Tyr
  misc_feature (62)..(62): Xaa is Ile, Leu, Met, or Val
  misc_feature (63)..(63): Xaa is Gly, Asn, Arg, Ser, Thr, or Trp
  misc_feature (64)..(64): Xaa is Ala, Asp, Glu, Phe, Gly, Met, Asn, Pro, Arg, Ser, Thr, Val, Trp, or Tyr
  misc_feature (65)..(65): Xaa is Asp, Phe, Gly, Met, Asn, Arg, Ser, Thr, Val, Tyr, or absent
  misc_feature (66)..(66): Xaa is Asp, Gly, Ser, Thr, or Tyr
  misc_feature (67)..(67): Xaa is Ala, Asp, Glu, Gly, Ile, Lys, Asn, Arg, Ser, Thr, or Val
  misc_feature (68)..(68): Xaa is Ala, Asp, Glu, Gly, Lys, Asn, Pro, Arg, Ser, Thr, or Val
  misc_feature (69)..(69): Xaa is Asn, Ser, or absent
  misc_feature (70)..(70): Xaa is Thr, Tyr, or absent
  misc_feature (71)..(71): Xaa is Ala, Gly, Ile, Asn, Thr, or absent
  misc_feature (72)..(72): Xaa is Asp, Phe, His, Ile, Lys, Leu, Asn, Arg, Ser, Thr, Val, Tyr, or absent
  misc_feature (73)..(73): Xaa is Tyr or absent
  misc_feature (74)..(74): Xaa is Ala, Gly, Ile, Arg, Ser, Thr, or Val
  misc_feature (75)..(75): Xaa is Ala, Asp, Glu, Gly, Lys, Met, Ser, or Tyr
  misc_feature (76)..(76): Xaa is Phe, Ile, or Ser
  misc_feature (77)..(77): Xaa is Ala or Val
  misc_feature (78)..(78): Xaa is Ala, Lys, Asn, or Val
  misc_feature (79)..(9): Xaa is Ala or Gly
  misc_feature (79)..(79): Xaa can be any naturally occurring amino acid
  misc_feature (82)..(82): Xaa is Ala or Thr
  misc_feature (83)..(83): Xaa is Phe, Ile, Leu, Thr, or Val
  misc_feature (85)..(85): Xaa is Arg or Thr
  misc_feature (87)..(87): Xaa is Asp, Gly, or Asn
  misc_feature (88)..(88): Xaa is Ala, Asp, Glu, Gly, or Thr
  misc_feature (89)..(89): Xaa is Glu, Lys, Gln, or Arg
  misc_feature (90)..(90): Xaa is Asp, His, Lys, Asn, Ser, Thr, or absent
  misc_feature (91)..(91): Xaa is Ala, Glu, Lys, Leu, Met, or Thr
  misc_feature (92)..(92): Xaa is Ala, Ile, Leu, or Val
  misc_feature (93)..(93): Xaa is Asp, Phe, His, Ser, Thr, or Tyr
  misc_feature (95)..(95): Xaa is Glu, Gly, Gln, or Thr
  misc_feature (96)..(96): Xaa is Met or Val
  misc_feature (97)..(97): Xaa is Asp, Asn, Arg, Ser, or Thr
  misc_feature (98)..(98): Xaa is Asp, Ile, Asn, or Ser
  misc_feature (100)..(100): Xaa is Glu, Gly, Lys, Asn, or Arg
  misc_feature (101)..(101): Xaa is Ala, Leu, Pro, or Ser
  misc_feature (102)..(102): Xaa is Asp or Glu
  misc_feature (105)..(105): Xaa is Ala or Gly
  misc_feature (106)..(106): Xaa is Ala, Asp, Leu, or Val
  misc_feature (107)..(107): Xaa is Phe or Tyr
  misc_feature (108)..(108): Xaa is Phe, Ile, Gln, Arg, Thr, Val, or Tyr
  misc_feature (110)..(110): Xaa is Ala, His, Asn, Gln, Ser, Thr, Trp, or Tyr
  misc_feature (111)..(111): Xaa is Ala, Ile, Lys, Leu, Met, Arg, Ser, Thr, or Tyr
  misc_feature (112)..(112): Xaa is Ala, Asp, Glu, Gly, His, Lys, Asn, Arg, Ser, Thr, Val, or Tyr
  misc_feature (113)..(113): Xaa is Ala, Asp, Glu, Phe, Gly, His, Ile, Lys, Asn, Pro, Gln, Arg, Ser, Thr, Tyr, or absent
  misc_feature (114)..(114): Xaa is Ala, Asp, Phe, Gly, Ile, Leu, Pro, Gln, Arg, Ser, Val, Trp, or absent
  misc_feature (115)..(115): Xaa is Ala, Asp, Glu, Phe, Gly, Leu, Met, Arg, Ser, Thr, Val, Tyr, or absent
  misc_feature (116)..(116): Xaa is Phe, Gly, His, Leu, Ser, Thr, Trp, Tyr, or absent
  misc_feature (117)..(117): Xaa is Cys, Gly, His, Asn, Arg, Ser, Thr, Tyr, or absent
  misc_feature (118)..(118): Xaa is Ala, Asp, Phe, Gly, Arg, Ser, Thr, Trp, or absent
  misc_feature (119)..(119): Xaa is Ala, Cys, Asp, Gly, Ile, Leu, Asn, Pro, Arg, Ser, Tyr, or absent
  misc_feature (120)..(120): Xaa is Cys, Gly, Ile, Asn, Pro, Arg, Ser, Thr, Val, Trp, Tyr, or absent
  misc_feature (121)..(121): Xaa is Asp, Phe, Gly, Leu, Ser, Trp, Tyr, or absent
  misc_feature (122)..(122): Xaa is Ala, Asp, Gly, Ile, Leu, Thr, Val, Trp, Tyr, or absent
  misc_feature (123)..(123): Xaa is Cys, Phe, Asn, Val, or absent
  misc_feature (124)..(124): Xaa is Phe, Gly, Ile, Leu, Gln, Ser, Tyr, or absent
  misc_feature (125)..(125): Xaa is Ala, Cys, Asp, Gly, Pro, Arg, Ser, Thr, or absent
  misc_feature (126)..(126): Xaa is Ala, Phe, Gly, Leu, Met, Pro, Gln, Arg, Ser, Thr, Val, Trp, Tyr, or absent
  misc_feature (127)..(127): Xaa is Ala, Asp, Glu, Gly, His, Ile, Pro, Gln, Arg, Ser, Thr, Trp, or absent
  misc_feature (128)..(128): Xaa is Ala, Asp, Gly, Ile, Lys, Met, Asn, Arg, Ser, Thr, Val, Trp, Tyr, or absent
  misc_feature (129)..(129): Xaa is Ala, Asp, Glu, Gly, His, Lys, Leu, Pro, Gln, Arg, Ser, Thr, or absent
  misc_feature (130)..(130): Xaa is Glu, Phe, Gly, His, Ile, Lys, Leu, Met, Asn, Gln, Arg, Ser, Thr, or Tyr
  misc_feature (131)..(131): Xaa is Ala, Asp, Glu, Gly, Ile, Leu, Asn, Pro, Ser, Thr, Val, or Tyr
  misc_feature (132)..(132): Xaa is Ala, Asp, Phe, His, Asn, Ser, Thr, Val, Trp, or Tyr
  misc_feature (133)..(133): Xaa is Arg or Trp
  misc_feature (135)..(135): Xaa can be any naturally occurring amino acid
XXXLXXSXGGXVXXGXSLXLSCXXXXXXXXXXXXXXXRQXXXXXGXXXXXXXXXXXXXXXXXXXXXXXXXXXXXXXXXXRFXXSXDXXXXXXXLXXXXLXXXDTXXXXCXXXXXXXXXXXXXXXXXXXXXXXXGXGTQVTVSS

SEQ ID NO: 184 - protein, 133 aa, Artificial Sequence
  VHH consensus sequence
  misc_feature (1)..(1): Xaa is Ala, Glu, or Gln
  misc_feature (6)..(6): Xaa is Asp, Glu, or Gln
  misc_feature (8)..(8): Xaa is Ala or Gly
  misc_feature (11)..(11): Xaa is Leu or Ser
  misc_feature (13)..(13): Xaa is Gln or Thr
  misc_feature (14)..(14): Xaa is Ala, Pro, or Thr
  misc_feature (19)..(19): Xaa is Arg or Thr
  misc_feature (23)..(23): Xaa is Ala, Thr, or Val
  misc_feature (24)..(24): Xaa is Ala, Thr, or Val
  misc_feature (25)..(25): Xaa is Ile or Ser
  misc_feature (26)..(26): Xaa is Glu or Gly
  misc_feature (27)..(27): Xaa is Phe, Arg, Ser, or Thr
  misc_feature (28)..(28): Xaa is Ile, Arg, or Thr
  misc_feature (29)..(29): Xaa is Phe, Gly, or Leu
  misc_feature (30)..(30): Xaa is Asp, Ala, Gly, Asn, Pro, or Ser
  misc_feature (31)..(31): Xaa is Phe, Asn, Arg, Ser, or Tyr
  misc_feature (32)..(32): Xaa is His, Lys, Asn, or Tyr
  misc_feature (33)..(33): Xaa is Ala, His, Ser, or Thr
  misc_feature (34)..(34): Xaa is Ile, Met, or Val
  misc_feature (35)..(35): Xaa is Ala or Gly
  misc_feature (37)..(37): Xaa is Phe, His, or Tyr
  misc_feature (40)..(40): Xaa is Ala or Ser
  misc_feature (43)..(43): Xaa is Lys or Asn
  misc_feature (44)..(44): Xaa is Glu or Gln
  misc_feature (46)..(46): Xaa is Asp or Glu
  misc_feature (47)..(47): Xaa is Phe, Gly, Leu, or Pro
  misc_feature (48)..(48): Xaa is Ala or Val
  misc_feature (49)..(49): Xaa is Ala or Ser
  misc_feature (50)..(50): Xaa is Ala, Cys, Gly, or Ile
  misc_feature (52)..(52): Xaa is Arg, Ser, or Thr
  misc_feature (53)..(53): Xaa is Gly, Met, Arg, Ser, Thr, or Tyr
  misc_feature (54)..(54): Xaa is Gly, Asn, or Ser
  misc_feature (55)..(55): Xaa is Asp, Gly, or absent
  misc_feature (57)..(57): Xaa is Ala, Gly, Lys, Asn, Ser, or Val
  misc_feature (58)..(58): Xaa is Ala, Ile, Thr, or Val
  misc_feature (59)..(59): Xaa is Asp, His, Ile, Asn, Thr, or Tyr
  misc_feature (61)..(61): Xaa is Ala, Gly, or absent
  misc_feature (62)..(62): Xaa is Asp or absent
  misc_feature (63)..(63): Xaa is Tyr or absent
  misc_feature (64)..(64): Xaa is Ala or absent
  misc_feature (65)..(65): Xaa is Asp or Ser
  misc_feature (66)..(66): Xaa is Phe or Ser
  misc_feature (67)..(67): Xaa is Ala or Val
  misc_feature (80)..(80): Xaa is Asn or Ser
  misc_feature (81)..(81): Xaa is Ala, Leu, Met, or Thr
  misc_feature (82)..(82): Xaa is Leu or Val
  misc_feature (83)..(83): Xaa is Ser or Tyr
  misc_feature (85)..(85): Xaa is Glu, Gln, or Thr
  misc_feature (87)..(87): Xaa is Asp or Asn
  misc_feature (90)..(90): Xaa is Lys or Asn
  misc_feature (91)..(91): Xaa is Leu or Pro
  misc_feature (92)..(92): Xaa is Asp or Glu
  misc_feature (96)..(96): Xaa is Leu or Val
  misc_feature (97)..(97): Xaa is Phe or Tyr
  misc_feature (100)..(100): Xaa is Ala, Asn, or Tyr
  misc_feature (101)..(101): Xaa is Ala, Met, Arg, or Thr
  misc_feature (102)..(102): Xaa is Asp, Ala, Gly, Asn, or Thr
  misc_feature (103)..(103): Xaa is Ala, Gly, Arg, Ser, or absent
  misc_feature (104)..(104): Xaa is Phe, Ile, Leu, Trp, or absent
  misc_feature (105)..(105): Xaa is Asp, Glu, Ala, Arg, Ser, Val, Tyr, or absent
  misc_feature (106)..(106): Xaa is Gly, Ser, Thr, Tyr, or absent
  misc_feature (107)..(107): Xaa is Cys, Asn, Arg, Thr, or absent
  misc_feature (108)..(108): Xaa is Asp, Ser, Trp, or absent
  misc_feature (109)..(109): Xaa is Asp, Asn, Ser, or absent
  misc_feature (110)..(110): Xaa is Thr, Val, Trp, Tyr, or absent
  misc_feature (111)..(111): Xaa is Glu, Phe, Arg, Tyr, or absent
  misc_feature (112)..(112): Xaa is Gly or absent
  misc_feature (113)..(113): Xaa is Ala, Cys, or absent
  misc_feature (114)..(114): Xaa is Cys, Ser, or absent
  misc_feature (115)..(115): Xaa is Ala, Ile, Leu, Pro, Arg, Ser, or absent
  misc_feature (116)..(116): Xaa is Gln, Leu, Met, Pro, Thr, Tyr, or absent
  misc_feature (117)..(117): Xaa is Ala, Gly, Ile, Pro, or absent
  misc_feature (118)..(118): Xaa is Ala, Gly, Leu, Met, Pro, Ser, Trp, or absent
  misc_feature (119)..(119): Xaa is Gln, Ala, Gly, Pro, Arg, or absent
  misc_feature (120)..(120): Xaa is Ile, Met, Arg, Ser, Thr, Tyr, or absent
  misc_feature (121)..(121): Xaa is Asp, Ile, Asn, Pro, or absent
  misc_feature (122)..(122): Xaa is Phe, His, Asn, Thr, or Tyr
  misc_feature (123)..(123): Xaa is Arg or Trp
  misc_feature (125)..(125): Xaa is Gln, Lys, or Asn
XVQLVXSXGGXVXXGGSLXLSCXXXXXXXXXXXXXWXRQXPGXXRXXXXXIXXXXGXXXYXXXXXXXKGRFTISRDNAKXXXXLXMXSLXXXDTAXXYCXXXXXXXXXXXXXXXXXXXXXXXXGXGTQVTVSS

SEQ ID NO: 185 - protein, 137 aa, Artificial Sequence
  VHH consensus sequence
  misc_feature (1)..(1): Xaa is Ala or Pro
  misc_feature (5)..(5): Xaa is Pro or Val
  misc_feature (6)..(6): Xaa is Gln, Ala, or Pro
  misc_feature (11)..(11): Xaa is Lys or Ser
  misc_feature (14)..(14): Xaa is Asp or Asn
  misc_feature (19)..(19): Xaa is Arg or Thr
  misc_feature (24)..(24): Xaa is Asp, Asn, Thr, or Val
  misc_feature (26)..(26): Xaa is Asp, Phe, Thr, or Val
  misc_feature (27)..(27): Xaa is Cys, Gly, His, Lys, Arg, Ser, or Thr
  misc_feature (28)..(28): Xaa is His, Asn, Ser, Thr, or Val
  misc_feature (29)..(29): Xaa is Cys, Lys, Val, or Tyr
  misc_feature (30)..(30): Xaa is Asp, Phe, Ile, Met, Arg, or Ser
  misc_feature (31)..(31): Xaa is Gln, Phe, His, Lys, Met, Arg, Ser, or Val
  misc_feature (32)..(32): Xaa is Cys, His, Met, Asn, Pro, Ser, or Tyr
  misc_feature (33)..(33): Xaa is Asp, Gln, Phe, Lys, Ser, Thr, or Val
  misc_feature (34)..(34): Xaa is His, Leu, or Thr
  misc_feature (35)..(35): Xaa is Asp, Ala, Phe, Met, or Ser
  misc_feature (36)..(36): Xaa is Trp or Tyr
  misc_feature (37)..(37): Xaa is Cys, Gly, Arg, or Tyr
  misc_feature (43)..(43): Xaa is Ile or Met
  misc_feature (44)..(44): Xaa is Ala, Phe, or Pro
  misc_feature (45)..(45): Xaa is Gly, Lys, or absent
  misc_feature (46)..(46): Xaa is Ala or absent
  misc_feature (47)..(47): Xaa is Lys or absent
  misc_feature (48)..(48): Xaa is Val or absent
  misc_feature (49)..(49): Xaa is Asp or absent
  misc_feature (50)..(50): Xaa is Phe or absent
  misc_feature (51)..(51): Xaa is Ile or absent
  misc_feature (52)..(52): Xaa is Pro or absent
  misc_feature (53)..(53): Xaa is Arg or absent
  misc_feature (54)..(54): Xaa is Gln or Ala
  misc_feature (55)..(55): Xaa is Cys, Phe, Lys, Leu, or Trp
  misc_feature (57)..(57): Xaa is Asp or Ser
  misc_feature (58)..(58): Xaa is Asp, Glu, Gly, Ser, Thr, or Val
  misc_feature (59)..(59): Xaa is His, Leu, or Val
  misc_feature (60)..(60): Xaa is Met, Arg, Ser, or absent
  misc_feature (61)..(61): Xaa is Ala, Ser, Thr, or Trp
  misc_feature (62)..(62): Xaa is Asp, Gln, Phe, Arg, Ser, or Thr
  misc_feature (63)..(63): Xaa is Gln or absent
  misc_feature (64)..(64): Xaa is Arg or absent
  misc_feature (65)..(65): Xaa is Gln, Phe, Arg, or Ser
  misc_feature (66)..(66): Xaa is Gln, Phe, or Ser
  misc_feature (67)..(67): Xaa is Asp, Gln, Phe, Met, Ser, or Thr
  misc_feature (68)..(68): Xaa is Asp, Ile, Asn, Arg, or Thr
  misc_feature (69)..(69): Xaa is Ile, Met, Ser, Thr, or Tyr
  misc_feature (71)..(71): Xaa is Asp, Arg, or Thr
  misc_feature (75)..(75): Xaa is Ile or Met
  misc_feature (76)..(76): Xaa is Asp or Phe
  misc_feature (79)..(79): Xaa is Asp or Thr
  misc_feature (84)..(84): Xaa is Gln or Met
  misc_feature (85)..(85): Xaa is Asp or Ala
  misc_feature (87)..(87): Xaa is Gly, Met, or absent
  misc_feature (88)..(88): Xaa is Ala or Thr
  misc_feature (89)..(89): Xaa is Asp, His, Lys, or Val
  misc_feature (90)..(90): Xaa is Cys, Thr, or Tyr
  misc_feature (92)..(92): Xaa is Phe or Pro
  misc_feature (93)..(93): Xaa is Leu or Val
  misc_feature (94)..(94): Xaa is Gln, Met, or Ser
  misc_feature (95)..(95): Xaa is His or Ser
  misc_feature (97)..(97): Xaa is Ala, Phe, Ile, or Arg
  misc_feature (98)..(98): Xaa is Lys or Asn
  misc_feature (103)..(103): Xaa is Lys or Val
  misc_feature (105)..(105): Xaa is Pro, Thr, or Tyr
  misc_feature (107)..(107): Xaa is Asp, Gly, Met, or Tyr
  misc_feature (108)..(108): Xaa is Asp, ile, Lys, Leu, Thr, or Tyr
  misc_feature (109)..(109): Xaa is Asp, Ala, Phe, Gly, Ile, Arg, Thr, Val, or Tyr
  misc_feature (110)..(110): Xaa is Asp, Gln, Ala, Cys, His, Ile, Met, Asn, Ser, or absent
  misc_feature (111)..(111): Xaa is Phe, His, Lys, Asn, Arg, Ser, Val, Tyr, or absent
  misc_feature (112)..(112): Xaa is Gln, Cys, Leu, Ser, Tyr, or absent
  misc_feature (113)..(113): Xaa is Phe, Ser, Thr, Trp, Tyr, or absent
  misc_feature (114)..(114): Xaa is Phe, Gly, Ser, or absent
  misc_feature (115)..(115): Xaa is Phe, Arg, Ser, Thr, or absent
  misc_feature (116)..(116): Xaa is Asp, Gln, Phe, or absent
  misc_feature (117)..(117): Xaa is Thr, Tyr, or absent
  misc_feature (118)..(118): Xaa is Glu, His, Ser, Tyr, or absent
  misc_feature (119)..(119): Xaa is Lys, Ser, Val, or absent
  misc_feature (120)..(120): Xaa is Ser, Thr, Val, Tyr, or absent
  misc_feature (121)..(121): Xaa is Asp, Gln, Cys, His, Asn, Trp, Tyr, or absent
  misc_feature (122)..(122): Xaa is Gln, Ile, Lys, Asn, Arg, Ser, Thr, Trp, or Tyr
  misc_feature (123)..(123): Xaa is Asp, Phe, Lys, Arg, Ser, or Thr
  misc_feature (124)..(124): Xaa is Cys, Phe, His, Ile, Lys, Pro, Arg, Ser, or Tyr
  misc_feature (125)..(125): Xaa is Asp, Gln, Lys, Met, Asn, Ser, Thr, or Val
  misc_feature (126)..(126): Xaa is Asp, Cys, Met, Thr, Trp, or Tyr
  misc_feature (127)..(127): Xaa is Arg or Trp
  misc_feature (129)..(129): Xaa is Ile or Pro
XVQLXXSGGGXVQXGGSLXLSCAXSXXXXXXXXXXXXRQAPGXXXXXXXXXXXXXVXXXXXXXXXXXXXYXDSVXXRFXISRDXXKXXXXLXXXXLXXEDTAXYXCXXXXXXXXXXXXXXXXXXXXXGXGTQVTVSS

SEQ ID NO: 186 - protein, 133 aa, Artificial Sequence
  VHH consensus sequence
  misc_feature (1)..(1): Xaa is Asp, Ala, or Pro
  misc_feature (2)..(2): Xaa is Lys or Val
  misc_feature (3)..(3): Xaa is Ile or Pro
  misc_feature (5)..(5): Xaa is Lys, Pro, or Val
  misc_feature (6)..(6): Xaa is Gln, Ala, Pro, or Ser
  misc_feature (11)..(11): Xaa is Lys or Ser
  misc_feature (13)..(13): Xaa is Ala, Lys, or Pro
  misc_feature (14)..(14): Xaa is Asp, Asn, or Thr
  misc_feature (16)..(16): Xaa is Ala, Phe, or Arg
  misc_feature (19)..(19): Xaa is Arg or Thr
  misc_feature (23)..(23): Xaa is Asp, Ala, His, Thr, or Val
  misc_feature (24)..(24): Xaa is Asp, Thr, or Val
  misc_feature (25)..(25): Xaa is Asp or Ser
  misc_feature (26)..(26): Xaa is Phe or Val
  misc_feature (27)..(27): Xaa is Cys, His, Lys, Met, Arg, Ser, Thr, or Val
  misc_feature (28)..(28): Xaa is His, Asn, Ser, or Thr
  misc_feature (29)..(29): Xaa is Cys, His, Lys, or Ser
  misc_feature (30)..(30): Xaa is Gln, Phe, Met, Arg, or Ser
  misc_feature (31)..(31): Xaa is Gln, Phe, Gly, His, Met, Pro, Arg, Ser, or Tyr
  misc_feature (32)..(32): Xaa is Lys, Met, Ser, Val, or Tyr
  misc_feature (33)..(33): Xaa is Asp, Met, Ser, Thr, or Val
  misc_feature (34)..(34): Xaa is His, Leu, or Val
  misc_feature (35)..(35): Xaa is Asp, Phe, Gly, Met, or Ser
  misc_feature (37)..(37): Xaa is Cys, Val, Trp, or Tyr
  misc_feature (40)..(40): Xaa is Asp, His, Ser, or Thr
  misc_feature (41)..(41): Xaa is Asn or absent
  misc_feature (42)..(42): Xaa is Pro or absent
  misc_feature (43)..(43): Xaa is Asp or absent
  misc_feature (44)..(44): Xaa is Asn, Arg, or Ser
  misc_feature (46)..(46): Xaa is Asp, Ile, or Pro
  misc_feature (47)..(47): Xaa is Gln, Ala, Phe, Asn, or Pro
  misc_feature (48)..(48): Xaa is Gly, Lys, Arg, or Thr
  misc_feature (49)..(49): Xaa is Asp, Gln, or Ala
  misc_feature (50)..(50): Xaa is Asp, Cys, Phe, Lys, Arg, Val, or Trp
  misc_feature (51)..(51): Xaa is His, Lys, or Val
  misc_feature (52)..(52): Xaa is Asp, Ser, or Thr
  misc_feature (53)..(53): Xaa is Asp, Glu, Cys, Phe, His, Ser, Thr, Val, or Tyr
  misc_feature (54)..(54): Xaa is His, Lys, Leu, or Val
  misc_feature (55)..(55): Xaa is Phe, Met, Ser, Thr, or Trp
  misc_feature (56)..(56): Xaa is Cys, Phe, Met, Asn, Arg, Ser, Thr, Val, Trp, or absent
  misc_feature (57)..(57): Xaa is Phe, Met, Arg, Ser, Tyr, or absent
  misc_feature (58)..(58): Xaa is Gln, Cys, Phe, Leu, Arg, Ser, Thr, Val, or Tyr
  misc_feature (59)..(59): Xaa is Gln, Ala, Phe, Arg, Ser, or Thr
  misc_feature (60)..(60): Xaa is Asp, Gln, Ala, His, Met, Arg, Ser, or Thr
  misc_feature (61)..(61): Xaa is Asp, Phe, His, Met, Arg, Ser, or Thr
  misc_feature (62)..(62): Xaa is Gln, Cys, Gly, His, Lys, Met, Arg, Ser, Thr, Val, or Tyr
  misc_feature (64)..(64): Xaa is Asp, Phe, His, Ser, Thr, or Val
  misc_feature (65)..(65): Xaa is Asp, Gln, Ala, Phe, Ile, Leu, or Tyr
  misc_feature (66)..(66): Xaa is His or Ser
  misc_feature (67)..(67): Xaa is Asp or Val
  misc_feature (68)..(68): Xaa is Asp, Ile, or Arg
  misc_feature (73)..(73): Xaa is Cys, His, Lys, Thr, or Val
  misc_feature (75)..(75): Xaa is Arg or Thr
  misc_feature (77)..(77): Xaa is Gln, Phe, or Met
  misc_feature (78)..(78): Xaa is Asp, Gln, Phe, or Thr
  misc_feature (79)..(79): Xaa is Ala, Ile, Pro, or Arg
  misc_feature (80)..(80): Xaa is Gln, Ile, Met, Ser, or Thr
  misc_feature (81)..(81): Xaa is Asp, Ile, or Thr
  misc_feature (82)..(82): Xaa is Asp, His, Lys, or Val
  misc_feature (83)..(83): Xaa is Gln, Cys, Gly, Ser, Thr, or Tyr
  misc_feature (87)..(87): Xaa is Gln, Met, Arg, Ser, or Thr
  misc_feature (88)..(88): Xaa is Gln, Met, or Ser
  misc_feature (90)..(90): Xaa is Ala, Ile, Met, or Arg
  misc_feature (91)..(91): Xaa is Asp, Asn, or Ser
  misc_feature (92)..(92): Xaa is Gln or Ala
  misc_feature (95)..(95): Xaa is Asp or Phe
  misc_feature (96)..(96): Xaa is Asp, Gln, or Val
  misc_feature (98)..(98): Xaa is Cys, His, Arg, Val, or Tyr
  misc_feature (100)..(100): Xaa is Asp, Gly, Met, Pro, Ser, Thr, Trp, or Tyr
  misc_feature (101)..(101): Xaa is Asp, His, Ile, Lys, Ser, or Thr
  misc_feature (102)..(102): Xaa is Asp, Gln, Phe, Gly, Arg, Ser, Val, or Tyr
  misc_feature (103)..(103): Xaa is Phe, Gly, Met, Asn, Pro, Arg, Ser, Tyr, or absent
  misc_feature (104)..(104): Xaa is Asp, Gln, Phe, Asn, Pro, Arg, Val, or absent
  misc_feature (105)..(105): Xaa is Asp, Gln, Cys, Phe, Lys, Leu, Arg, Ser, Thr, Val, Tyr, or absent
  misc_feature (106)..(106): Xaa is Cys, Gly, Lys, Met, Arg, Ser, Val, Tyr, or absent
  misc_feature (107)..(107): Xaa is Asp, Ala, Arg, Ser, Val, Tyr, or absent
  misc_feature (108)..(108): Xaa is Cys, Phe, Lys, Leu, Ser, Tyr, or absent
  misc_feature (109)..(109): Xaa is Gln, Phe, Arg, or absent
  misc_feature (110)..(110): Xaa is Glu, Met, Asn, Arg, Ser, or absent
  misc_feature (111)..(111): Xaa is Cys, Lys, Ser, Thr, Tyr, or absent
  misc_feature (112)..(112): Xaa is Asp, Phe, Lys, Trp, Tyr, or absent
  misc_feature (113)..(113): Xaa is Glu, Cys, Met, Asn, Val, Tyr, or absent
  misc_feature (114)..(114): Xaa is Cys, Phe, His, Lys, Pro, Ser, Tyr, or absent
  misc_feature (115)..(115): Xaa is Glu, Phe, Arg, Ser, Thr, or absent
  misc_feature (116)..(116): Xaa is Asp, Asn, Arg, Ser, Trp, Tyr, or absent
  misc_feature (117)..(117): Xaa is Asp, Gln, Ala, Phe, Gly, Asn, Pro, Arg, Thr, Val, Tyr, or absent
  misc_feature (118)..(118): Xaa is Asp, Gln, Phe, His, Met, Arg, Ser, Thr, Val, or absent
  misc_feature (119)..(119): Xaa is Asp, Gln, Ala, Phe, Gly, Ile, Lys, Pro, Arg, Ser, Thr, or absent
  misc_feature (120)..(120): Xaa is Phe, Gly, Leu, Met, Ser, Tyr, or absent
  misc_feature (121)..(121): Xaa is Gln, Ala, Phe, Lys, Met, Ser, Thr, or Tyr
  misc_feature (122)..(122): Xaa is Gln, Ser, Thr, Val, or Tyr
  misc_feature (123)..(123): Xaa is Arg or Trp
  misc_feature (125)..(125): Xaa is Ile or Pro
XXXLXXSGGGXVXXGXSLXLSCXXXXXXXXXXXXXWXRQXXXXXGXXXXXXXXXXXXXXXXXYXXXXXGRFTXSXDXXXXXXXLQMXXLXXXDTXXYXCXXXXXXXXXXXXXXXXXXXXXXXXGXGTQVTVSS

(19) United States
(12) Patent Application Publication
(10) Pub. No.: US 2024/0270826 A1
(43) Pub. Date: Aug. 15, 2024

(54) ANTIBODIES THAT BIND SARS-COV-2 SPIKE PROTEIN

(71) Applicant: National Research Council of Canada, Ottawa (CA)

(72) Inventors: Jamshid TANHA, Orleans (CA); Martin A. ROSSOTTI, Ottawa (CA)

(73) Assignee: National Research Council of Canada, Ottawa, ON (CA)

(21) Appl. No.: 18/556,415

(22) PCT Filed: Apr. 22, 2022
(86) PCT No.: PCT/IB2022/053756

§ 371 Date: Oct. 20, 2023

(30) Foreign Application Priority Data
Apr. 22, 2021 (CA) 3115877

(51) Int. Cl. C07K 16/10; A61K 39/00; A61P 31/14; G01N 33/569

(52) U.S. Cl. CPC C07K 16/1003; A61P 31/14; G01N 33/56983; A61K 2039/505; C07K 2317/22; C07K 2317/76; C07K 2317/92; C07K 2317/94; G01N 2333/165; G01N 2469/10

(57) ABSTRACT

Described herein are antibodies that specifically recognize the SARS-CoV-2 spike (S) polypeptide, compositions comprising said antibodies, uses thereof, and methods employing said antibodies. Each antibody specifically recognizes the S1-RBD domain, S1-NTD domain, or S2 subunit of the SARS-CoV-2 spike polypeptide. Some antibodies are cross-reactive with variants of SARS-CoV-2 and other coronavirus spike polypeptides, such as SARS-CoV S, pangolin CoV S, bat SARS-like CoV S, and civet SARS-CoV S.

### FIELD

The present disclosure relates to antibodies that specifically bind a coronavirus spike polypeptide, particularly the spike polypeptide of SARS-CoV-2 and variants thereof, and to the use of such antibodies for various applications including the detection of a coronavirus and/or treatment or prevention of a coronavirus infection.

### BACKGROUND

Coronavirus is a single-stranded enveloped RNA virus belonging to the subfamily Coronavirinae in the order Nidovirales. Based on genomic structure, coronaviruses have been classified into four genera; Alphacoronavirus, Betacoronavirus, Gammacoronavirus, and Deltacoronavirus; two of which (alphacorona viruses and betacoronaviruses) infect mammals. Seven coronaviruses are known to cause human disease: HCoV 229E, HCov OC43, HCoVNL63, HCoVHKU1, SARS-CoV, MERS-CoV, and SARS-CoV-2. Three coronaviruses, SARS-CoV, MERS-CoV, and SARS-CoV-2, cause serious illness in humans, whereas the remaining four human coronaviruses are associated with mild illness.

Since 2002, there have been three coronavirus outbreaks causing serious human illness. The first outbreak, caused by SARS-CoV, originated in China with the first case reported in November 2002. By July 2003, there were 8098 cases and 774 deaths in 29 countries (Arora et al., 2020). The second outbreak, caused by MERS-CoV, originated in Saudi Arabia, with the first case reported in June 2012. The disease was ultimately identified in 26 countries, with 1621 confirmed cases and 584 deaths (Arora et al., 2020). The third outbreak, caused by SARS-CoV-2, originated in China with the first case reported in December 2019. On Mar. 11, 2020, the World Health Organization (WHO) declared the outbreak a pandemic. According to information provided by the Johns Hopkins Coronavirus Resource Center, as of Apr. 22, 2021, the global case count was 144 million and there had been 3.06 million deaths worldwide.

Coronavirus entry into host cells is mediated by the coronavirus spike protein (S), which is a homotrimeric glycoprotein. The spike polypeptide includes three segments, an ectodomain, a single-pass transmembrane anchor, and an intracellular tail. The spike ectodomain is made up of a receptor-binding subunit (S1) and a membrane-fusion subunit (S2). S1 includes two major domains, an N-terminal domain (NTD) and a C-terminal domain (CTD), which is also known as the receptor binding domain (RBD). Following the RBD, S1 contains two subdomains (SD1 and S1-5D2) as described in Lan et al., 2020.

During virus entry, S1 binds to a host cell surface receptor and S2 fuses the host and viral membranes (Li, 2016). The host cell surface receptor bound by both SARS-CoV and SARS-CoV-2 is a zinc peptidase angiotensin-converting enzyme 2 (ACE2), whereas MERS-CoV recognizes a serine peptidase (DPP4) (Li, 2016; Zhou et al, 2020). The receptor binding domain (RBD) of SARS-CoV-2 has been characterized and the binding mode of the SARS-CoV-2 RBD to ACE2 has been found to be nearly identical to that observed for SARS-CoV (Lan et al., 2020).

There are currently few treatments available for SARS-CoV-2 infection or other coronavirus infections and, while vaccines for SARS-CoV-2 are now coming onto the market, vaccine distribution is far from complete. Additionally, the duration and breadth of protection offered by SARS-CoV-2 vaccines is not yet known, meaning that vaccinated individuals may become increasingly susceptible to subsequent infection with time. Further, vaccination may be ineffective for immunocompromised individuals, leaving them susceptible to life-threatening coronavirus infections. Antibodies that neutralize coronaviruses, such as SARS-CoV-2, have significant potential as therapeutic agents. Further antibodies with high affinity for coronaviruses, such as SARS-CoV-2, may allow for detection, quantification, or capture of coronaviruses with high sensitivity and specificity.

### SUMMARY

Provided is an isolated or purified antibody that specifically recognizes at least one coronavirus spike polypeptide, wherein the antibody comprises an antigen binding portion of an antibody heavy chain, wherein the antigen binding portion comprises a first complementarity determining region (CDR1), a second complementarity determining region (CDR2), and a third complementarity determining region (CDR3), and wherein CDR1, CDR2, and CDR3, respectively, comprise the amino acid sequence set forth in:
SEQ ID NO: 1, SEQ ID NO: 45, and SEQ ID NO: 90;
SEQ ID NO: 2, SEQ ID NO: 45, and SEQ ID NO: 91;
SEQ ID NO: 1, SEQ ID NO: 46, and SEQ ID NO: 92;
SEQ ID NO: 3, SEQ ID NO: 47, and SEQ ID NO: 93;
SEQ ID NO: 4, SEQ ID NO: 48, and SEQ ID NO: 94;
SEQ ID NO: 5, SEQ ID NO: 49, and SEQ ID NO: 95;
SEQ ID NO: 6, SEQ ID NO: 50, and SEQ ID NO: 96;
SEQ ID NO: 7, SEQ ID NO: 51, and SEQ ID NO: 97;
SEQ ID NO: 8, SEQ ID NO: 52, and SEQ ID NO: 98;
SEQ ID NO: 9, SEQ ID NO: 53, and SEQ ID NO: 99;
SEQ ID NO: 10, SEQ ID NO: 54, and SEQ ID NO: 100;
SEQ ID NO: 11, SEQ ID NO: 55, and SEQ ID NO: 101;
SEQ ID NO: 12, SEQ ID NO: 56, and SEQ ID NO: 102;
SEQ ID NO: 13, SEQ ID NO: 57, and SEQ ID NO: 103;
SEQ ID NO: 14, SEQ ID NO: 58, and SEQ ID NO: 104;
SEQ ID NO: 15, SEQ ID NO: 59, and SEQ ID NO: 105;
SEQ ID NO: 16, SEQ ID NO: 60, and SEQ ID NO: 106;
SEQ ID NO: 17, SEQ ID NO: 61, and SEQ ID NO: 107;
SEQ ID NO: 18, SEQ ID NO: 62, and SEQ ID NO: 108;
SEQ ID NO: 19, SEQ ID NO: 63, and SEQ ID NO: 109;
SEQ ID NO: 20, SEQ ID NO: 64, and SEQ ID NO: 110;
SEQ ID NO: 21, SEQ ID NO: 65, and SEQ ID NO: 111;
SEQ ID NO: 22, SEQ ID NO: 66, and SEQ ID NO: 112;
SEQ ID NO: 23, SEQ ID NO: 67, and SEQ ID NO: 113;
SEQ ID NO: 24, SEQ ID NO: 68, and SEQ ID NO: 114;
SEQ ID NO: 25, SEQ ID NO: 69, and SEQ ID NO: 115;
SEQ ID NO: 26, SEQ ID NO: 70, and SEQ ID NO: 116;
SEQ ID NO: 27, SEQ ID NO: 71, and SEQ ID NO: 117;
SEQ ID NO: 28, SEQ ID NO: 72, and SEQ ID NO: 118;
SEQ ID NO: 29, SEQ ID NO: 73, and SEQ ID NO: 119;
SEQ ID NO: 30, SEQ ID NO: 74, and SEQ ID NO: 120;
SEQ ID NO: 31, SEQ ID NO: 75, and SEQ ID NO: 121;
SEQ ID NO: 32, SEQ ID NO: 76, and SEQ ID NO: 122;
SEQ ID NO: 33, SEQ ID NO: 77, and SEQ ID NO: 123;
SEQ ID NO: 34, SEQ ID NO: 78, and SEQ ID NO: 124;
SEQ ID NO: 35, SEQ ID NO: 79, and SEQ ID NO: 125;
SEQ ID NO: 36, SEQ ID NO: 80, and SEQ ID NO: 125;
SEQ ID NO: 20, SEQ ID NO: 81, and SEQ ID NO: 126;
SEQ ID NO: 20, SEQ ID NO: 81, and SEQ ID NO: 127;
SEQ ID NO: 37, SEQ ID NO: 82, and SEQ ID NO: 128;
SEQ ID NO: 38, SEQ ID NO: 83, and SEQ ID NO: 129;
SEQ ID NO: 39, SEQ ID NO: 84, and SEQ ID NO: 130;
SEQ ID NO: 40, SEQ ID NO: 85, and SEQ ID NO: 131;
SEQ ID NO: 41, SEQ ID NO: 86, and SEQ ID NO: 132;
SEQ ID NO: 42, SEQ ID NO: 87, and SEQ ID NO: 133;
SEQ ID NO: 43, SEQ ID NO: 88, and SEQ ID NO: 134; or
SEQ ID NO: 44, SEQ ID NO: 89, and SEQ ID NO: 135.

In an embodiment, the antibody is a neutralizing antibody and CDR1, CDR2, and CDR3, respectively, comprise the amino acid sequence set forth in:
SEQ ID NO: 1, SEQ ID NO: 45, and SEQ ID NO: 90;
SEQ ID NO: 1, SEQ ID NO: 46, and SEQ ID NO: 92;
SEQ ID NO: 2, SEQ ID NO: 45, and SEQ ID NO: 91;
SEQ ID NO: 3, SEQ ID NO: 47, and SEQ ID NO: 93;
SEQ ID NO: 4, SEQ ID NO: 48, and SEQ ID NO: 94;
SEQ ID NO: 5, SEQ ID NO: 49, and SEQ ID NO: 95;
SEQ ID NO: 6, SEQ ID NO: 50, and SEQ ID NO: 96;
SEQ ID NO: 7, SEQ ID NO: 51, and SEQ ID NO: 97;
SEQ ID NO: 8, SEQ ID NO: 52, and SEQ ID NO: 98;
SEQ ID NO: 10, SEQ ID NO: 54, and SEQ ID NO: 100;
SEQ ID NO: 11, SEQ ID NO: 55, and SEQ ID NO: 101;
SEQ ID NO: 13, SEQ ID NO: 57, and SEQ ID NO: 103;
SEQ ID NO: 14, SEQ ID NO: 58, and SEQ ID NO: 104;
SEQ ID NO: 15, SEQ ID NO: 59, and SEQ ID NO: 105;
SEQ ID NO: 16, SEQ ID NO: 60, and SEQ ID NO: 106;
SEQ ID NO: 17, SEQ ID NO: 61, and SEQ ID NO: 107;
SEQ ID NO: 19, SEQ ID NO: 63, and SEQ ID NO: 109;
SEQ ID NO: 20, SEQ ID NO: 81, and SEQ ID NO: 127;
SEQ ID NO: 21, SEQ ID NO: 65, and SEQ ID NO: 111;
SEQ ID NO: 22, SEQ ID NO: 66, and SEQ ID NO: 112;
SEQ ID NO: 23, SEQ ID NO: 67, and SEQ ID NO: 113;
SEQ ID NO: 25, SEQ ID NO: 69, and SEQ ID NO: 115;
SEQ ID NO: 26, SEQ ID NO: 70, and SEQ ID NO: 116;
SEQ ID NO: 27, SEQ ID NO: 71, and SEQ ID NO: 117;
SEQ ID NO: 32, SEQ ID NO: 76, and SEQ ID NO: 122;
SEQ ID NO: 33, SEQ ID NO: 77, and SEQ ID NO: 123; or
SEQ ID NO: 36, SEQ ID NO: 80, and SEQ ID NO: 125.

In an embodiment, the antibody specifically binds the S1-NTD domain of the coronavirus spike polypeptide and CDR1, CDR2, and CDR3, respectively, comprise the amino acid sequence set forth in:
SEQ ID NO: 21, SEQ ID NO: 65, and SEQ ID NO: 111;
SEQ ID NO: 22, SEQ ID NO: 66, and SEQ ID NO: 112;
SEQ ID NO: 23, SEQ ID NO: 67, and SEQ ID NO: 113;
SEQ ID NO: 24, SEQ ID NO: 68, and SEQ ID NO: 114;
SEQ ID NO: 25, SEQ ID NO: 69, and SEQ ID NO: 115;
SEQ ID NO: 26, SEQ ID NO: 70, and SEQ ID NO: 116;
SEQ ID NO: 20, SEQ ID NO: 81, and SEQ ID NO: 126;
SEQ ID NO: 37, SEQ ID NO: 82, and SEQ ID NO: 128; or
SEQ ID NO: 38, SEQ ID NO: 83, and SEQ ID NO: 129.

In an embodiment, the antibody specifically binds the S2 subunit of the coronavirus spike polypeptide and CDR1, CDR2, and CDR3, respectively, comprise the amino acid sequence set forth in:
SEQ ID NO: 27, SEQ ID NO: 71, and SEQ ID NO: 117;
SEQ ID NO: 28, SEQ ID NO: 72, and SEQ ID NO: 118;
SEQ ID NO: 29, SEQ ID NO: 73, and SEQ ID NO: 119;
SEQ ID NO: 30, SEQ ID NO: 74, and SEQ ID NO: 120;
SEQ ID NO: 31, SEQ ID NO: 75, and SEQ ID NO: 121;
SEQ ID NO: 32, SEQ ID NO: 76, and SEQ ID NO: 122;
SEQ ID NO: 33, SEQ ID NO: 77, and SEQ ID NO: 123;
SEQ ID NO: 34, SEQ ID NO: 78, and SEQ ID NO: 124;
SEQ ID NO: 39, SEQ ID NO: 84, and SEQ ID NO: 130;
SEQ ID NO: 40, SEQ ID NO: 85, and SEQ ID NO: 131;
SEQ ID NO: 41, SEQ ID NO: 86, and SEQ ID NO: 132;
SEQ ID NO: 42, SEQ ID NO: 87, and SEQ ID NO: 133;
SEQ ID NO: 43, SEQ ID NO: 88, and SEQ ID NO: 134; or
SEQ ID NO: 44, SEQ ID NO: 89, and SEQ ID NO: 135.

In an embodiment, the antibody specifically binds the S1-RBD domain of the coronavirus spike polypeptide and CDR1, CDR2, and CDR3, respectively, comprise the amino acid sequence set forth in:
SEQ ID NO: 1, SEQ ID NO: 45, and SEQ ID NO: 90;
SEQ ID NO: 2, SEQ ID NO: 45, and SEQ ID NO: 91;
SEQ ID NO: 1, SEQ ID NO: 46, and SEQ ID NO: 92;
SEQ ID NO: 3, SEQ ID NO: 47, and SEQ ID NO: 93;
SEQ ID NO: 4, SEQ ID NO: 48, and SEQ ID NO: 94;
SEQ ID NO: 5, SEQ ID NO: 49, and SEQ ID NO: 95;
SEQ ID NO: 6, SEQ ID NO: 50, and SEQ ID NO: 96;
SEQ ID NO: 7, SEQ ID NO: 51, and SEQ ID NO: 97;
SEQ ID NO: 8, SEQ ID NO: 52, and SEQ ID NO: 98;
SEQ ID NO: 9, SEQ ID NO: 53, and SEQ ID NO: 99;
SEQ ID NO: 10, SEQ ID NO: 54, and SEQ ID NO: 100;
SEQ ID NO: 11, SEQ ID NO: 55, and SEQ ID NO: 101;
SEQ ID NO: 12, SEQ ID NO: 56, and SEQ ID NO: 102;
SEQ ID NO: 13, SEQ ID NO: 57, and SEQ ID NO: 103;
SEQ ID NO: 14, SEQ ID NO: 58, and SEQ ID NO: 104;
SEQ ID NO: 15, SEQ ID NO: 59, and SEQ ID NO: 105;
SEQ ID NO: 16, SEQ ID NO: 60, and SEQ ID NO: 106;
SEQ ID NO: 17, SEQ ID NO: 61, and SEQ ID NO: 107;
SEQ ID NO: 18, SEQ ID NO: 62, and SEQ ID NO: 108;
SEQ ID NO: 19, SEQ ID NO: 63, and SEQ ID NO: 109;
SEQ ID NO: 20, SEQ ID NO: 64, and SEQ ID NO: 110;
SEQ ID NO: 35, SEQ ID NO: 79, and SEQ ID NO: 125;
SEQ ID NO: 36, SEQ ID NO: and SEQ ID NO: 125; or
SEQ ID NO: 20, SEQ ID NO: and SEQ ID NO: 127.

In an embodiment, the antibody is cross-reactive with the spike polypeptide of SARS-CoV-2 and SARS-CoV, and CDR1, CDR2, and CDR3, respectively, comprise the amino acid sequence set forth in:
SEQ ID NO: 1, SEQ ID NO: 45, and SEQ ID NO: 92;
SEQ ID NO: 9, SEQ ID NO: 53, and SEQ ID NO: 99;
SEQ ID NO: 21, SEQ ID NO: 65, and SEQ ID NO: 111;
SEQ ID NO: 22, SEQ ID NO: 66, and SEQ ID NO: 112;
SEQ ID NO: 8, SEQ ID NO: 52, and SEQ ID NO: 98;
SEQ ID NO: 11, SEQ ID NO: 55, and SEQ ID NO: 101;
SEQ ID NO: 13, SEQ ID NO: 57, and SEQ ID NO: 103;
SEQ ID NO: 18, SEQ ID NO: 62, and SEQ ID NO: 108;
SEQ ID NO: 19, SEQ ID NO: 63, and SEQ ID NO: 109;
SEQ ID NO: 36, SEQ ID NO: 80, and SEQ ID NO: 125;
SEQ ID NO: 28, SEQ ID NO: 72, and SEQ ID NO: 118;
SEQ ID NO: 30, SEQ ID NO: 74, and SEQ ID NO: 120;
SEQ ID NO: 31, SEQ ID NO: 75, and SEQ ID NO: 121;
SEQ ID NO: 32, SEQ ID NO: 76, and SEQ ID NO: 122;
SEQ ID NO: 33, SEQ ID NO: 77, and SEQ ID NO: 123;
SEQ ID NO: 40, SEQ ID NO: 85, and SEQ ID NO: 131;
SEQ ID NO: 41, SEQ ID NO: 86, and SEQ ID NO: 132; or
SEQ ID NO: 42, SEQ ID NO: 87, and SEQ ID NO: 133.

In an embodiment, the antibody recognizes a linear epitope, and CDR1, CDR2, and CDR3, respectively, comprise the amino acid sequence set forth in:
SEQ ID NO: 21, SEQ ID NO: 65, and SEQ ID NO: 111;
SEQ ID NO: 24, SEQ ID NO: 68, and SEQ ID NO: 114;
SEQ ID NO: 27, SEQ ID NO: 71, and SEQ ID NO: 117;
SEQ ID NO: 28, SEQ ID NO: 72, and SEQ ID NO: 118;
SEQ ID NO: 29, SEQ ID NO: 73, and SEQ ID NO: 119;
SEQ ID NO: 31, SEQ ID NO: 75, and SEQ ID NO: 121;
SEQ ID NO: 32, SEQ ID NO: 76, and SEQ ID NO: 122;
SEQ ID NO: 30, SEQ ID NO: 74, and SEQ ID NO: 120;
SEQ ID NO: 39, SEQ ID NO: 84, and SEQ ID NO: 130;
SEQ ID NO: 40, SEQ ID NO: 85, and SEQ ID NO: 131; or
SEQ ID NO: 41, SEQ ID NO: 86, and SEQ ID NO: 132.

In an embodiment, the antibody comprises the amino acid sequence set forth in SEQ ID NO: 183.

In an embodiment of the antibody, CDR1, CDR2, and CDR3, respectively, comprise the amino acid sequence set forth in:
SEQ ID NO: 21, SEQ ID NO: 65, and SEQ ID NO: 111;
SEQ ID NO: 23, SEQ ID NO: 67, and SEQ ID NO: 113; or
SEQ ID NO: 24, SEQ ID NO: 68, and SEQ ID NO: 114.

In an embodiment of the antibody, CDR1, CDR2, and CDR3, respectively, comprise the amino acid sequence set forth in: SEQ ID NO: 38, SEQ ID NO: 83, and SEQ ID NO: 129.

In an embodiment of the antibody, CDR1, CDR2, and CDR3, respectively, comprise the amino acid sequence set forth in:
SEQ ID NO: 20, SEQ ID NO: 81, and SEQ ID NO: 126 or
SEQ ID NO: 37, SEQ ID NO: 82, and SEQ ID NO: 128.

In an embodiment, the antibody comprises the amino acid sequence set forth in SEQ ID NO: 184.

In an embodiment, the antibody comprises the amino acid sequence set forth in SEQ ID NO: 158, SEQ ID NO: 157, SEQ ID NO: 172, SEQ ID NO: 175, SEQ ID NO: 176, SEQ ID NO: 160, SEQ ID NO: 161, SEQ ID NO: 159, or SEQ ID NO: 162, or an amino acid sequence having at least 75%, at least 80%, at least 85%, at least 90%, at least 95%, at least 96%, at least 97%, at least 98%, or at least 99% sequence identity to the full length of the amino acid sequence set forth in SEQ ID NO: 158, SEQ ID NO: 157, SEQ ID NO: 172, SEQ ID NO: 175, SEQ ID NO: 176, SEQ ID NO: 160, SEQ ID NO: 161, SEQ ID NO: 159, and/or SEQ ID NO: 162.

In an embodiment of the antibody, CDR1, CDR2, and CDR3, respectively, comprise the amino acid sequence set forth in:
SEQ ID NO: 31, SEQ ID NO: 75, and SEQ ID NO: 121;
SEQ ID NO: 40, SEQ ID NO: 85, and SEQ ID NO: 131;
SEQ ID NO: 41, SEQ ID NO: 86, and SEQ ID NO: 132;
SEQ ID NO: 42, SEQ ID NO: 87, and SEQ ID NO: 133;
SEQ ID NO: 43, SEQ ID NO: 88, and SEQ ID NO: 134; or
SEQ ID NO: 44, SEQ ID NO: 89, and SEQ ID NO: 135.

In an embodiment of the antibody, CDR1, CDR2, and CDR3, respectively, comprise the amino acid sequence set forth in SEQ ID NO: 33, SEQ ID NO: 77, and SEQ ID NO: 123.

In an embodiment of the antibody, CDR1, CDR2, and CDR3, respectively, comprise the amino acid sequence set forth in SEQ ID NO: 32, SEQ ID NO: 76, and SEQ ID NO: 122.

In an embodiment of the antibody, CDR1, CDR2, and CDR3, respectively, comprise the amino acid sequence set forth in SEQ ID NO: 27, SEQ ID NO: 71, and SEQ ID NO: 117.

In an embodiment of the antibody, CDR1, CDR2, and CDR3, respectively, comprise the amino acid sequence set forth in SEQ ID NO: 28, SEQ ID NO: 72, and SEQ ID NO: 118.

In an embodiment, the antibody comprises the amino acid sequence set forth in SEQ ID NO: 185.

In an embodiment, the antibody comprises the amino acid sequence set forth in SEQ ID NO: 180, SEQ ID NO: 182, SEQ ID NO: 166, SEQ ID NO: 177, SEQ ID NO: 179, SEQ ID NO: 163, SEQ ID NO: 164, SEQ ID NO: 167, SEQ ID NO: 170, SEQ ID NO: 168, SEQ ID NO: 169, SEQ ID NO: 181, SEQ ID NO: 165, or SEQ ID NO: 178, or an amino acid sequence having at least 75%, at least 80%, at least 85%, at least 90%, at least 95%, at least 96%, at least 97%, at least 98%, or at least 99% sequence identity to the full length of the amino acid sequence set forth in SEQ ID NO: 180, SEQ ID NO: 182, SEQ ID NO: 166, SEQ ID NO: 177, SEQ ID NO: 179, SEQ ID NO: 163, SEQ ID NO: 164, SEQ ID NO: 167, SEQ ID NO: 170, SEQ ID NO: 168, SEQ ID NO: 169, SEQ ID NO: 181, SEQ ID NO: 165, and/or SEQ ID NO: 178.

In an embodiment of the antibody, CDR1, CDR2, and CDR3, respectively, comprise the amino acid sequence set forth in:
SEQ ID NO: 5, SEQ ID NO: 49, and SEQ ID NO: 95;
SEQ ID NO: 14, SEQ ID NO: 58, and SEQ ID NO: 104;
SEQ ID NO: 16, SEQ ID NO: 60, and SEQ ID NO: 106;
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in:
SEQ ID NO: 3, SEQ ID NO: 47, and SEQ ID NO: 93;
SEQ ID NO: 6, SEQ ID NO: 50, and SEQ ID NO: 96;
SEQ ID NO: 14, SEQ ID NO: 58, and SEQ ID NO: 104;
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in:
SEQ ID NO: 3, SEQ ID NO: 47, and SEQ ID NO: 93;
SEQ ID NO: 6, SEQ ID NO: 50, and SEQ ID NO: 96;
SEQ ID NO: 16, SEQ ID NO: 60, and SEQ ID NO: 106;
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in SEQ ID NO: 7, SEQ ID NO: 51, and SEQ ID NO: 97.

In an embodiment of the antibody, CDR1, CDR2, and CDR3, respectively, comprise the amino acid sequence set forth in:
SEQ ID NO: 4, SEQ ID NO: 48, and SEQ ID NO: 94;
SEQ ID NO: 9, SEQ ID NO: 53, and SEQ ID NO: 99;
SEQ ID NO: 11, SEQ ID NO: 55, and SEQ ID NO: 101; or
SEQ ID NO: 12, SEQ ID NO: 56, and SEQ ID NO: 102.

In an embodiment of the antibody, CDR1, CDR2, and CDR3, respectively, comprise the amino acid sequence set forth in:
SEQ ID NO: 3, SEQ ID NO: 47, and SEQ ID NO: 93;
SEQ ID NO: 6, SEQ ID NO: 50, and SEQ ID NO: 96;
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in:
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in:
SEQ ID NO: 14, SEQ ID NO: 58, and SEQ ID NO: 104;
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in:
SEQ ID NO: 16, SEQ ID NO: 60, and SEQ ID NO: 106;
SEQ ID NO: 10, SEQ ID NO: 54, and SEQ ID NO: 100;
SEQ ID NO: 15, SEQ ID NO: 59, and SEQ ID NO: 105; or
SEQ ID NO: 20, SEQ ID NO: 81, and SEQ ID NO: 127.

In an embodiment of the antibody, CDR1, CDR2, and CDR3, respectively, comprise the amino acid sequence set forth in:
SEQ ID NO: 1, SEQ ID NO: 45, and SEQ ID NO: 90;
SEQ ID NO: 1, SEQ ID NO: 46, and SEQ ID NO: 92;
SEQ ID NO: 2, SEQ ID NO: 45, and SEQ ID NO: 91;
SEQ ID NO: 8, SEQ ID NO: 52, and SEQ ID NO: 98;
SEQ ID NO: 13, SEQ ID NO: 57, and SEQ ID NO: 103;
SEQ ID NO: 17, SEQ ID NO: 61, and SEQ ID NO: 107;
SEQ ID NO: 19, SEQ ID NO: 63, and SEQ ID NO: 109;
SEQ ID NO: 35, SEQ ID NO: 79, and SEQ ID NO: 125; or
SEQ ID NO: 36, SEQ ID NO: 80, and SEQ ID NO: 125.

In an embodiment, the antibody comprises the amino acid sequence set forth in SEQ ID NO: 186.

In an embodiment, the antibody comprises the amino acid sequence set forth in SEQ ID NO: 171, SEQ ID NO: 173, SEQ ID NO: 149, SEQ ID NO: 155, SEQ ID NO: 147, SEQ ID NO: 148, SEQ ID NO: 139, SEQ ID NO: 142, SEQ ID NO: 154, SEQ ID NO: 144, SEQ ID NO: 145, SEQ ID NO: 156, SEQ ID NO: 174, SEQ ID NO: 137, SEQ ID NO: 136, SEQ ID NO: 138, SEQ ID NO: 152, SEQ ID NO: 153, SEQ ID NO: 140, SEQ ID NO: 143, SEQ ID NO: 141, SEQ ID NO: 146, SEQ ID NO: 150, or SEQ ID NO: 151, or an amino acid sequence having at least 75%, at least 80%, at least 85%, at least 90%, at least 95%, at least 96%, at least 97%, at least 98%, or at least 99% sequence identity to the full length of the amino acid sequence set forth in SEQ ID NO: 171, SEQ ID NO: 173, SEQ ID NO: 149, SEQ ID NO: 155, SEQ ID NO: 147, SEQ ID NO: 148, SEQ ID NO: 139, SEQ ID NO: 142, SEQ ID NO: 154, SEQ ID NO: 144, SEQ ID NO: 145, SEQ ID NO: 156, SEQ ID NO: 174, SEQ ID NO: 137, SEQ ID NO: 136, SEQ ID NO: 138, SEQ ID NO: 152, SEQ ID NO: 153, SEQ ID NO: 140, SEQ ID NO: 143, SEQ ID NO: 141, SEQ ID NO: 146, SEQ ID NO: 150, and/or SEQ ID NO: 151.

In an embodiment, the antibody is a single domain antibody. In a further embodiment, the antibody is a VHH.

In an embodiment the antibody is of camelid origin.

In an embodiment, the antibody is in a multivalent display format. In a further embodiment, the antibody is linked to an Fc fragment. In a further embodiment, the Fc-linked antibody is in a bivalent display format.

In an embodiment of the antibody, the at least one coronavirus spike polypeptide specifically binds an ACE2 receptor.

In an embodiment of the antibody, the at least one coronavirus spike polypeptide comprises a SARS-CoV-2 spike polypeptide.

In an embodiment of the antibody, the at least one coronavirus spike polypeptide is comprised within a homotrimer.

Another embodiment is an antibody cocktail composition comprising two or more of the antibodies as described herein. The composition may comprise two, three, four, five, or more different antibodies as described herein. The antibody cocktail composition may further comprise a pharmaceutically acceptable carrier and/or diluent.

Another embodiment is a nucleic acid molecule encoding an antibody as described herein. A further embodiment is a vector comprising the nucleic acid molecule. In an embodiment of the vector, the nucleic acid molecule is operably linked to at least one promoter and/or regulatory element to enable expression in a host cell. An additional embodiment is a host cell comprising the vector.

Another embodiment is a pharmaceutical composition comprising at least one antibody as defined herein and a pharmaceutically acceptable carrier and/or diluent. In an embodiment, the pharmaceutical composition is for delivery by inhalation or nebulization.

Another embodiment is a composition comprising at least one antibody as defined herein, linked to another molecule. In an embodiment, the other molecule is a label or polypeptide. In an embodiment, the other molecule is an ACE2 polypeptide or a fragment thereof.

Another embodiment is a composition or apparatus comprising at least one antibody as defined herein immobilized on a substrate. A further embodiment is a method for capturing a coronavirus or a coronavirus spike polypeptide or fragment thereof from a sample, the method comprising exposing the sample to the composition or apparatus. In an embodiment, the coronavirus is a coronavirus that specifically binds an ACE2 receptor or the coronavirus spike polypeptide is a coronavirus spike polypeptide that specifically binds an ACE2 receptor. In an embodiment, the coronavirus is SARS-CoV-2 or SARS-CoV.

Another embodiment is use of an antibody as described herein to treat or detect a coronavirus infection. In an embodiment, the coronavirus infection is caused by at least one coronavirus that specifically binds an ACE2 receptor. In an embodiment, the coronavirus infection is caused by SARS-CoV-2 and/or SARS-CoV.

Another embodiment is use of an antibody or composition as described herein to detect, quantify and/or capture a coronavirus; or to detect, quantify and/or capture a coronavirus spike polypeptide or fragment thereof. In an embodiment, the coronavirus is a coronavirus that specifically binds an ACE2 receptor or the coronavirus spike polypeptide is a coronavirus spike polypeptide that specifically binds an ACE2 receptor. In an embodiment, the coronavirus is SARS-CoV-2 or SARS-CoV.

Another embodiment is a method for treating or preventing a coronavirus infection, the method comprising administering at least one antibody or composition as described herein to a subject in need thereof. In an embodiment, the coronavirus infection is caused by at least one coronavirus that specifically binds an ACE2 receptor. In an embodiment, the coronavirus infection is caused by SARS-CoV-2 and/or SARS-CoV. In an embodiment, the administration is by inhalation or nebulization.

Another embodiment is a method for detecting the presence of a coronavirus or a coronavirus spike polypeptide or fragment thereof in a sample, the method comprising exposing the sample to at least one antibody or composition as described herein and assaying for specific binding between the at least one antibody and the sample, wherein specific binding indicates a presence of the at least one coronavirus or coronavirus spike polypeptide or fragment thereof in the sample.

In an embodiment of the methods described in the preceding paragraphs, the coronavirus is a coronavirus that specifically binds an ACE2 receptor or the coronavirus spike polypeptide is a coronavirus spike polypeptide that specifically binds an ACE2 receptor. In an embodiment, coronavirus is SARS-CoV-2 or SARS-CoV, or the coronavirus spike polypeptide or fragment thereof is a SARS-CoV-2 or SARS-CoV coronavirus spike polypeptide or fragment thereof.

Another embodiment is an antibody or composition as described herein for use to detect or treat a coronavirus infection. In an embodiment, the coronavirus infection is caused by at least one coronavirus that specifically binds an ACE2 receptor. In an embodiment, the at least one coronavirus is SARS-CoV-2 and/or SARS-CoV.

Another embodiment is an antibody composition as described herein for use to detect, quantify and/or capture a coronavirus; or to detect, quantify and/or capture a coronavirus spike polypeptide or fragment thereof. In an embodiment, the coronavirus is a coronavirus that specifically binds an ACE2 receptor, or the coronavirus spike polypeptide is a coronavirus spike polypeptide that specifically binds an ACE2 receptor. In an embodiment, the coronavirus is SARS-CoV-2 or SARS-CoV or the coronavirus spike polypeptide or fragment thereof is a SARS-CoV-2 or SARS-CoV spike polypeptide or fragment thereof.

Another embodiment is use of an antibody as described herein in the manufacture of a medicament for prevention or treatment of a coronavirus infection. In an embodiment, the coronavirus infection is caused by at least one coronavirus that specifically binds an ACE2 receptor. In an embodiment, the at least one coronavirus is SARS-CoV-2 and/or SARS-CoV. In an embodiment, the medicament is for delivery by inhalation or nebulization.

### BRIEF DESCRIPTION OF THE DRAWINGS

Throughout the present disclosure, including in the drawings, antibodies may be referred to by their full name, e.g. NRCoV2-1d, NRCoV2-02, NRCoV2-SR03, or NRCoV2-MRed02, or by an abbreviation in which the “NRCoV2-” portion of the antibody name is omitted, e.g. 1 d, 02, SR03, or MRed02. Further, “RBD” and “S1-RBD” are used interchangeably, as are “NTD” and “S1-NTD”.

FIGS. 1A and 1B describe antigen validation by ELISA. FIG. 1A shows the results of an ELISA assessing the binding of microtiter-well-adsorbed (S, S1, S2, S1-RBD) and microtiter-well-captured (AviTag-S1, AviTag-S1-RBD) SARS-CoV-2 spike protein fragments to cognate ACE2 receptor (ACE2-hFc). AviTag-S1 and AviTag-S1-RBD were captured on streptavidin-coated microtiter wells through their C-terminal biotins. FIG. 1B shows the results of an ELISA confirming the binding of microtiter-well-adsorbed SARS-CoV-2 spike protein fragments S, S1, S2 and S1-RBD to a commercial rabbit anti-SARS-CoV-2 S polyclonal antibody (pAb).

FIGS. 2A and 2B show the results of llama serology. FIG. 2A shows the results of an ELISA performed with pre-immune (day 0) and immune (day 21 and 28) sera, demonstrating that spike protein-immunized Maple Red and Eva Green llamas generated a strong immune response against target antigens S, S1, S2 and S1-RBD. ELISA performed with day 0, 21 and 28 sera showed spike protein-immunized llamas did not react with non-target antigens (casein and dipeptidase 1 [DPEP1]), demonstrating specificity of the immune response. FIG. 2B shows flow cytometry surrogate neutralization assays performed with pre-immune (day 0) and immune (day 21 and 28) sera demonstrating that the Eva Green llama mounted a polyclonal immune response that was more potent in inhibiting the binding of SARS-CoV-2 S to ACE2 than Maple Red's. Due to a lack of complete curves, inhibitory serum titers for Maple Red sera were estimated assuming similar upper plateaus as those for Eva Green sera.

FIG. 3 provides a schematic representation of three different antibody formats monomeric V_{H}H, bivalent V_{H}H-Fc and monovalent V_{H}H-Fc.

FIGS. 4A and 4B show size-exclusion chromatogram (SEC) profiles of anti-SARS-CoV-2 spike protein V_{H}Hs. FIG. 4A shows SEC profiles of Eva Green V_{H}Hs. FIG. 4B shows SEC profiles of Maple Red V_{H}Hs. Vₑ, elution volume: mAU, milliabsorbance unit.

FIGS. 5A and 5B show data on the thermostability of anti-SARS-CoV-2 spike protein V_{H}Hs. FIG. 5A provides representative examples showing the thermal unfolding of NRCoV2-1d, NRCoV2-02, NRCoV2-07 and NRCoV2-11, as determined using CD spectroscopy. FIG. 5B provides a summary of V_{H}H Tₘs. The dotted line across the graph in FIG. 5B represents the median Tₘ (70.4° C.).

FIGS. 6A, 6B, 6C, 6D and 6E show SPR/ELISA binding affinity, specificity and cross reactivity data for anti-SARS-CoV-2 V_{H}Hs and V_{H}H-Fcs. FIGS. 6A and 6B show the results of ELISA assessing the cross-reactivity of anti-SARS-CoV-2 V_{H}H-Fcs against to a collection of spike glycoproteins from various coronavirus genera and SARS-CoV-2 variants. Assays were performed at a fixed V_{H}H-Fc concentration (13 nM). The V_{H}H-72 (Wrapp et al., 2020) benchmark and human ACE-2 were included for comparison. The epitope bin numbers provided along the bottom of FIG. 6B correspond to the bins shown in FIG. 9G. FIG. 6C shows representative SPR sensorgrams showing single-cycle kinetic analysis of NRCoV2-02, NRCoV2-07, NRCoV2-SR03 and NRCoV2-S2A4 V_{H}H binding to SARS-CoV S and SARS-CoV-2 S, S1, S2 and S1-RBD. Spike proteins were captured on CM5 sensorchip surfaces, followed by flowing V_{H}Hs over the sensorchip surfaces at the concentration ranges shown in each panel. “NRCoV2-02/NRCoV2-07”, “SR03” and “S2A4” represent SPR binding profiles for V_{H}Hs specific to SARS-CoV-2 S1-RBD, S1-NTD and S2, respectively. “NRCoV2-07” also represents binding profiles for V_{H}Hs that cross-react with SARS-CoV. FIGS. 6D and 6E show the results of ELISA assessing the domain specificity of a set of anti-SARS-CoV-2 V_{H}H-Fcs. Assays were performed against SARS-CoV-2 S, S1, S1-NTD and S1-RBD at a fixed concentration (13 nM) (FIG. 6D) or varying concentrations (FIG. 6E) of V_{H}H-Fcs. In the graphs shown in FIG. 6E, NRCoV2-02 is included as an internal control (dashed line).

FIGS. 7A and 7B show on-/off-rate maps summarizing V_{H}H kinetic rate constants, kₐs and k_{d}s. Diagonal lines represent equilibrium dissociation constants, K_{D}s. Maps were constructed using the V_{H}H binding data against SARS-CoV-2 S (FIG. 7A) and SARS-CoV S (FIG. 7B). In FIG. 7A, V_{H}Hs are clustered based on subunit/domain specificity determined in Example 5. Anti-SARS-CoV V_{H}H-72, which cross-reacts with SARS-CoV-2 S1-RBD (Wrapp et al., 2020), and the monomeric ACE2 (ACE2-H6) are included as benchmark/reference binders.

FIGS. 8A and 8B show the results of flow cytometry assessing the binding of V_{H}H-Fcs to SARS-CoV-2 S-expressing CHO-S cells. FIG. 8A shows representative examples. FIG. 8B summarizes affinity values, i.e., EC₅₀s, determined from graphs in FIG. 8A. V_{H}H-72 (Wrapp et al., 2020; open circle) is included for comparison. The line through the data points is the median.

FIGS. 9A, 9B, 9C, 9D, 9E, 9F, and 9G show epitope typing and binning data obtained by SDS-PAGE/WB, sandwich ELISA and SPR. FIGS. 9A and 9B show the results of epitope typing of anti-SARS-CoV-2 V_{H}Hs by SDS-PAGE/WB. Binding of biotinylated V_{H}Hs or V_{H}H-Fcs to denatured SARS-CoV-2 S was detected using streptavidin-peroxidase conjugate (FIG. 9A) or anti human Ig Fc antibody-peroxidase conjugate (FIG. 9B), respectively. Presence of binding signals indicates V_{H}H recognizing a linear epitope. The absence of binding signals is an indirect indication of V_{H}Hs recognizing conformational epitopes. Toxin A-specific A20.1 V_{H}H (Hussack et al., 2011) was used as a negative antibody control. “PBS” and “A20.1” represent experiments where V_{H}H test articles were replaced with PBS and C. difficile toxin A-specific V_{H}H A20.1. FIG. 9C shows representative sensorgrams showing SPR epitope binning on SARS-CoV-2 S-immobilized surfaces. FIGS. 9D and 9E show epitope binning of S1-RBD-specific V_{H}Hs by competitive sandwich ELISA. ELISA binding results for pair-wise combinations of V_{H}Hs against S1 are presented as a heat map. Binding pairs giving binding signal (shaded) were considered as recognizing non-overlapping epitopes hence belonging to different epitope bins or V_{H}H clusters, while those giving no/week binding signals (colorless/pale shading) were considered to be recognizing overlapping epitopes belonging to the same epitope bins. ACE2-Fc and V_{H}H-72 V_{H}H/V_{H}H-Fc benchmark (Wrapp et al., 2020) were also included in assays. Wells captured with C. difficile toxin A-specific V_{H}H negative control, A20.1 (Hussack et al., 2011), did not give any binding. FIG. 9F provides a schematic summary of the initial epitope binning results. NRCoV2-1c and NRCoV2-MRed02 were assigned to bin 1 since their CDRs were essentially the same as to those of NRCoV2-1a/1d and NRCoV2-MRed04, respectively, with experimentally defined bins. FIG. 9G provides a schematic summary of binning results after further characterization. Unless specified otherwise, references to epitope bin numbers throughout the present disclosure refer to the bins identified in FIG. 9F. The bin numbers provided in FIG. 9E correspond to the bins shown in FIG. 9G.

FIG. 10 shows the results of ELISA assessing the ability of monomeric V_{H}Hs in blocking (“neutralizing”) the binding of human ACE2 receptor (ACE2-Fc) to its SARS-CoV-2 S1-RBD ligand (i.e., S). A_{450 nm} is a measure of blocking. V_{H}H-72 V_{H}H (Wrapp et al., 2020) and monomeric ACE2-H6 served as positive antibody controls, while toxin A-specific A20.1 V_{H}H (Hussack et al., 2011) was a negative antibody control. “PBS” represents assays in which V_{H}H was substituted with PBS and, similar to the A20.1 control, provides a reference binding signal for lack of any blocking (“min inhibition”). The “-ACE2-Fc” control represents an assay in which ACE2-Fc is omitted and provides a reference binding signal for 100% blocking (“max inhibition”).

FIG. 11 shows sensorgrams showing the ability of monomeric V_{H}Hs in blocking (“neutralizing”) the binding of ACE2 receptor to its ligand SARS-CoV-2 S. A tandem SPR in two different orientation formats were performed where injection of V_{H}H (orientation #1) or ACE2 (orientation #2) at 20-40×K_{D} concentration (V_{H}H) or 1 μM (ACE2) over sensor chip-immobilized S was followed by injection of V_{H}H+ACE2 mix at the same V_{H}H and ACE2 concentrations. Solid and dashed profiles represent binding results with the two orientation formats. “NRCoV2-02:ACE2” represents profiles for blocking (neutralizing) V_{H}Hs where the addition of the V_{H}H or ACE2 results in no significant increase in binding over that achieved by the injection of the ACE2 or V_{H}H over the antigen surface. “NRCoV2-11:ACE2” represents profiles for non-blocking (non-neutralizing V_{H}Hs where the addition of the V_{H}H or ACE2 results in significant increase in binding over that achieved by the injection of the ACE2 or V_{H}H over the antigen surface. ΔRUs, representing binding differences between the first and second injection, were calculated from the sensorgrams and used to identify V_{H}Hs that block (neutralize) the binding of ACE2 receptor to its ligand S1-RBD. ACE2 is provided as an abbreviation for monomeric ACE2-H₆.

FIGS. 12A and 12B show the results of flow cytometry assessing the ability of monomeric V_{H}Hs in blocking (“neutralizing”) the binding of SARS-CoV-2 S to ACE2-expressing Vero E6 cells at 100 nM (FIG. 12A) or increasing (FIG. 12B) V_{H}H concentrations. FIG. 12B provides plots showing inhibition of SARS-CoV-2 S binding to Vero E6 cells as a function of V_{H}H concentration. The NRCoV2-1d, NRCoV2-02, NRCoV2-05, and NRCoV2-11 V_{H}Hs are S1-RBD, SR13, S1-NTD-specific. Monomeric ACE2 (ACE2-H6) serves as positive “antibody” control and reference, and V_{H}H-72 V_{H}H (Wrapp et al., 2020) is included as benchmark. “A20.1” and “PBS” represent negative control assays in which V_{H}Hs were replaced with C. difficile toxin A-specific A20.1 V_{H}H (Hussack et al., 2011) and PBS, respectively.

FIGS. 13A and 13B show virus-neutralizing potential of V_{H}H-Fcs in flow cytometry-based surrogate virus neutralization assays. FIG. 13A shows flow cytometry assessing the ability of bivalent V_{H}H-Fcs in blocking (“neutralizing”) the binding of SARS-CoV-2 S to ACE2-expressing Vero E6 cells at 250 nM V_{H}H-Fc concentrations. FIG. 13B shows flow cytometry assessing the ability of bivalent V_{H}H-Fcs in blocking (“neutralizing”) the binding of SARS-CoV-2 S to ACE2-expressing Vero E6 cells at increasing V_{H}H-Fc concentrations. NRCoV2-1d, NRCoV2-02, NRCoV2-04, NRCoV2-05, NRCoV2-11, and NRCoV2-20 V_{H}H-Fcs are S1-RBD-specific, while NRCoV2-SR01 and NRCoV2-SR13 V_{H}H-Fcs are S1-NTD-specific. V_{H}H-72 V_{H}H-Fc (Wrapp et al., 2020) is included as a benchmark. “A20.1” and “PBS” represent negative control assays in which V_{H}Hs were replaced with C. difficile toxin A-specific A20.1 V_{H}H (Hussack et al., 2011) and PBS, respectively.

FIGS. 14A and 14B show the results of a V_{H}H-Fc in vitro live-virus microneutralization assay. Antibody concentrations that gave 100% neutralization, i.e., MN₁₀₀s, were used to rank the neutralizing potency of V_{H}H-Fcs. A lower Woo means a higher neutralization potency. V_{H}H-72 (Wrapp et al., 2020) is included as benchmark. FIG. 14A provides a plot showing the MN₁₀₀s of bivalent V_{H}H-Fcs. The inset shows MN₁₀₀s of monomeric NRCoV2-02 and V_{H}H-72 V_{H}Hs. *The MN₁₀₀ of NRCoV2-02 bivalent V_{H}H-Fc is ≤0.01 nM, since its potency was not tested below the 0.01 nM concentration. FIG. 14B provides a plot comparing the MN₁₀₀s of bivalent V_{H}H-Fcs to monovalent V_{H}H-Fcs. Monovalent V_{H}H-72-Fc did not show Woo at the highest concentration tested (350 nM). In monovalent V_{H}H-Fc constructs, one heavy chain displays an S-specific V_{H}H, while the other displays a C. difficile toxin A-specific, mock V_{H}H (A26.8) (Hussack et al., 2011).

FIGS. 15A, 15B, 15C, 15D, and 15E show the results of V_{H}H-Fc in vitro live-virus neutralization assay. FIG. 15A shows inhibition capability of S1-RBD-specific V_{H}H-Fcs at high (312.5 nM) and low (2.5 nM) V_{H}H-Fc concentrations. As expected, NRCoV2-08, NRCoV2-19 and NRCoV2-21 which showed no binding to spike protein-expressing CHO cells (CHO-S), do not neutralize either. V_{H}H-72 (Wrapp et al., 2020) and C. difficile toxin A-specific V_{H}H A20.1 (Hussack et al., 2011) are included as benchmark and negative control, respectively. FIGS. 15B-D provide representative examples showing inhibition capability of V_{H}H-Fcs as a function of V_{H}H-Fc concentration, for select S-RBD specific antibodies (FIG. 15B), S1-NTD-specific antibodies (FIG. 15C), and S2-specific antibodies (FIG. 15D). Antibody concentrations that gave 50% neutralization, i.e., IC₅₀s, were calculated from graphs and used to rank the neutralizing potency of V_{H}H-Fcs. Bin ud, epitope bin undetermined. FIG. 15E shows a summary of IC₅₀ categorized based on subunit/domain specificity and epitope bin. A lower IC₅₀ means a higher neutralization potency. V_{H}H-72 is shown as open circle in bin 1. Bin ud, epitope bin undetermined. The line through the data points is the median.

FIGS. 16A, 16B, 16C, and 16D show data on the stability of V_{H}Hs against aerosolization treatment. FIG. 16A shows SEC profiles of pre- vs post-aerosolized V_{H}Hs, for representative V_{H}Hs. NRCoV2-1d, NRCoV2-02 and NRCoV2-07 represent the vast majority of V_{H}Hs which were resistant to aerosolization-induced aggregation, showing a homogenously monomeric peak. In contrast, the V_{H}H-72 benchmark forms a significant amount of soluble aggregates following aerosolization. NRCoV2-11 on the other hand represents the few V_{H}Hs that formed visible, precipitating aggregates reflected in significant reduction of their monomeric peak areas (compare monomeric peak for pre- vs post-aerosolized NRCoV2-11). Vₑ, elution volume. FIG. 16B summarizes the % recovery of all V_{H}Hs and FIG. 16C summarizes the % recovery of a subset of V_{H}Hs. % recovery represents the proportion of a V_{H}H that remained monomerically soluble following aerosolization. The open circle in FIG. 16B represents benchmark V_{H}H-72. The line through the data points is the median. FIG. 16D shows the results of ELISA assessing the effect of aerosolization on the functionality of V_{H}Hs by comparing the binding activity of pre- vs post-aerosolized V_{H}Hs against SARS-CoV-2 S. Essentially identical EC₅₀s for pre- vs post-aerosolized V_{H}Hs clearly indicate aerosolization had no effect on the functional activity of V_{H}Hs. Pre, pre-aerosolized V_{H}H; post, post-aerosolized V_{H}H.

FIG. 17 provides the results of sandwich ELISA demonstrating the potential utility of V_{H}Hs in detecting/capturing SARS-CoV-2, SARS-CoV and related viruses, as well as their spike proteins. SARS-CoV-2 S, S1 and S1-RBD antigens were used as surrogates for viruses. Specific detection of S, S1 and S1-RBD was achieved using NRCoV2-02 V_{H}H as the capture antibody and NRCoV2-1d, NRCoV2-02, NRCoV2-04, NRCoV2-07, or NRCoV2-11 V_{H}H-Fcs as detecting antibodies. SC₅₀ is the concentration of antigen that gives 50% binding and were calculated from graphs.

FIG. 18 shows an alignment of amino acid sequences of S-specific V_{H}H antibodies described herein.

FIG. 19 shows an alignment of amino acid sequences of S1-NTD-specific V_{H}H antibodies described herein.

FIG. 20 shows an alignment of amino acid sequences of S2-specific V_{H}H antibodies described herein.

FIG. 21 shows an alignment of amino acid sequences of S1-RBD-specific V_{H}H antibodies described herein.

FIGS. 22A, 22B, 22C, and 22D show the results of efficacy tests of V_{H}H-Fcs in hamsters challenged with SARS-CoV-2. FIG. 22A shows lung viral load in V_{H}H-Fc-treated (V_{H}H-72 benchmark, 1d, 05, MRed05, SR01, S2A3, 1d/MRed05, 1d/SR01) and control groups treated with PBS or isotype A20.1 V_{H}H-Fc at 5 dpi. PFU, plaque-forming unit. FIG. 22B shows the percent body weight change for antibody-treated and control groups. FIG. 22C shows the percent body weight change at 5 dpi. In FIG. 22A and FIG. 22C, treatment effects, assessed by one-way ANOVA with Dunnett's multiple comparison post hoc test, were significant (*p<0.05, **p<0.01, ***p<0.001 or ****p<0.0001). Dunnett's test was performed by comparing treatment groups against the isotype control. ns, not significant. FIG. 22D shows a correlation curve of body weight change vs viral titer at 5 dpi. A strong negative correlation (r=−0.9436, p<0.0001) between body weight change and lung viral titer was observed.

FIG. 23 shows immunohistochemical demonstration of SARS-CoV-2 nucleocapsid (N) protein in the lungs of V_{H}H-Fcs-treated animals. Untreated (PBS) and A20.1 isotype-treated animals showed strong viral N protein immunoreactivity which was mainly found in large multifocal patches of consolidated areas. Black arrow indicates the presence of viral N protein in bronchiolar epithelial cells. Omission of anti-nucleocapsid antibody eliminated the staining (Negative). Shown also is the absence of staining in healthy animals (Naïve). A marked reduction in viral N protein staining was seen in all lung tissues examined from V_{H}H-Fc-treated animals (middle and bottom panels). While no staining was observed in 05, MRed05, 1d/SR01 and 1d/MRed05, small foci of viral N protein was detected in V_{H}H-72, 1d, SR01 and S2A3. Representative images are shown from a single experiment.

FIG. 24 shows immunohistochemical detection of infiltrating macrophages in the lungs of V_{H}H-Fc-treated animals. Untreated (PBS) and A20.1 isotype-treated animals showed an intense immune reaction to anti-Iba-1 antibody and an increased number of Iba-1-positive macrophagesin the consolidated areas. A substantial reduction in the number of Iba-1-positive macrophages was seen in the perivascular areas and pulmonary interstitium in the lungs of V_{H}H-Fc-treated animals. Representative images are shown from a single experiment.

FIG. 25 shows immunohistochemical detection of T lymphocytes in the lungs of V_{H}H-Fc-treated animals. Untreated (PBS) and A20.1 isotype-treated animals showed an increased number of T lymphocytes in the pulmonary interstitium. A dramatic decrease in the number of T lymphocytes was seen in the lungs of V_{H}H-Fc-treated animals. Representative images are shown from a single experiment.

FIG. 26 shows immunohistochemical detection of apoptotic cells in the lungs of V_{H}H-Fc-treated animals. Untreated (PBS) and A20.1 isotype-treated animals showed an increase in the number of TUNEL-positive cells with classical features of apoptotic cells in the pulmonary interstitium. The large grey frame in the corner of PBS panel shows the magnification of the region (small grey frame) in the lung parenchyma, scale bar=50 μm. A marked reduction in the TUNEL-positive cells was seen in the lungs of NRCoV2-05- and NRCoV2-MRed05-treated animals. Black arrows indicate occasional TUNEL-positive cells. Representative images are shown from a single experiment.

FIG. 27 shows on-/off-rate maps summarizing V_{H}H kinetic rate constants, kas and k_{d}s determined by SPR for the binding of V_{H}Hs to SARS-CoV S.

FIGS. 28A and 28B show on-/off-rate maps summarizing V_{H}H kinetic rate constants, kas and k_{d}s determined by SPR for the binding of V_{H}Hs to SARS-CoV-2 Alpha S (FIG. 28A) and SARS-CoV-2 Beta S (FIG. 28B).

FIG. 29 shows representative SPR sensorgrams showing single-cycle kinetics analysis of NRCoV2-02, NRCoV2-15 and NRCoV2-MRed05 binding to Wuhan, Alpha and Beta S (NRCoV2-02, NRCoV2-15) and RBD (NRCoV2-MRed05).

FIG. 30 shows a summary of IC₅₀s obtained by live virus neutralization assays (LVNAs) for V_{H}H-Fcs against Wuhan, Alpha, and Beta SARS-CoV-2 variants. The epitope bin numbers provided in FIG. 30 correspond to the bins shown in FIG. 9G.

FIGS. 31A, 31B, 31C, and 31D show results from live virus neutralization assays assessing the ability of SARS-CoV-2 V_{H}H-Fcs in blocking the infection of ACE2-expressing Vero E6 cells by SARS-CoV-2 Alpha (FIG. 31A and FIG. 31C) and Beta (FIG. 31B and FIG. 31D) variants at fixed (FIG. 31A and FIG. 31B) or varying (FIG. 31C and FIG. 31D) V_{H}H-Fc concentrations. Inhibition assays shown in FIG. 31A and FIG. 31B were performed at 312, 12.5 or 0.5 nM V_{H}H-Fc concentrations. IC₅₀s calculated from graphs in FIG. 31C and FIG. 31D are recorded in Table 19. V_{H}H-72 and C. difficile toxin A-specific V_{H}H A20.1 are included as a benchmark and negative antibody control, respectively. The epitope bin numbers provided in FIGS. 31C and 31D correspond to the bins shown in FIG. 9G.

FIG. 32 shows in vivo stability and persistence of V_{H}Hs. Stability and persistence were determined by monitoring the concentration of a representative V_{H}H-Fc (NRCoV2-1 d) in hamster blood at various days post-injection by ELISA. V_{H}H-72 V_{H}H-Fc was used as the benchmark.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure.

Terms defined below may have the meanings ascribed to them, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

### Definitions

The “coronavirus spike polypeptide” or “coronavirus spike protein” (S) is the major coronavirus surface protein, and is a glycosylated homotrimer that binds to a host cell receptor and mediates coronavirus entry into a host cell. The coronavirus may be SARS-CoV-2, SARS-CoV, or another coronavirus. “SARS-CoV-2” may be used herein to refer to any strain or variant of the SARS-CoV-2 virus. Similarly, “SARS-CoV” may be used to refer to any strain or variant of the SARS-CoV virus. A SARS-CoV-2 variant is a strain of SARS-CoV-2 that comprises one or more mutations relative to the Wuhan strain of SARS-CoV-2. A variant may be, but need not be, a variant that has been designated as a variant of concern or a variant of interest by the World Health Organization.

As used herein, the term “polypeptide” refers to a molecule comprising two or more amino acid residues linked by peptide bonds. A polypeptide may have primary, secondary, and/or tertiary structure. A “protein” comprises at least one polypeptide and may have primary, secondary, tertiary, and/or quaternary structure. The terms “polypeptide” and “protein” are often used interchangeably, and a polypeptide may be comprised by a protein. For example, a protein may be a homo- or hetero-multimer that comprises two or more polypeptides, or a protein may comprise a single polypeptide. A polypeptide or protein may include one or more post-translational modifications, such as, but not limited to, glycosylation, phosphorylation, lipidation, S-nitrosylation, N-acetylation, or methylation.

As used herein, the term “fragment”, in the context of a polypeptide, refers to a portion of a polypeptide comprising a series of consecutive amino acid residues from a parent polypeptide. In a specific embodiment, the term “fragment” refers to an amino acid sequence of at least 5, at least 10, at least 15, at least 20, at least 25, at least 30, at least 35, at least 40, at least 45, or at least 50 consecutive amino acid residues from a parent polypeptide. In embodiments, a fragment may comprise an epitope or binding domain from a parent polypeptide. In embodiments, a fragment may be a biologically active fragment that retains one or more functional characteristics of a parent polypeptide.

The term “antibody”, as used herein, refers to an antigen binding protein comprising at least a heavy chain variable region (V_{H}) that binds a target epitope. The term antibody includes monoclonal antibodies comprising immunoglobulin heavy and light chain molecules, single heavy chain variable domain antibodies, and variants and derivatives thereof, including chimeric variants of monoclonal and single heavy chain variable domain antibodies. The antibody may be a naturally-occurring antibody, it may be obtained by manipulation of a naturally-occurring antibody, or it may be produced using recombinant methods. For example, an antibody may include, but is not limited to a Fv, single-chain Fv (scFv; a molecule consisting of V_{L} and V_{H} connected with a peptide linker), Fab, F(ab′)2, single domain antibody (sdAb; an antibody composed of a single V_{L} or V_{H}), or a multivalent presentation of any of these. Antibodies such as those just described may require linker sequences, disulfide bonds, or other types of covalent bond to link different portions of the antibody. Those of skill in the art will be familiar with the requirements of the different types of antibodies and various approaches for their construction.

In a non-limiting example, the antibody may be a single domain antibody derived from a naturally-occurring source. Heavy chain antibodies of camelid origin (Hamers-Casterman et al, 1993) lack light chains and thus their antigen binding sites consist of one domain, termed V_{H}H. sdAbs have also been observed in shark and are termed VNAR (Nuttall et al, 2003). Other sdAbs may be engineered based on human Ig heavy and light chain sequences (Jespers et al, 2004; To et al, 2005). As used herein, the term “single domain antibody” includes single domain antibodies directly isolated from V_{H}, V_{H}H, V_{L}, or VNAR reservoir of any origin through phage display or other technologies, single domain antibodies derived from the aforementioned single domain antibodies, recombinantly produced single domain antibodies, as well as single domain antibodies generated through further modification of such single domain antibodies by humanization, affinity maturation, stabilization, solubilization, camelization, or other methods of antibody engineering. Also encompassed by the disclosure are homologues, derivatives, or fragments that retain the antigen-binding function and specificity of the single domain antibody.

Single domain antibodies possess desirable properties for antibody molecules, such as high thermostability, high detergent resistance, relatively high resistance to proteases (Dumoulin et al, 2002) and high production yield (Arbabi-Ghahroudi et al, 1997). They can also be engineered to have very high affinity by isolation from an immune library (Li et al, 2009) or by in vitro affinity maturation (Davies & Riechmann, 1996). Further modifications to increase stability, such as the introduction of non-canonical disulfide bonds (Hussack et al, 2011; Kim et al, 2012), may also be brought to a single domain antibody.

A person of skill in the art would be well-acquainted with the structure of a single-domain antibody. A single domain antibody comprises a single immunoglobulin domain that retains the immunoglobulin fold; most notably, only three CDR/hypervariable loops form the antigen-binding site. However, and as would be understood by one of skill in the art, not all CDRs may be required for binding the antigen. For example, and without wishing to be limiting, one, two, or three of the CDRs may contribute to binding and recognition of the antigen by a single domain antibody. The CDRs of the single domain antibody or variable domain are referred to herein as CDR1, CDR2, and CDR3, and numbered as defined by Lefranc et al., 2003.

As described herein, the amino acid sequence and structure of a heavy chain variable domain, including a V_{H}H, can be considered-without however being limited thereto to be comprised of four framework regions or ‘FR’, which are referred to in the art and herein as Framework region 1‘ or’FR1′; as Framework region 2‘ or’FR2′; as Framework region 3′ or FR3′; and as Framework region 4‘ or’FR4′, respectively; which framework regions are interrupted by three complementarity determining regions or ‘CDR's’, which are referred to in the art as ‘Complementarity Determining Region 1’ or ‘CDR1’; as ‘Complementarity Determining Region 2’ or ‘CDR2’; and as ‘Complementarity Determining Region 3’ or ‘CDR3’, respectively. CDRs described in the present disclosure have been defined using the IMGT numbering system (Lefranc et al, 2003).

The term “binding” as used herein in the context of binding between an antibody, such as a V_{H}H, and a coronavirus spike protein epitope as a target, refers to the process of a non-covalent interaction between molecules. Preferably, said binding is specific. The terms specific‘ or specificity’ or grammatical variations thereof refer to the number of different types of antigens or their epitopes to which a particular antibody such as a V_{H}H can bind. The specificity of an antibody, also referred to as “specific binding”, can be determined based on affinity. A specific antibody preferably has a binding affinity (Kd) for its epitope of less than 10⁻⁷ M, preferably less than 10⁻⁸ M.

The term “affinity”, as used herein, refers to the strength of a binding reaction between a binding domain of an antibody and an epitope. It is the sum of the attractive and repulsive forces operating between the binding domain and the epitope. The term “affinity”, as used herein, refers to the equilibrium dissociation constant, Kd.

The term “epitope” or “antigenic determinant”, as used herein, refers to a part of an antigen that is recognized by an antibody. The term epitope includes linear epitopes and conformational epitopes. A linear epitope is an epitope that is recognized by an antibody based on its primary structure, and a stretch of contiguous amino acids is sufficient for binding. A conformational epitope is based on 3-D surface features and shape and/or tertiary structure of the antigen.

The term “neutralizing antibody”, as used herein, refers to an antibody that, when bound to an epitope, interferes with at least one of the steps leading to the release of a virus genome, such as a coronavirus genome, into a host cell.

The term “subject”, as used herein, refers to an animal that is susceptible to infection by a coronavirus. The subject may be an animal that is susceptible to infection by a coronavirus that binds an ACE2 receptor, such as SARS-CoV-2 or SARS-CoV. The subject may be a human or non-human animal. Preferably the subject is a human or non-human mammal. Correspondingly, the ACE2 receptor may be a human ACE2 receptor or an animal ACE2 receptor.

The term “administering”, as used herein, refers to the introduction into a subject of a therapeutic agent. Many administration routes are known in the art, and include, but are not limited to, parenteral (intravenous, intramuscular, and subcutaneous), oral, nasal, ocular, transmucosal (buccal, vaginal, and rectal), transdermal, and pulmonary administration.

The terms “strong interaction” and “strong binding”, as used herein, refer to the presence of salt bridges and cation-pi interactions between amino acid residues, as is known to the skilled person.

The terms “weak interaction” and “weak binding”, as used herein, refer to the presence of hydrogen bonds and non-bonded/hydrophobic interactions, as is known to the skilled person.

The term “purified,” as used herein, refers to a molecule, e.g. a polypeptide or protein that has been identified and substantially separated and/or recovered from the components of its natural environment. The term “isolated antibody”, as used herein, refers to an antibody that is substantially freed from other antibody molecules having different antigenic specificities. Further, a purified or isolated antibody may be substantially free of one or more other cellular and/or chemical substances. Absolute purity is not required for a molecule to be considered purified or isolated.

The term “pharmaceutically acceptable”, as used herein, means generally regarded as safe when administered to humans. Preferably, as used herein, the term “pharmaceutically acceptable” is approved by a federal or state government regulatory agency for use in animals, more preferably in humans. The term “carrier” means a diluent, adjuvant, excipient, or vehicle with which a compound is formulated and/or administered. Such pharmaceutical carriers can be water and sterile liquids, such as petroleum, animal, vegetable or synthetically derived oils such as peanut oil, soybean oil, mineral oil, sesame oil. Water or saline solutions and aqueous dextrose and glycerol solutions are preferably used as carriers for injectable solutions. Suitable pharmaceutical carriers are, for example, described in “Remington (23^{rd} edition), The Science and Practice of Pharmacy”.

As used herein the term “linked” or “linkage” includes covalent and non-covalent linkage (bonding). As used herein, the term “linker” refers to a chemical group or molecule that can be used to join one molecule to another. An antibody may be linked to another molecule by a linker or an antibody may be directly linked (aka joined, fused, or bonded) to another molecule, without the use of a linker. Suitable linkers are known in the art and may be selected based on the chemical nature of the molecules being joined. Examples of linkers include peptide linkers and chemical cross-linkers. Peptide linkers may comprise a single amino acid residue or a plurality of amino acid residues. An antibody and a polypeptide may, for example, be linked by chemical conjugation, with or without the use of a linker, or produced as a fusion, for example by recombinant protein expression.

As used herein the term “label” refers to a molecule or compound that can be used to label a molecule, such as an antibody, to allow detection of the molecule. Suitable labels will be known to one skilled in the art and include, but are not limited to, radioisotopes; enzymes, such as horse radish peroxidase (HRP) or calf intestinal alkaline phosphate (AP); fluorophores; antigen binding fragments from cleaved antibodies (Fabs); and colloidal gold. Covalent linkage is commonly used to link a label to a molecule of interest, however, non-covalent linkage is also possible, for example, when the label is a Fab.

As used herein, the term “nucleic acid molecule” refers to any nucleic acid-containing molecule including, but not limited to, DNA, RNA, and DNA/RNA hybrids, in any form and/or conformation. The term encompasses nucleic acids that include any of the known base analogs of DNA and RNA. For example, single-stranded, double-stranded, nuclear, extranuclear, extracellular, and isolated nucleic acids are all contemplated.

As used herein, the term “vector” refers to a synthetic nucleotide sequence used for manipulation of genetic material, including but not limited to cloning, subcloning, sequencing, or introduction of exogenous genetic material into cells, tissues or organisms. It is understood by one skilled in the art that vectors may contain synthetic DNA sequences, naturally occurring DNA sequences, or both. Examples of commonly used vectors include plasmids, viral vectors, cosmids, and artificial chromosomes.

As used herein, the term “regulatory sequence” includes promoters, enhancers and other expression control elements, such as polyadenylation sequences, matrix attachment sites, insulator regions for expression of multiple genes on a single construct, ribosome entry/attachment sites, introns that are able to enhance expression, and silencers. Promoters may be cell-specific or tissue-specific to facilitate expression in a desired target.

When referring to two nucleotide sequences, one being a regulatory sequence, the term “operably linked” is used herein to mean that the two sequences are associated in a manner that allows the regulatory sequence to affect expression of the other nucleotide sequence. It is not required that the operably-linked sequences be directly adjacent to one another with no intervening sequence(s).

As used herein, the term “host cell” refers to a cell into which a nucleic acid molecule or vector may be introduced, for example to allow for replication of the nucleic acid molecule or vector by the host cell and/or to allow for expression of the nucleic acid molecule, or of a nucleic acid molecule comprised by the vector, by the host cell to produce a product of interest, such as an RNA or protein. In a specific embodiment, the nucleic acid molecule may encode an antibody as described herein, and introduction of the nucleic acid molecule into the host cell may allow the antibody to be expressed by the host cell. A host cell may be any suitable cell, such as a bacterial cell or eukaryotic cell. Commonly used host cells include E. coli, yeast, and mammalian cells, such as, but not limited to, Chinese hamster ovary (CHO) cells, mouse myeloma cells, and human embryonic kidney (HEK) cells.

The term “treatment” and variations thereof, such as “treat” or “treating”, as used herein, refer to the administration of a therapeutic molecule or composition to a subject to reduce or eliminate one or more symptoms of an illness or disease in the subject and/or to reduce the duration of the illness or disease in the subject.

The term “prevention” and variations thereof, such as “prevent” or “preventing”, as used herein, refer to the prophylactic administration of a therapeutic molecule or composition to a subject to prevent the occurrence of, or to reduce the severity of, an illness or disease in the subject.

The term “sample” as used herein, refers to a sample in which a coronavirus presence is suspected or expected. For example, the sample may be a biological sample from a subject, such as, but not limited to, blood or a fraction thereof, saliva, cellular material, urine, or feces; a sample from a bioreactor; or an environmental sample.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e. % identity=(number of identical overlapping positions/total number of positions)×100). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, modified as in Karlin and Altschul, 1993. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present invention. To obtain gapped alignments for comparison purposes, Gapped BLAST can be utilized as described in Altschul et al., 1997. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein the singular forms “a”, “an”, and “the” include plural references unless the context clearly dictates otherwise.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

### Description

The present disclosure relates to SARS-CoV-2 spike protein-specific antibodies and uses thereof. Provided are isolated or purified antibodies comprising complementarity determining region (CDR) 1, CDR2, and CDR3 sequences as outlined in Table 6. The antibodies described herein recognize a variety of spike protein epitopes in different subunit and domains of the coronavirus spike protein, specifically S2, the N-terminal domain of S1 (S1-NTD), and the receptor binding domain of S1 (S1-RBD). Within these subunits/domains, antibodies described herein recognize several different epitopes. Because of this epitopic diversity, antibodies described herein may be used in combination, for example for combination therapy, or as bispecific or multi-specific antibodies.

An antibody as described herein comprises an antigen binding portion of an antibody heavy chain, wherein the antigen binding portion comprises a first complementarity determining region (CDR1), a second complementarity determining region (CDR2), and a third complementarity determining region (CDR3), and wherein CDR1, CDR2, and CDR3, respectively, comprise the amino acid sequence set forth in: SEQ ID NO: 1, SEQ ID NO: 45, and SEQ ID NO: 90; SEQ ID NO: 2, SEQ ID NO: 45, and SEQ ID NO: 91; SEQ ID NO: 1, SEQ ID NO: 46, and SEQ ID NO: 92; SEQ ID NO: 3, SEQ ID NO: 47, and SEQ ID NO: 93; SEQ ID NO: 4, SEQ ID NO: 48, and SEQ ID NO: 94; SEQ ID NO: 5, SEQ ID NO: 49, and SEQ ID NO: 95; SEQ ID NO: 6, SEQ ID NO: 50, and SEQ ID NO: 96; SEQ ID NO: 7, SEQ ID NO: 51, and SEQ ID NO: 97; SEQ ID NO: 8, SEQ ID NO: 52, and SEQ ID NO: 98; SEQ ID NO: 9, SEQ ID NO: 53, and SEQ ID NO: 99; SEQ ID NO: 10, SEQ ID NO: 54, and SEQ ID NO: 100; SEQ ID NO: 11, SEQ ID NO: 55, and SEQ ID NO: 101; SEQ ID NO: 12, SEQ ID NO: 56, and SEQ ID NO: 102; SEQ ID NO: 13, SEQ ID NO: 57, and SEQ ID NO: 103; SEQ ID NO: 14, SEQ ID NO: 58, and SEQ ID NO: 104; SEQ ID NO: 15, SEQ ID NO: 59, and SEQ ID NO: 105; SEQ ID NO: 16, SEQ ID NO: 60, and SEQ ID NO: 106; SEQ ID NO: 17, SEQ ID NO: 61, and SEQ ID NO: 107; SEQ ID NO: 18, SEQ ID NO: 62, and SEQ ID NO: 108; SEQ ID NO: 19, SEQ ID NO: 63, and SEQ ID NO: 109; SEQ ID NO: 20, SEQ ID NO: 64, and SEQ ID NO: 110; SEQ ID NO: 21, SEQ ID NO: 65, and SEQ ID NO: 111; SEQ ID NO: 22, SEQ ID NO: 66, and SEQ ID NO: 112; SEQ ID NO: 23, SEQ ID NO: 67, and SEQ ID NO: 113; SEQ ID NO: 24, SEQ ID NO: 68, and SEQ ID NO: 114; SEQ ID NO: 25, SEQ ID NO: 69, and SEQ ID NO: 115; SEQ ID NO: 26, SEQ ID NO: 70, and SEQ ID NO: 116; SEQ ID NO: 27, SEQ ID NO: 71, and SEQ ID NO: 117; SEQ ID NO: 28, SEQ ID NO: 72, and SEQ ID NO: 118; SEQ ID NO: 29, SEQ ID NO: 73, and SEQ ID NO: 119; SEQ ID NO: 30, SEQ ID NO: 74, and SEQ ID NO: 120; SEQ ID NO: 31, SEQ ID NO: 75, and SEQ ID NO: 121; SEQ ID NO: 32, SEQ ID NO: 76, and SEQ ID NO: 122; SEQ ID NO: 33, SEQ ID NO: 77, and SEQ ID NO: 123; SEQ ID NO: 34, SEQ ID NO: 78, and SEQ ID NO: 124; SEQ ID NO: 35, SEQ ID NO: 79, and SEQ ID NO: 125; SEQ ID NO: 36, SEQ ID NO: 80, and SEQ ID NO: 125; SEQ ID NO: 20, SEQ ID NO: 81, and SEQ ID NO: 126; SEQ ID NO: 20, SEQ ID NO: 81, and SEQ ID NO: 127; SEQ ID NO: 37, SEQ ID NO: 82, and SEQ ID NO: 128; SEQ ID NO: 38, SEQ ID NO: 83, and SEQ ID NO: 129; SEQ ID NO: 39, SEQ ID NO: 84, and SEQ ID NO: 130; SEQ ID NO: 40, SEQ ID NO: 85, and SEQ ID NO: 131; SEQ ID NO: 41, SEQ ID NO: 86, and SEQ ID NO: 132; SEQ ID NO: 42, SEQ ID NO: 87, and SEQ ID NO: 133; SEQ ID NO: 43, SEQ ID NO: 88, and SEQ ID NO: 134; or SEQ ID NO: 44, SEQ ID NO: 89, and SEQ ID NO: 135. In an embodiment, the antibody comprises the amino acid sequence set forth in SEQ ID NO: 183, 184, 185, or 186.

In an embodiment, an antibody as described herein comprises the amino acid sequence set forth in SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, or SEQ ID NO: 182. In another embodiment, the antibody comprises an amino acid sequence having at least 75%, at least 80%, at least 85%, at least 90%, at least 95%, at least 96%, at least 97%, at least 98%, or at least 99% sequence identity to the full length of the amino acid sequence set forth in SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, and/or SEQ ID NO: 182 and comprises CDR1, CDR2, and CDR3 sequences that, respectively, comprise the amino acid sequence set forth in: SEQ ID NO: 1, SEQ ID NO: 45, and SEQ ID NO: 90; SEQ ID NO: 2, SEQ ID NO: 45, and SEQ ID NO: 91; SEQ ID NO: 1, SEQ ID NO: 46, and SEQ ID NO: 92; SEQ ID NO: 3, SEQ ID NO: 47, and SEQ ID NO: 93; SEQ ID NO: 4, SEQ ID NO: 48, and SEQ ID NO: 94; SEQ ID NO: 5, SEQ ID NO: 49, and SEQ ID NO: 95; SEQ ID NO: 6, SEQ ID NO: 50, and SEQ ID NO: 96; SEQ ID NO: 7, SEQ ID NO: 51, and SEQ ID NO: 97; SEQ ID NO: 8, SEQ ID NO: 52, and SEQ ID NO: 98; SEQ ID NO: 9, SEQ ID NO: 53, and SEQ ID NO: 99; SEQ ID NO: 10, SEQ ID NO: 54, and SEQ ID NO: 100; SEQ ID NO: 11, SEQ ID NO: 55, and SEQ ID NO: 101; SEQ ID NO: 12, SEQ ID NO: 56, and SEQ ID NO: 102; SEQ ID NO: 13, SEQ ID NO: 57, and SEQ ID NO: 103; SEQ ID NO: 14, SEQ ID NO: 58, and SEQ ID NO: 104; SEQ ID NO: 15, SEQ ID NO: 59, and SEQ ID NO: 105; SEQ ID NO: 16, SEQ ID NO: 60, and SEQ ID NO: 106; SEQ ID NO: 17, SEQ ID NO: 61, and SEQ ID NO: 107; SEQ ID NO: 18, SEQ ID NO: 62, and SEQ ID NO: 108; SEQ ID NO: 19, SEQ ID NO: 63, and SEQ ID NO: 109; SEQ ID NO: 20, SEQ ID NO: 64, and SEQ ID NO: 110; SEQ ID NO: 21, SEQ ID NO: 65, and SEQ ID NO: 111; SEQ ID NO: 22, SEQ ID NO: 66, and SEQ ID NO: 112; SEQ ID NO: 23, SEQ ID NO: 67, and SEQ ID NO: 113; SEQ ID NO: 24, SEQ ID NO: 68, and SEQ ID NO: 114; SEQ ID NO: 25, SEQ ID NO: 69, and SEQ ID NO: 115; SEQ ID NO: 26, SEQ ID NO: 70, and SEQ ID NO: 116; SEQ ID NO: 27, SEQ ID NO: 71, and SEQ ID NO: 117; SEQ ID NO: 28, SEQ ID NO: 72, and SEQ ID NO: 118; SEQ ID NO: 29, SEQ ID NO: 73, and SEQ ID NO: 119; SEQ ID NO: 30, SEQ ID NO: 74, and SEQ ID NO: 120; SEQ ID NO: 31, SEQ ID NO: 75, and SEQ ID NO: 121; SEQ ID NO: 32, SEQ ID NO: 76, and SEQ ID NO: 122; SEQ ID NO: 33, SEQ ID NO: 77, and SEQ ID NO: 123; SEQ ID NO: 34, SEQ ID NO: 78, and SEQ ID NO: 124; SEQ ID NO: 35, SEQ ID NO: 79, and SEQ ID NO: 125; SEQ ID NO: 36, SEQ ID NO: 80, and SEQ ID NO: 125; SEQ ID NO: 20, SEQ ID NO: 81, and SEQ ID NO: 126; SEQ ID NO: 20, SEQ ID NO: 81, and SEQ ID NO: 127; SEQ ID NO: 37, SEQ ID NO: 82, and SEQ ID NO: 128; SEQ ID NO: 38, SEQ ID NO: 83, and SEQ ID NO: 129; SEQ ID NO: 39, SEQ ID NO: 84, and SEQ ID NO: 130; SEQ ID NO: 40, SEQ ID NO: 85, and SEQ ID NO: 131; SEQ ID NO: 41, SEQ ID NO: 86, and SEQ ID NO: 132; SEQ ID NO: 42, SEQ ID NO: 87, and SEQ ID NO: 133; SEQ ID NO: 43, SEQ ID NO: 88, and SEQ ID NO: 134; or SEQ ID NO: 44, SEQ ID NO: 89, and SEQ ID NO: 135.

Another embodiment is a nucleic acid molecule encoding an antibody as described herein. A further embodiment is a vector comprising the nucleic acid molecule. Optionally, the nucleic acid molecule may be operably linked to at least one promoter and/or regulatory element to enable expression in a host cell. A further embodiment is a host cell comprising the nucleic acid or vector.

An antibody as described herein may be comprised within a composition. For example, the antibody may be comprised within a pharmaceutical composition that comprises a pharmaceutically acceptable carrier and/or diluent, the antibody may be linked to another molecule, or the antibody may be immobilized on a substrate. In an embodiment, the pharmaceutical composition may be for delivery by inhalation or nebulization.

Antibodies and compositions as described herein may be used, or for use, to treat or prevent a coronavirus infection, including an infection caused by at least one coronavirus that specifically binds an ACE2 receptor. Antibodies as described herein may also be used in the manufacture of a medicament for prevention or treatment of a coronavirus infection. In a specific embodiment, the at least one coronavirus is SARS-CoV-2 and/or SARS-CoV. Further provided is a method for prevention or treatment of a coronavirus infection comprising administering an antibody or composition as described herein to a subject in need thereof. In an embodiment, the administration is by inhalation or nebulization.

Antibodies and compositions as described herein may also be used, or for use, to detect, quantify, and/or capture a coronavirus, a coronavirus spike polypeptide or a coronavirus spike polypeptide fragment. Further provided are methods for detecting, quantifying, and/or capturing a coronavirus, a coronavirus spike polypeptide or a coronavirus spike polypeptide fragment using an antibody or composition as described herein. In an embodiment, the coronavirus or spike polypeptide is a coronavirus or spike polypeptide that specifically binds an ACE2 receptor. The ACE2 receptor may be a human ACE2 receptor or an animal ACE2 receptor. In a specific embodiment, the coronavirus is SARS-CoV-2 or SARS-CoV, or the spike polypeptide or fragment thereof is from SARS-CoV-2 or SARS-CoV

Several of the antibodies described herein have the characteristics of neutralizing antibodies, and some have been demonstrated to be cross-reactive with the spike protein of other coronaviruses, such as SARS-CoV and related coronaviruses that infect bats, pangolin, and civet, suggesting that antibodies described herein may be useful for binding the spike protein of more than one coronavirus; including coronaviruses that bind an ACE2 receptor, such as SARS-CoV-2 and SARS-CoV. Antibodies described herein have also been demonstrated to bind various SARS-CoV-2 spike protein variants, such as the Wuhan-Hu-1 variant that was first identified in China; the B.1.1.7 variant that was first identified in the United Kingdom (also referred to herein as the UK variant, or the Alpha variant); the B.1.352 variant that was first identified in South Africa (also referred to herein as the South Africa variant, or the Beta variant), the B.1.617.1 variant that was first detected in India (also referred to herein as Kappa); the B.1.617.2 variant that was first detected in India (also referred to herein as Delta); and the B.1.1.529 variant that was first detected in South Africa (also referred to herein as Omicron).

Antibodies described herein may be linked to another molecule or substrate. For example, they may be linked to a detectable label to allow detection, quantification, and/or visualization; they may be linked to a molecule that extends antibody half-life, such as polyethylene glycol (PEG), Ig Fc, serum albumin, serum-albumin-specific antibody, serum-albumin-specific peptide, or Fc-specific peptides, proteins or antibodies; they may be linked to a therapeutic molecule; they may be immobilized onto a substrate, such as a plastic surface, a magnetic bead or a protein sheet or bead; and/or they may be linked to a polypeptide. In a specific embodiment, antibodies described herein may be linked to an ACE2 polypeptide or a fragment thereof.

Antibodies described herein may also be employed in various formats and combinations. For example, antibodies described herein may be monoparatropic or multiparatropic (including biparatropic), or monospecific or multispecific (including bispecific). Antibodies described herein may be in a monovalent format or in a multivalent format (including a bivalent format). Antibodies described herein that are specific for the same or different epitopes, or for the same or different spike protein subunit or domains, may be linked, for example to produce antibodies with different affinities and/or specificities. Further, antibodies described herein may be linked to one or more other antibodies or antibody fragments. In addition, antibodies described herein may be used individually or in combination. A combination may comprise any two or more antibodies described herein, or it may comprise at least one antibody described herein and another antibody. In some embodiments, the antibodies are V_{H}H antibodies or V_{H}H-Fc antibodies.

Antibodies described herein may be useful for a variety of applications. For example, they may be useful for detecting the presence of a coronavirus or a coronavirus spike polypeptide or fragment thereof, for capturing a coronavirus or a coronavirus spike polypeptide or fragment thereof, for quantifying the amount of a coronavirus or a coronavirus spike polypeptide or fragment thereof in a sample; for treatment or prevention of a coronavirus infection; for diagnosing a coronavirus infection; for monitoring the production of a coronavirus spike protein or fragment thereof, for purification of a coronavirus spike protein or fragment thereof, for detecting the level of expression of a coronavirus spike protein or fragment thereof, and/or for quantifying the amount of a coronavirus. Antibodies described herein have been shown to be stable against aerosolization, indicating that they may be suitable for delivery to the lungs by inhalation or nebulization. Further, cross-reactive antibodies may have general applicability for the treatment, prevention, detection, quantification or capture of coronaviruses, in addition to SARS-CoV-2, or coronavirus spike polypeptides or fragments thereof from coronaviruses in addition to SARS-CoV-2. In specific embodiments, cross-reactive antibodies may be used to bind coronaviruses or coronavirus spike polypeptides that bind an ACE2 receptor, including fragments of such coronavirus spike polypeptides.

Antibodies described herein may be classified based on the spike protein subunit or domain to which they bind. Nine antibodies were generated that bind to the S1-NTD domain, 24 antibodies were generated that bind to the S1-RBD domain, and 14 antibodies were generated that bind to the S2 subunit (see Tables 5 and 6). Neutralization assays, as described in the Examples, identified antibodies with neutralizing properties within each of these three groups. To the inventors' knowledge, this is the first known observation of single domain antibodies neutralizing the SARS-CoV-2 virus by targeting a non-S1-RBD region of S, i.e., S1-NTD or S2.

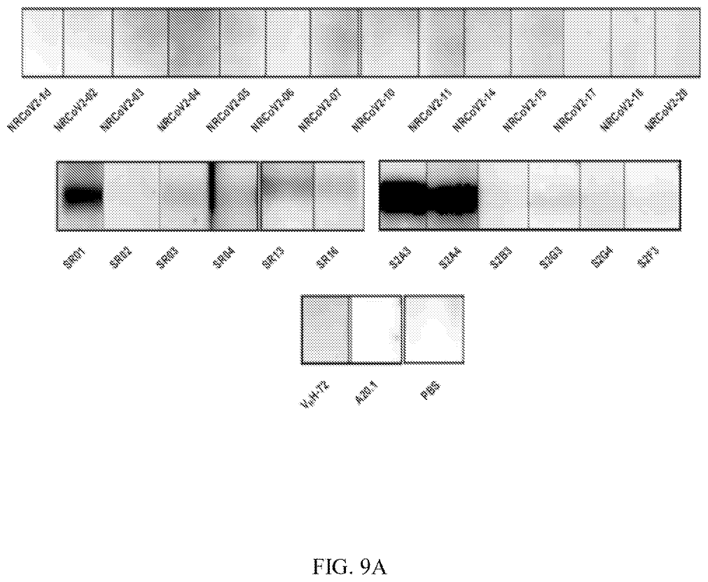

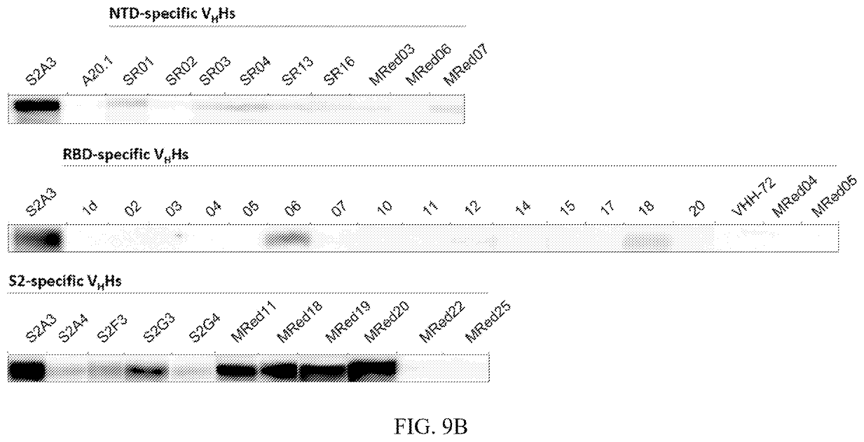

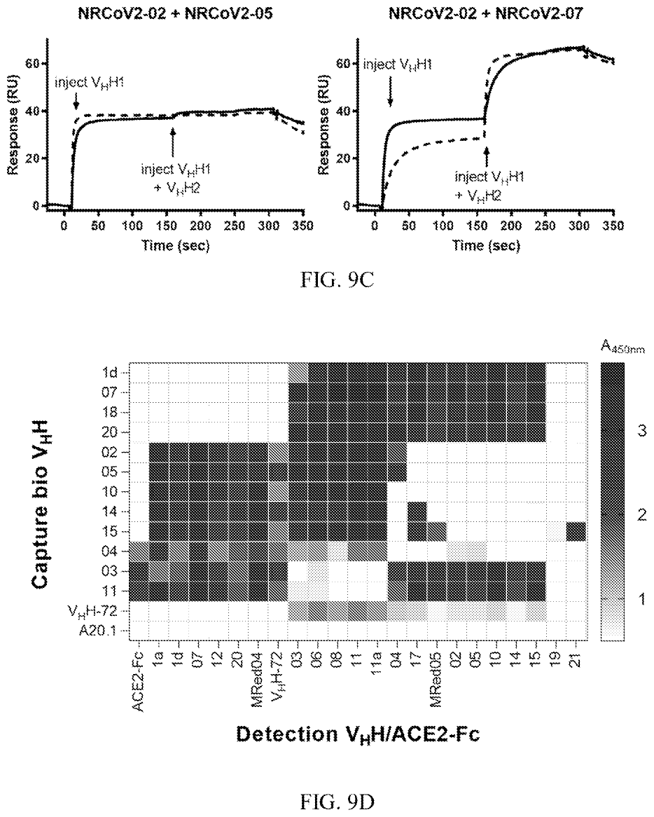

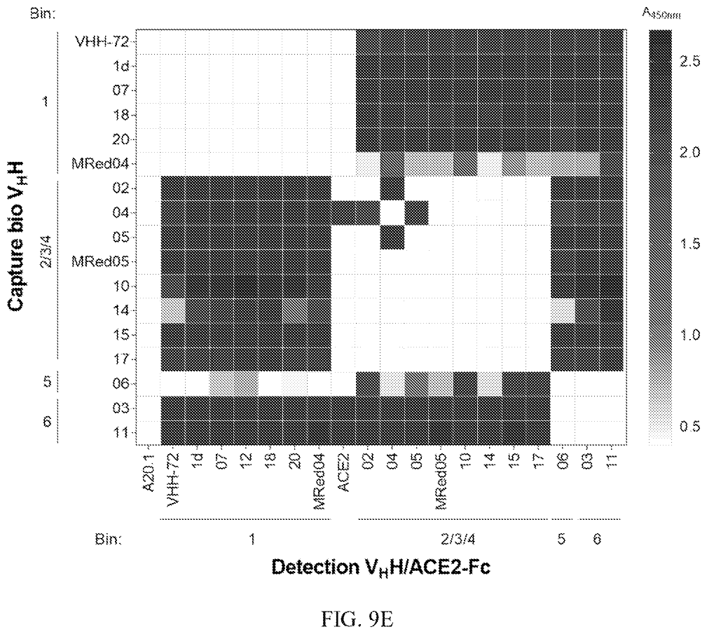

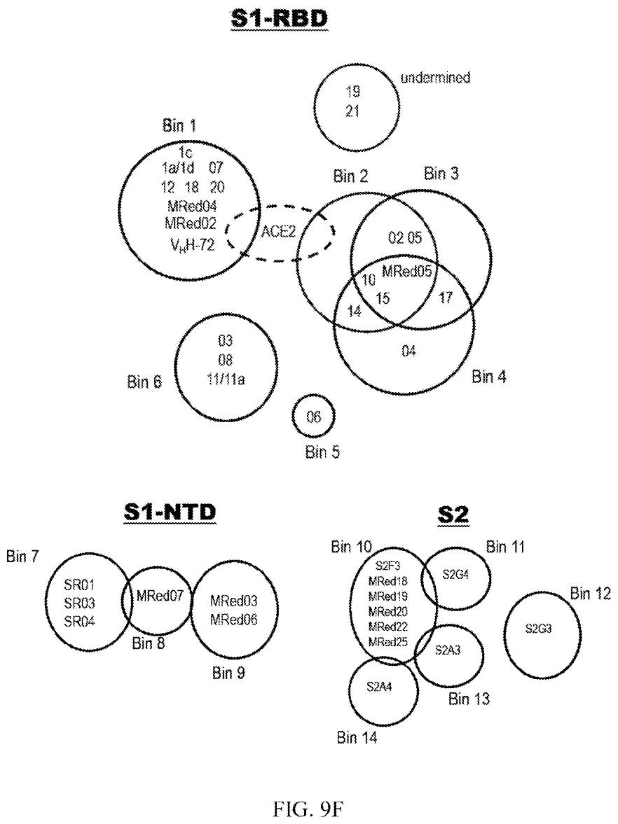

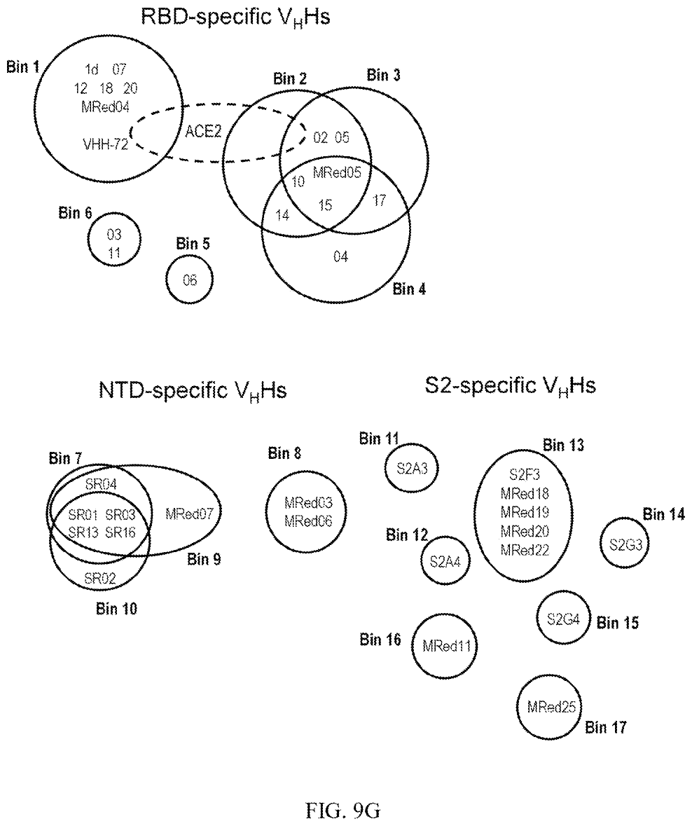

Within the three groups of antibodies identified above, further classification is possible based on epitope specificity, which was determined by epitope binning experiments (see Example 7). Preliminary results showed that antibodies binding to S1-NTD may be grouped into three epitope bins; antibodies binding S1-RBD may be grouped into six epitope bins, with some overlap between bins; and antibodies binding S2 may be grouped into five epitope bins (FIG. 9F). Further characterization identified additional epitope bins, so that antibodies binding to S1-NTD may be grouped into four epitope bins; antibodies binding S1-RBD may be grouped into six epitope bins, with some overlap between bins; and antibodies binding S2 may be grouped into seven epitope bins (FIG. 9G).

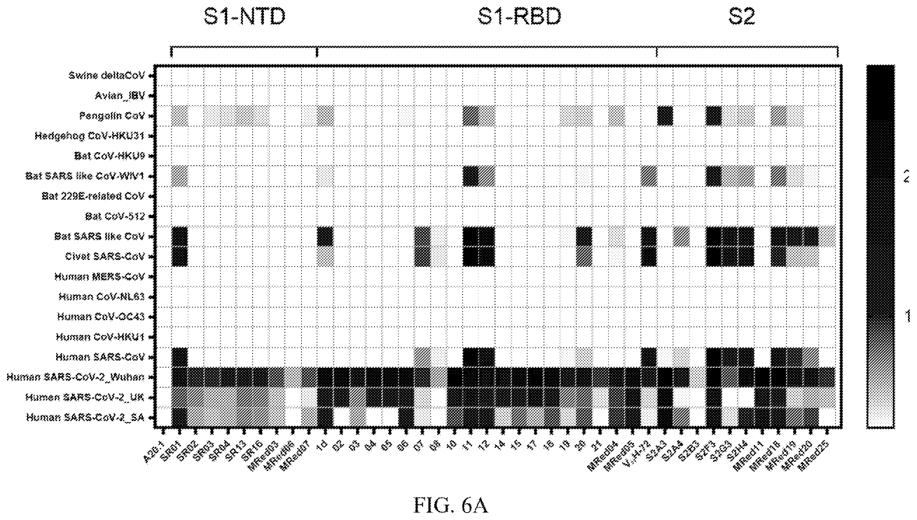

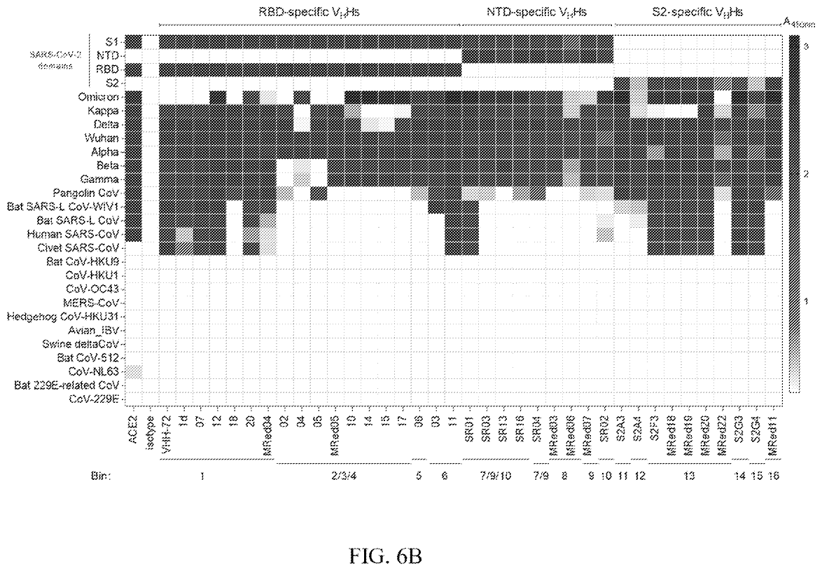

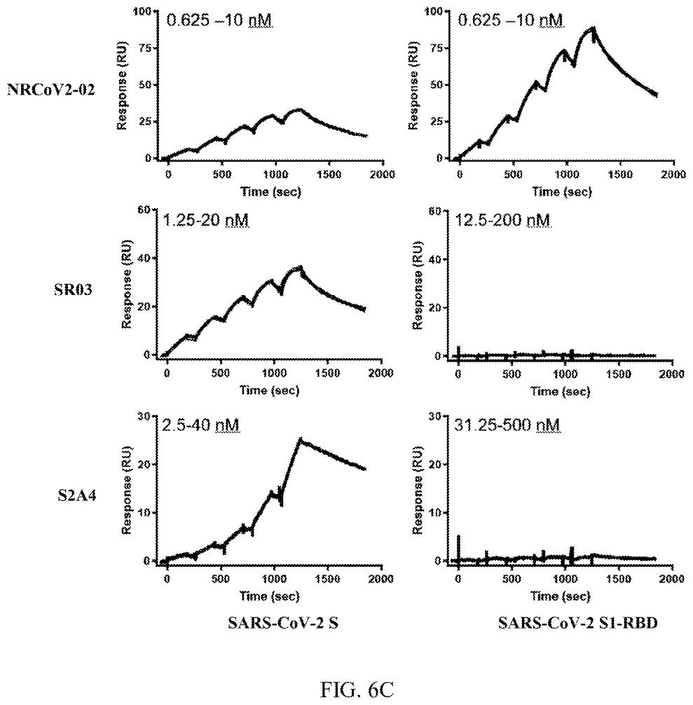

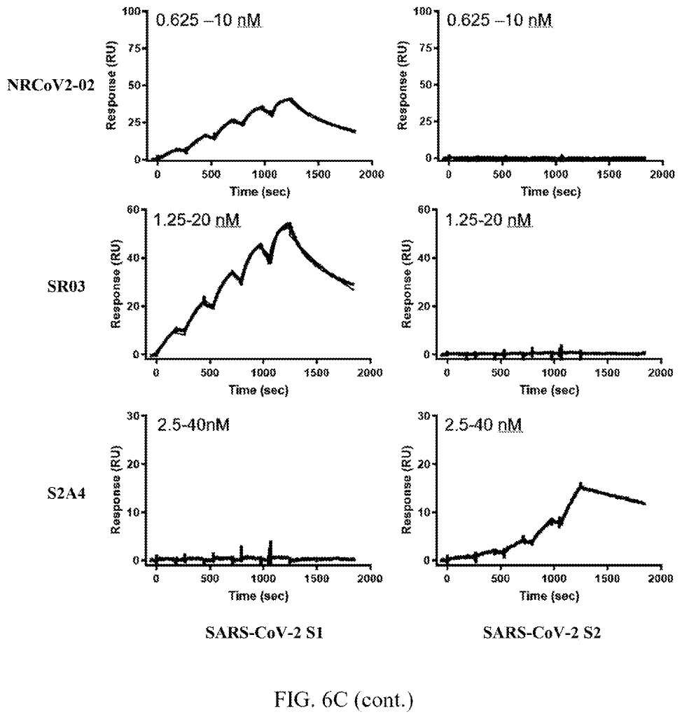

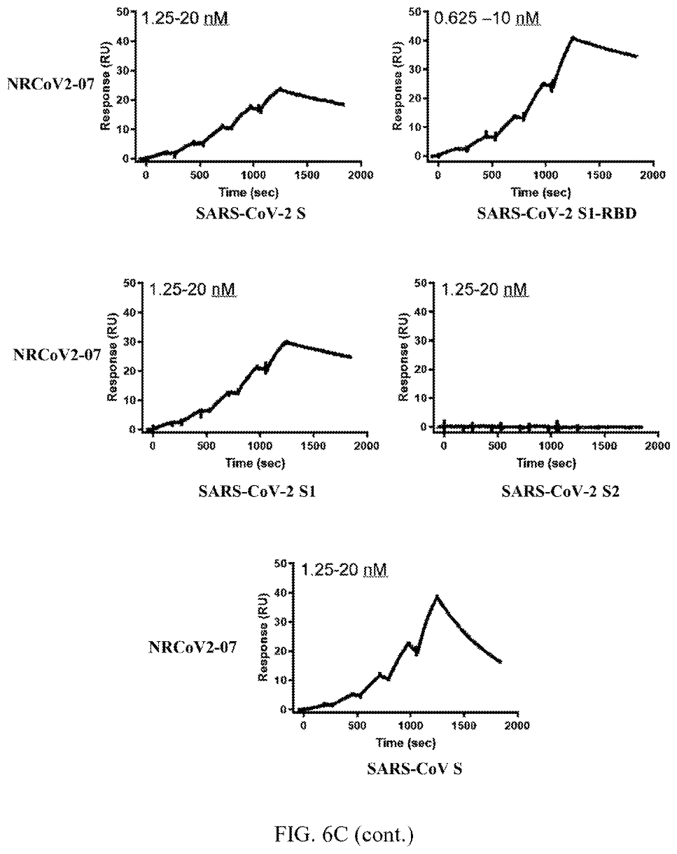

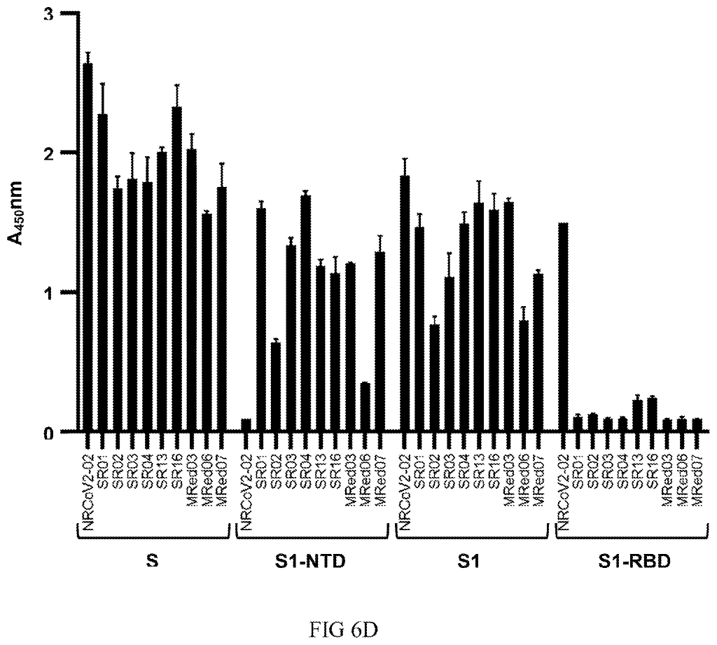

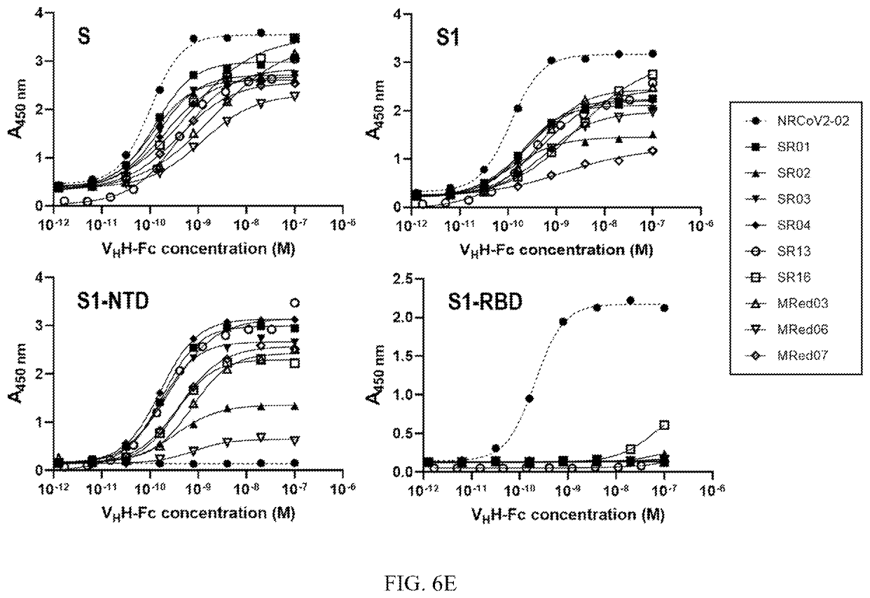

Antibodies described herein may also be classified based on their pattern of cross-reactivity with different coronavirus spike proteins and/or spike protein variants, as shown in FIGS. 6A and 6B. Antibodies that recognize the same set of spike proteins and/or spike protein variants may be viewed as a single group.

As demonstrated in the Examples, several of the antibodies described herein have substantially increased binding affinity in comparison to a benchmark V_{H}H spike protein antibody, V_{H}H-72 (Wrapp et al., 2020). Further, many of the antibodies described herein are demonstrated to outperform V_{H}H-72 in neutralization assays, and some are demonstrated to be more broadly neutralizing than V_{H}H-72. Additionally, some antibodies described herein are demonstrated to be more broadly cross-reactive than V_{H}H-72.

The antibodies described in the following examples may be modified, while still retaining antigen specificity. For example, changes may be introduced into the amino acid sequence of the framework regions, or the antibodies may be humanized. The antibodies may also be linked to other molecule(s). Antibodies and compositions resulting from such modifications are contemplated and encompassed by the present disclosure.

### EXAMPLES

The following non-limiting examples are illustrative of the present disclosure and/or outline studies conducted pertaining to the present disclosure.

Several coronavirus spike protein fragments (spike protein antigens) were used in the Examples described below. Table 1 provides a list of spike protein fragments used in these studies.

TABLE 1
Coronavirus spike protein fragments used for library
selection, binding and epitope study experiments

| | | | | Reference |
|---|---|---|---|---|
| | | | | describing |
| | Accession | | | expression & |
| Description | number | Source | Tag | purification |
| S1 (aa16-685) | QHD43416.1^{b} | National Research | FLAG, 6xHis | Akache et al., |
| (Wuhan) | | Council of | | 2021 |
| | | Canada (NRC) | | |
| S1 (aa16-685) | QHD43416.1^{b} | ACROBiosystems | AviTag, 6xHis | na |
| (Wuhan) | | | | |
| S1 (aa16-685) | QHD43416.1^{b} | ACROBiosystems | Human IgG1 | na |
| (Wuhan) | | | Fc | |
| NTD (aa16-305) | QHD43416.1^{b} | NRC | FLAG, 6xHis | Akache et al., |
| (Wuhan) | | | | 2021 |
| RBD/SD1 (aa319-591) | QHD43416.1^{b} | NRC | Human IgG1 | Wrapp et al., |
| (Wuhan) | | | Fc | 2020 |
| RBD/SD1 (aa319-591) | QHD43416.1^{b} | NRC | 6xHis | Wrapp et al., |
| (Wuhan) | | | | 2020 |
| RBD_short (aa331-521) | QHD43416.1^{b} | NRC | 6xHis | Akache et al., |
| (Wuhan) | | | | 2021 |
| RBD (aa319-541) | QHD43416.1^{b} | NRC | FLAG-6xHis | Akache et al., |
| (Wuhan) | | | (N-term), E5 | 2021 |
| | | | (C-term) | |
| RBD_α (aa319-541) | QHD43416.1^{b} | NRC | FLAG, 6xHis | Colwill et al., |
| (B.1.1.7) | | | | 2021 |
| RBD_β (aa319-541) | QHD43416.1^{b} | NRC | FLAG, 6xHis | Colwill et al., |
| (B.1.351) | | | | 2021 |
| RBD_Wuhan | QHD43416.1^{b} | ACROBiosystems | AviTag, 6xHis | na |
| (aa319-541) | | | | |
| RBD_SARS-CoV | AAP13442.1^{b} | NRC | FLAG, 6xHis | Sulea et al., |
| (aa306-527) | | | | 2022 |
| S2 (aa686-1208) | QHD43416.1^{b} | NRC | FLAG, 6xHis | Akache et al., |
| (Wuhan) | | | | 2021 |
| Swine deltaCoV S^{a} | AIH06857.1^{b} | NRC | FLAG-Dual | Galipeau et al., |
| | | | Strep-6xHis | 2021 |
| Avian_IBV S^{a} | AAP92675.1^{b} | NRC | FLAG-Dual | Galipeau et al., |
| | | | Strep-6xHis | 2021 |
| Pangolin CoV S^{a} | QIA48632.1^{b} | NRC | FLAG-Dual | Galipeau et al., |
| | | | Strep-6xHis | 2021 |
| Hedgehog CoV | QGA70702.0^{b} | NRC | FLAG-Dual | Galipeau et al., |
| HKU31 S^{a} | | | Strep-6xHis | 2021 |
| Bat CoV HKU9 S^{a} | YP_001039971.1^{c} | NRC | FLAG-Dual | Galipeau et al., |
| | | | Strep-6xHis | 2021 |
| Bat SARS like CoV- | AGZ48828.1^{b} | NRC | FLAG-Dual | Galipeau et al., |
| WIVI S^{a} | | | Strep-6xHis | 2021 |
| Bat 229E-related CoV | APD51507.1^{b} | NRC | FLAG-Dual | Galipeau et al., |
| S^{a} | | | Strep-6xHis | 2021 |
| Bat CoV 512 S^{a} | YP_001351684.1^{c} | NRC | FLAG-Dual | Galipeau et al., |
| | | | Strep-6xHis | 2021 |
| Bat SARS like CoV^{a} | ATO98157.1^{b} | NRC | FLAG-Dual | Galipeau et al., |
| | | | Strep-6xHis | 2021 |
| Civet SARS-CoV¹ S^{a} | AAU04646.1^{b} | NRC | FLAG-Dual | Galipeau et al., |
| | | | Strep-6xHis | 2021 |
| Human MERS-CoV S^{a} | AGH58717.1^{b} | NRC | FLAG-Dual | Galipeau et al., |
| | | | Strep-6xHis | 2021 |
| Human CoV-NL63 S | APF29071.1^{b} | Sino Biological | 6xHis | na |
| Human CoV-OC43 S^{a} | AGT51431.1^{b} | NRC | FLAG-Dual | Galipeau et al., |
| | | | Strep-6xHis | 2021 |
| Human CoV-HKU1 S | Q0ZME7.1^{b} | Sino Biological | 6xHis | na |
| Human CoV-229E S^{a} | AAK32191.1^{b} | Sino Biological | 6xHis | na |
| Human SARS-CoV S^{a} | AAP13442.1^{b} | NRC | FLAG-Dual | Galipeau et al., |
| | | | Strep-6xHis | 2021 |
| Human SARS-CoV- | QHD43416.1^{b} | NRC | FLAG, 6xHis | Colwill et al., |
| 2_Wuhan S^{a} (SmT1) | | | | 2021 |
| Human SARS-CoV- | — | NRC | FLAG, 6xHis | Galipeau et al., |
| 2_α (B.1.351) S^{a} | | | | 2021 |
| Human SARS-CoV- | — | NRC | FLAG, 6xHis | Galipeau et al., |
| 2_β (B.1.1.7) S^{a} | | | | 2021 |
| Human SARS-CoV- | — | NRC | none | Galipeau et al., |
| 2_γ (P.1) S^{a} | | | | 2021 |
| Human SARS-CoV- | — | NRC | FLAG, 6xHis | Stuible et al., |
| 2_Δ (B.1.617.2) S^{a} | | | | 2021 |
| Human SARS-CoV- | — | NRC | FLAG, 6xHis | Stuible et al., |
| 2_κ(B.1.617.1) S^{a} | | | | 2021 |
| Human SARS-CoV- | — | NRC | FLAG, 6xHis | Sulea et al., |
| 2_Omicron | | | | 2022 |
| (B.1.1.529) S^{a} | | | | |
| Human ACE2 | Q9BYF1-1^{d} | ACROBiosystems | Human IgG1 | na |
| (aa18-740) | | | Fc | |
| Human ACE2 | Q9BYF1-1^{d} | NRC | 6xHis | Wrapp et al., |
| (aa18-615) | | | | 2020 |

^{a}Proteins are C-terminally fused to the resistin trimerization domain.
^{b}GenBank;
^{c}NCBI;
^{d}UniProt. na, not applicable

### Example 1: Antigen Validation

### Introduction

Prior to use in library selection (panning) experiments, four SARS-CoV-2 spike protein antigens (S, S1, S1-RBD, and S2, as described in Table 1) were validated for structural integrity and functionality in adsorbed/captured states on microtiter wells by standard ELISA. Unless stated otherwise, all spike protein fragments used in the following Examples were produced as described in Stuible et al., 2021.

### Materials and Methods

### Binding to Cognate Human Angiotensin Converting Enzyme (ACE2) Receptor

ELISA was performed to determine if spike proteins were able to bind to human ACE2 when passively adsorbed (S, S1, S1-RBD and S2) or captured (S1, S1-RBD) on microtiter wells. For passive adsorption, wells of NUNC® Immulon 4 HBX MaxiSorp™ microtiter plates (Thermo Fisher, Cat #3855) were coated with 50 ng of SARS-CoV-2 spike proteins (S, S1, S2, S1-RBD) in 100 μL PBS overnight at 4° C. Following removal of protein solutions and three washes with PBST (PBS supplemented with 0.05% [v/v] Tween® 20), wells were blocked with PBSC (1% [w/v] casein [SIGMA, Cat #E3414] in PBS) at room temperature for 1 h. For capturing, in vivo biotinylated fragments harboring the AviTag™ (AviTag-S1, AviTag-S1-RBD) were diluted in PBS and added at 50 ng/well (100 μL) to pre-blocked Streptavidin Coated High Capacity Strip wells (Thermo Fisher, Cat #15501). After 1 h incubation at room temperature, wells were washed five times with PBST and incubated for an additional hour with 100 μL/well of 2-fold serially diluted ACE2-Fc (human ACE2 fused to human IgG1 Fc domain; ACROBiosystems, Cat #AC2-H5257) in PBSTC (PBS/0.2% casein/0.1% Tween® 20). Wells were washed five times and incubated for 1 h with 1 μg/mL HRP-conjugated goat anti-human IgG (SIGMA, Cat #A0170). Wells were washed 10 times and incubated with 100 μL peroxidase substrate solution (SeraCare, Cat #50-76-00) at room temperature for 15 min. Reactions were stopped by adding 50 μL 1 M H₂SO₄ to wells, and absorbance were subsequently measured at 450 nm using a Multiskan™ FC photometer (Thermo Fisher).

### Binding to Cognate Anti-Spike Protein Polyclonal Antibody

The four spike antigens were passively adsorbed as described above. After blocking with PBSC, wells were emptied, washed five times and incubated at room temperature for 1 h with 100 μL of 1 μg/mL anti-SARS-CoV-2 spike rabbit polyclonal antibody (Sino Biologicals, Cat #40589-T62) in PBSCT. Following 10 washes with PBST, wells were incubated with 100 μL 1/2500 dilution (320 ng/mL) of goat anti-rabbit:HRP (Jackson Immunoresearch, Cat #111-035-144) in PBSCT for 1 h at room temperature. After 1 h incubation and final five washes with PBST, the peroxidase activity was determined as described above.

### Results and Discussion

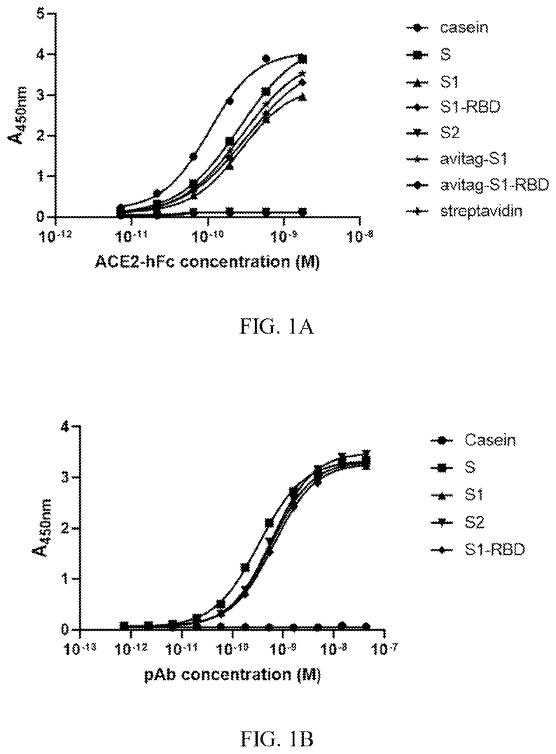

The passively adsorbed spike fragments, S, S1, S1-RBD, as well as the streptavidin-captured fragments, AviTag-S1-RBD and AviTag-S1, were found to bind to ACE2 with similarly high affinities (EC₅₀=0.10-0.32 nM; FIG. 1A, Table 2). As expected, the S2 subunit of the spike protein did not bind to ACE2. Additionally, as shown in FIG. 1B and Table 3, all four spike fragments in passively-adsorbed states (S, S1, S2, and S1-RBD), bound with high affinity (EC₅₀=0.34-0.65 nM) to a polyclonal antibody known to be specific for SARS-CoV-2 spike protein; confirming the structural integrity/identity of the spike protein fragments. The ELISA data demonstrate that the various spike fragments tested should maintain their natural and intact structures in passively-adsorbed and captured states during panning experiments.

TABLE 2
Binding affinity (EC₅₀) of passively absorbed spike fragments
and streptavidin-captured spike fragments to ACE2

| Antigen | S | S1 | S2 | S1-RBD | AviTag-S1-RBD | AviTag-S1 |
|---|---|---|---|---|---|---|
| EC₅₀ | 0.26 | 0.28 | nb | 0.32 | 0.10 | 0.28 |
| (nM) | | | | | | |

“nb” indicates lack or no binding.

TABLE 3
Binding affinity (EC₅₀) of passively absorbed
spike fragments to a polyclonal antibody known
to be specific for SARS-CoV-2 spike protein

| Antigen | S | S1 | S2 | S1-RBD |
|---|---|---|---|---|
| EC₅₀ (nM) | 0.34 | 0.59 | 0.6 | 0.65 |

### Example 2: Llama Immunization and Serum Analyses

### Introduction

As described below, two llamas were immunized with SARS-CoV-2 S or S/S1-RBD to trigger the generation of a diverse pool of antibodies targeting manifold sites over the surface of S, and targeting the S1-RBD sub-domain of S which is used by the virus to start the process of host cell infection through interaction with the ACE2 receptor. Llama sera were assessed by ELISAs for generation of immune responses against SARS-CoV-2 spike proteins, and by flow cytometry surrogate neutralization assays for generation of neutralizing antibodies.

### Materials and Methods

### Llama Immunization

Immunizations were performed at Cedarlane Laboratories (Burlington, ON, Canada) and essentially as described (Hussack et al., 2011). One llama (Eva Green) was immunized with 100 μg of S in 500 μL PBS combined with 500 μL of Freund's complete adjuvant on day 0, followed by immunization with 70 μg of S1-RBD (ACROBiosystems, Cat #SPD-552H6) in Freund's incomplete adjuvant on each of days 7, 14, and 21. Bleeds were taken at days 0, 21, and 28. A second llama (Maple Red) was immunized with 100 μg of S in 500 μL PBS combined with 500 μL of Freund's complete adjuvant on day 0, followed by immunization with 100 μg of S mixed with Freund's incomplete adjuvant on day 7, and immunization with 50 μg of S mixed with Freund's incomplete adjuvant on each of days 14 and 21.

### Serum ELISA

Llama sera were tested for antigen-specific immune response by ELISA essentially as described (Hussack et al., 2011; Henry et al., 2016). Briefly, dilutions of sera in PBST were added to wells pre-coated with S, S1, S2 or S1-RBD. Negative antigen control wells were pre-coated with casein (100 μL of 1% v/w) or recombinant human dipeptidase 1 ectodomain, DPEP1 (50 ng/well; Sino Biological, Cat #13543-H08H). Following 1 h incubation at room temperature, wells were washed 10 times with PBST and incubated with HRP-conjugated polyclonal goat anti-llama IgG heavy and light chain antibody (Bethyl, Cat #A160-100P) for 1 h at room temperature. After 10 washes, the peroxidase activity was determined as described above.

### Serum Surrogate Neutralization Assay by Flow Cytometry

Trimeric SARS-CoV-2 S was chemically biotinylated using EZ-Link™ NHS-LC-LC-Biotin following manufacturer's instructions (Thermo Fisher, Cat #21343). Vero E6 cells (ATCC, Cat #CRL-1586) were maintained according to ATCC protocols. Briefly, cells were grown to confluency in DMEM medium (Thermo Fisher, Cat #11965084) supplemented with 10% heat inactivated FBS (Thermo Fisher, Cat #10438034) and 2 mM Glutamax™ (Thermofisher, Cat #35050061) at 37° C. in a humidified 5% CO₂ atmosphere in T75 flasks. For flow cytometry experiments, cells were harvested by Accutase™ (Thermo Fisher, Cat #A111050) treatment, washed once by centrifugation, and resuspended at 1×10⁶ cells/mL in PBSB (PBS containing 1% BSA) and 0.05% [v/v] sodium azide [SIGMA, Cat #52002]). Cells were kept on ice until use. To determine the presence of neutralizing antibodies in the immune sera of llamas, 400 ng of chemically biotinylated trimeric SARS-CoV-2 S was mixed with 5×10⁴ Vero E6 cells in the presence of 2-fold dilutions of sera (pre immune, day 21 and day 28 serum) in a final volume of 150 μL. Following 1 h of incubation on ice, cells were washed twice with PBSB by centrifugation for 5 min at 1200 rpm and then incubated for an additional hour with 50 μL of Streptavidin, R-Phycoerythrin Conjugate (SAPE, ThermoFisher, Cat #S866) at 250 ng/mL diluted in PBSB. After a final wash, cells were resuspended in 100 μL PBSB and data were acquired on a CytoFLEX® S flow cytometer (Beckman Coulter, Brea, CA) and analyzed by FlowJo™ software (FlowJo LLC, v10.6.2, Ashland, OR). Percent inhibition (neutralization) was calculated according to the following formula: % inhibition=100×[1−(Fₙ−Fₘᵢₙ)/(Fₘₐₓ−Fₘᵢₙ)], where, Fₙ is the measured fluorescence at any given competitor serum dilution, Fₘᵢₙ is the baseline fluorescence measured in the presence of cells and SAPE only, and Fₘₐₓ is the maximum fluorescence, measured in the absence of competitor serum.

### Results and Discussion

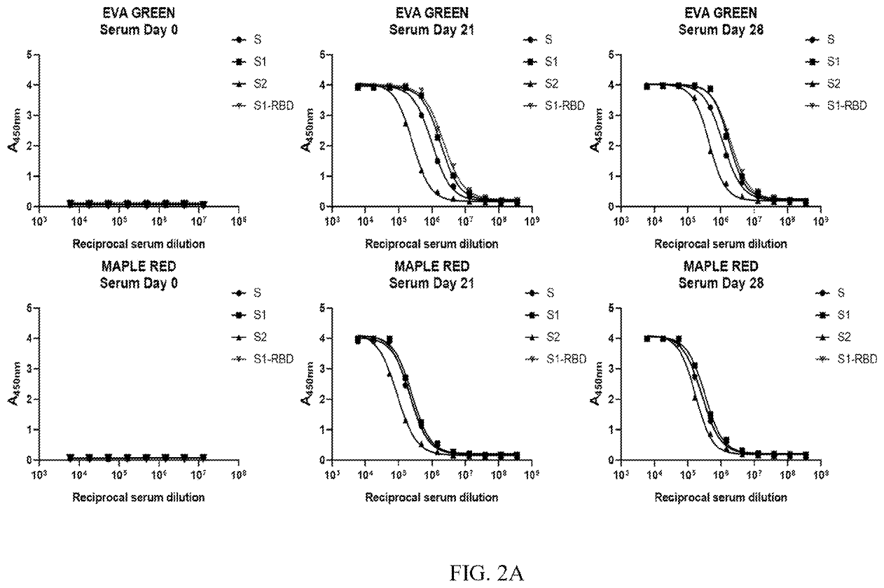

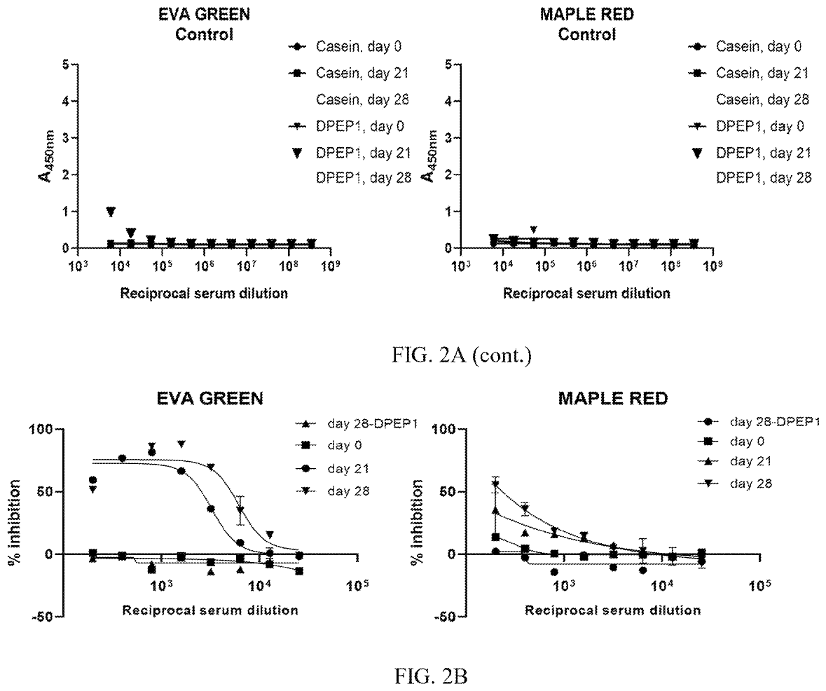

The results of ELISAs performed with pre-immune (day 0) and immune (day 21 and 28) sera demonstrate that both llamas generated a strong immune response against target immunogens S, S1, S2 and S1-RBD (FIG. 2A). Based on EC₅₀ values, which indicate the strength of immune responses, Eva Green generated a stronger immune response, up to 10-fold stronger, than Maple Red consistently across all four spike fragments (FIG. 2A; Table 4). Further, the immune responses were specific for SARS-CoV-2 antigens, as sera from day 0, 21 and 28 did not react with casein or DPEP1. Interestingly, one initial injection of S was enough to develop a strong, maximum immune response against S2 by Eva Green. Llama sera were also assessed by flow cytometry surrogate neutralization assays for generation of neutralizing antibodies, i.e., antibodies that block the interaction between the trimeric SARS-CoV-2 S and ACE2 displayed on the surface of Vero E6 cells. As shown in FIG. 2B and Table 5, inhibition serum titers of 3300 (Day 21) and 6200 (Day 28) reciprocal serum dilution (RSD) were obtained in the case of Eva Green sera whereas weaker inhibition serum titers, <200 (Day 21) and <400 RSD (Day 28), were obtained for Maple Red.

TABLE 4
ELISA results summarizing day 0, 21 and 28 binding serum
titers (EC₅₀s) of Eva Green and Maple Red llamas against
spike protein fragments S, S1, S2 and S1-RBD

| | | Binding serum titer, EC₅₀ | | | |
|---|---|---|---|---|---|
| | | (reciprocal serum dilution) | | | |
| Llama | Day | S | S1 | S2 | S1-RBD |
| Eva Green | 0 | — | — | — | — |
| | 21 | 1.0 × 10⁶ | 1.8 × 10⁶ | 0.3 × 10⁶ | 2.2 × 10⁶ |
| | 28 | 1.2 × 10⁶ | 1.8 × 10⁶ | 0.4 × 10⁶ | 2.0 × 10⁶ |
| Maple Red | 0 | — | — | — | — |
| | 21 | 0.2 × 10⁶ | 0.3 × 10⁶ | 0.1 × 10⁶ | 0.2 × 10⁶ |
| | 28 | 0.3 × 10⁶ | 0.3 × 10⁶ | 0.2 × 10⁶ | 0.3 × 10⁶ |

Dashes indicate lack of binding.

TABLE 5
Flow cytometry-based surrogate virus neutralization
assay results summarizing day 21 and 28 inhibition serum titers
(IC₅₀s) of Eva Green and Maple Red llamas using spike
protein S as surrogate for the virus

Inhibition serum titer, IC₅₀
(reciprocal serum dilution)

| Llama | Day 21 | Day 28 |
|---|---|---|
| Eva Green | 3300 | 6200 |
| Maple Red | <200 | <400 |

### Example 3: Phage Display Library Construction, Selection and Screening

### Introduction

Two libraries (Eva Green and Maple Red) were constructed and subjected to selection against spike protein fragments. Selection and screening efforts were aimed at isolating not only S1-RBD binders, but also S1-NTD and S2 binders, as recent findings indicate that in addition to S1-RBD binders, S1-NTD and S2 binders could also be neutralizing (Rogers et al., 2020; Ravichandran et al., 2020). To this end, two libraries were generated and were selected under six different conditions to maximize the number and epitopic diversity of hits against S1-RBD, S1-NTD and S2. After two rounds of selection, monoclonal phages ELISA and DNA sequencing were performed to identify antigen-specific hits.

### Materials and Methods

### Phage Display Library Construction

On day 28, 100 mL of blood from each of the two llamas was drawn and peripheral blood mononuclear cells (PBMCs) were purified by Ficoll® gradient at Cedarlane Laboratories (Burlington, ON, Canada). Two independent phage-displayed V_{H}/V_{H}H libraries were constructed from ˜5×10⁷ PBMCs as described previously (Henry et al., 2016; Rossotti et al., 2015; Henry et al., 2015). Total RNA was extracted from PBMCs using TRIzol™ Plus RNA Purification Kit (Thermo Fisher, Cat #12183555) following manufacturer's instructions and used to reverse transcribe cDNA with SuperScript™ IV VILO™ Master Mix supplemented with random hexamer (Thermofisher, Cat #SO142) and oligo (dT) (Thermofisher, Cat #AM5730G) primers. V_{H}/V_{H}H genes were amplified using semi-nested PCR and cloned into the phagemid vector pMED1, followed by transformation of E. coli TG1 to construct two libraries with sizes of 1×10⁷ and 2×10⁷ independent transformants for Eva Green and Maple Red, respectively. Both libraries showed an insert rate of ˜95%, as verified by DNA sequencing. Phage particles displaying the V_{H}s/V_{H}Hs were rescued from E. coli cell libraries using M13K07 helper phage (New England Biolabs, Cat #N0315S) as described in Hussack et al., 2011 and used for selection experiments described below.

### Library Selection and Screening

Library selections (pannings) and screenings were performed essentially as described (Hussack et al., 2011; Rossotti et al., 2015). Library selections were performed on microtiter wells under 6 different phage binding/elution conditions designated P1-P6. Briefly, for the phage binding step, library phages were diluted at 1×10¹¹ colony-forming units (cfu)/mL in PBSBT [PBS supplemented with 1% [w/v] BSA and 0.05% Tween® 20] and incubated in antigen-coated microtiter wells for 2 h at 4° C. For P1-P4, phages were added to wells with passively-adsorbed S (10 μg/well; P1), passively-adsorbed S2 (10 μg/well; P2), streptavidin-captured biotinylated S1 (0.5 μg/well; P3) and streptavidin-captured biotinylated S1-RBD (0.5 μg/well; P4). For P5, phages were pre-absorbed on passively-adsorbed S1-RBD wells (10 μg/well) for 1 h at 4° C. and then the unbound phage in the solution was transferred to wells with streptavidin-captured biotinylated S1 (0.5 μg/well) in the presence of non-biotinylated S1-RBD competitor in solution (10 μg/well). Following the binding stage (P1-P5), wells were washed 10 times with PBST and bound phages were eluted by treatment with 100 mM glycine pH 2.2 for 10 min at room temperature, followed by immediate neutralization of phages with 2 M Tris. Similar to P4, in P6, phages were bound on streptavidin-captured biotinylated S1-RBD but elution of bound phages were carried out competitively with 50 nM ACE2-Fc following the washing step. For all pannings, a small aliquot of eluted phage was used to determine their titer on LB-agar/ampicillin plates and the remaining were used for their subsequent amplification in E. coli TG1 strain (Hussack et al., 2011). The amplified phages were used as input for the next round of selection as described above.

After two rounds of selection, 16 (Eva Green) or 12 (Maple Red) colonies from each of the P1-P6 selections were screened for antigen binding by monoclonal phage ELISA against S, S1, S2 and S1-RBD. Individual colonies from eluted-phage titer plates were grown in 96 deep well plates in 0.5 mL 2YT media/100 μg/mL-carbenicillin/1% (w/v) glucose at 37° C. and 250 rpm to an OD₆₀₀ of 0.5. Then, 10¹⁰ cfu M13K07 helper phage was added to each well and incubation continued for another 30 min under the same conditions. Cells were subsequently pelleted by centrifugation, the supernatant was discarded and the bacterial pellets were resuspended in 500 μL 2YT/100 μg/mL carbencillin/50 μg/mL kanamycin and incubated overnight at 28° C. Next day, phage supernatants were recovered by centrifugation, diluted 3-fold in PBSTC and used in subsequent screening assays by ELISA. To this end, antigens were coated onto microtiter wells at 50 ng/well overnight at 4° C. Next day, plates were blocked with PBSC, washed five times with PBSTC, and 100 μL of phage supernatants prepared above were added to wells, followed by incubation for 1 h at room temperature in an orbital shaking platform. After 10 washes, binding of phages was detected by adding 100 μL/well of anti-M13:HRP (Santa Cruz, Cat #SC-53004HRP) at 40 ng/mL in PBSTC and incubating as above. After 10 washes, the peroxidase activity was determined as described previously. Following confirmation of success of library panning as determined by monoclonal phages ELISA, a total of ≈1200 individual clones (2100 clones per panning strategy; ≈600 clones per library) were colony-PCRed and subsequently sequenced, resulting in the identification of 35 (Eva Green) and 12 (Maple Red) potential spike-specific V_{H}H antibodies.

### Results and Discussion

Eva Green and Maple Red libraries were constructed with functional sizes (library sizes corrected for insert rate) of ˜1×10⁷ and ˜2×10⁷, respectively. Two rounds of selection under six different panning conditions (P1-P6) were subsequently performed for both libraries. To confirm the success of selection in enriching for binders, samples of 12-16 clones per panning condition were tested for binding against S, S1, S2 and S1-RBD by phage ELISA. The frequent occurrence of positive clones determined by monoclonal phage ELISA confirmed selections efficiently enriched for binders. Specificity patterns observed, i.e., binding against S vs S1 vs. S2 vs S1-RBD, in sample sets reflected the selection strategy as well as the immunization strategy (Eva Green was immunized with S once but predominantly [three times] with S1-RBD). In P3, P4 and P6, as expected based on the selection strategy, essentially all binders were S1-RBD specific. For Maple Red, the immunization with the whole spike S generated a strong bias against non-S1-RBD-specific antibodies, an observation recently seen with patients recovered from SARS-CoV-2 natural infection (Rogers et al., 2020) and rabbits immunized with SARS-CoV-2 S (Ravichandran et al. 2020). Panning against S (P1) essentially produced S2 binders as opposed to S1-RBD binders seen in the case of Eva Green library. Additionally, in contrast to what was observed in the case of the Eva Green, for the Maple Red P3 strategy, where panning was performed against S1, half of the binders tested were specific for non-S1-RBD region of S1. Nonetheless when selections were specifically directed towards S1-RBD binders, as in the P4 and P6 selection strategies, all tested binders were S1-RBD specific. Additionally, the P5 strategy almost exclusively selected for V_{H}Hs specific to non-RBD region of the S1 subunit. In summary, the immunization strategy was a key determinant of the outcome of in vivo generated V_{H}Hs with respect to spike subunit/domain specificity, and in vitro directed selection strategies effectively yielded intended binding specificities. Subsequently, a larger number of clones, >600 clones per library, were screened by DNA sequencing to obtain a large pool of potential binders. The unique sequences were subjected to binding validation, as described below.

### Example 4: V_{H}H Cloning/Expression in E. coli, Stability/Affinity Validation and Cross-Reactivity Studies

### Introduction

Hits identified by monoclonal phage ELISA and DNA sequencing were cloned into the expression vector pMRo.BAP.H₆ (Rossotti et al., 2019), produced as His6-tagged V_{H}Hs in the periplasmic space of E. coli BL21(DE3) and purified by immobilized metal-ion affinity chromatography (IMAC). V_{H}Hs were subsequently validated for binding and further explored for cross-reactivity soluble ELISA against SARS-CoV-2, SARS-CoV and MERS-CoV spike proteins. Additionally, V_{H}Hs were validated for aggregation resistance by size exclusion chromatography (SEC) and thermostability by circular dichroism Tₘ measurement assays. Lead V_{H}Hs were produced in mammalian cells in fusion with human IgG1 Fc and were subsequently tested in a comprehensive cross-reactivity ELISA against a collection of various coronavirus spike proteins (S).

### Materials and Methods

DNA Sequence Analysis and V_{H}H Production in E. coli

Colonies were analyzed by DNA sequencing and identified V_{H}H sequences were aligned using IMGT system. V_{H}Hs were subsequently cloned into pET expression vector (Novagen, Madison, WI) for their production in BL21(DE3) E. coli as monomeric soluble protein (Rosotti et al., 2019). Briefly, individual colonies were cultured overnight in 10 mL of LB supplemented with 50 μg/mL of kanamycin (LB/Kan) at 37° C. and 250 rpm. After 16 h, cultures were added to 250 mL LB/Kan and grown to an OD₆₀₀ of 0.6. Expression of V_{H}Hs was induced with 10 μM of IPTG (isopropyl β-D-1-thiogalactopyranoside) overnight at 28° C. and 250 rpm. The following day, bacterial pellets were harvested by centrifugation at 6,000 rpm for 15 min at 4° C. and V_{H}/V_{H}Hs were extracted by sonication and purified by IMAC as described previously (Rosotti et al., 2019). In addition, for ELISA (see below), a small fraction was biotinylated by incubating 1 mg of purified V_{H}Hs with 10 μM of ATP (Alfa Aesar, Cat #CAAAJ61125-09), 100 μM of D-(+)-biotin (VWR, Cat #97061-446) and a bacterial cell extract overexpressing E. coli BirA as described previously (Rossotti et al., 2015b). The same procedure was followed to produce a biotinylated V_{H}H-72 benchmark V_{H}H (Wrapp et al., 2020), a SARS-CoV spike protein-specific V_{H}H that cross-reacts with the SARS-CoV-2 spike protein receptor binding domain.

### V_{H}H Binding Validation and Preliminary Cross-Reactivity Studies by ELISA

Binding validation studies were performed with S1-RBD-specific clones. Briefly, microtiter well plates were coated with 50 ng/well SARS-CoV-2 S1-RBD in 100 μL PBS overnight at 4° C. Plates were blocked with PBSC for 1 h at room temperature, then washed five times with PBST and incubated with decreasing concentrations of biotinylated V_{H}Hs. After 1 h incubation, plates were washed 10 times with PBST and binding of V_{H}Hs was probed using HRP-streptavidin (Jackson ImmunoResearch, Cat #016-030-084). Finally, plates were washed 10 times with PBST and peroxidase activity was determined as described above.

### Stability Determinations by Size Exclusion Chromatography (SEC) and Circular Dichroism

Purified V_{H}Hs were subjected to SEC to validate their aggregation resistance. Briefly, 2 mg of each affinity purified V_{H}H was injected into Superdex™ 75 GL column (Cytiva) connected to an ÄKTA FPLC protein purification system (Cytiva) as previously described (Henry et al., 2017). PBS was used as running buffer at 0.8 mL/min. Fractions corresponding to the monomeric peak were pooled and stored at 4° C. until use. To determine thermostability, V_{H}H Ts were measured by circular dichroism as previously described (Henry et al., 2017). Ellipticity of V_{H}Hs were determined at 200 μg/mL V_{H}H concentrations and 205 nm wavelength in 100 mM sodium phosphate buffer, pH 7.4. Ellipticity measurements were normalized to percentage scale and Tₘs were determined from plot of % folded vs temperature and fitting the data to a Boltzmann distribution.

Production of V_{H}Hs in Mammalian Cells Infusion with Human IgG1 Fc

Codon-optimized genes for bivalent V_{H}H-Fcs were synthesized (GenScript). For heterodimeric monovalent V_{H}H-Fcs, V_{H}H genes were PCR amplified as described previously and cloned into pTT5-hIgG1Fc between the genes for human V_{H} leader sequence and the human IgG1 hinge/Fc sequences, using NarI/HindIII restriction sites. Bivalent V_{H}H-Fcs were produced by transient transfection of HEK293-6E cells followed by protein A affinity chromatography as previously described (Rosotti et al., 2019). Heterodimeric monovalent V_{H}H-Fcs were produced by co-transfection of HEK293-6E cells with two pTT5 vectors, one encoding for a 6×His-tagged heavy chain (V_{H}H1-hinge-C_{H}2-C_{H}3-His₆), the other for a non-tagged heavy chain of a different V_{H}H (V_{H}H2-hinge-C_{H}2-C_{H}3). The heterodimeric antibodies were purified by sequential protein A affinity chromatography and IMAC. For IMAC, antibodies were eluted using a linear 0-0.5 M imidazole gradient over 20 column volumes to separate species bearing one 6×His tag (heterodimeric, monovalent) from those bearing two 6×His tags (homodimeric, bivalent). Proteins were buffer exchanged using Amicon® Ultra-15 Centrifugal Filter Units (Millipore, Cat #UFC905024) with phosphate-buffered saline (PBS), pH 7.4. The same procedure was applied for the generation of the reference bio-V_{H}H-72 and V_{H}H-72-Fc using the sequence published by Wrapp et al., 2020. The sequence of the V_{H}H was ordered as GeneBlock (IDT DNA) flanked by SfiI sites for cloning into pMRo.BAP.H6, and NarI/HindIII for cloning into pTT5-hIgG1Fc. Protein purity was evaluated by SDS-PAGE using 4-20% Mini-PROTEAN® TGX Stain-Free™ Gels (Bio-Rad, Cat #17000435).

### V_{H}H-Fc Comprehensive Cross-Reactivity Studies by ELISA

Recombinant coronavirus spike proteins S (Table 1) were coated overnight onto NUNC® MaxiSorp™ 4BX plates (Thermo Fisher) at 50 ng/well in 100 μL of PBS, pH 7.4. The next day, plates were blocked with 200 μL PBSC for 1 h at room temperature, then washed five times with PBST and incubated at room temperature for 1 h on rocking platform at 80 rpm with 1 μg/mL V_{H}H-Fc diluted in PBSTC. Plates were washed five times with PBSTC and binding of V_{H}H-Fcs was detected using 1 μg/mL HRP-conjugated goat anti-human IgG. Finally plates were washed five times and peroxidase (HRP) activity was measured as described above.

### Results and Discussion

A total of ˜1200 colonies were analyzed by DNA sequencing. Forty seven potential V_{H}H binders were identified from the two libraries (35 from the Eva Green and 12 from the Maple Red library) by phage ELISA and DNA sequencing, with the vast majority (35 V_{H}Hs) coming from the Eva Green library (Tables 6 and 7). Some V_{H}Hs may be clonally related due to their high sequence identity in their CDRs. Examples include NRCoV2-1a, NRCoV2-1c and NRCoV2-1d from the Eva Green library (Table 6) and NRCoV2-MRed02 and NRCoV2-MRed04 from the Maple Red library (Table 7). V_{H}H hits were cloned in E. coli, confirmed by DNA sequencing, and expressed and purified by IMAC. Following expression of V_{H}Hs, the binding of a sample set of V_{H}Hs was validated by ELISA. Affinities, expressed as EC₅₀s, were high, ranging from 0.4 to 7.2 nM (data not shown). V_{H}Hs were also tested for aggregation resistance and stability, and cross-reactivity.

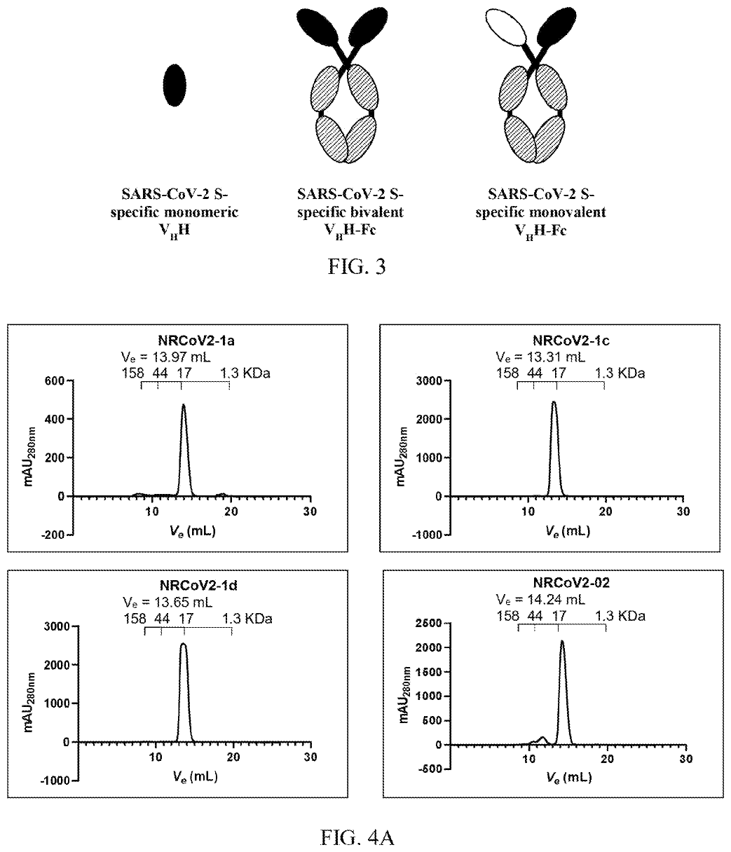

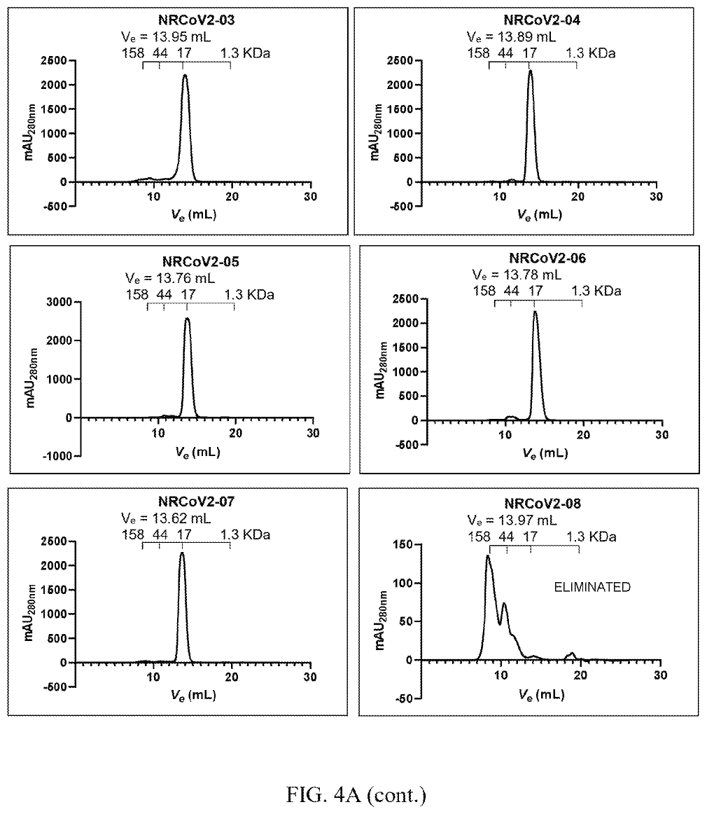

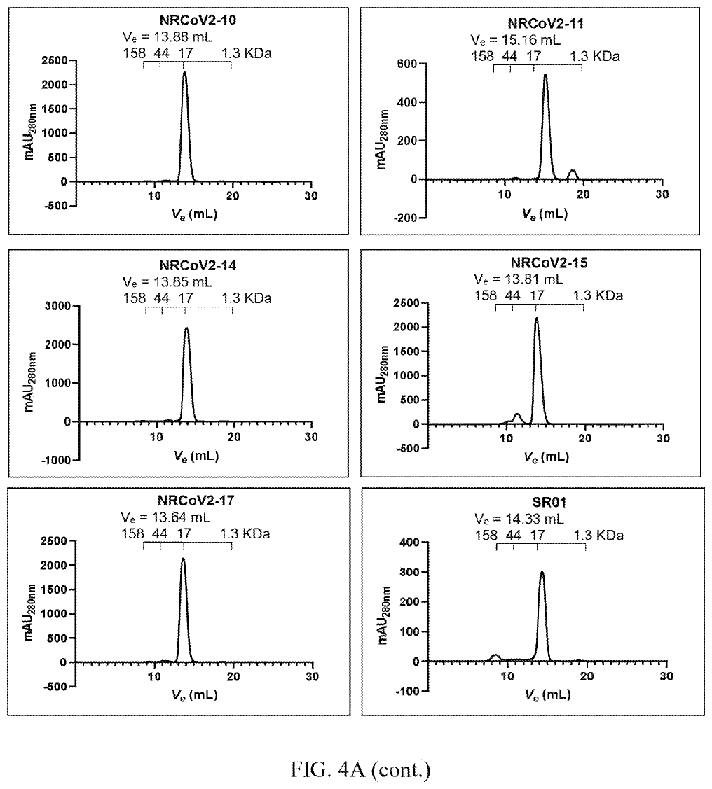

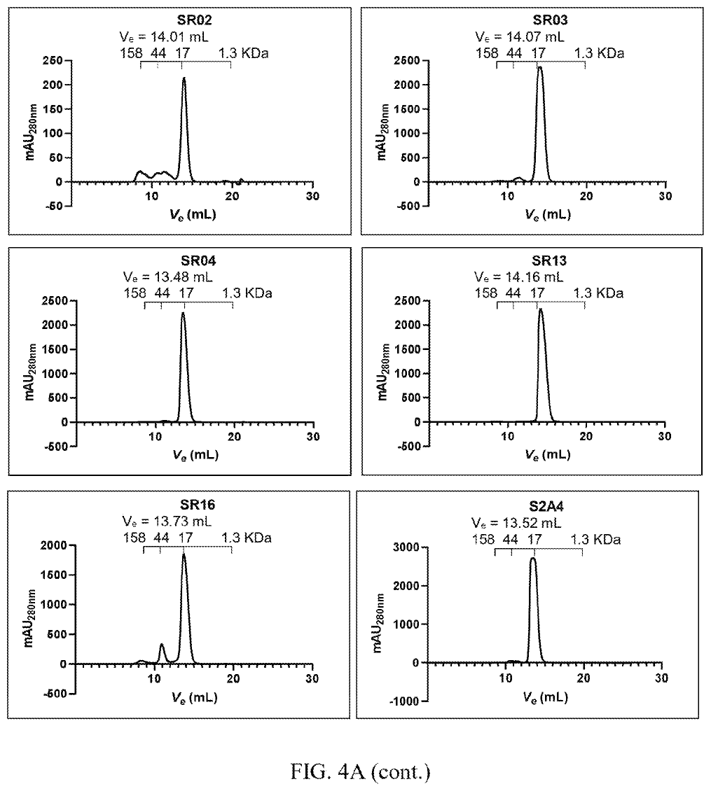

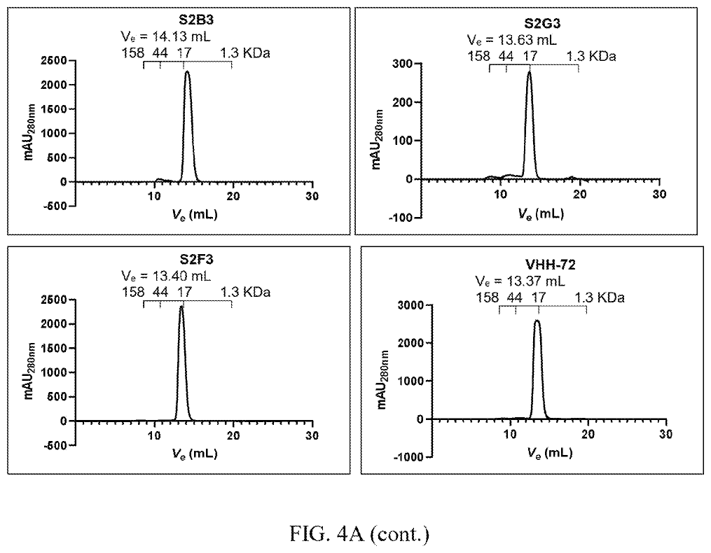

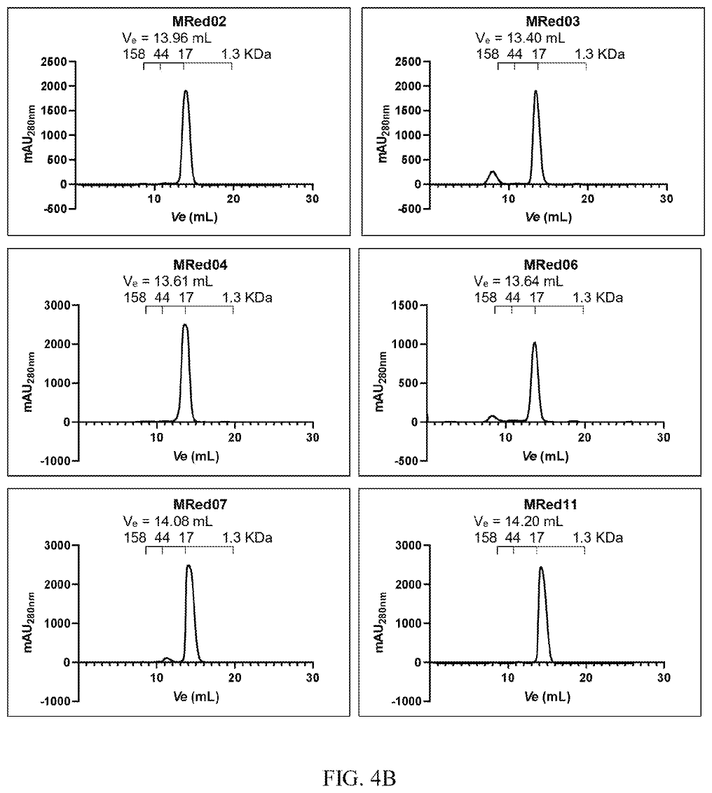

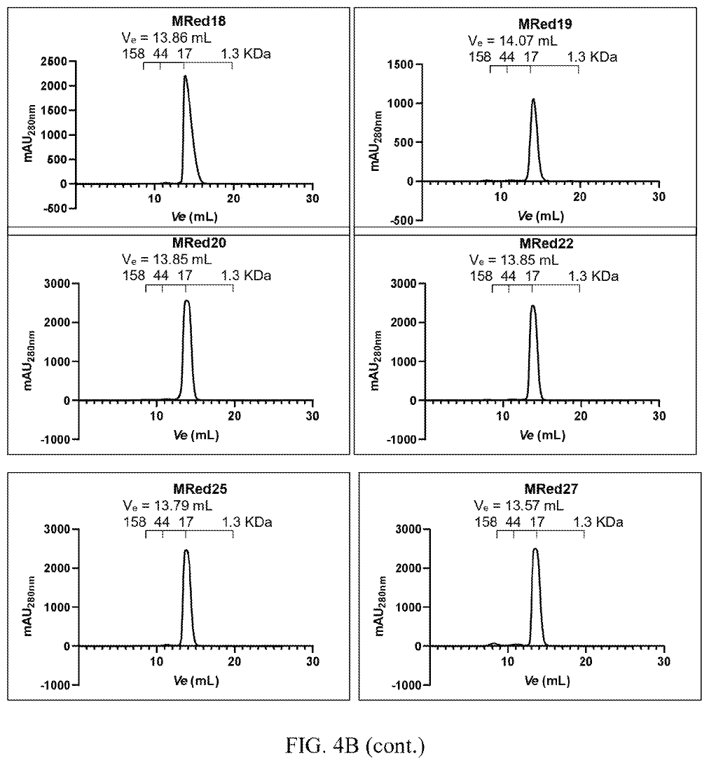

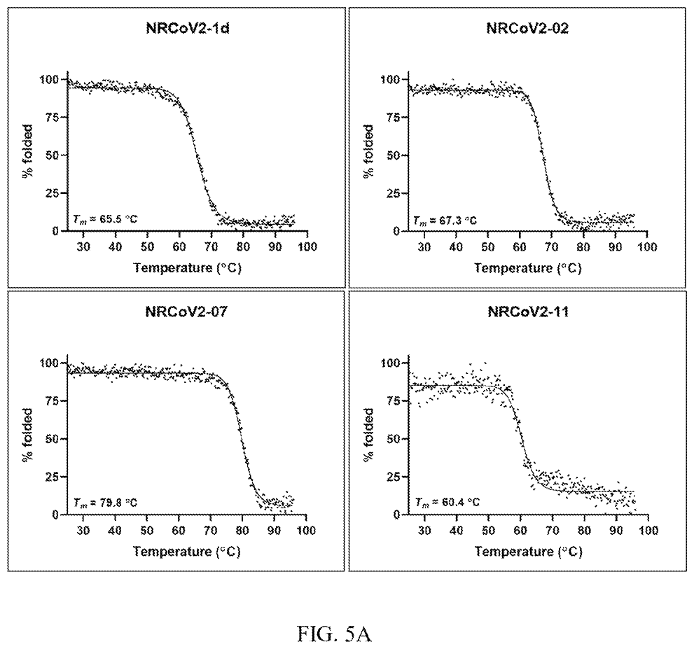

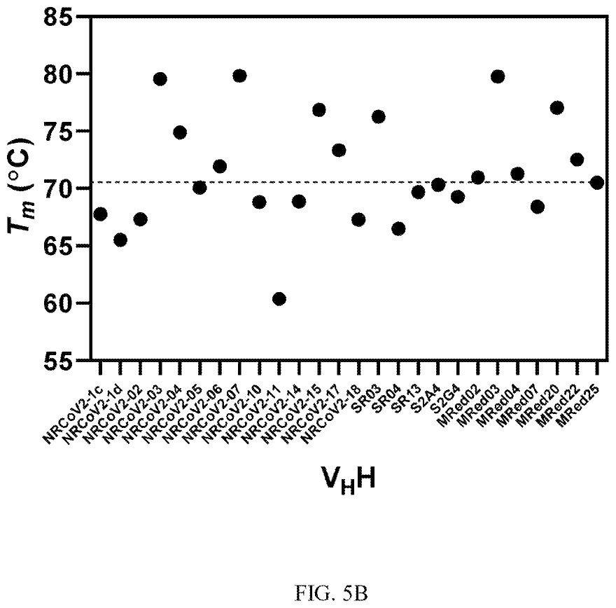

Aggregation resistance and stability are desirable attributes of biotherapeutics, as they affect both efficacy and manufacturability. By size exclusion chromatography, all V_{H}Hs tested were found to be aggregation resistant (FIGS. 4A and 4B), except for NRCoV2-08, which showed some degree of aggregation The V_{H}Hs were also tested for thermal stability and found to be highly thermostable. With the exception of NRCoV2-11, which had a relatively lower Tₘ of 60.4° C., the remaining 25 V_{H}Hs tested had Tₘs higher than 65° C., with a Tₘ range and median of 65.5-79.8° C. and 70.4° C., respectively (FIGS. 5A and 5B). Many V_{H}Hs had Tₘs that were higher than that of the V_{H}H-72 benchmark (73.0° C.). V_{H}Hs with antigen binding activity were produced as monomeric and dimeric V_{H}H-Fcs for subsequent binding and neutralization assays. The schematic formats of these fusion molecules are depicted in FIG. 3.

The results of cross-reactivity studies using SARS-CoV-2 variants and various coronaviruses are shown in FIGS. 6A and 6B. Initial experiments showed that for the UK (Alpha) and South Africa (Beta) variants of SARS-CoV-2, eight out of nine S1-NTD-specific V_{H}Hs tested were cross-reactive to both variants (FIG. 6A). In the case of S1-RBD-specific V_{H}Hs, 15/20 cross-reacted to both variants and an additional four cross-reacted with the UK variant. Only one (NRCoV2-08) V_{H}H was not cross-reactive at all. Additionally, one S1-NTD-specific V_{H}H, six S1-RBD-specific and eight S2-specific V_{H}Hs cross-reacted with SARS-CoV. Many antibodies also cross-reacted with pangolin CoV, with fewer, but still significant, numbers cross-reacting to SARS-like CoV W1V1, bat SARS-like CoV and civet SARS-CoV with similar cross-reactivity patterns. None of the antibodies tested cross-reacted with Swine deltaCoV, Avian IBV, hedgehog CoV HKU31, Bat CoV HKU9, Bat 229E-related CoV, bat CoV 512, human MERS betaCoV Jordan, human CoV-NL63, Human CoV-OC43 or human CoV-HKU1.

In a subsequent experiment (results shown in FIG. 6B), V_{H}Hs were examined for cross reactivity to a collection of spike glycoproteins from various coronavirus genera and SARS-CoV-2 variants by ELISA (FIG. 6B) and SPR (Tables 11 and 12), many V_{H}H-Fcs cross-reacted with the S protein from variants Alpha, Beta, Gamma, Delta and Kappa (B.1.617.1; Variant Being Monitored [VBM]). The exceptions were: 1) RBD-specific V_{H}Hs NRCoV2-02/NRCoV2-05 did not cross-react with Beta and Gamma and NRCoV2-04/NRCoV2-14/NRCoV2-15, did not cross-react with Kappa and 2) S2-specific V_{H}Hs NRCoV2-MRed18 and NRCoV2-MRed19 did not cross-react with Kappa. All nine NTD-specific V_{H}Hs cross-reacted with all variants tested. Additionally, many V_{H}Hs cross-reacted with pangolin CoV, with fewer cross-reacting to SARS-CoV, SARS-like CoV WIV1, bat SARS-like CoV and civet SARS CoV. These viruses, including variants, are all of the Betacoronavirus Sarbecovirus subgenus. None of the antibodies tested cross-reacted with the remaining 11 non-Sarbecovirus Betacoronavirus, or with Alphacoronavirus, Deltacoronavirus or Gammacoronavirus. 29 V_{H}Hs cross-reacted with the Omicron variant (FIG. 6B). The broadly cross-reactive antibodies included V_{H}Hs targeting all three regions of the S protein (RBD, NTD, S2). The most broadly cross-reactive V_{H}Hs recognizing 10-12 viruses, including SARS-CoV-2 variants, were two NTD binders (NRCoV2-SR01, NRCoV2-SR02), six RBD binders (NRCoV2-1 d, NRCoV2-07, NRCoV2-11, NRCoV2-12, NRCoV2-20, NRCoV2-MRed04) and six S2 binders (NRCoV2-S2F3, NRCoV2-S2G3, NRCoV2-S2G4, NRCoV2-MRed18, NRCoV2-MRed19, NRCoV2-MRed20). The V_{H}H-72 benchmark was also broadly cross-reactive. The panel of V_{H}Hs had similar cross-reactivity profiles to human ACE2, except that ACE2 did not bind civet SARS-CoV S and, unsurprisingly, bound HCoV-NL63 S.

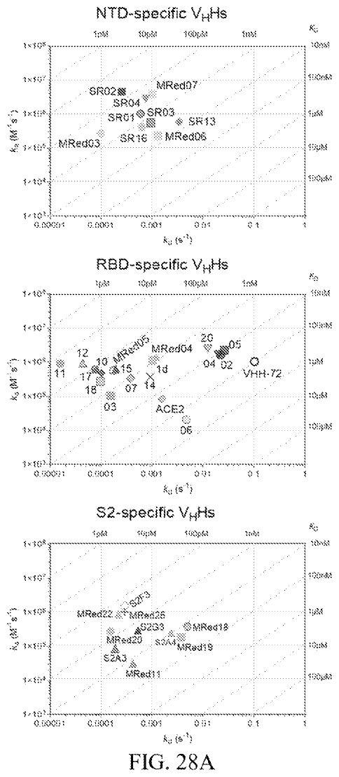

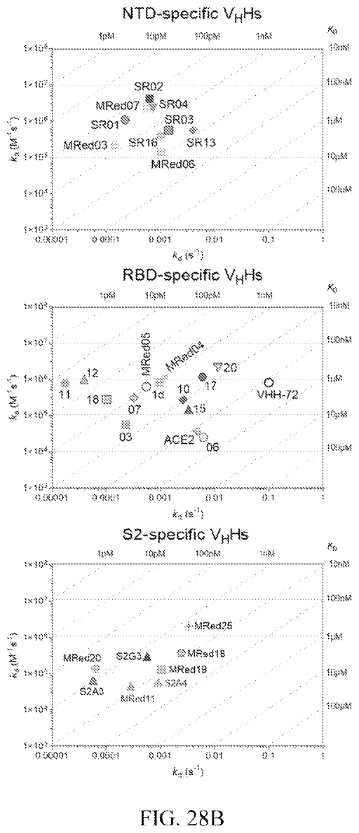

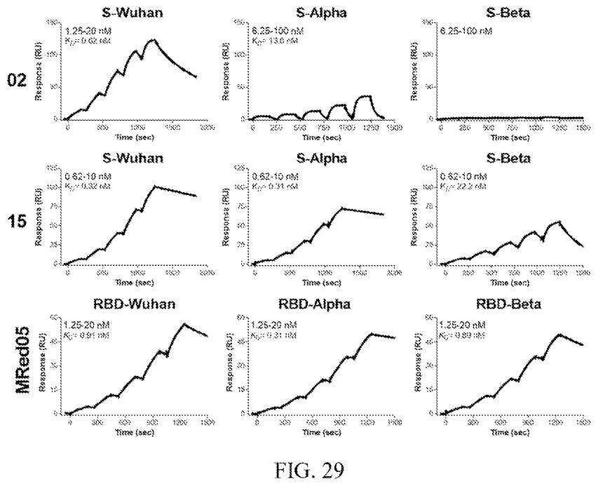

When tested by SPR against SARS-CoV, 12 out of 14 ELISA-positive V_{H}Hs cross-reacted with SARS-CoV S, most with comparably high affinities (Table 11. Seven of these V_{H}Hs were S2-specific, four were RBD-specific and one was NTD-specific. Against the Alpha and Beta variants, the SPR cross-reactivity data, performed with 37 V_{H}Hs, were consistent with ELISA, except for NRCoV2-04 and NRCoV2-14, which were negative or very weak for binding to the Beta variant by SPR (Tables 11 and 12). All 37 V_{H}Hs tested bound the Alpha variant S protein, and 34 were also cross-reactive to the Beta variant S protein (FIGS. 28A (Alpha) and 28B (Beta); FIG. 29; Table 11; Table 12). Thirteen out of 17 RBD-specific V_{H}Hs bound all three variants with similar affinities, except for V_{H}Hs NRCoV2-10, NRCoV2-15 and NRCoV2-17 which bound to the Beta variant with 40-50-fold weaker affinity; the remaining four that did not bind the Beta variant showed cross-reactivity with the Alpha variant with similar (NRCoV2-04, NRCoV2-14) or reduced (˜5-fold [NRCoV2-05] and ˜20-fold [NRCoV2-02]) affinity relative to the Wuhan variant. All NTD-specific and S2-specific V_{H}Hs cross-reacted with the three variants with essentially the same or similar affinities.

The cross-reactivity of the V_{H}Hs and V_{H}H-Fcs is significant, as it is believed that the progenitor of SARS-CoV was generated by recombination among bat SARS-like coronaviruses that spread to humans via civet cat as an intermediate host (Zheng et al, 2020). Further, most new emerging viruses are derived from strains circulating in zoonotic reservoirs. Antibodies that can cross-react against a variety of animal and human coronaviruses have potential to be used for detection and/or treatment of emerging coronavirus outbreaks.

TABLE 6
CDR-IMGT sequences of anti-spike protein V_{H}Hs
obtained from Eva Green phage display library

| | | | | | | | Sub- | |
|---|---|---|---|---|---|---|---|---|
| | | | | | | | unit/ | |
| | | | | | | | do- | Selec- |
| | | SEQ | | SEQ | | SEQ | main | tion |
| | CDR | ID | | ID | | ID | speci- | stra- |
| V_{H}H | 1 | NO: | CDR 2 | NO: | CDR 3 | NO: | ficity | tegy |
| NRCoV2- | GSTL | 1 | VSSSDGST | 45 | AADYSMRP | 90 | S1-RBD | P1, |
| 1a² | DYYA | | | | LWVSRWHR | | | P3, |
| | | | | | DYEY | | | P4, |
| | | | | | | | | P6 |
| NRCoV2- | GSIL | 2 | VSSSDGST | 45 | AADYSMRR | 91 | S1-RBD | P1, |
| 1c² | DYYA | | | | FAVGRWHR | | | P3, |
| | | | | | DYEY | | | P4, |
| | | | | | | | | P6 |
| NRCoV2- | GSTL | 1 | VSSSDGNT | 46 | AADYSMRP | 92 | S1-RBD | P1, |
| 1d¹ | DYYA | | | | FAVGRWHR | | | P3, |
| | | | | | DYEY | | | P4, |
| | | | | | | | | P6 |
| NRCoV2- | GFTF | 3 | ISGRGDDT | 47 | TKGPDLYY | 93 | S1-RBD | P1, |
| 02¹ | SNYA | | | | FGSGYSD | | | P3, |
| | | | | | | | | P4, |
| | | | | | | | | P5, |
| | | | | | | | | P6 |
| NRCoV2- | GITF | 4 | MSNMDST | 48 | NIYGPTYS | 94 | S1-RBD | P4, |
| 03¹ | SYYA | | | | TRRNEY | | | P6 |
| NRCoV2- | GSPF | 5 | ISGGGIA | 49 | WSSYEST | 95 | S1-RBD | P1, |
| 04¹ | SNVV | | | | | | | P3, |
| | | | | | | | | P4, |
| | | | | | | | | P6 |
| NRCoV2- | GFIF | 6 | INSGGGDT | 50 | SKGPVSSY | 96 | S1-RBD | P1, |
| 05¹ | SNYA | | | | YGSGYDY | | | P3, |
| | | | | | | | | P4, |
| | | | | | | | | P6 |
| NRCoV2- | VSTF | 7 | IGFVGAT | 51 | NARHYGGS | 97 | S1-RBD | P4, |
| 06¹ | SSYA | | | | EY | | | P6 |
| NRCoV2- | GVTL | 8 | ISSNGRRN | 52 | AAVQDVHG | 98 | S1-RBD | P1, |
| 07¹ | DYYA | | | | DNYYCTSP | | | P3, |
| | | | | | NEYNV | | | P4, |
| | | | | | | | | P6 |
| NRCoV2- | GFTL | 9 | ISRSGTTT | 53 | AADYQYST | 99 | S1-RBD | P4, |
| 08² | DDYA | | | | YCLGYDAH | | | P6 |
| | | | | | YEY | | | |
| NRCoV2- | GNTF | 10 | ISSRGIS | 54 | YAADDLGD | 100 | S1-RBD | P1, |
| 10¹ | SRSN | | | | Y | | | P3, |
| | | | | | | | | P4 |
| NRCoV2- | GSSL | 11 | ISRYYSST | 55 | AARSRDFS | 101 | S1-RBD | P1, |
| 11¹ | DSYS | | | | SPFSATDT | | | P5 |
| | | | | | YTS | | | |
| NRCoV2- | GFTL | 12 | ISRYYEST | 56 | AARSRDFS | 102 | S1-RBD | P5 |
| 11a² | DSYN | | | | SPISATDK | | | |
| | | | | | YGS | | | |
| NRCoV2- | GRTF | 13 | VAAISWGG | 57 | AADRGLSY | 103 | S1-RBD | P1, |
| 12² | RNYV | | TEI | | YYTRTTEY | | | P4, |
| | | | | | NY | | | P6 |
| NRCoV2- | GTTF | 14 | ISVFGST | 58 | HAVNADIG | 104 | S1-RBD | P3 |
| 14¹ | SHYA | | | | GDY | | | |
| NRCoV2- | GSTS | 15 | VSTSGAT | 59 | YAAYGGGG | 105 | S1-RBD | P1, |
| 15¹ | GRNT | | | | DY | | | P3, |
| | | | | | | | | P4, |
| | | | | | | | | P6 |
| NRCoV2- | GSPF | 16 | ISPTGNR | 60 | QAANVNGG | 106 | S1-RBD | P4 |
| 17¹ | SQLA | | | | DY | | | |
| NRCoV2- | GITI | 17 | INSGGST | 61 | SLHTSHDY | 107 | S1-RBD | P1, |
| 18¹ | SGYN | | | | | | | P3, |
| | | | | | | | | P4, |
| | | | | | | | | P5, |
| | | | | | | | | P6 |
| NRCoV2- | GLTL | 18 | LTSGGTG | 62 | AADRARLR | 108 | S1-RBD | P1, |
| 19² | NSYA | | | | FGCSLNFR | | | P4 |
| | | | | | REVAYDY | | | |
| NRCoV2- | GRTF | 19 | VAVISGSD | 63 | AADRGMSY | 109 | S1-RBD | P4 |
| 20¹ | SNYV | | TET | | YYTRATEY | | | |
| | | | | | YY | | | |
| NRCoV2- | GFTL | 20 | ISSGGST | 64 | AADHRGRS | 110 | S1-RBD | P1, |
| 21² | DYYA | | | | LRFGCSSS | | | P4, |
| | | | | | TTDYLY | | | P5 |
| NRCoV2- | GFTF | 21 | ISGNGGVT | 65 | AATGIRST | 111 | S1-NTD | P3, |
| SR01³ | DNYA | | | | WSVYGCSR | | | P5 |
| | | | | | LAGPYDY | | | |
| NRCoV2- | EFTL | 22 | IRYSGGGI | 66 | AADRLYSR | 112 | S1-NTD | P5 |
| SR02³ | NYYS | | | | ACPTAGGR | | | |
| | | | | | NY | | | |
| NRCoV2- | GSIF | 23 | ISSGGKT | 67 | NRGGWEYR | 113 | S1-NTD | P3, |
| SR03³ | SNNH | | | | SSYYIMGP | | | P5 |
| | | | | | H | | | |
| NRCoV2- | GRTF | 24 | ISMGGNTN | 68 | NTAALVGN | 114 | S1-NTD | P5 |
| SR04³ | SSHT | | YA | | RLLPMATI | | | |
| | | | | | T | | | |
| NRCoV2- | GSRF | 25 | ISSGGST | 69 | NMGGWDYR | 115 | S1-NTD | P3, |
| SR13³ | GSKH | | | | SNTYIPGS | | | P5 |
| | | | | | RSDY | | | |
| NRCoV2- | GTTF | 26 | ISTSGAV | 70 | NTGGWDYR | 116 | S1-NTD | P3, |
| SR16³ | SRYH | | | | SSTFIMGL | | | P5 |
| | | | | | N | | | |
| NRCoV2- | GRPY | 27 | KQRELVAA | 71 | NTGSLSYG | 117 | S2 | P2 |
| S2A3¹ | SNYA | | ISSGGTT | | GSVYYPSY | | | |
| | | | | | DN | | | |
| NRCoV2- | GSPF | 28 | ISTGGSR | 72 | HAAARDSH | 118 | S2 | P2 |
| S2A4¹ | RSNV | | | | GIYLLDT | | | |
| NRCoV2- | ASTF | 29 | ISTGSNT | 73 | NYRSIYYG | 119 | S2 | P2 |
| S2B3² | GDSA | | | | QNF | | | |
| NRCoV2- | GFTF | 30 | INSGDRDS | 74 | ALVFGYTS | 120 | S2 | P2 |
| S2H4² | NLYS | | TT | | RDYCLTPK | | | |
| | | | | | RGNY | | | |
| NRCoV2- | VRIL | 31 | ITSGGST | 75 | NLRDILSQ | 121 | S2 | P2 |
| S2F3¹ | SVPA | | | | PF | | | |
| NRCoV2- | GSTF | 32 | ITSGGAT | 76 | YTTKRDDA | 122 | S2 | P2 |
| S2G3¹ | GIFL | | | | SVY | | | |
| NRCoV2- | GSTF | 33 | ISSDGDK | 77 | NKHWWTGD | 123 | S2 | P2 |
| S2G4¹ | SGYA | | | | W | | | |
| NRCoV2- | GITV | 34 | ISAGGST | 78 | NYGPGYRK | 124 | S2 | P2 |
| S202² | SRIG | | | | AA | | | |

Subunit/domain specificities were determined by ¹SPR, ²ELISA or ³both.
SPR assays and ELISAs were perfomed against various spike protein subunit/domains using V_{H}Hs and V_{H}H-Fcs, respectively.

TABLE 7
CDR-IMGT sequences of anti-spike protein V_{H}Hs
obtained from Maple Red V_{H}H phage display
library

| | | | | | | | Sub- | |
|---|---|---|---|---|---|---|---|---|
| | | | | | | | unit/ | |
| | | | | | | | do- | Selec- |
| | | SEQ | | SEQ | | SEQ | main/ | tion |
| | CDR | ID | | ID | | ID | speci- | strat- |
| V_{H}H | 1 | NO: | CDR 2 | NO: | CDR 3 | NO: | ficity | egy |
| NRCoV2- | GNIF | 35 | IWSDS | 79 | AADRGFVV | 125 | S1-RBD | P1, |
| MRed02² | SINS | | RT | | RGQYDY | | | P3, |
| | | | | | | | | P4, |
| | | | | | | | | P6 |
| NRCoV2- | GNSF | 36 | IWSDT | 80 | AADRGFVV | 125 | S1-RBD | P4, |
| MRed04¹ | SINT | | TT | | RGQYDY | | | P6 |
| NRCoV2- | GFTL | 20 | ISSSD | 81 | ATDAFATC | 126 | S1-NTD | P3, |
| MRed03³ | DYYA | | GST | | DSWYAQIA | | | P5 |
| | | | | | QYDF | | | |
| NRCoV2- | GFTL | 20 | ISSSD | 81 | ATGPQAYY | 127 | S1-RBD | P4, |
| MRed05² | DYYA | | GST | | SGSYYFQC | | | P6 |
| | | | | | PQAGMDY | | | |
| NRCoV2- | GFTL | 37 | ISSSD | 82 | ATDSFSSC | 128 | S1-NTD | P3 |
| MRed06³ | AYYA | | GSA | | SDYESGMD | | | |
| | | | | | F | | | |
| NRCoV2- | GSIG | 38 | ITRGG | 83 | YANYGWAI | 129 | S1-NTD | P3 |
| MRed07³ | PFNT | | VT | | PY | | | |
| NRCoV2- | GFTF | 39 | INSGG | 84 | ATTISDGS | 130 | S2 | P1, |
| MRed11¹ | SSYA | | GST | | SWSTKSY | | | P2 |
| NRCoV2- | TTVF | 40 | VSDGG | 85 | NYYNYYYG | 131 | S2 | P2 |
| MRed18¹ | GRNA | | TP | | RNF | | | |
| NRCoV2- | TIIF | 41 | MTTSG | 86 | YMHSVYYG | 132 | S2 | P2 |
| MRed19¹ | KGQT | | SA | | IDY | | | |
| NRCoV2- | GLSF | 42 | IRESG | 87 | AAKPPFYG | 133 | S2 | P2 |
| MRed20¹ | SSYD | | SGT | | SGTYSTPR | | | |
| | | | | | AYLY | | | |
| NRCoV2- | GSVF | 43 | ISSRG | 88 | NAREFTGF | 134 | S2 | P2 |
| MRed22¹ | ASNA | | ST | | DY | | | |
| NRCoV2- | GHTF | 44 | ISWRG | 89 | AAEMWGTA | 135 | S2 | P2 |
| MRed25¹ | SRYG | | DST | | TIVASRYT | | | |
| | | | | | Y | | | |

Domain/subunit specificities were determined by ¹SPR, ²ELISA or ³both.
SPR assays and ELISAs were performed against various spike protein domains/subunit using V_{H}Hs and V_{H}H-Fcs, respectively.

### Example 5: Binding Characteristics of V_{H}Hs and V_{H}H-Fcs: Surface Plasmon Resonance (SPR) and ELISA Binding Studies

### Introduction

Binding of anti-SARS-CoV-2 V_{H}Hs against various SARS-CoV-2 spike protein fragments (Wuhan) was assayed using SPR and ELISA to determine their affinity and domain/sub-domain specificity. Binding of V_{H}Hs against SARS-CoV, SARS-CoV-2 UK (Alpha) variant and SARS-CoV-2 South African (Beta) variant spike protein S was also carried out to determine their virus cross-reactivity patterns.

### Materials and Methods

### Affinity/Specificity Detennination of V_{H}Hs Against SARS-CoV Spike (S), SARS-CoV-2 Spike (S) and SARS-CoV-2 Spike Fragments by SPR

Standard SPR techniques were used for binding studies. All SPR assays were performed on a Biacore™ T200 instrument (Cytiva) at 25° C. with HBS-EP running buffer (10 mM HEPES, 150 mM NaCl, 3 mM EDTA, 0.005% [v/v] Tween® 20, pH 7.4) and CM5 sensor chips (Cytiva). Prior to SPR analyses all analytes in flow (V_{H}Hs, ACE2 receptor) were SEC-purified on a Superdex™ 75 Increase 10/300 GL column (Cytiva) in HBS-EP buffer at a flow rate of 0.8 mL/min to obtain monomeric proteins. SARS-CoV spike (S), SARS-CoV-2 spike trimer (S) and various SARS-CoV-2 spike fragments were immobilized on CM5 sensor chips through standard amine coupling (10 mM acetate buffer, pH 4.0; Cytiva). On the first sensor chip, 1983 response units (RUs) of SARS-CoV spike (Sino Biologicals, Cat #40634-V08B), 843 RUs of SARS-CoV-2 S1-RBD fused to human Fc (S1-RBD-Fc) and 972 RUs of EGFR (irrelevant control surface) were immobilized. On a second sensor chip, 2346 RUs of SARS-CoV-2 S, 1141 RUs of SARS-CoV-2 S1 subunit and 1028 RUs of SARS-CoV-2 S2 subunit were immobilized. The theoretical maximum binding response for V_{H}Hs binding to these surfaces ranged from 224-262 RUs. An ethanolamine blocked surface on each sensor chip served as a reference. Single cycle kinetics was used to determine V_{H}H and ACE2 binding kinetics and affinities. V_{H}Hs at various concentration ranges (from 0.25-4 nM to 125-2000 nM) were flowed over all surfaces at a flow rate of 40 μL/min with 180 s of contact time and 600 s of dissociation time. Surfaces were regenerated with a 12 s pulse of 10 mM glycine, pH 1.5, at a flow rate of 100 μL/min. Injection of EGFR-specific V_{H}H EK2 served as a negative control for the SARS-CoV and SARS-CoV-2 surfaces and as a positive control for the EGFR surface. The ACE2 affinity was determined using similar conditions by flowing a range of monomeric ACE2 concentrations (31.53-500 nM). All affinities were calculated by fitting reference flow cell-subtracted data to a 1:1 interaction model using BIA evaluation Software v3.0 (Cytiva).

For V_{H}H 12 and MRed05, V_{H}H-Fc formats were used in SPR experiments. Approximately 200 RUs of V_{H}H-Fcs (2 μg/mL) were captured on goat anti-human IgG surfaces (4000 RUs, Jackson ImmunoResearch, Cat #109-005-098) at a flow rate of 10 μL/min for 30 s. A range of SEC-purified RBD fragments (Table 1; SARS-CoV, Wuhan, Alpha and Beta) at 0.62-10 nM were flowed over the captured V_{H}H-Fc at a flow rate of 40 μL/min with 180 s of contact time and 300 s of dissociation. Surfaces were regenerated with a 120 s pulse of 10 mM glycine, pH 1.5, at a flow rate of 50 μL/min. Affinities were calculated from reference flow cell subtracted sensorgrams as described above.

### Domain Specificity Determination of V_{H}Hs by ELISA.

V_{H}Hs that bound to the S1 subunit but not its S1-RBD domain in SPR assays, were further examined by ELISA to determine if they were binding to the S1-NTD domain of S1. Briefly, S, S1, S1-NTD and S1-RBD were coated onto NUNC® MaxiSorp™ 4BX plates (Thermo Fisher) at 100 ng/well in 100 μL PBS, pH 7.4. The next day, plates were blocked with 200 μL PBSC for 1 h at room temperature, then washed five times with PBST and incubated with fixed (13 nM) or decreasing concentrations of V_{H}H-Fcs diluted in PBSTC. After 1 h, plates were washed 10 times with PBSTC and binding of V_{H}H-Fc fusions was detected by incubating wells with 100 μL of 1 μg/mL HRP-conjugated goat anti-human IgG. Finally, plates were washed 10 times with PBST and peroxidase activity was determined as described above. EC₅₀s for the binding of V_{H}H-Fcs to S and S fragments were obtained from the plot of A450. (binding) vs V_{H}H-Fc concentration. S1-NTD covering amino acids 16-305 of SARS-CoV-2 S (GenBank accession number: QHD43416.1) was expressed in CHO cells.

Affinity/Specificity Determination of V_{H}Hs Against Spike Protein S from SARS-CoV-2 Wuhan, UK (Alpha) and South African (Beta) Variants by SPR

Affinity and specificity of V_{H}Hs against spike protein S from SARS-CoV-2 Wuhan, UK and South African variants by SPR was determined essentially as described above.

### Results and Discussion

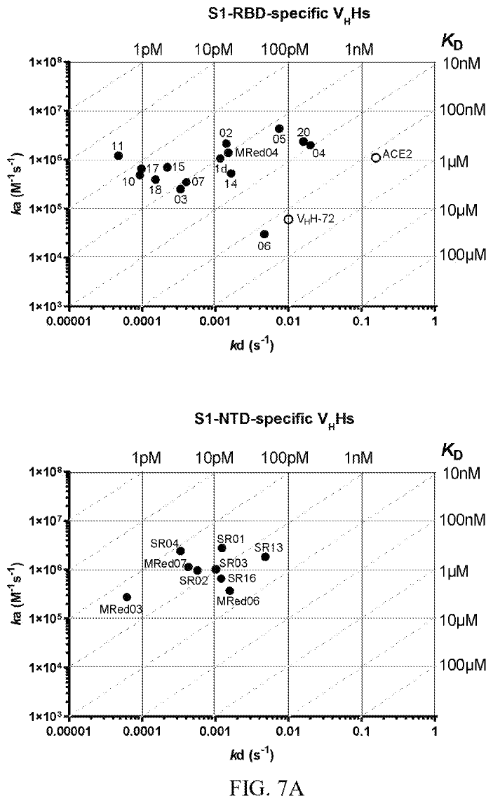

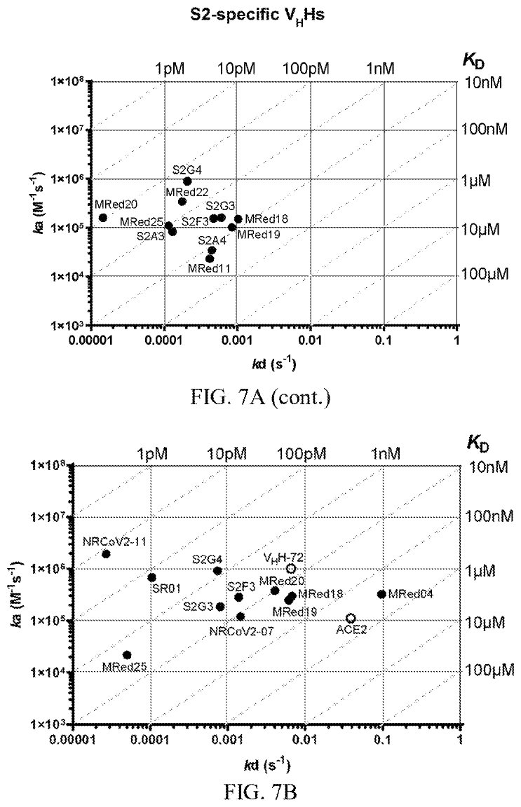

V_{H}Hs were tested by SPR against SARS-CoV-2 S, S1, S1-RBD and S2 to determine their affinity and domain/sub-domain specificity. Binding data are presented in FIG. 6C, FIGS. 7A-B, Table 8 and Table 9. In SPR binding assays, NRCoV2-5R01, NRCoV2-SR02, NRCoV2-SR03, NRCoV2-SR04, NRCoV2-SR13, NRCoV2-SR16, NRCoV2-MRed03, NRCoV2-MRed06 and NRCoV2-MRed07 bound to the S1 subunit but not to its S1-RBD domain. Subsequent ELISAs performed against SARS-CoV-2 S, S1, S1-NTD and S1-RBD showed these V_{H}Hs were S1-NTD-specific (FIGS. 6D and 6E and Table 10). V_{H}Hs displayed high affinity towards their target (i.e., S) with the vast majority having Kos in the range of single-digit-nM to pM. Three clusters of V_{H}Hs based on domain/subdomain specificity were identified: (i) S1-RBD-specific V_{H}Hs; (ii) S1-NTD-specific V_{H}Hs; and (iii) S2-specific V_{H}Hs (FIG. 7A).

As for the S1-RBD-specific V_{H}Hs, with the exception of NRCoV2-06, which had an affinity of 223 nM (Table 11), the remaining 16 cluster members displayed high affinities ranging from 0.02-10 nM, all vastly outperforming the benchmark V_{H}H-72 V_{H}H, which had a K_{D} of 86.2 nM. Nine V_{H}Hs were S1-NTD-specific and, similar to S1-RBD-specific V_{H}Hs, displayed high affinities (K_{D}s) in the range of 0.1-5.2 nM. Lastly, 11 V_{H}Hs were S2 subunit-specific, with similarly high affinities (K_{D}s) ranging from 0.09-12.8 nM.

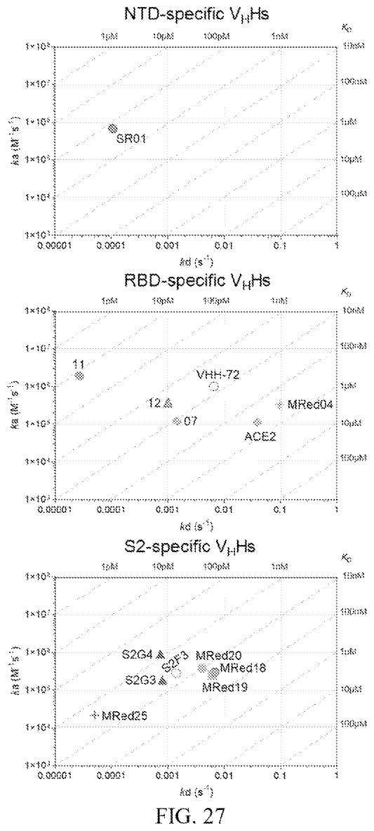

V_{H}Hs were tested against SARS-CoV (S) in SPR assays for quantitative determination of cross-reactivity. V_{H}Hs were first screened for cross-reactivity at fixed concentrations. Twelve out of 37 V_{H}Hs screened showed cross-reactivity to SARS-CoV S. These 12 V_{H}Hs were subsequently subjected to comprehensive binding analysis against both SARS-CoV S and SARS-CoV-2 S at multiple V_{H}H concentrations. The SPR cross-reactivity results, which agreed with those from ELISAs, are presented in FIG. 27 and Table 11. Seven out of the 12 V_{H}Hs tested were S2-specific, four were S1-RBD-specific and one was S1-NTD-specific. NRCoV2-MRed04 showed weak binding to SARS-CoV S compared to SARS-CoV-2 S (300 nM for SARS-CoV S vs 1 nM for SARS-CoV-2 S), but the remaining V_{H}Hs cross-reacted with high/comparable affinities to both SARS-CoV-2 S and SARS-CoV S. NRCoV2-07, NRCoV2-12, NRCoV2-MRed18, NRCoV2-MRed19 and NRCoV2-MRed20 cross-reacted with SARS-CoV S with relatively lower affinities in comparison to SARS-CoV-2 S, but nonetheless with high absolute affinities in the low nanomolar K_{D} range. The S1-NTD-specific V_{H}H, NRCoV2-SR01, cross-reacted with SARS-CoV S with high affinity (0.15 nM for SARS-CoV S vs 0.56 nM for SARS-CoV-2 S); one S1-RBD-specific V_{H}H, NRCoV2-11, cross-reacted with SARS-CoV S with very high affinity (0.014 nM for SARS-CoV S vs 0.018 nM for SARS-CoV-2 S); and four S2-specific V_{H}Hs demonstrated high, comparable affinities to SARS-CoV and SARS-CoV-2 S in the single-digit-nM to pM K_{D} range.

Against the Alpha and Beta variants, SPR cross-reactivity data performed with 37 V_{H}Hs, were consistent with ELISA, except for NRCoV2-04 and NRCoV2-14 which were negative or very weak for binding to the Beta variant by SPR. All 37 V_{H}Hs tested bound the Alpha variant S protein, 34 of which were also cross-reactive to the Beta variant S protein (FIG. 28A, FIG. 28B and Table 12). Thirteen out of 17 RBD-specific V_{H}Hs bound all three variants with similar affinities, except for V_{H}Hs NRCoV2-10, NRCoV2-15 and NRCoV2-17 which bound to the Beta variant with 40-50-fold weaker affinity; the remaining four that did not bind the Beta variant showed cross-reactivity with the Alpha variant with similar (NRCoV2-04, NRCoV2-14) or reduced (˜5-fold [NRCoV2-05] and ˜20-fold [NRCoV2-02]) affinity relative to the Wuhan variant. All NTD-specific and S2-specific V_{H}Hs cross-reacted with the three variants with essentially the same or similar affinities.

TABLE 8
Kinetic and equilibrium constants for the binding of Eva
Green V_{H}Hs to SARS-CoV-2 Wuhan spike protein fragments

| | S1-RBD-Fc¹ | | | S¹ | | |
|---|---|---|---|---|---|---|
| V_{H}H/ | kₐ | k_{d} | K_{D} | kₐ | k_{d} | K_{D} |
| ACE2 | (1/Ms) | (1/s) | (M) | (1/Ms) | (1/s) | (M) |
| NRCoV2-1d | 5.62E+05 | 1.18E−03 | 2.10E−09 | 1.06E+06 | 1.17E−03 | 1.10E−09 |
| NRCoV2-02 | 1.83E+06 | 1.41E−03 | 7.73E−10 | 2.14E+06 | 1.41E−03 | 6.61E−10 |
| NRCoV2-03 | 1.66E+05 | 2.55E−04 | 1.53E−09 | 2.50E+05 | 3.34E−04 | 1.34E−09 |
| NRCoV2-04 | 1.53E+06 | 1.92E−02 | 1.25E−08 | 1.97E+06 | 1.98E−02 | 1.00E−08 |
| NRCoV2-05 | 2.51E+06 | 5.85E−03 | 2.33E−09 | 4.33E+06 | 7.43E−03 | 1.72E−09 |
| NRCoV2-06 | 5.94E+04 | 4.15E−03 | 6.99E−08 | 3.02E+04 | 4.69E−03 | 1.55E−07 |
| NRCoV2-07 | 3.18E+05 | 2.84E−04 | 8.94E−10 | 3.50E+05 | 4.03E−04 | 1.15E−09 |
| NRCoV2-10 | 3.51E+05 | 9.32E−05 | 2.66E−10 | 4.83E+05 | 9.27E−05 | 1.92E−10 |
| NRCoV2-11 | 8.92E+05 | 2.26E−04 | 2.53E−10 | 1.21E+06 | 4.73E−05 | 3.91E−11 |
| NRCoV2-12 | 7.60E+05 | 3.69E−05 | 4.86E−11 | nd | | |
| NRCoV2-14 | 2.61E+05 | 9.44E−04 | 3.61E−09 | 5.28E+05 | 1.64E−03 | 3.10E−09 |
| NRCoV2-15 | 6.82E+05 | 2.33E−04 | 3.42E−10 | 7.06E+05 | 2.21E−04 | 3.13E−10 |
| NRCoV2-17 | 5.67E+05 | 2.24E−04 | 3.95E−10 | 6.59E+05 | 9.79E−05 | 1.49E−10 |
| NRCoV2-18 | 2.68E+05 | 1.97E−04 | 7.36E−10 | 3.95E+05 | 1.52E−04 | 3.84E−10 |
| NRCoV2-20 | 1.43E+06 | 1.23E−02 | 8.61E−09 | 2.37E+06 | 1.59E−02 | 6.73E−09 |
| NRCoV2-SR01 | − | − | − | 2.77E+06 | 1.23E−03 | 4.45E−10 |
| NRCoV2-SR02 | − | − | − | 9.67E+05 | 5.71E−04 | 5.90E−10 |
| NRCoV2-SR03 | − | − | − | 1.01E+06 | 1.02E−03 | 1.01E−09 |
| NRCoV2-SR04 | − | − | − | 2.39E+06 | 3.35E−04 | 1.40E−10 |
| NRCoV2-SR13 | − | − | − | 1.83E+06 | 4.81E−03 | 2.62E−09 |
| NRCoV2-SR16 | − | − | − | 6.57E+05 | 1.20E−03 | 1.82E−09 |
| NRCoV2-S2A3 | − | − | − | 8.40E+04 | 1.30E−04 | 1.55E−09 |
| NRCoV2-S2A4 | − | − | − | 3.49E+04 | 4.46E−04 | 1.28E−08 |
| NRCoV2-S2G3 | − | − | − | 1.62E+05 | 6.07E−04 | 3.74E−09 |
| NRCoV2-S2G4 | − | − | − | 8.93E+05 | 2.07E−04 | 2.32E−10 |
| NRCoV2-S2F3 | − | − | − | 1.56E+05 | 4.73E−04 | 3.03E−09 |
| V_{H}H-72² | 6.67E+05 | 1.34E−01 | 2.00E−07 | 1.10E+06 | 1.56E−01 | 1.42E−07 |
| ACE2-H6² | 3.71E+04 | 1.18E−02 | 3.17E−07 | 6.02E+04 | 9.96E−03 | 1.65E−07 |
| NRCsdAb022² | − | − | − | − | − | − |
| | S1¹ | | | S2¹ | | |
| V_{H}H/ | kₐ | k_{d} | K_{D} | kₐ | k_{d} | K_{D} |
| ACE2 | (1/Ms) | (1/s) | (M) | (1/Ms) | (1/s) | (M) |
| NRCoV2-1d | 8.67E+05 | 1.14E−03 | 1.32E−09 | − | − | − |
| NRCoV2-02 | 2.10E+06 | 1.38E−03 | 6.59E−10 | − | − | − |
| NRCoV2-03 | 2.41E+05 | 2.63E−04 | 1.09E−09 | − | − | − |
| NRCoV2-04 | 1.61E+06 | 1.80E−02 | 1.12E−08 | − | − | − |
| NRCoV2-05 | 4.03E+06 | 8.06E−03 | 2.00E−09 | − | − | − |
| NRCoV2-06 | 1.04E+05 | 1.33E−02 | 1.29E−07 | − | − | − |
| NRCoV2-07 | 3.01E+05 | 3.08E−04 | 1.02E−09 | − | − | − |
| NRCoV2-10 | 4.48E+05 | 9.54E−05 | 2.13E−10 | − | − | − |
| NRCoV2-11 | 1.08E+06 | 1.80E−04 | 1.67E−10 | − | − | − |
| NRCoV2-12 | nd | | | − | − | |
| NRCoV2-14 | 3.19E+05 | 1.12E−03 | 3.49E−09 | − | − | − |
| NRCoV2-15 | 6.75E+05 | 2.25E−04 | 3.33E−10 | − | − | − |
| NRCoV2-17 | 6.29E+05 | 9.66E−05 | 1.53E−10 | − | − | − |
| NRCoV2-18 | 3.08E+05 | 1.65E−04 | 5.36E−10 | − | − | − |
| NRCoV2-20 | 1.96E+06 | 1.89E−02 | 9.63E−09 | − | − | − |
| NRCoV2-SR01 | 3.63E+06 | 1.58E−03 | 4.37E−10 | − | − | − |
| NRCoV2-SR02 | 1.05E+06 | 5.55E−04 | 5.30E−10 | − | − | − |
| NRCoV2-SR03 | 9.05E+05 | 1.03E−03 | 1.13E−09 | − | − | − |
| NRCoV2-SR04 | 2.18E+06 | 4.93E−04 | 2.26E−10 | − | − | − |
| NRCoV2-SR13 | 2.25E+06 | 5.45E−03 | 2.43E−09 | − | − | − |
| NRCoV2-SR16 | 7.39E+05 | 1.15E−03 | 1.55E−09 | − | − | − |
| NRCoV2-S2A3 | − | − | − | 4.60E+04 | 1.03E−04 | 2.23E−09 |
| NRCoV2-S2A4 | − | − | − | 2.81E+04 | 4.14E−04 | 1.47E−08 |
| NRCoV2-S2G3 | − | − | − | 1.47E+05 | 6.27E−04 | 4.28E−09 |
| NRCoV2-S2G4 | − | − | − | 9.25E+05 | 3.82E−04 | 4.13E−10 |
| NRCoV2-S2F3 | − | − | − | 1.01E+05 | 6.29E−04 | 6.22E−09 |
| V_{H}H-72² | 9.40E+05 | 1.46E−01 | 1.56E−07 | − | − | − |
| ACE2-H6² | 6.21E+04 | 1.24E−02 | 2.00E−07 | − | − | − |
| NRCsdAb022² | − | − | − | − | − | − |

¹For any given V_{H}H, K_{D} values across different spike fragments, S1-RBD-Fc, S1, S2 and S were in agreement. Lack of V_{H}H binding for certain spike fragments is consistent with V_{H}Hs' subunit/domain specificities. Binding parameters were determined by flowing monomeric V_{H}Hs over sensorchip surfaces coated with various spike fragments, except for binding parameters for NRCoV2-12 which were obtained by flowing monomeric RBDs over V_{H}H-Fc-captured surfaces. Dashes indicate lack of binding. “nd”, not done,
²V_{H}H-72 (Wrapp et al., 2020) and ACE2-H6 are positive binder controls, EGFR-specific V_{H}H, NRCsdAb022 (Rossotti et al., 2019) is a negative control.

TABLE 9
Kinetic and equilibrium dissociation constants for the binding of
Maple Red V_{H}Hs to various SARS-CoV-2 Wuhan spike protein fragments

| | S1-RBD-Fc¹ | | | S¹ | | |
|---|---|---|---|---|---|---|
| V_{H}H/ | ka | kd | KD | ka | kd | KD |
| ACE2¹ | (1/Ms) | (1/s) | (M) | (1/Ms) | (1/s) | (M) |
| NRCoV2-MRed03 | − | − | − | 1.57E+05 | 8.14E−05 | 5.20E−10 |
| NRCoV2-MRed04 | 8.55E+05 | 1.76E−03 | 2.06E−09 | 1.39E+06 | 1.50E−03 | 1.09E−09 |
| NRCoV2-MRed05 | 6.50E+05 | 4.96E−04 | 7.63E−10 | nd | | |
| NRCoV2-MRed06 | − | − | − | 1.85E+05 | 8.87E−04 | 4.80E−09 |
| NRCoV2-MRed07 | − | − | − | 1.60E+06 | 3.78E−04 | 2.36E−10 |
| NRCoV2-MRed11 | − | − | − | 2.34E+04 | 4.18E−04 | 1.78E−08 |
| NRCoV2-MRed18 | − | − | − | 2.02E+05 | 1.53E−03 | 7.56E−09 |
| NRCoV2-MRed19 | − | − | − | 1.59E+05 | 7.99E−04 | 5.01E−09 |
| NRCoV2-MRed20 | − | − | − | 1.60E+05 | 1.46E−05 | 9.18E−11 |
| NRCoV2-MRed22 | − | − | − | 3.47E+05 | 1.76E−04 | 5.06E−10 |
| NRCoV2-MRed25 | − | − | − | 1.12E+05 | 1.15E−04 | 1.02E−09 |
| V_{H}H-72² | 6.82E+05 | 1.26E−01 | 1.85E−07 | 1.06E+06 | 1.50E−01 | 1.42E−07 |
| ACE2-H6² | 3.86E+04 | 1.06E−02 | 2.74E−07 | 8.63E+04 | 1.18E−02 | 1.37E−07 |
| NRCsdAb022² | − | − | − | − | − | − |
| | S1¹ | | | S2¹ | | |
| V_{H}H/ | ka | kd | KD | ka | kd | KD |
| ACE2¹ | (1/Ms) | (1/s) | (M) | (1/Ms) | (1/s) | (M) |
| NRCoV2-MRed03 | 2.76E+05 | 6.15E−05 | 2.23E−10 | − | − | − |
| NRCoV2-MRed04 | 1.09E+06 | 1.83E−03 | 1.68E−09 | − | − | − |
| NRCoV2-MRed05 | nd | | | nd | | |
| NRCoV2-MRed06 | 3.71E+05 | 1.57E−03 | 4.22E−09 | − | − | − |
| NRCoV2-MRed07 | 1.13E+06 | 4.27E−04 | 3.78E−10 | − | − | − |
| NRCoV2-MRed11 | − | − | − | 4.09E+04 | 2.13E−04 | 5.21E−09 |
| NRCoV2-MRed18 | − | − | − | 1.52E+05 | 1.03E−03 | 6.80E−09 |
| NRCoV2-MRed19 | − | − | − | 1.02E+05 | 8.40E−04 | 8.20E−09 |
| NRCoV2-MRed20 | − | − | − | 1.13E+05 | 3.38E−05 | 2.99E−10 |
| NRCoV2-MRed22 | − | − | − | 2.70E+05 | 2.84E−04 | 1.05E−09 |
| NRCoV2-MRed25 | − | − | − | 1.22E+05 | 1.90E−04 | 1.56E−09 |
| V_{H}H-72² | 8.30E+05 | 1.61E−01 | 1.94E−07 | − | − | − |
| ACE2-H6² | 7.29E+04 | 1.32E−02 | 1.82E−07 | − | − | − |
| NRCsdAb022² | − | − | − | − | − | − |

¹For any given V_{H}H, K_{D} values across different spike fragments, S1-RBD-Fc, S1, S2 and S were in agreement. Lack of V_{H}H binding for certain spike fragments was consistent with V_{H}Hs' subunit/domain specificities. Binding parameters were determined by flowing monomeric V_{H}Hs over sensorchip surfaces coated with various spike fragments, except for NRCoV2-MRed05, for which binding parameters were obtained by flowing monomeric RBDs over V_{H}H-Fc-captured surfaces. Dashes indicate lack of binding, “nd”, not done.
²V_{H}H-72 (Wrapp et al., 2020) and ACE2-H6 are positive binder controls, EGFR-specific V_{H}H, NRCsdAb022 (Rossotti et al., 2019) is a negative control.

TABLE 10
ELISA data for the binding of V_{H}H-Fcs
to various spike protein fragments

| | EC₅₀app (nM)² | | | | Subdomain |
|---|---|---|---|---|---|
| V_{H}H-Fc¹ | S | S1 | S1-NTD | S1-RBD | specificity |
| NRCoV2-SR01 | 0.13 | 0.23 | 0.19 | — | S1-NTD |
| NRCoV2-SR02 | 0.11 | 0.13 | 0.34 | — | S1-NTD |
| NRCoV2-SR03 | 0.16 | 0.20 | 0.17 | — | S1-NTD |
| NRCoV2-SR04 | 0.20 | 0.25 | 0.17 | — | S1-NTD |
| NRCoV2-SR13 | 0.43 | 0.43 | 0.23 | — | S1-NTD |
| NRCoV2-SR16 | 0.59 | 2.70 | 0.36 | — | S1-NTD |
| NRCoV2-MRed03 | 0.20 | 0.48 | 0.70 | — | S1-NTD |
| NRCoV2-MRed06 | 0.40 | 0.59 | 0.40 | — | S1-NTD |
| NRCoV2-MRed07 | 1.20 | 1.50 | 0.80 | — | S1-NTD |
| NRCoV2-02 | 0.10 | 0.11 | — | 0.21 | S1-RBD |

¹S1-specfic V_{H}Hs that did not bind to S1-RBD by SPR (Table 8 and Table 9), were tested for specificity against S1-NTD. The S1-RBD-specific NRCoV2-02 internal control gave the expected specificity binding profile.
²EC₅₀app, apparent EC₅₀.

TABLE 11
Kinetic and equilibrium dissociation constants for the
binding of V_{H}Hs to SARS-CoV-2 Wuhan, SARS-CoV-2 Alpha,
SARSCoV-2 Beta and SARS-CoV spike glycoproteins

| | SARS-CoV-2 | | | SARS-CoV-2 | | |
|---|---|---|---|---|---|---|
| | Wuhan^{a} | | | Alpha^{a} | | |
| V_{H}H/ | kₐ | k_{d} | K_{D} | kₐ | k_{d} | K_{D} |
| ACE2 | (1/Ms) | (1/s) | (M) | (1/Ms) | (1/s) | (M) |

RBD-specific V_{H}H

| | | | | | | |
|---|---|---|---|---|---|---|
| NRCoV2-1d | 1.39E+06 | 1.05E−03 | 7.50E−10 | 1.13E+06 | 1.02E−03 | 9.07E−10 |
| NRCoV2-02 | 2.04E+06 | 1.27E−03 | 6.24E−10 | 1.64E+06 | 2.22E−02 | 1.36E−08 |
| NRCoV2-03 | 1.16E+05 | 1.81E−04 | 1.56E−09 | 1.03E+05 | 1.54E−04 | 1.49E−09 |
| NRCoV2-04 | 1.84E+06 | 1.88E−02 | 1.02E−08 | 1.67E+06 | 1.96E−02 | 1.17E−08 |
| NRCoV2-05 | 2.76E+06 | 7.09E−03 | 2.57E−09 | 2.28E+06 | 2.61E−02 | 1.14E−08 |
| NRCoV2-06 | 2.05E+04 | 4.56E−03 | 2.23E−07 | 2.05E+04 | 4.68E−03 | 2.29E−07 |
| NRCoV2-07 | 3.78E+05 | 3.55E−04 | 9.39E−10 | 3.39E+05 | 3.78E−04 | 1.12E−09 |
| NRCoV2-10 | 5.81E+05 | 1.16E−04 | 1.99E−10 | 4.84E+05 | 1.02E−04 | 2.10E−10 |
| NRCoV2-11 | 9.64E+05 | 1.71E−05 | 1.77E−11 | 9.28E+05 | 1.59E−05 | 1.71E−11 |
| NRCoV2-12^{a} | 7.47E+05 | 3.50E−05 | 4.69E−11 | 9.47E+05 | 4.38E−05 | 4.63E−11 |
| NRCoV2-14 | 3.89E+05 | 1.01E−03 | 2.60E−09 | 3.75E+05 | 9.17E−04 | 2.44E−09 |
| NRCoV2-15 | 6.91E+05 | 2.22E−04 | 3.21E−10 | 6.37E+05 | 1.94E−04 | 3.05E−10 |
| NRCoV2-17 | 6.14E+05 | 9.45E−05 | 1.54E−10 | 6.14E+05 | 7.64E−05 | 1.25E−10 |
| NRCoV2-18 | 2.95E+05 | 9.39E−05 | 3.18E−10 | 2.78E+05 | 9.79E−05 | 3.53E−10 |
| NRCoV2-20 | 2.64E+06 | 1.16E−02 | 4.39E−09 | 2.48E+06 | 1.23E−02 | 4.97E−09 |
| NRCoV2- | 1.51E+06 | 1.31E−03 | 8.63E−10 | 1.34E+06 | 1.22E−03 | 9.09E−10 |
| MRed04 | | | | | | |
| NRCoV2- | 5.62E+05 | 5.09E−04 | 9.05E−10 | 5.73E+05 | 1.76E−04 | 3.07E−10 |
| MRed05^{a} | | | | | | |

NTD-specific V_{H}H

| | | | | | | |
|---|---|---|---|---|---|---|
| NRCoV2-SR01 | 1.43E+06 | 8.05E−04 | 5.64E−10 | 1.01E+06 | 5.94E−04 | 5.91E−10 |
| NRCoV2-SR02 | 4.98E+06 | 6.69E−04 | 1.35E−10 | 4.54E+06 | 2.54E−04 | 5.59E−11 |
| NRCoV2-SR03 | 6.70E+05 | 1.13E−03 | 1.69E−09 | 5.55E+05 | 9.55E−04 | 1.72E−09 |
| NRCoV2-SR04 | 3.68E+06 | 5.15E−04 | 1.40E−10 | 2.80E+06 | 7.50E−04 | 2.67E−10 |
| NRCoV2-SR13 | 1.06E+06 | 3.76E−03 | 3.56E−09 | 5.92E+05 | 3.44E−03 | 5.82E−09 |
| NRCoV2-SR16 | 4.88E+05 | 9.57E−04 | 1.96E−09 | 4.04E+05 | 6.33E−04 | 1.57E−09 |
| NRCoV2- | 2.37E+05 | 1.21E−04 | 5.08E−10 | 2.71E+05 | 9.89E−05 | 3.64E−10 |
| MRed03 | | | | | | |
| NRCoV2- | 1.92E+05 | 9.96E−04 | 5.19E−09 | 2.27E+05 | 1.30E−03 | 5.72E−09 |
| MRed06 | | | | | | |
| NRCoV2- | 4.58E+06 | 4.81E−04 | 1.05E−10 | 3.90E+06 | 1.03E−03 | 2.64E−10 |
| MRed07 | | | | | | |

S2-specific V_{H}H

| | | | | | | |
|---|---|---|---|---|---|---|
| NRCoV2-S2A3 | 9.83E+04 | 5.51E−05 | 5.61E-10 | 8.70E+04 | 1.89E−04 | 2.18E−09 |
| NRCoV2-S2A4 | 3.49E+04 | 4.46E−04 | 1.28E−08 | 2.48E+05 | 2.36E−03 | 9.52E−09 |
| NRCoV2-S2F3 | 1.56E+05 | 4.73E−04 | 3.03E−09 | | + | |
| NRCoV2-S2G3 | 3.24E+05 | 6.06E−04 | 1.87E−09 | 2.98E+05 | 5.30E−04 | 1.78E−09 |
| NRCoV2-S2G4 | 8.93E+05 | 2.07E−04 | 2.32E−10 | 1.56E+06 | 2.92E−04 | 1.87E−10 |
| NRCoV2- | 4.57E+04 | 2.83E−04 | 6.20E−09 | 3.11E+04 | 4.26E−04 | 1.37E−08 |
| MRed11 | | | | | | |
| NRCoV2- | 1.97E+05 | 1.19E−03 | 6.03E−09 | 3.82E+05 | 4.93E−03 | 1.29E−08 |
| MRed18 | | | | | | |
| NRCoV2- | 1.31E+05 | 1.18E−03 | 9.07E−09 | 1.83E+05 | 3.70E−03 | 2.02E−08 |
| MRed19 | | | | | | |
| NRCoV2- | 1.60E+05 | 1.46E−05 | 9.18E−11 | 2.78E+05 | 1.53E−04 | 5.50E−10 |
| MRed20 | | | | | | |
| NRCoV2- | 3.47E+05 | 1.76E−04 | 5.06E−10 | 8.96E+05 | 2.20E−04 | 2.46E−10 |
| MRed22 | | | | | | |
| NRCoV2- | 1.12E+05 | 1.15E−04 | 1.02E−09 | 1.02E+06 | 2.89E−04 | 2.83E−10 |
| MRed25 | | | | | | |

Control

| ACE2-H₆^{b} | 6.38E+04 | 9.79E−03 | 1.53E−07 | 8.52E+04 | 1.56E−03 | 1.83E−08 |
|---|---|---|---|---|---|---|
| VHH-72^{b} | 1.23E+06 | 1.06E−01 | 8.62E−08 | 1.05E+06 | 1.01E−01 | 9.60E−08 |
| NRCsdAb022^{b} | − | − | − | − | − | − |
| | SARS-CoV-2 | | | | | |
| | Beta^{a} | | | SARS-CoV^{a} | | |
| V_{H}H/ | kₐ | k_{d} | K_{D} | kₐ | k_{d} | K_{D} |
| ACE2 | (1/Ms) | (1/s) | (M) | (1/Ms) | (1/s) | (M) |

RBD-specific V_{H}H

| | | | | | | |
|---|---|---|---|---|---|---|
| NRCoV2-1d | 8.12E+05 | 9.56E−04 | 1.18E−09 | − | − | − |
| NRCoV2-02 | − | − | − | − | − | − |
| NRCoV2-03 | 5.54E+04 | 2.26E−04 | 4.08E−09 | − | − | − |
| NRCoV2-04 | − | − | − | − | − | − |
| NRCoV2-05 | − | − | − | − | − | − |
| NRCoV2-06 | 2.48E+04 | 6.14E−03 | 2.48E−07 | − | − | − |
| NRCoV2-07 | 3.05E+05 | 3.21E−04 | 1.05E−09 | 1.20E+05 | 1.46E−03 | 1.22E−08 |
| NRCoV2-10 | 2.70E+05 | 2.62E−03 | 9.73E−09 | − | − | − |
| NRCoV2-11 | 7.57E+05 | 1.72E−05 | 2.27E−11 | 1.93E+06 | 2.70E−05 | 1.40E−11 |
| NRCoV2-12^{a} | 9.70E+05 | 3.89E−05 | 4.01E−11 | 3.76E+05 | 1.01E−03 | 2.69E−09 |
| NRCoV2-14 | − | − | − | − | − | − |
| NRCoV2-15 | 1.48E+05 | 3.28E−03 | 2.22E−08 | − | − | − |
| NRCoV2-17 | 1.14E+06 | 5.88E−03 | 5.14E−09 | − | − | − |
| NRCoV2-18 | 2.76E+05 | 1.01E−04 | 3.65E−10 | − | − | − |
| NRCoV2-20 | 2.08E+06 | 1.14E−02 | 5.47E−09 | − | − | − |
| NRCoV2- | 1.14E+06 | 1.23E−03 | 1.07E−09 | 3.23E+05 | 9.70E−02 | 3.00E−07 |
| MRed04 | | | | | | |
| NRCoV2- | 6.12E+05 | 5.44E−04 | 8.88E−10 | | − | |
| MRed05^{a} | | | | | | |

NTD-specific V_{H}H

| | | | | | | |
|---|---|---|---|---|---|---|
| NRCoV2-SR01 | 1.09E+06 | 2.21E−04 | 2.02E−10 | 6.81E+05 | 1.05E−04 | 1.54E−10 |
| NRCoV2-SR02 | 4.19E+06 | 6.28E−04 | 1.50E−10 | − | − | − |
| NRCoV2-SR03 | 5.64E+05 | 1.40E−03 | 2.49E−09 | − | − | − |
| NRCoV2-SR04 | 2.24E+06 | 7.23E−04 | 3.24E−10 | − | − | − |
| NRCoV2-SR13 | 5.71E+05 | 4.01E−03 | 7.02E−09 | − | − | − |
| NRCoV2-SR16 | 3.95E+05 | 1.01E−03 | 2.57E−09 | − | − | − |
| NRCoV2- | 2.13E+05 | 1.43E−04 | 6.72E−10 | − | − | − |
| MRed03 | | | | | | |
| NRCoV2- | 1.39E+05 | 1.01E−03 | 7.24E−09 | − | − | − |
| MRed06 | | | | | | |
| NRCoV2- | 2.38E+06 | 5.52E−04 | 2.31E−10 | − | − | − |
| MRed07 | | | | | | |

S2-specific V_{H}H

| | | | | | | |
|---|---|---|---|---|---|---|
| NRCoV2-S2A3 | 6.69E+04 | 5.71E−05 | 8.53E−10 | − | − | − |
| NRCoV2-S2A4 | 5.88E+4 | 8.98E−4 | 1.53E−8 | − | − | − |
| NRCoV2-S2F3 | | + | | 2.82E+05 | 1.39E−03 | 4.91E−09 |
| NRCoV2-S2G3 | 3.04E+05 | 5.63E−04 | 1.85E−09 | 1.87E+05 | 8.00E−04 | 4.27E−09 |
| NRCoV2-S2G4 | | + | | 9.20E+05 | 7.35E−04 | 7.99E−10 |
| NRCoV2- | 4.54E+04 | 2.82E−04 | 6.21E−09 | − | − | − |
| MRed11 | | | | | | |
| NRCoV2- | 3.69E+05 | 2.39E−03 | 6.48E−09 | 3.00E+05 | 6.77E−03 | 2.25E−08 |
| MRed18 | | | | | | |
| NRCoV2- | 1.29E+05 | 1.04E−03 | 8.07E−09 | 2.49E+05 | 6.14E−03 | 2.46E−08 |
| MRed19 | | | | | | |
| NRCoV2- | 1.39E+05 | 6.33E−05 | 4.55E−10 | 3.80E+05 | 4.06E−03 | 1.07E−08 |
| MRed20 | | | | | | |
| NRCoV2- | | + | | − | − | − |
| MRed22 | | | | | | |
| NRCoV2- | 2.05E+06 | 3.29E−03 | 1.60E−09 | 2.18E+04 | 5.01E−05 | 2.29E−09 |
| MRed25 | | | | | | |

Control

| | | | | | | |
|---|---|---|---|---|---|---|
| ACE2-H₆^{b} | 3.66E+04 | 4.78E−03 | 1.31E−07 | 1.11E+05 | 3.89E−02 | 3.51E−07 |
| VHH-72^{b} | 8.01E+05 | 9.92E−02 | 1.24E−07 | 1.01E+06 | 6.56E−03 | 6.52E−09 |
| NRCsdAb022^{b} | − | − | − | − | − | − |

^{a}Binding parameters were determined by flowing monomeric V_{H}Hs over sensorchip surfaces coated with S, except for V_{H}H NRCoV2-12 and MRed05, which were obtained by flowing monomeric RBDs (aa319-541 [SARS-CoV-2]; aa306-527 [SARS-CoV]) over V_{H}H-Fc-captured surfaces. Dashes indicate lack of binding. nd, not determined.
^{b}ACE2-H₆ and V_{H}H-72 (Wrapp et al., 2020), positive controls, EGFR-specific V_{H}H NRCsdAb022 (Rossotti et al., 2019) negative control.

TABLE 12
SPR affinity (K_{D}) of V_{H}Hs against trimeric spikes protein
S from the Wuhan-Hu-1 (Wuhan), UK B.1.1.7 (Alpha) and
South Africa B.1.351 (Beta) SARS-CoV-2 variants

| | Subunit/domain | K_{D} (nM) | | |
|---|---|---|---|---|
| V_{H}H/ACE2 | specificity/epitope bin | Wuhan | Alpha | Beta |
| ACE2 | S1-RBD | 153 | 18.3 | 131 |
| NRCoV2-1d | S1-RBD/Bin 1 | 0.75 | 0.91 | 1.2 |
| NRCoV2-07 | | 0.94 | 1.1 | 1.1 |
| NRCoV2-12 | | 0.047 | 0.046 | 0.04 |
| NRCoV2-18 | | 0.32 | 0.35 | 0.37 |
| NRCoV2-20 | | 4.39 | 4.97 | 5.47 |
| NRCoV2-MRed04 | | 0.86 | 0.91 | 1.07 |
| V_{H}H-72¹ | | 86.2 | 96 | 124 |
| NRCoV2-02 | S1-RBD/Bin 2, 3 | 0.62 | 13.6 | —³ |
| NRCoV2-05 | | 2.6 | 11.4 | — |
| NRCoV2-10 | S1-RBD/Bin 2, 3, 4 | 0.2 | 0.21 | 9.73 |
| NRCoV2-15 | | 0.32 | 0.31 | 22.2 |
| NRCoV2-MRed05 | | 0.91 | 0.31 | 0.89 |
| NRCoV2-14 | S1-RBD/Bin 2, 4 | 2.6 | 2.44 | weak |
| | | | | binding |
| NRCoV2-17 | S1-RBD/Bin 3, 4 | 0.15 | 0.13 | 5.1 |
| NRCoV2-04 | S1-RBD/Bin 4 | 10.2 | 11.7 | — |
| NRCoV2-06 | S1-RBD/Bin 5 | 223 | 229 | 248 |
| NRCoV2-11 | | 0.018 | 0.017 | 0.023 |
| NRCoV2-03 | S1-RBD/Bin 6 | 1.56 | 1.49 | 4.08 |
| NRCoV2-SR01 | S1-NTD/Bin 7, 9, 10 | 0.56 | 0.59 | 0.2 |
| NRCov2-SR03 | | 1.69 | 1.72 | 2.49 |
| NRCoV2-SR13 | | 3.6 | 5.8 | 7 |
| NRCoV2-SR16 | | 2 | 1.6 | 2.6 |
| NRCoV2-MRed03 | S1-NTD/Bin 8 | 0.51 | 0.36 | 0.67 |
| NRCov2-MRed06 | | 5.2 | 5.72 | 7.24 |
| NRCoV2-MRed07 | S1-NTD/Bin 9 | 0.11 | 0.26 | 0.23 |
| NRCoV2-SR04 | S1-NTD/Bin 7, 9 | 0.14 | 0.27 | 0.32 |
| NRCoV2-SR02 | S1-NTD/Bin 10 | 0.47 | 0.11 | 0.53 |
| NRCoV2-S2A3 | S2/Bin 11 | 0.56 | 2.18 | 0.85 |
| NRCoV2-S2A4 | S2/Bin 12 | 12.8 | 9.5 | 15.3 |
| NRCoV2-S2F3 | S2/Bin 13 | 3.03 | +⁴ | +⁴ |
| NRCoV2-MRed18 | | 6.03 | 12.9 | 6.48 |
| NRCoV2-MRed19 | | 9.07 | 20.2 | 8.07 |
| NRCoV2-MRed20 | | 0.092 | 0.55 | 0.45 |
| NRCoV2-MRed22 | | 0.51 | 0.25 | +⁴ |
| NRCoV2-S2G3 | S2/Bin 14 | 1.87 | 1.78 | 1.85 |
| NRCoV2-S2G4 | S2/Bin 15 | 0.23 | 0.19 | +⁴ |
| NRCoV2-MRed11 | S2/Bin 16 | 6.2 | 13.7 | 6.2 |
| NRCoV2-MRed25 | S2/Bin 17 | 1.02 | 0.28 | 1.6 |

¹V_{H}H-72 is the benchmark (Wrapp et al., 2020);
²nd, not determined;
³“—”, no binding;
⁴“+”, V_{H}H bound, but poor fitting precluded K_{D} determination. Epitope bin numbers correspond to the bins shown in FIG. 9G.

### Example 6: Cell Binding Assays by Flow Cytometry

### Introduction

In the previous Examples, lead V_{H}Hs were shown to be binding to SARS-CoV-2 S in its purified form. In this Example, it was confirmed whether the V_{H}Hs also bind to SARS-CoV-2 S in its more natural context, i.e., displayed on the cell membrane of CHO cells.

### Materials and Methods

A stable Chinese hamster ovary (CHO) cell line CHO^{BRI TM}/55E1 (Stuible et al., 2021) overexpressing SARS-CoV-2 S (CHO-S) was grown in BalanCD™ CHO Growth A medium (Irvine Scientific) supplemented with 50 μM of methionine sulfoximine (MSX) at 120 rpm and 37° C. in a humidified 5% CO₂ atmosphere. When the cell count reached 2×10⁶/mL, the expression of the membrane anchored SARS-CoV-2 trimeric spike protein (SmT1, described in Stuible et al, 2021) was induced by adding cumate at 2 μg/mL. Expression was carried out for 48 h at 32° C. For flow cytometry experiments, cells were harvested by centrifugation and resuspended at 1×10⁶ cells/mL in PBSB (1% PBS containing 1% BSA and 0.05 [v/v] sodium azide). Cells were kept on ice until use. Serially, three-fold dilutions of V_{H}H-Fcs were prepared in V-Bottom 96-well microtest plates (Globe Scientific, Cat #120130) and mixed with 50 μL of CHO-S cells. Plates were incubated for 1 h on ice, washed twice with PBSB by centrifugation 5 min at 1200 rpm and then incubated for an additional hour with 50 μL of R-Phycoerythrin AffiniPure F(ab′)₂ Fragment Goat Anti-Human IgG (Jackson Immunoresearch, Cat #109-116-170) at 250 ng/mL diluted in PBSB. After a final wash, cells were resuspended in 100 μL PBSB and data were acquired on a Beckman Culter CytoFlex S and analyzed by FlowJo™ (FlowJo LLC, vi 0.6.2, Ashland).

### Results and Discussion

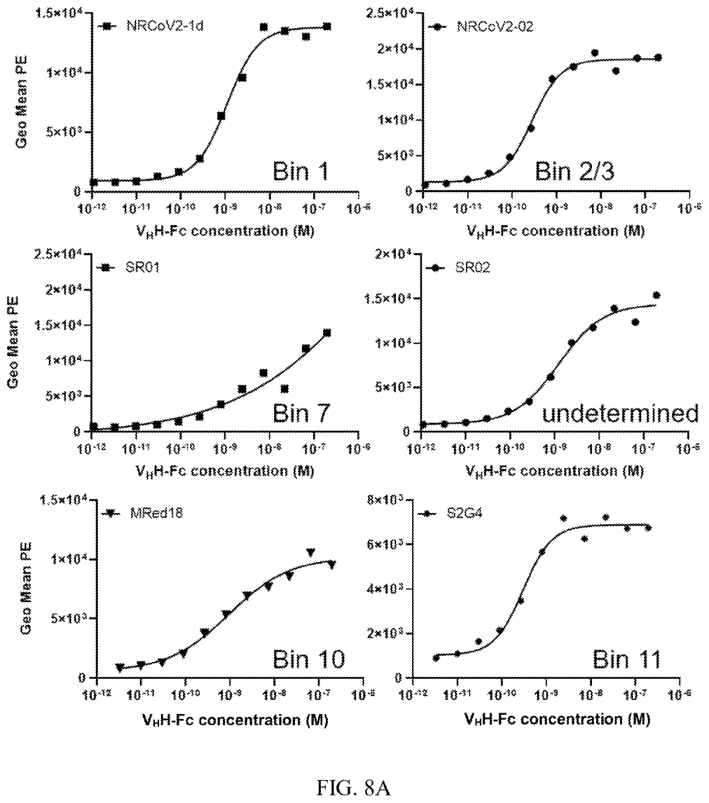

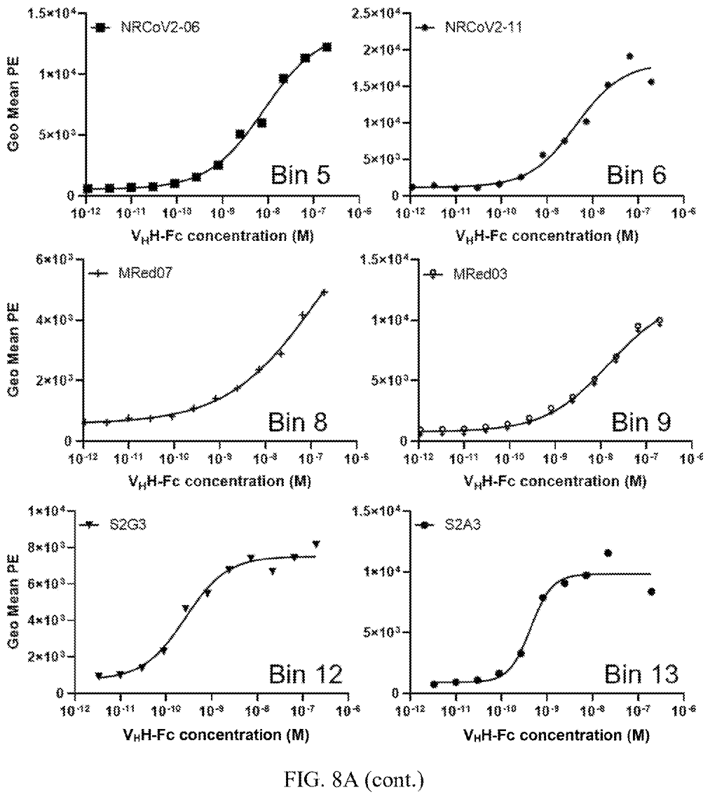

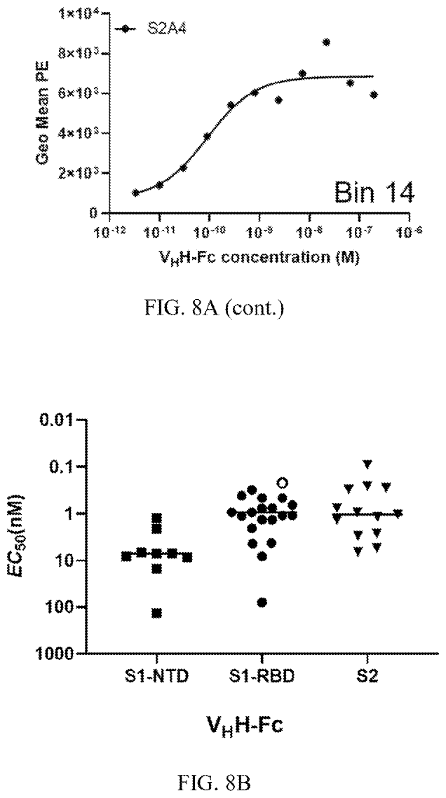

Interestingly, four V_{H}H-Fcs (NRCoV2-08, NRCoV2-19, NRCoV2-21, NRCoV2-S202) which bound to SARS-CoV-2 S in purified form did not bind to SARS-CoV-2 S-displaying target cells. The remaining 41 V_{H}H-Fcs, however, bound to cells in a dose dependent manner (FIG. 8A-B; Table 13). Aside from NRCoV2-03 which had a modest apparent affinity (EC₅₀app) of ˜80 nM, the remaining 18 S1-RBD-specific V_{H}H-Fcs bound to S-displaying CHO-S cells with high affinities (EC₅₀ range: 0.3-8.1 nM; EC₅₀ median: 1 nM). For S1-NTD-binders, excluding the outlier NRCoV2-MRed07 (EC₅₀=132 nM), the apparent EC₅₀s for the remaining V_{H}Hs were also high (range: 1.2-15.1 nM; median: 7 nM). Similarly, affinities for S2-specific V_{H}H-Fcs were also high (EC₅₀ range: 0.1-6.5; EC₅₀ median: 1 nM). V_{H}H-72 benchmark with an EC₅₀ of 0.2 nM ranked amongst the strongest S1-RBD-specific binders.

TABLE 13
Summary of V_{H}H-Fc bindings to SARS-CoV-2 S expressing CHO—S cells

| S1-RBD-specific | | | S1-NTD-specific | | | S2-specific | | |
|---|---|---|---|---|---|---|---|---|
| | EC₅₀ | | | EC₅₀ | | | EC₅₀ | |
| V_{H}H-Fc | (nM) | Bₘₐₓ | V_{H}H-Fc | (nM) | Bₘₐₓ | V_{H}H-Fc | (nM) | Bₘₐₓ |
| NRCoV2- | 1.1 | 17186 | NRCoV2- | 6.6 | 15389 | NRCoV2- | 0.44 | 9819 |
| 1a | | | SR01 | | | S2A3 | | |
| NRCoV2- | 1.1 | 13817 | NRCoV2- | 1.2 | 14416 | NRCoV2- | 0.1 | 6858 |
| 1d | | | SR02 | | | S2A4 | | |
| NRCoV2- | 0.3 | 18553 | NRCoV2- | 7.1 | 19857 | NRCoV2- | 6.5 | 6665 |
| 02 | | | SR03 | | | S2B3 | | |
| NRCoV2- | 78.9 | 16242 | NRCoV2- | 8.2 | 10119 | NRCoV2- | 2.7 | 6953 |
| 03 | | | SR04 | | | S2F3 | | |
| NRCoV2- | 1.3 | 15489 | NRCoV2- | 7.0 | 17808 | NRCoV2- | 0.3 | 7529 |
| 04 | | | SR13 | | | S2G3 | | |
| NRCoV2- | 0.5 | 17419 | NRCoV2- | 1.4 | 11553 | NRCoV2- | 0.3 | 6897 |
| 05 | | | SR16 | | | S2G4 | | |
| NRCoV2- | 8.1 | 13615 | NRCoV2- | 15.1 | 11990 | NRCoV2- | 1.0 | 7906 |
| 06 | | | MRed03 | | | S2H4 | | |
| NRCoV2- | 1.1 | 10620 | NRCoV2- | 8.5 | 9023 | NRCoV2- | − | − |
| 07 | | | MRed06 | | | S202 | | |
| NRCoV2- | − | − | NRCoV2- | 132.4 | 8673 | NRCoV2- | 2.9 | 3249 |
| 08 | | | MRed07 | | | MRed11 | | |
| NRCoV2- | 1.3 | 28044 | | | | NRCoV2- | 0.9 | 10205 |
| 10 | | | | | | MRed18 | | |
| NRCoV2- | 4.3 | 18230 | | | | NRCoV2- | 1.3 | 5482 |
| 11 | | | | | | MRed19 | | |
| NRCoV2- | 2.0 | 11963 | | | | NRCoV2- | 6.5 | 6665 |
| 11a | | | | | | MRed20 | | |
| NRCoV2- | 0.8 | 21821 | | | | NRCoV2- | 0.2 | 5324 |
| 12 | | | | | | MRed22 | | |
| NRCoV2- | 0.9 | 18801 | | | | NRCoV2- | 0.8 | 4421 |
| 14 | | | | | | MRed25 | | |
| NRCoV2- | 0.8 | 21053 | | | | | | |
| 15 | | | | | | | | |
| NRCoV2- | 0.4 | 19019 | | | | | | |
| 17 | | | | | | | | |
| NRCoV2- | 0.9 | 18266 | | | | | | |
| 18 | | | | | | | | |
| NRCoV2- | − | − | | | | | | |
| 19 | | | | | | | | |
| NRCoV2- | 0.7 | 12056 | | | | | | |
| 20 | | | | | | | | |
| NRCoV2- | − | − | | | | | | |
| 21 | | | | | | | | |
| NRCoV2- | 4.2 | 14916 | | | | | | |
| MRed04 | | | | | | | | |
| NRCoV2- | 0.5 | 17349 | | | | | | |
| MRed05 | | | | | | | | |
| VHH-72 | 0.2 | 8026 | | | | | | |

“−”, No detectable binding observed.

### Example 7: Epitope Studies

### Introduction

Western blotting experiments were performed to determine if V_{H}Hs bind to conformational or linear epitopes. Additionally, competitive sandwich ELISA as well as SPR were performed to differentiate V_{H}Hs with respect to recognizing non-overlapping epitopes.

### Materials and Methods

### Epitope Typing by Sodium Dodecyl Sulphate-Polyacrylamide Gel Electrophoresis/Western Blotting (SDS-PAGE/WB)

A standard SDS-PAGE/WB was performed to detect the binding of V_{H}Hs to nitrocellulose-immobilized, denatured SARS-CoV-2 S. Briefly, 10 μg/lane of S was run on 4-20% Mini-PROTEAN® TGX Stain-Free™ Protein Gels (Bio-Rad, Cat #4568081), transferred to nitrocellulose (Sigma, Cat #GE10600002) and blocked with 1% PBSC overnight at 4° C. Then, 0.5-cm nitrocellulose strips containing the denatured S were placed on Mini Incubation Trays (Bio-Rad, Cat #1703902) and incubated with 1 mL of 1 μg/mL V_{H}H-Fcs or biotinylated V_{H}Hs (V_{H}H-BAP-His6). After 1 h incubation at room temperature, strips were washed 10 times with PBST and the binding of V_{H}H-Fcs or biotinylated V_{H}Hs to denatured S was probed, respectively, by incubating strips with 1 mL of 100 ng/mL anti-human Ig Fc antibody-peroxidase conjugate or streptavidin-peroxidase conjugate (Jackson ImmunoResearch, Cat #016-030-084) at room temperature for 1 h. Finally, strips were washed 10 times with PBST and peroxidase activity was detected using chemiluminescent reagent (SuperSignal™ West Pico PLUS Chemiluminescent Substrate, ThermoFisher, Cat #34580). Images of developed strips were acquired on Molecular Imager® Gel Doc™ XR System (Bio-Rad, Cat #1708195EDU).

### Epitope Binning by SPR

Standard SPR techniques were used for binding studies. All SPR assays were performed on a Biacore™ T200 instrument (Cytiva) at 25° C. with HBS-EP running buffer (10 mM HEPES, 150 mM NaCl, 3 mM EDTA, 0.005% [v/v] Tween® 20, pH 7.4) and CM5 sensor chips (Cytiva). Prior to SPR analyses all analytes in flow (V_{H}Hs, ACE2 receptor) were SEC-purified on a Superdex® 75 Increase 10/300 GL column (Cytiva) in HBS-EP buffer at a flow rate of 0.8 mL/min to obtain monomeric proteins. V_{H}H epitope binning was performed by SPR dual injection experiments on the SARS-CoV-2 S at a flow rate of 40 μL/min in HBS-EP buffer. Dual injections consisted of injection of V_{H}H1 (at 50-100×K_{D} concentration) for 150 s, followed by immediate injection of a mixture of V_{H}H1+V_{H}H2 (both at 50-100×K_{D} concentration) for 150 s. The opposite orientation was also performed (V_{H}H2 followed by V_{H}H2+V_{H}H1) (FIG. 9C). Surfaces were regenerated using a 12 s pulse of 10 mM glycine, pH 1.5, at a flow rate of 100 μL/min. All pairwise combinations of V_{H}Hs were analyzed and distinct or overlapping epitope bins determined.

### Epitope Binning by ELISA

The pairwise ability of V_{H}Hs to bind to their antigen in a sandwich ELISA format was evaluated as described previously (Rosotti et al., 2015a; Delfin-Riela et al., 2020), (FIG. 9D). Briefly, a matrix of 14 wells (row)×23 wells (column) was generated using six NUNC® MaxiSorp™ 4BX plates (Thermo Fisher) and coated overnight at 4° C. with 4 μg/mL streptavidin (Jackson ImmunoResearch, Cat #016-000-113) in 100 μL PBS, pH 7.4. Wells were blocked with 200 μL PBSC for 1 h at room temperature and then biotinylated V_{H}Hs (10 μg/mL in 100 μL PBSCT) were captured in each row (same V_{H}H in each row; 14 rows for a total of 14 V_{H}Hs) for 1 h at room temperature. Wells were washed 5 times with PBST and incubated with 100 ng/mL of SARS-CoV-2 S1 diluted in PBSCT for 1 h. Wells were washed and each column was incubated with the pairing, V_{H}H-Fcs/ACE2-Fc at 1 μg/mL used as detector antibodies (same V_{H}H-Fc in each column; 23 column for a total of 22 V_{H}H-Fcs and ACE2-Fc). The binding of V_{H}H-Fcs/ACE2-Fc to S1 was detected using 100 μL 1 μg/mL HRP-conjugated goat anti-human IgG (SIGMA, Cat #A0170). Finally, plates were washed 10 times with PBST and peroxidase activity was determined as described above. The same procedure was carried out performing a matrix of 17 well (row)×20 wells (column) as shown in FIG. 9E.

### Results and Discussion

To determine whether V_{H}Hs recognize conformational or linear epitopes, they were subjected to binding analysis against SARS-CoV-2 S by denaturing, SDS-PAGE/Western blot. As shown in FIG. 9A using the monomeric V_{H}Hs as probe, three out of 26 V_{H}Hs tested bound to denatured S, indicating they were recognizing linear epitopes, while the remaining V_{H}Hs appeared to be conformational epitope-specific based on their lack of significant binding to denatures S. In assays where V_{H}H-Fc was used instead of V_{H}H, 15 out of 37 V_{H}H-Fcs tested were determined to bind to linear epitopes (FIG. 9B). These linear epitope-specific V_{H}Hs give the option of virus detection against denatured S by robust diagnostic techniques such as SDS-PAGE/Western blot, where the additional molecular weight information provided by the SDS-PAGE would serve as a second, confirmatory piece of information to eliminate/reduce false positives obtained by binding data alone.

To identify the number of distinct (non-overlapping) epitopes, V_{H}Hs were subjected to epitope binning experiments by SPR and sandwich ELISA. In SPR epitope binning assays, the first V_{H}H (“V_{H}H1”) was flowed over a spike protein-immobilized sensorchip and allowed to saturate its epitope, followed by the addition of the second, V_{H}H2 applied as a mixture of V_{H}H1+V_{H}H2 to keep the V_{H}H1 epitope saturated during the binding of V_{H}H2. Assays were performed in a second orientation as well to cross-confirm results: V_{H}H2+(V_{H}H2+V_{H}H1). FIG. 9C (left panel) exemplifies a V_{H}H pair (NRCoV2-02/NRCoV2-05) binding to an overlapping epitope, hence belonging to the same epitope bin, as the addition of the second V_{H}H does not result in any increased binding (i.e., increase in RU) over that obtained for the addition of the first V_{H}H. FIG. 9C (right panel), on the other hand, exemplifies a V_{H}H pair (NRCoV2-02/NRCoV2-07) binding to non-overlapping epitopes, hence belonging to different epitope bins, as the addition of the second V_{H}H results in significant increase in binding over that already achieved by the addition of the first V_{H}H. SPR assays were performed with combination pairs of nine V_{H}Hs, including V_{H}H-72 against S1-RBD, six V_{H}Hs against S1 and 10 V_{H}Hs against S2. A conceptually similar assay to SPR was performed for 14 more S1-RBD-specific V_{H}Hs by a sandwich ELISA to further expand on epitope bins identified by SPR for the S1-RBD-specific V_{H}Hs (FIGS. 9D (initial results) and 9E (further results)). The sandwich ELISA allowed for the rapid identification of antibody pairs that simultaneously bound to the antigen, hence to non-overlapping epitopes. ACE2 and the benchmark V_{H}H, V_{H}H-72, were also included in the epitope binning experiments. The ELISA experiments confirmed the results of epitope binning by SPR, expanded the number of binders within each epitope bin, and identified new epitope bins. The epitope binning results obtained by SPR and ELISA are summarized in FIGS. 9F (initial results), 9G (further results) and Table 14. Initial binning results identified 14 non-overlapping/partially overlapping bins: six for S1-RBD-specific V_{H}Hs, three for S1-NTD-specific V_{H}Hs and five for S2-specific V_{H}Hs. Benchmark V_{H}H-72 binned with S1-RBD-specific V_{H}Hs NRCoV2-1a/1c/1d, NRCoV2-07, NRCoV2-12, NRCoV2-18, NRCoV2-20, NMed02 and NRCov2-MRed04. Thirteen out of 22 RBD-specific V_{H}Hs tested, binned with ACE2 (FIG. 9F). Further characterization led to the identification of 17 non-overlapping/partially overlapping bins: six for S1-RBD-specific V_{H}Hs, four for S1-NTD-specific V_{H}Hs and seven for S2-specific V_{H}Hs (as shown in FIG. 9G).

TABLE 14
Summary of epitope binning results

| | | | | | | | Do- | |
|---|---|---|---|---|---|---|---|---|
| | | | | | | | main/ | |
| | | | | | | | sub- | |
| | | SEQ | | SEQ | | SEQ | unit | Epi- |
| | CDR | ID | | ID | | ID | speci- | tope |
| V_{H}H | 1 | NO: | CDR 2 | NO: | CDR 3 | NO: | ficity | Bin |
| NRCoV2- | GFTF | 21 | ISGNG | 65 | AATGIRST | 111 | S1-NTD | 7/9/ |
| SR01 | DNYA | | GVT | | WSVYGCSR | | | 10 |
| | | | | | LAGPYDY | | | |
| NRCoV2- | GSIF | 23 | ISSGG | 67 | NRGGWEYR | 113 | S1-NTD | 7/9/ |
| SR03 | SNNH | | KT | | SSYYIMGP | | | 10 |
| | | | | | H | | | |
| NRCoV2- | GSRF | 25 | ISSGG | 69 | NMGGWDYR | 115 | S1-NTD | 7/9/ |
| SR13 | GSKH | | ST | | SNTYIPGS | | | 10 |
| | | | | | RSDY | | | |
| NRCoV2- | GTTF | 26 | ISTSG | 70 | NTGGWDYR | 116 | S1-NTD | 7/9/ |
| SR16 | SRYH | | AV | | SSTFIMGL | | | 10 |
| | | | | | N | | | |
| NRCoV2- | GRTF | 24 | ISMGG | 68 | NTAALVGN | 114 | S1-NTD | 7/9 |
| SR04 | SSHT | | NTNYA | | RLLPMATI | | | |
| | | | | | T | | | |
| NRCoV2- | GFTL | 20 | ISSSD | 81 | ATDAFATC | 126 | S1-NTD | 8 |
| MRed03 | DYYA | | GST | | DSWYAQIA | | | |
| | | | | | QYDF | | | |
| NRCoV2- | GFTL | 37 | ISSSD | 82 | ATDSFSSC | 128 | S1-NTD | 8 |
| MRed06 | AYYA | | GSA | | SDYESGMD | | | |
| | | | | | F | | | |
| NRCoV2- | GSIG | 38 | ITRGG | 83 | YANYGWAI | 129 | S1-NTD | 9 |
| MRed07 | PFNT | | VT | | PY | | | |
| NRCoV2- | EFTL | 22 | IRYSG | 66 | AADRLYSR | 112 | S1-NTD | 10 |
| SR02 | NYYS | | GGI | | ACPTAGGR | | | |
| | | | | | NY | | | |
| NRCoV2- | GSTL | 1 | VSSSD | 45 | AADYSMRP | 90 | S1-RBD | 1 |
| 1a | DYYA | | GST | | LWVSRWHR | | | |
| | | | | | DYEY | | | |
| NRCoV2- | GSIL | 2 | VSSSD | 45 | AADYSMRR | 91 | S1-RBD | 1 |
| 1c | DYYA | | GST | | FAVGRWHR | | | |
| | | | | | DYEY | | | |
| NRCoV2- | GSTL | 1 | VSSSD | 46 | AADYSMRP | 92 | S1-RBD | 1 |
| 1d | DYYA | | GNT | | FAVGRWHR | | | |
| | | | | | DYEY | | | |
| NRCoV2- | GVTL | 8 | ISSNG | 52 | AAVQDVHG | 98 | S1-RBD | 1 |
| 07 | DYYA | | RRN | | DNYYCTSP | | | |
| | | | | | NEYNV | | | |
| NRCoV2- | GRTF | 13 | VAAIS | 57 | AADRGLSY | 103 | S1-RBD | 1 |
| 12 | RNYV | | WGGTE | | YYTRTTEY | | | |
| | | | I | | NY | | | |
| NRCoV2- | GITI | 17 | INSGG | 61 | SLHTSHDY | 107 | S1-RBD | 1 |
| 18 | SGYN | | ST | | | | | |
| NRCoV2- | GRTF | 19 | VAVIS | 63 | AADRGMSY | 109 | S1-RBD | 1 |
| 20 | SNYV | | GSDTE | | YYTRATEY | | | |
| | | | T | | YY | | | |
| NRCoV2- | GNIF | 35 | IWSDS | 79 | AADRGFVV | 125 | S1-RBD | 1 |
| MRed02 | SINS | | RT | | RGQYDY | | | |
| NRCoV2- | GNSF | 36 | IWSDT | 80 | AADRGFVV | 125 | S1-RBD | 1 |
| MRed04 | SINT | | TT | | RGQYDY | | | |
| NRCoV2- | GSPF | 5 | ISGGG | 49 | WSSYEST | 95 | S1-RBD | 4 |
| 04 | SNVV | | IA | | | | | |
| NRCoV2- | VSTF | 7 | IGFVG | 51 | NARHYGGS | 97 | S1-RBD | 5 |
| 06 | SSYA | | AT | | EY | | | |
| NRCoV2- | GITF | 4 | MSNMD | 48 | NIYGPTYS | 94 | S1-RBD | 6 |
| 03 | SYYA | | ST | | TRRNEY | | | |
| NRCoV2- | GFTL | 9 | ISRSG | 53 | AADYQYST | 99 | S1-RBD | 6 |
| 08 | DDYA | | TTT | | YCLGYDAH | | | |
| | | | | | YEY | | | |
| NRCoV2- | GSSL | 11 | ISRYY | 55 | AARSRDFS | 101 | S1-RBD | 6 |
| 11 | DSYS | | SST | | SPFSATDT | | | |
| | | | | | YTS | | | |
| NRCoV2- | GFTL | 12 | ISRYY | 56 | AARSRDFS | 102 | S1-RBD | 6 |
| 11a | DSYN | | EST | | SPISATDK | | | |
| | | | | | YGS | | | |
| NRCoV2- | GFTF | 3 | ISGRG | 47 | TKGPDLYY | 93 | S1-RBD | 2,3 |
| 02 | SNYA | | DDT | | FGSGYSD | | | |
| NRCoV2- | GFIF | 6 | INSGG | 50 | SKGPVSSY | 96 | S1-RBD | 2,3 |
| 05 | SNYA | | GDT | | YGSGYDY | | | |
| NRCoV2- | GNTF | 10 | ISSRG | 54 | YAADDLGD | 100 | S1-RBD | 2,3, |
| 10 | SRSN | | IS | | Y | | | 4 |
| NRCoV2- | GSTS | 15 | VSTSG | 59 | YAAYGGGG | 105 | S1-RBD | 2,3, |
| 15 | GRNT | | AT | | DY | | | 4 |
| NRCoV2- | GFTL | 20 | ISSSD | 81 | ATGPQAYY | 127 | S1-RBD | 2,3, |
| MRed05 | DYYA | | GST | | SGSYYFQC | | | 4 |
| | | | | | PQAGMDY | | | |
| NRCoV2- | GTTF | 14 | ISVFG | 58 | HAVNADIG | 104 | S1-RBD | 2,4 |
| 14 | SHYA | | ST | | GDY | | | |
| NRCoV2- | GSPF | 16 | ISPTG | 60 | QAANVNGG | 106 | S1-RBD | 3,4 |
| 17 | SQLA | | NR | | DY | | | |
| NRCoV2- | GLTL | 18 | LTSGG | 62 | AADRARLR | 108 | S1-RBD | ud |
| 19 | NSYA | | TG | | FGCSLNFR | | | |
| | | | | | REVAYDY | | | |
| NRCoV2- | GFTL | 20 | ISSGG | 64 | AADHRGRS | 110 | S1-RBD | ud |
| 21 | DYYA | | ST | | LRFGCSSS | | | |
| | | | | | TTDYLY | | | |
| NRCoV2- | GRPY | 27 | KQREL | 71 | NTGSLSYG | 117 | S2 | 11 |
| S2A3 | SNYA | | VAAIS | | GSVYYPSY | | | |
| | | | SGGTT | | DN | | | |
| NRCoV2- | GSPF | 28 | ISTGG | 72 | HAAARDSH | 118 | S2 | 12 |
| S2A4 | RSNV | | SR | | GIYLLDT | | | |
| NRCoV2- | VRIL | 31 | ITSGG | 75 | NLRDILSQ | 121 | S2 | 13 |
| S2F3 | SVPA | | ST | | PF | | | |
| NRCoV2- | TTVF | 40 | VSDGG | 85 | NYYNYYYG | 131 | S2 | 13 |
| MRed18 | GRNA | | TP | | RNF | | | |
| NRCoV2- | TIIF | 41 | MTTSG | 86 | YMHSVYYG | 132 | S2 | 13 |
| MRed19 | KGQT | | SA | | IDY | | | |
| NRCoV2- | GLSF | 42 | IRESG | 87 | AAKPPFYG | 133 | S2 | 13 |
| MRed20 | SSYD | | SGT | | SGTYSTPR | | | |
| | | | | | AYLY | | | |
| NRCoV2- | GSVF | 43 | ISSRG | 88 | NAREFTGF | 134 | S2 | 13 |
| MRed22 | ASNA | | ST | | DY | | | |
| NRCoV2- | GSTF | 33 | ISSDG | 77 | NKHWWTGD | 123 | S2 | 14 |
| S2G4 | SGYA | | DK | | W | | | |
| NRCoV2- | GSTF | 32 | ITSGG | 76 | YTTKRDDA | 122 | S2 | 15 |
| S2G3 | GIFL | | AT | | SVY | | | |
| NRCoV2- | GFTF | 39 | INSGG | 84 | ATTISDGS | 130 | S2 | 16 |
| MRed11 | SSYA | | GST | | SWSTKSY | | | |
| NRCoV2- | GHTF | 44 | ISWRG | 89 | AAEMWGTA | 135 | S2 | 17 |
| MRed25 | SRYG | | DST | | TIVASRYT | | | |
| | | | | | Y | | | |
| NRCoV2- | ASTF | 29 | ISTGS | 73 | NYRSIYYG | 119 | S2 | nd |
| S2B3 | GDSA | | NT | | QNF | | | |
| NRCoV2- | GFTF | 30 | INSGD | 74 | ALVFGYTS | 120 | S2 | nd |
| S2H4 | NLYS | | RDSTT | | RDYCLTPK | | | |
| | | | | | RGNY | | | |
| NRCoV2- | GITV | 34 | ISAGG | 78 | NYGPGYRK | 124 | S2 | nd |
| S202 | SRIG | | ST | | AA | | | |

nd = no data, ud = undetermined. Epitope bin numbers correspond to the bins shown in FIG. 9G.

### Example 8: Surrogate Virus Neutralization Assays

### Introduction

Surrogate neutralization assays were performed to identify potential neutralizing V_{H}Hs/V_{H}H-Fcs, i.e., V_{H}Hs/V_{H}H-Fcs inhibiting SARS-CoV-2 viruses from entering host cells. Three different surrogate assays were performed: ELISA, SPR and flow cytometry. In ELISA and SPR, ACE2 and SARS-CoV-2 S acted as surrogates for an ACE2-containing host cell and an S-containing invading virus, respectively. In flow cytometry assays, which were performed directly against the host cell (Vero E6), S1-RBD or S served as surrogate virus. Antibodies that interfered with the binding of spike fragment proteins to ACE2 in the surrogate assays were considered to be neutralizing antibodies.

### Materials and Methods

### ACE2 Competition Assay by ELISA

Wells of NUNC® MaxiSorp™ microtiter plates (Thermo Fisher) were coated overnight at 4° C. with 50 ng/well of S in 100 μL PBS, pH 7.4. Next day, plates were blocked with 250 μL PBSC for 1 h at room temperature. For ACE2/V_{H}H competition binding to SARS-CoV-2 S, 50 μL of ACE2-Fc (ACROBiosystems, Cat #AC2-H5257) at 400 ng/mL was mixed with 50 μL of V_{H}H at 1 μM, and then transferred to SARS-CoV-2 S coated microtiter plate wells. After 1 h incubation at room temperature, plates were washed 10 times with PBST and the ACE2-Fc binding was detected using 1 μg/mL goat anti-human IgG (Fc specific)-peroxidase antibody (SIGMA, Cat #A0170) in 100 μL PBSCT. After 10 washes with PBST, the peroxidase activity was determined as described above.

ACE2 competition assay by SPR

Standard SPR techniques were used for binding studies. All SPR assays were performed on a Biacore™ T200 instrument (Cytiva) at 25° C. with HBS-EP running buffer (10 mM HEPES, 150 mM NaCl, 3 mM EDTA, 0.005% [v/v] Tween® 20, pH 7.4) and CM5 sensor chips (Cytiva). Prior to SPR analyses, all analytes in flow (V_{H}Hs, ACE2 receptor) were SEC-purified on a Superdex™ 75 Increase 10/300 GL column (Cytiva) in HBS-EP buffer at a flow rate of 0.8 mL/min to obtain monomeric proteins. V_{H}Hs were analyzed for their ability to block the SARS-CoV-2 spike trimer (S) interaction with ACE2 using SPR dual injection experiments. V_{H}Hs and ACE2 were flowed over the SARS-CoV-2 S surface at 40 μL/min in HBS-EP buffer. Dual injections consisted of injection of ACE2 (1 μM) for 150 s, followed by immediate injection of a mixture of ACE2 (1 μM)+V_{H}H (at 20-40×K_{D} concentration) for 150 s. The opposite orientation was also performed (V_{H}H followed by V_{H}H+ACE2). Surfaces were regenerated using a 12 s pulse of 10 mM glycine, pH 1.5, at a flow rate of 100 μL/min. All pairwise combinations of V_{H}Hs and ACE2 were analyzed. V_{H}Hs that competed with ACE2 for SARS-CoV-2 spike trimer binding showed no increase in binding response during the second injection. Conversely, a binding response was seen during the second injection for V_{H}Hs that did not compete with ACE2.

### ACE2 Competition Assay by Flow Cytometry

Experiments were performed essentially as described in Example 2. Briefly, 400 ng of chemically biotinylated trimeric SARS-CoV-2 S was mixed with 5×10⁴ Vero E6 cells in the presence of decreasing concentrations of V_{H}Hs or V_{H}H-Fcs in a final volume of 150 μL. Following 1 h incubation on ice, cells were washed twice with PBSB by centrifugation at 1200 rpm for 5 min and then incubated for an additional hour with 50 μL Streptavidin, R-Phycoerythrin Conjugate (SAPE, ThermoFisher, Cat #S866) at 250 ng/mL diluted in PBSB. After a final wash, cells were resuspended in 100 μL PBSB and data were acquired on a CytoFlex™ S flow cytometer (Beckman Culter) and analyzed by FlowJo™ (FlowJo LLC, v10.6.2, Ashland, OR). As an internal reference for competition experiments, a competition assay with recombinant human ACE2-His6 in lieu of V_{H}H was also included. A20.1, a C. difficile toxin A-specific V_{H}H (Hussack et al., 2011) was used as negative control V_{H}H. Percent inhibition (neutralization) was calculated according to the following formula: % inhibition=100×[1−(Fₙ−Fₘᵢₙ)/(Fₘₐₓ−Fₘᵢₙ)], where, Fₙ is the measured fluorescence at any given competitor V_{H}H concentration, Fₘᵢₙ is the background fluorescence measured in the presence of cells and SAPE only, and Fₘₐₓ is the maximum fluorescence, measured in the absence of V_{H}H competitor.

### Results and Discussion

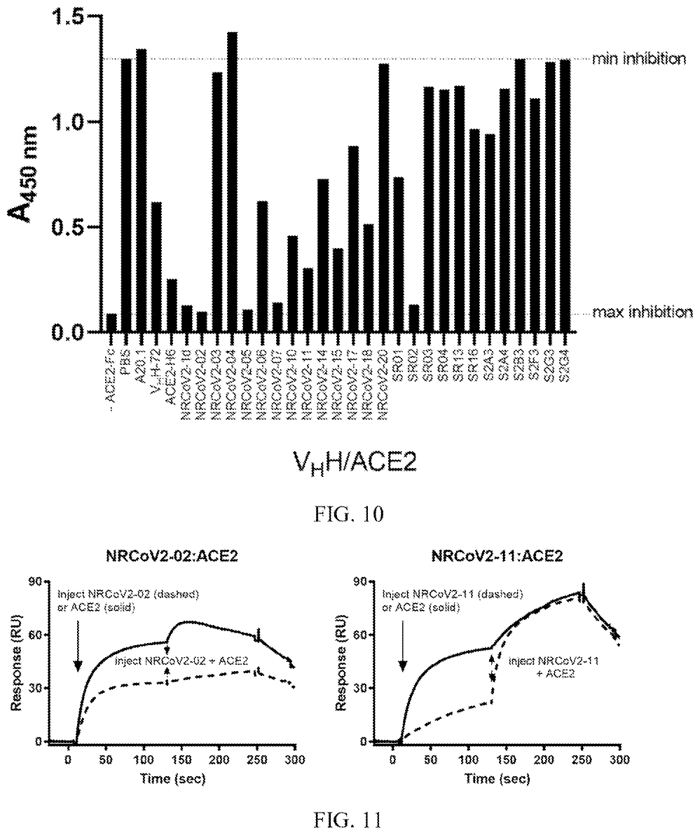

Initially, a total of 26 V_{H}Hs (14 S1-RBD-specific, 6 S1-NTD-specific and 6 S2-specific) were subjected to competitive ELISA, to identify those that are neutralizing, i.e., reduce the binding of ACE2-Fc to S. As shown in FIG. 10, the majority of S1-RBD binders were significantly neutralizing, with NRCoV2-1d, NRCoV2-02, NRCoV2-05 and NRCoV2-07 displaying essentially 100% inhibition and outperforming the V_{H}H-72 benchmark. Two of the S1-NTD-specific V_{H}Hs (NRCoV2-SR01, NRCoV2-SR02) showed significant neutralization, with NRCoV2-SR02 essentially neutralizing at 100%. None of the S2 binders showed significant neutralizing activity. A conceptually similar assay to ELISA was performed by a competitive SPR. The results are shown in FIG. 11 and Table 16. The four lead neutralizers identified by ELISA, i.e., NRCoV2-01 d, NRCoV2-02, NRCoV2-05 and NRCoV2-07, were confirmed by SPR to be complete neutralizers (“blockers”). NRCoV2-14, NRCoV2-15, NRCoV2-18 and NRCoV2-20 showed partial neutralization (“+/−”; Table 16). The remaining V_{H}Hs tested were judged to be non-neutralizing. Although the ELISA and SPR results agreed in the case of the majority of V_{H}Hs, there was some disagreement. For example, while NRCoV2-SR02, NRCoV2-06, NRCoV2-10, and NRCoV2-11 were neutralizing by ELISA, they were not found to be neutralizing by SPR. Conversely, NRCoV2-20 was judged to be somewhat neutralizing by SPR, but non-neutralizing by ELISA.

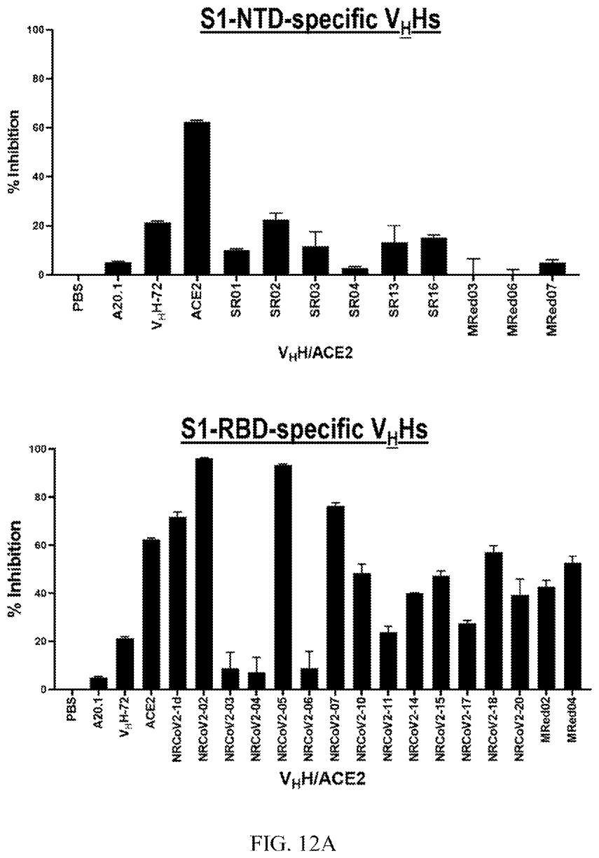

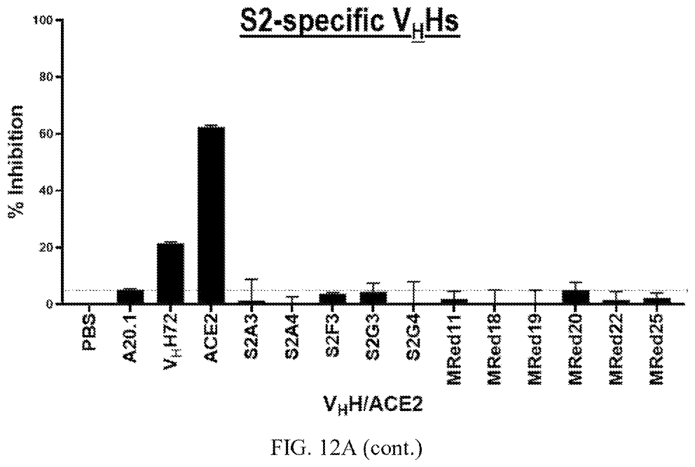

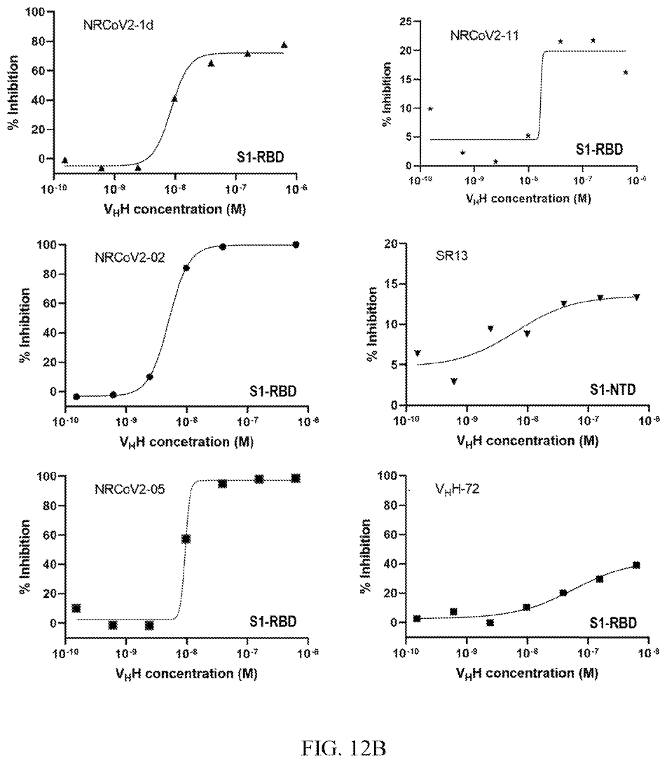

Finally, a quantitative surrogate neutralization assay was performed by flow cytometry, where antibodies were assessed based on their ability to block the interaction of trimeric SARS-CoV-2 S with ACE2 on the surface of Vero E6 cells (African green monkey kidney cells). (Vero E6 cells are known to be highly susceptible to infection by SARS-CoV-2 and SARS-CoV.) Both monomeric V_{H}Hs and bivalent V_{H}H-Fcs were assessed. IC₅₀s, IC₉₉s and Iₘₐₓ% values, measures of potency and efficacy were used to rank neutralizing antibodies. A preliminary screen performed at a single concentration with S1-RBD-, S1-NTD- and S2-specific V_{H}Hs showed that many of the S1-RBD-specific V_{H}Hs were potent neutralizers (FIG. 12A). Assays were also performed at multiple V_{H}H concentrations allowing determination of IC₅₀s and Iₘₐₓ% values (FIG. 12B; Table 17). The Iₘₐₓ% for NRCoV2-5R13 was too low to warrant a reliable IC₅₀ determination for this V_{H}H. In agreement with the preliminary results, all of the neutralizers were S1-RBD-specific with many exhibiting high neutralization potencies and efficacies. In particular, NRCoV2-1d, NRCoV2-02, NRCoV2-05 and NRCoV2-07 led others with IC₅₀/Iₘₐₓ% values of 8.6 nM/72%, 5.1 nM/100%, 9.5 nM/97%, and 7.5 nM/86%, respectively. A second group of V_{H}Hs, including NRCoV2-10, NRCoV2-14, NRCoV2-15, NRCoV2-18, NRCoV2-20 and NRCoV2-MRed04 were also potent/efficacious neutralizers (Table 17). All of these antibodies outperformed the benchmark V_{H}H-72, which had a far higher IC₅₀ (59 nM). NRCoV2-11 and NRCoV2-17 although showing high potencies (IC₅₀s of 16.8 nM and 9.4 nM, respectively), had weak efficacies (Iₘₐₓ% values of 20% and 18%, respectively). None of the S1-NTD or S2 binders was neutralizing. The results obtained by flow cytometry correlated well with those obtained by ELISA and SPR

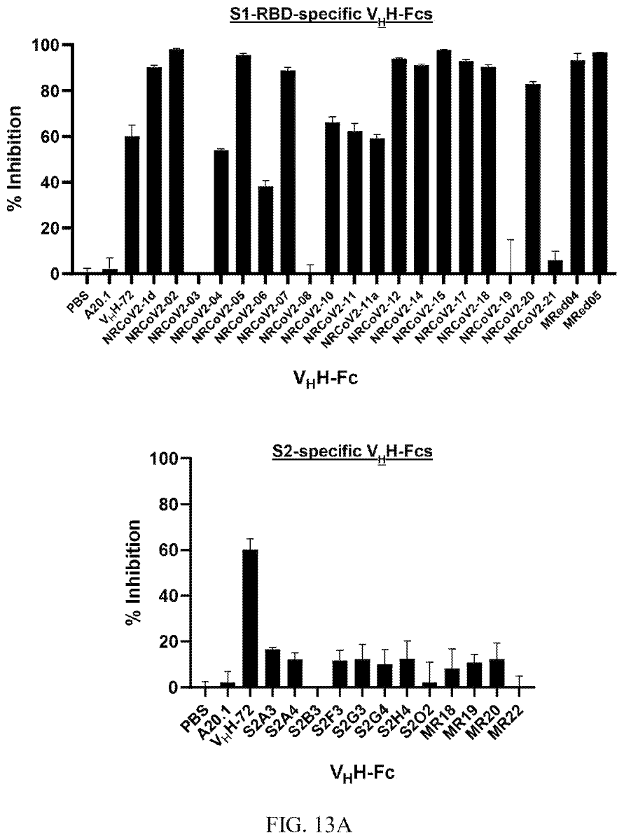

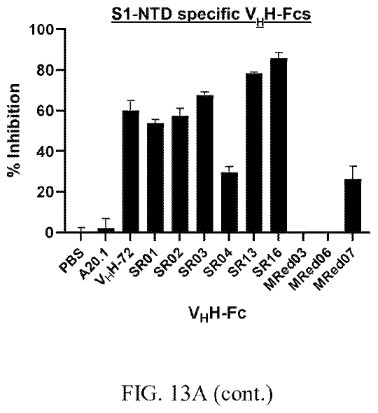

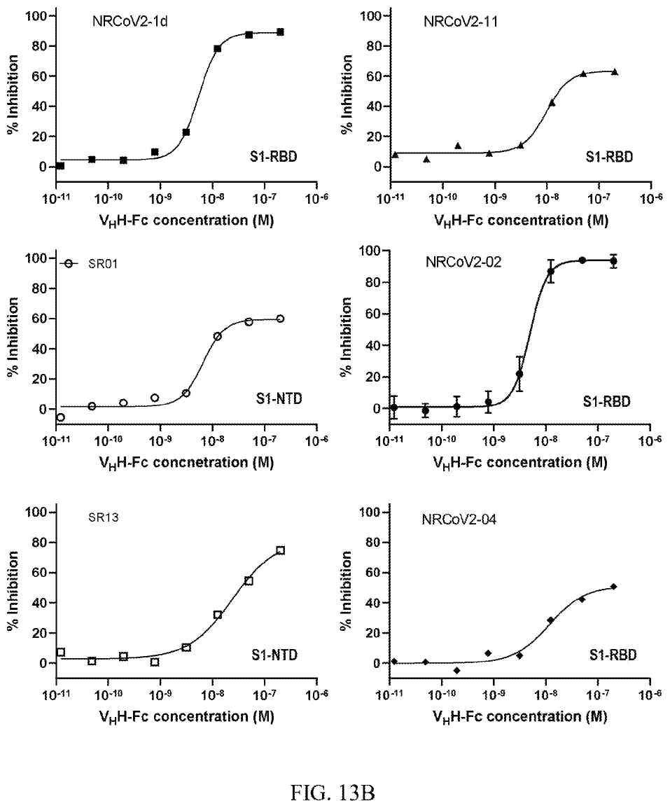

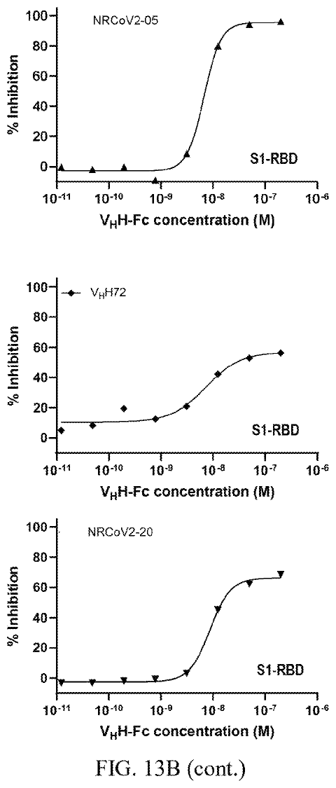

To increase the neutralization potency and efficacy of the V_{H}Hs, they were reformatted as bivalent V_{H}H-Fcs. The increase in size (from 16 kDa V_{H}H to 80 kDa V_{H}H-Fc) as well as avidity (from monovalent V_{H}H to bivalent V_{H}H-Fc) could sterically hinder the binding of S to ACE2 and increase V_{H}Hs' apparent affinity leading to their improved neutralization potency and efficacy. Thus, V_{H}H-Fcs were generated and tested in flow cytometry surrogate neutralization assays as described above. The majority of V_{H}H-Fcs demonstrated high potencies and efficacies (FIGS. 13A-B; Table 17). Reformatting had a significant effect on the neutralization potencies/efficacies of V_{H}Hs. As for S1-RBD-specific V_{H}Hs, reformatting imparted neutralization capability to NRCoV2-04, and significantly improved the neutralization potency/efficacy of NRCoV2-11, NRCoV2-14, NRCoV2-15, NRCoV2-17, and NRCoV2-18, as well as the V_{H}H-72 benchmark. The potency and efficacy of NRCoV2-1d, NRCoV2-02, NRCoV2-05 and NRCoV2-07 were not essentially affected with reformatting, except for NRCoV2-1d whose Iₘₐₓ% was increased from 72% (V_{H}H) to 89% (V_{H}H-Fc). Reformatting had a more profound effect on S1-NTD-specific V_{H}Hs, transforming six V_{H}Hs (NRCoV2-SR01, NRCoV2-SR02, NRCoV2-SR03, NRCoV2-SR04, NRCoV2-5R16, and NRCoV2-MRed07) into neutralizing antibodies, with some displaying strong potencies/efficacies (NRCoV2-SR01, NRCoV2-SR02, and NRCoV2-SR13). As for S2-specific V_{H}H-Fcs, none was found to be neutralizing. Based on IC₉₉/Iₘₐₓ% values, many V_{H}H-Fcs outperformed the V_{H}H-72 benchmark. These included S1-RBD-specific V_{H}H-Fcs NRCoV2-1a, NRCoV2-1d, NRCoV2-02, NRCoV2-05, NRCoV2-07, NRCoV2-11, NRCoV2-11a, NRCoV2-12, NRCoV2-14, NRCoV2-15, NRCoV2-17, NRCoV2-20, NRCoV2-MRed04, NRCoV2-MRed05 and the S1-NTD-specific V_{H}H-Fcs NRCoV2-SR02 and NRCoV2-SR03.

The surrogate neutralization assays were then extended to variants Alpha, Beta, Gamma, Delta, Kappa and Omicron using all of the RBD-specific and a subset of NTD-specific V_{H}H-Fcs (Table 15). In this assay Wuhan was included and performed again as an internal reference. Several observations were made. First, for cross-neutralizing V_{H}Hs, the IC₅₀s across variants did not change significantly. Second, while all Wuhan neutralizers also remained Alpha neutralizers, some lost their capability to inhibit Beta, Gamma, Delta and Kappa with variable cross-neutralizing patterns. In particular, with respect to the RBD-specific V_{H}Hs, the cross-neutralization profiles for Beta vs Gamma and Delta vs Kappa were identical, which is likely reflective of the key escape mutations in these variants (K417N, E484K and N501Y for Beta vs K417T, E484K and N501Y for Gamma; L452R and T478K for Delta vs L452R and E484Q for Kappa). Third, and importantly, 12 out of 20 V_{H}H-Fcs (10 RBD-specific, two NTD-specific) were Delta neutralizers, nine of which (eight RBD-specific, one NTD-specific) neutralized across all variants. Fourth, the majority of these nine pan-neutralizers (six RBD-specific, one NTD-specific) also neutralized SARS-CoV. Fifth, Omicron mutations had a major impact on antibodies targeting bin 1, from which only NRCoV2-12 and 20 were able to neutralize with comparable potency to Wuhan or the other variants tested. The neutralization ability of the benchmark V_{H}H-72 was abolished by Omicron mutations. Antibodies from bin 2/3/4 were able to neutralize Omicron with comparable IC₅₀ to Wuhan, except for NRCoV-2-02/05 and MRed05, which were negative. NRCoV2-11 (anti-RBD) and SR01 (anti-NTD) were also efficient, achieving neutralization as potent as was observed against Wuhan spike protein. From the list of antibodies tested NRCoV2-12, -20, -11 and -SR01 are the leads, showing efficient pan-neutralization against the SARS-CoV-2 variants generated so far, and outperforming the benchmark V_{H}H-72.

TABLE 15
Flow cytometry SVNAs against SARS-CoV-2 variants and SARS-CoV

| | SVNA IC₅₀ (nM) | | | | | | | | |
|---|---|---|---|---|---|---|---|---|---|
| | | SARS-CoV-2 S | | | | | | | |
| | V_{H}H-Fc | Wuhan | Alpha | Beta | Gamma | Delta | Kappa | Omicron | SARS-CoV S |
| bin | RBD-specific V_{H}H | | | | | | | | |
| 1 | VHH-72 | 5.6 | 10.6 | 5.1 | 3.3 | 10.5 | 8.5 | − | 7.8 |
| | 1d | 4.7 | 6.1 | 13.1 | 4.8 | 6.4 | 5.5 | − | − |
| | 07 | 4.7 | 5.7 | 3.6 | 3.2 | 2.3 | 3.6 | − | 4.2 |
| | 12 | 3.5 | 5.2 | 8 | 3.4 | 6.4 | 6.2 | 17.2 | 3.1 |
| | 18 | 9.1 | 12.2 | 16.6 | 12.1 | 10.2 | 17.4 | − | 12.7 |
| | 20 | 6.5 | 5.2 | 11.9 | 7.5 | 12.6 | 4.1 | 13.1 | 10.3 |
| | MRed04 | 5 | 6.4 | 24.4 | 8.7 | 11.8 | 10.4 | − | 24.6 |
| 2, 3 | 02 | 4.7 | 4.2 | − | − | 8.4 | 7.2 | − | − |
| | 05 | 4.9 | 4.8 | − | − | 7.6 | 6.8 | − | − |
| 2, 4 | 14 | 7.5 | 18 | 58 | 177 | − | − | 5.2 | − |
| 3, 4 | 17 | 8.6 | 10.6 | 26.4 | 214 | − | − | 3.4 | − |
| 2, 3, 4 | 10 | 8.8 | 11.3 | 10.8 | 21.8 | − | − | 4.3 | − |
| | 15 | 5.8 | 9.8 | 12.2 | 10.8 | − | − | 3 | − |
| | MRed05 | 4.3 | 4.2 | 4.4 | 4.7 | 4.8 | 4.8 | − | − |
| 4 | 04 | 10.8 | 21.9 | − | − | − | − | 18.4 | − |
| 5 | 06 | − | − | − | − | − | − | − | − |
| 6 | 03 | − | − | − | − | − | − | − | − |
| | 11 | 3.2 | 6.6 | 10.7 | 4.7 | 7.7 | 3.3 | 8.8 | 7.7 |
| bin | NTD-specific V_{H}H | | | | | | | | |
| 7, 9, 10 | SR01 | 4.2 | 3.1 | 8.8 | 2.1 | 3.1 | 2.3 | 4.4 | 5.1 |
| | SR13 | 7.7 | 22.4 | − | 16.5 | − | 12.2 | − | 15 |
| 10 | SR02 | 1.7 | 7.3 | − | 4.7 | 6.1 | 3.1 | − | − |

“−” dash indicates lack of surrogate neutralization

TABLE 16
Eva Green V_{H}H binding data obtained by tandem SPR surrogate virus neutralization assays against surface-immobilized SARS-CoV-2 S.

| Summary orientation #1: V_{H}H followed by V_{H}H + ACE2 | | | | | | | Summary orientation #2: ACE2 followed by ACE2 + V_{H}H | | | | | | |
|---|---|---|---|---|---|---|---|---|---|---|---|---|---|
| | | | End | End | | | | | | End | End | | |
| | | | 1^{st} | 2^{nd} | inj | | | | | 1^{st} | 2^{nd} | inj | |
| | Solution | Solution | inj¹ | inj¹ | 2 − 1 | | | Solution | Solution | inj¹ | inj¹ | 2 − 1 | |
| Cycle | 1 | 2 | (RU) | (RU) | (ΔRU) | Blocker | Cycle | 1 | 2 | (RU) | (RU) | (ΔRU) | Blocker |
| 1 | Buffer | ACE2 | −3.1 | 56 | 59.1 | No | 1 | − | − | − | − | − | − |
| 2 | NRCoV2- | NRCoV2-1d + | 26.3 | 40 | 13.7 | Yes | 2 | ACE2 | NRCoV2-1d + | 57.1 | 56.4 | −0.7 | Yes |
| | 1d | ACE2 | | | | | | | ACE2 | | | | |
| 3 | NRCoV2- | NRCoV2-02 + | 33 | 39.3 | 6.3 | Yes | 3 | ACE2 | NRCoV2-02 + | 55.8 | 59.6 | 3.8 | Yes |
| | 02 | ACE2 | | | | | | | ACE2 | | | | |
| 4 | NRCoV2- | NRCoV2-03 + | 18.1 | 82.3 | 64.2 | No | 4 | ACE2 | NRCoV2-03 + | 52.8 | 75.3 | 22.5 | No |
| | 03 | ACE2 | | | | | | | ACE2 | | | | |
| 5 | NRCoV2- | NRCoV2-04 + | 41.4 | 95.8 | 54.4 | No | 5 | ACE2 | NRCoV2-04 + | 52.4 | 98.8 | 46.4 | No |
| | 04 | ACE2 | | | | | | | ACE2 | | | | |
| 6 | NRCoV2- | NRCoV2-05 + | 36.9 | 46.8 | 9.9 | Yes | 6 | ACE2 | NRCoV2-05 + | 52.2 | 60.5 | 8.3 | Yes |
| | 05 | ACE2 | | | | | | | ACE2 | | | | |
| 7 | NRCoV2- | NRCoV2-06 + | 38.5 | 92.2 | 53.7 | No | 7 | ACE2 | NRCoV2-06 + | 52.3 | 94.4 | 42.1 | No |
| | 06 | ACE2 | | | | | | | ACE2 | | | | |
| 8 | NRCoV2- | NRCoV2-07 + | 19.1 | 37.5 | 18.4 | Yes | 8 | ACE2 | NRCoV2-07 + | 52 | 59.1 | 7.1 | Yes |
| | 07 | ACE2 | | | | | | | ACE2 | | | | |
| 10 | NRCoV2- | NRCoV2-10 + | 9.6 | 58.8 | 49.2 | No | 10 | ACE2 | NRCoV2-10 + | 52.1 | 64.1 | 12 | No |
| | 10 | ACE2 | | | | | | | ACE2 | | | | |
| 11 | NRCoV2- | NRCoV2-11 + | 21.8 | 80.7 | 58.9 | No | 11 | ACE2 | NRCoV2-11 + | 52.3 | 83.6 | 31.3 | No |
| | 11 | ACE2 | | | | | | | ACE2 | | | | |
| 12 | NRCoV2- | NRCoV2-SR13 + | 36.9 | 86.8 | 49.9 | No | 12 | ACE2 | NRCoV2-SR13 + | 52.3 | 92.8 | 40.5 | No |
| | SR13 | ACE2 | | | | | | | ACE2 | | | | |
| 13 | NRCoV2- | NRCoV2-14 + | 26.9 | 70.4 | 43.5 | +/− | 13 | ACE2 | NRCoV2-14 + | 52.3 | 75.9 | 23.6 | No |
| | 14 | ACE2 | | | | | | | ACE2 | | | | |
| 14 | NRCoV2- | NRCoV2-15 + | 10.1 | 50.7 | 40.6 | +/− | 14 | ACE2 | NRCoV2-15 + | 50 | 59.7 | 9.7 | No |
| | 15 | ACE2 | | | | | | | ACE2 | | | | |
| 15 | NRCoV2- | NRCoV2-SR16 + | 37.4 | 88.8 | 51.4 | No | 15 | ACE2 | NRCoV2-SR16 + | 52.1 | 95.7 | 43.6 | No |
| | SR16 | ACE2 | | | | | | | ACE2 | | | | |
| 16 | NRCoV2- | NRCoV2-17 + | 16.7 | 69.5 | 52.8 | No | 16 | ACE2 | NRCoV2-17 + | 52 | 71.7 | 19.7 | No |
| | 17 | ACE2 | | | | | | | ACE2 | | | | |
| 17 | NRCoV2- | NRCoV2-18 + | 12.6 | 52.5 | 39.9 | +/− | 17 | ACE2 | NRCoV2-18 + | 50.9 | 61.7 | 10.8 | +/− |
| | 18 | ACE2 | | | | | | | ACE2 | | | | |
| 18 | NRCoV2- | NRCoV2-20 + | 27.1 | 60.5 | 33.4 | +/− | 18 | ACE2 | NRCoV2-20 + | 51.3 | 66.2 | 14.9 | +/− |
| | 20 | ACE2 | | | | | | | ACE2 | | | | |
| 19 | NRCoV2- | NRCoV2-SR01 + | 39.4 | 92 | 52.6 | No | 19 | ACE2 | NRCoV2-SR01 + | 51.4 | 97.2 | 45.8 | No |
| | SR01 | ACE2 | | | | | | | ACE2 | | | | |
| 20 | NRCoV2- | NRCoV2-SR02 + | 10.3 | 60.2 | 49.9 | No | 20 | ACE2 | NRCoV2-SR02 + | 49.1 | 67.8 | 18.7 | No |
| | SR02 | ACE2 | | | | | | | ACE2 | | | | |
| 21 | NRCoV2- | NRCoV2-SR03 + | 18.5 | 70.7 | 52.2 | No | 21 | ACE2 | NRCoV2-SR03 + | 50 | 76.4 | 26.4 | No |
| | SR03 | ACE2 | | | | | | | ACE2 | | | | |
| 22 | NRCoV2- | NRCoV2-SR04 + | 10.9 | 63.6 | 52.7 | No | 22 | ACE2 | NRCoV2-SR04 + | 50.5 | 68.5 | 18 | No |
| | SR04 | ACE2 | | | | | | | ACE2 | | | | |
| 23 | NRCoV2- | NRCoV2-S2A3 + | 15 | 82 | 67 | No | 23 | ACE2 | NRCoV2-S2A3 + | 50.8 | 72.2 | 21.4 | No |
| | S2A3 | ACE2 | | | | | | | ACE2 | | | | |
| 24 | NRCoV2- | NRCoV2-S2A4 + | 11.6 | 73.3 | 61.7 | No | 24 | ACE2 | NRCoV2-S2A4 + | 50.9 | 71.1 | 20.2 | No |
| | S2A4 | ACE2 | | | | | | | ACE2 | | | | |
| 25 | NRCoV2- | NRCoV2-S2G3 + | 59 | 113.2 | 54.2 | No | 25 | ACE2 | NRCoV2-S2G3 + | 51 | 114.1 | 63.1 | No |
| | S2G3 | ACE2 | | | | | | | ACE2 | | | | |
| 26 | NRCoV2- | NRCoV2-S2G4 + | 21.3 | 81.7 | 60.4 | No | 26 | ACE2 | NRCoV2-S2G4 + | 50.9 | 79.3 | 28.4 | No |
| | S2G4 | ACE2 | | | | | | | ACE2 | | | | |
| 27 | NRCoV2- | NRCoV2-S2F3 + | 50 | 111.1 | 61.1 | No | 27 | ACE2 | NRCoV2-S2F3 + | 50.7 | 106.9 | 56.2 | No |
| | S2F3 | ACE2 | | | | | | | ACE2 | | | | |
| 28 | V_{H}H-72 | V_{H}H-72 + | 25.8 | 37.8 | 12 | Yes | 28 | ACE2 | V_{H}H-72 + | 51 | 51.2 | 0.2 | Yes |
| | | ACE2 | | | | | | | ACE2 | | | | |

V_{H}Hs were used at 20-40x K_{D} concentrations, ACE2 at 1 μM.
¹inj, injection.

TABLE 17
Neutralization capabilities of SARS-CoV-2-specific
V_{H}Hs/V_{H}H-Fcs obtained by surrogate virus neutralization
flow cytometry assays against SARS-CoV-2 S (Wuhan)

| | Domain/ | V_{H}H/ACE2-H₆² | | | V_{H}H-Fc/ACE2-Fc² | | |
|---|---|---|---|---|---|---|---|
| V_{H}H/ | subdomain | IC₅₀³ | IC₉₉³ | | IC₅₀³ | IC₉₉³ | |
| ACE2 | specificity | (nM) | (nM) | Iₘₐₓ %³ | (nM) | (nM) | Iₘₐₓ %³ |
| ACE2 | S1-RBD | 24.5 | 1506 | 94% | 5.1 | 328.1 | 97% |
| V_{H}H-72¹ | S1-RBD | 59 | 9409 | 43% | 7.2 | 238.5 | 57% |
| NRCoV2-1a | S1-RBD | nd | nd | nd | 9.2 | 89.4 | 94% |
| NRCoV2-1d | S1-RBD | 8.6 | 40.8 | 72% | 5.4 | 42 | 89% |
| NRCoV2-02 | S1-RBD | 5.1 | 15.5 | 100% | 5 | 30 | 94% |
| NRCoV2-03 | S1-RBD | − | − | − | − | − | − |
| NRCoV2-04 | S1-RBD | − | − | − | 11.7 | 420.4 | 51% |
| NRCoV2-05 | S1-RBD | 9.5 | 14.2 | 97% | 6.7 | 37 | 95% |
| NRCoV2-06 | S1-RBD | − | − | − | − | − | − |
| NRCoV2-07 | S1-RBD | 7.5 | 65.3 | 86% | 6.8 | 98.9 | 89% |
| NRCoV2-08 | S1-RBD | nd | nd | nd | − | − | − |
| NRCoV2-10 | S1-RBD | 16.1 | 121 | 50% | 7.7 | 77.3 | 91% |
| NRCoV2-11 | S1-RBD | − | − | − | 9.7 | 99.8 | 63% |
| NRCoV2-11a | S1-RBD | nd | nd | nd | 9.7 | 67.6 | 46% |
| NRCoV2-12 | S1-RBD | nd | nd | nd | 7.3 | 839.9 | 90% |
| NRCoV2-14 | S1-RBD | 21.3 | 17352 | 54% | 9.9 | 160.5 | 83% |
| NRCoV2-15 | S1-RBD | 12.1 | 233 | 44% | 8.1 | 71.5 | 95% |
| NRCoV2-17 | S1-RBD | − | − | − | 8.6 | 100.6 | 88% |
| NRCoV2-18 | S1-RBD | 8.9 | 133 | 51% | 12 | 680.5 | 90% |
| NRCoV2-19 | S1-RBD | nd | nd | nd | − | − | − |
| NRCoV2-20 | S1-RBD | 5.1 | 1371 | 43% | 8.7 | 70.3 | 66% |
| NRCoV2-21 | S1-RBD | nd | nd | nd | − | − | − |
| NRCoV2-MRed04 | S1-RBD | 6.1 | 124 | 53% | 4.6 | 262 | 73% |
| NRCoV2-MRed05 | S1-RBD | 15 | 184 | 88% | 6.1 | 36.3 | 95% |
| NRCoV2-SR01 | S1-NTD | − | − | − | 6.6 | 56.9 | 59% |
| NRCoV2-SR02 | S1-NTD | − | − | − | 5.8 | 49.8 | 53% |
| NRCoV2-SR03 | S1-NTD | − | − | − | − | − | − |
| NRCoV2-SR04 | S1-NTD | − | − | − | − | − | − |
| NRCoV2-SR13 | S1-NTD | − | − | − | 23.8 | 1974.5 | 82% |
| NRCoV2-SR16 | S1-NTD | − | − | − | − | − | − |
| NRCoV2-MRed03 | S1-NTD | − | − | − | − | − | − |
| NRCoV2-MRed06 | S1-NTD | − | − | − | − | − | − |
| NRCoV2-MRed07 | S1-NTD | − | − | − | − | − | − |
| NRCoV2-S2A3 | S2 | − | − | − | − | − | − |
| NRCoV2-S2A4 | S2 | − | − | − | − | − | − |
| NRCoV2-S2F3 | S2 | − | − | − | − | − | − |
| NRCoV2-S2G3 | S2 | − | − | − | − | − | − |
| NRCoV2-S2G4 | S2 | − | − | − | − | − | − |
| NRCoV2-S2H4 | S2 | nd | nd | nd | − | − | − |
| NRCoV2-S202 | S2 | nd | nd | nd | − | − | − |
| NRCoV2-MRed11 | S2 | − | − | − | − | − | − |
| NRCoV2-MRed18 | S2 | − | − | − | − | − | − |
| NRCoV2-MRed19 | S2 | − | − | − | − | − | − |
| NRCoV2-MRed20 | S2 | − | − | − | − | − | − |
| NRCoV2-MRed22 | S2 | − | − | − | − | − | − |
| NRCoV2-MRed25 | S2 | − | − | − | − | − | − |

¹V_{H}H-72 benchmark is SARS-CoV S-specific V_{H}H that cross-reacts with SARS-CoV-2 S (Wrapp et al., 2020).
²ACE2-H₆ is His₆-tagged monomeric ACE2, ACE2-Fc, human Ig Fc-fused dimeric ACE2.
³IC₅₀, concentration of V_{H}H/V_{H}H/Fc giving 50% neutralization; IC₉₉, concentration of V_{H}H/V_{H}H/Fc giving 99% neutralization; Iₘₐₓ %, maximal inhibitory effect; IC₅₀, IC₉₉ and Iₘₐₓ % values were extracted from graphs exemplified in FIGS. 12B and FIGS. 13B. Dash indicate V_{H}H/V_{H}H-Fc does not neutralize the interaction between Vero E6 cell-displayed ACE2 and soluble S.
⁴ICs cannot be determined with certainty due to low Iₘₐₓ % values. nd, not determined, due to lack of sufficient quantities and/or neutralization as V_{H}H-Fc.

### Example 9: Live-Virus Neutralization Assays

### Introduction

V_{H}H-Fcs were subjected to authentic-virus neutralizations assays, i.e., microneutralization assays, to identify those that neutralized infection of host cells by the invading SARS-CoV-2 virus.

### Materials and Methods

### Authentic-Virus Neutralizations Assays

Neutralization activity of antibodies to SARS-CoV-2 was determined with the microneutralization assay. In brief, antibody (V_{H}H-Fc and V_{H}H) stocks were prepared at 1 mg/mL in PBS and sterilized by passing through 0.22 μM filters. 1:5 serial dilutions of 50 μg/mL of each antibody was carried out in DMEM, high glucose media supplemented with 1 mM sodium pyruvate, 1 mM non-essential amino acids, 100 U/ml penicillin-streptomycin, and 1% heat-inactivated fetal bovine serum. SARS-CoV-2 (strain SARS-CoV-2/Canada/VIDO-01/2020) was incubated at 250 pfu with antibody dilution in 1:1 ratio at 37° C. for 1 h. Vero E6 cells seeded in 96-well plates were infected with virus/antibody mix and incubated at 37° C. in humidified/5% CO₂ incubator for 72 hours post-infection (hpi). Cells were then fixed in 10% formaldehyde overnight and virus infection was detected with mouse anti-SARS-CoV-2 nucleocapsid antibody (R&D Systems, clone #1035111) and counterstained with rabbit anti-mouse IgG-HRP (Rockland Inc.). Colorimetric development was obtained with o-phenylenediamine dihydrochloride peroxidate substrate (Sigma-Aldrich) and detected on Biotek Synergy H1 plate reader at 490 nm. IC₅₀ was determined from non-linear regression on GraphPad Prism 9. For determining neutralization potencies by measuring cytopathic effect (CPE), infected Vero E6 cells were incubated at 37° C. for 96 h until the virus-only control wells had nearly 100% CPE (cell-only controls were also included). Neutralization was scored by MN₁₀₀, lowest antibody concentration that gave no CPE, i.e., 100% neutralization. Assays were performed in technical duplicates.

### Results and Discussion

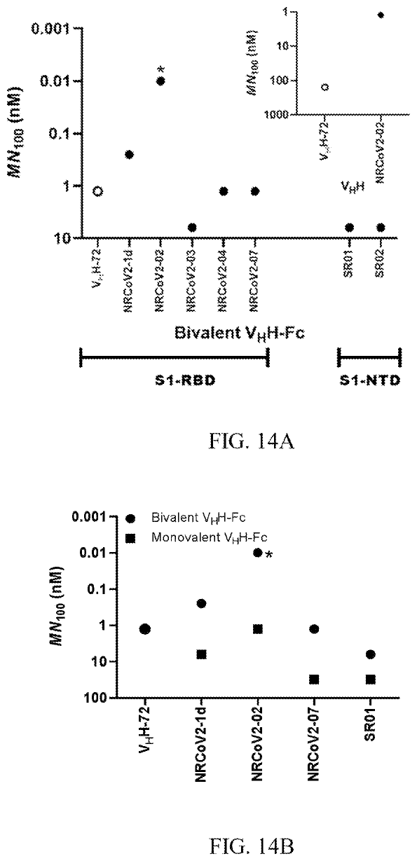

A select set of lead V_{H}H-Fcs were subjected to preliminary authentic-virus microneutralization assays to assess their SARS-CoV-2 virus-neutralizing activity. These included five S1-RBD-specific V_{H}Hs and two S1-NTD-specfic V_{H}Hs. Neutralization was scored by MN₁₀₀, the lowest antibody concentration that gave no cytopathic effect (100% neutralization). Results are shown in FIGS. 14A-B and Table 18. All V_{H}H-Fcs demonstrated significant neutralization capabilities, with MN₁₀₀s ranging from 6.25 nM (lowest neutralization capability) to 50.01 nM (highest neutralization capability). The most potent neutralizers were amongst the S1-RBD binders: NRCoV2-02 (MN₁₀₀≤0.01 nM); NRCoV2-1d (MN₁₀₀ 0.25 nM); NRCoV2-04 and NRCoV2-07 (MN₁₀₀ 1.25 nM); NRCoV2-03 (MN₁₀₀ 6.25 nM). NRCoV2-02 and NRCoV2-1 d were far more potent neutralizers than the benchmark (V_{H}H-72), by five- and 125-fold, respectively. S1-NTD binders had MN₁₀₀s of 6.25 nM (NRCoV2-SR01, NRCoV2-SR02). The lead antibody, NRCoV2-02 also outperformed the benchmark in V_{H}H format by 125-fold (FIG. 14A inset). To explore the contribution of bivalency to the neutralization potency of V_{H}H-Fcs, monovalent V_{H}H-Fc versions of select V_{H}H-Fcs were generated. Based on MN₁₀₀ values, neutralization potencies were decreased by five-fold for NRCoV2-SR01, 25-fold for NRCoV2-1d and NRCoV2-07 and more than 125-fold for NRCoV2-02, with their conversion from bivalent to monovalent V_{H}H-Fcs, demonstrating the sizable contribution of bivalency to their neutralization potency. In the case of NRCoV2-02, the identical MN₁₀₀ for its monovalent V_{H}H and monovalent V_{H}H-Fc versions indicates that the observed dramatic increase in neutralization potency in going from V_{H}H to bivalent V_{H}H-Fc was likely due solely to an increase in valency, not size (steric hindrance). The loss of bivalency also had drastic effect on V_{H}H-72, rendering it non-neutralizing at the highest concentration tested.

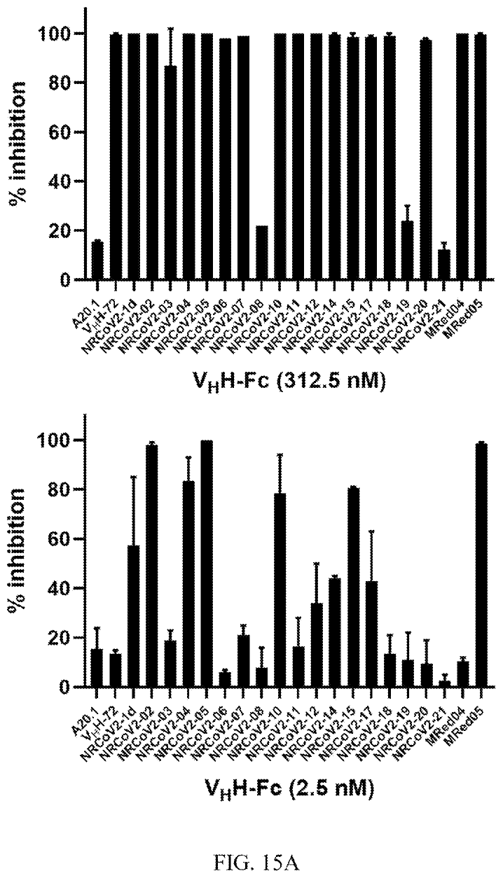

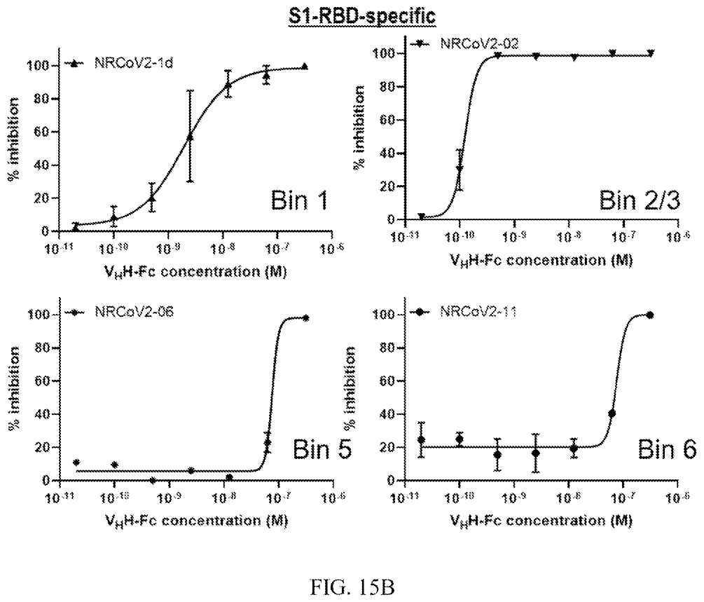

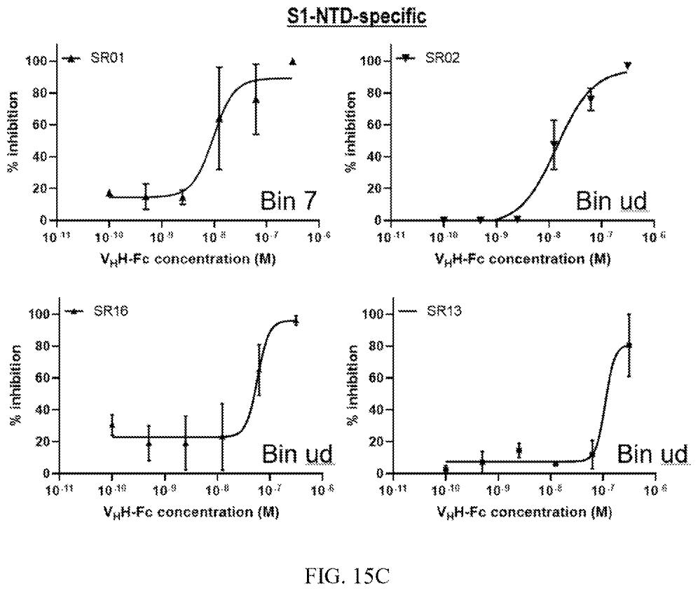

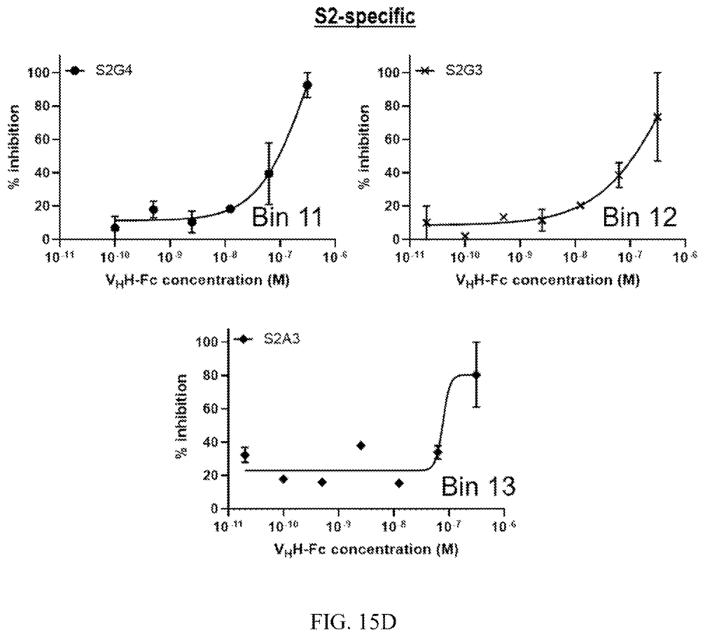

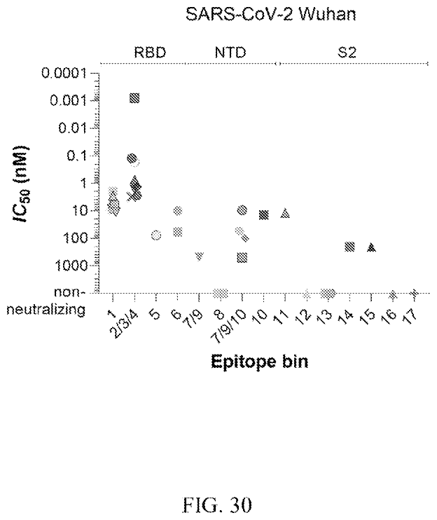

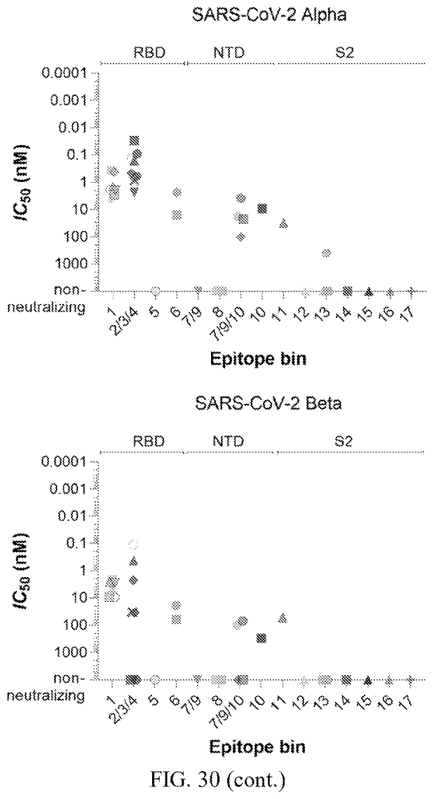

A more comprehensive authentic neutralization assay was performed to determine the IC₅₀ of V_{H}H-Fcs (FIG. 15 A-D; Table 19). Most potent neutralizers were amongst the S1-RBD binders with 17 out of 20 V_{H}H-Fcs tested being neutralizing. The most potent V_{H}H-Fcs recognized epitopes 2/3/4 and had IC₅₀s of 0.0008-3.1 nM (FIG. 15E and FIG. 30; Table 19). The leads were NRCoV-05 (IC₅₀ 0.0008 nM) followed closely by NRCoV-02 (IC₅₀ 0.12 nM) and NRCoV2-MRed 05 (IC₅₀ 0.17 nM). V_{H}H-Fcs recognizing epitope 1 showed intermediate potencies with IC₅₀s of 1.94-9.6 nM, with V_{H}H-72 (belonging to the same bin 1) having similar IC₅₀ (8.46 nM). V_{H}H-Fcs recognizing epitope 5 and 6 showed IC₅₀s of 9.96-76 nM. As for S1-NTD-specific V_{H}H, six out of nine V_{H}H-Fcs tested were neutralizing, with the lead V_{H}H-Fcs having IC₅₀s of 9.42, 14.31 and 54.2 nM. The remaining two had IC₅₀s in the high nM—micromolar range. Out of 13 S2-specific V_{H}H-Fcs tested, three, NRCoV2-S2A3, NRCoV2-S2G3 and NRCoV2-S2G4, were neutralizing with IC₅₀s from 12.2 nM for S2A3 to high nM—micromolar range for S2G3 and S2G4. These belonged to three different epitope bins. Nine V_{H}H-Fcs outperformed the V_{H}H-72 benchmark by 2.5-10,000-fold. In particular, the NRCoV2-05, NRCoV2-02 and NRCoV2-MRed05 leads showed 10,000-fold, 70-fold and 50-fold higher potency than V_{H}H-72, respectively. We provide the first examples of single domain antibodies that neutralize the SARS-CoV-2 virus by targeting the non-S1-RBD region of S, i.e., S1-NTD and S2.

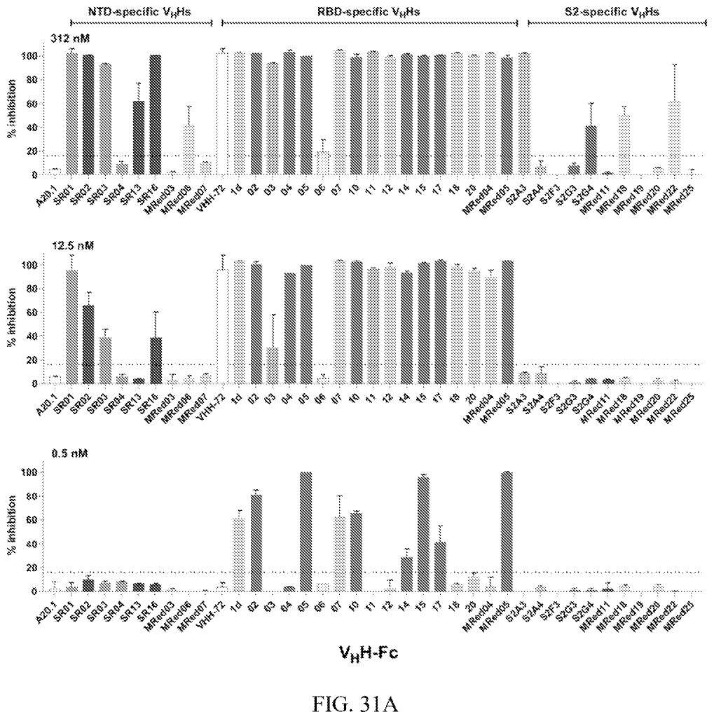

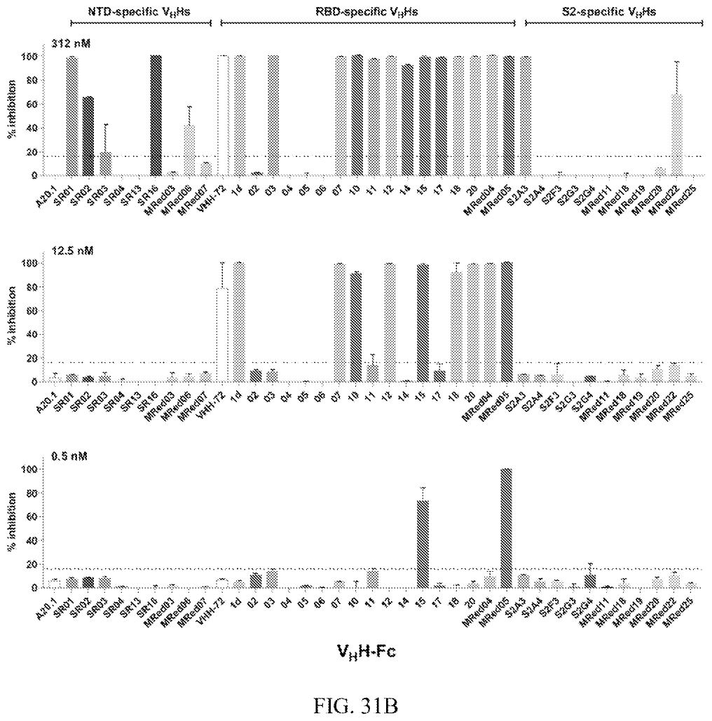

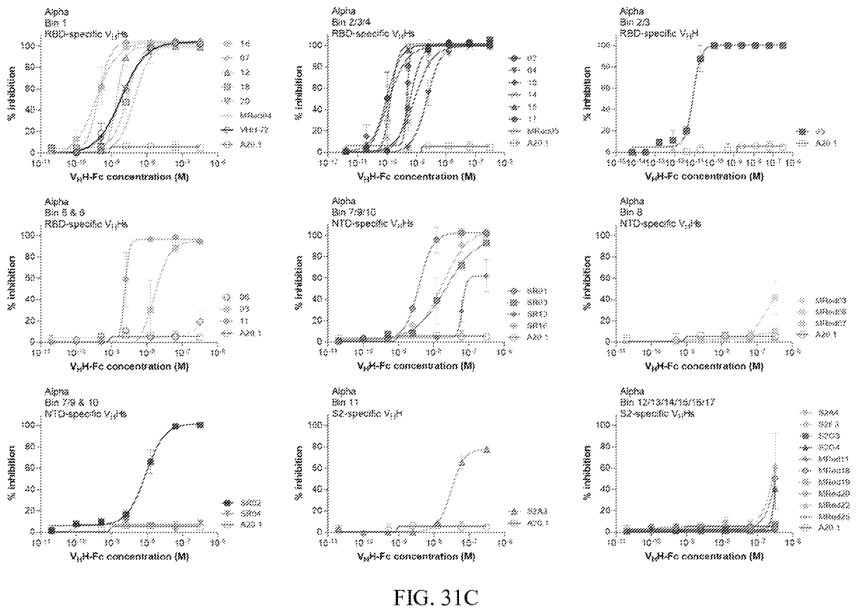

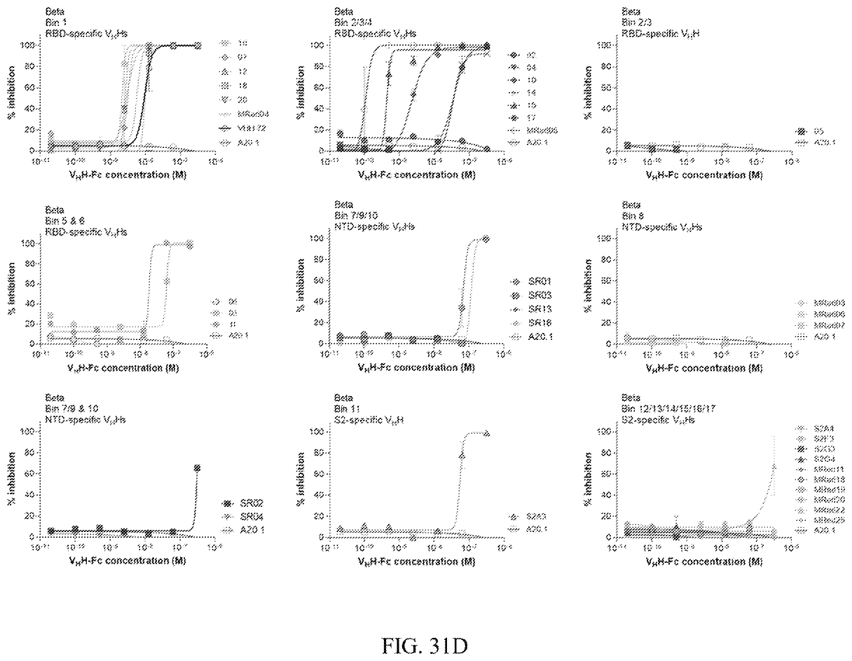

The live virus neutralization assays were then extended to include Alpha and Beta variants. With the exception of V_{H}H-Fc NRCoV2-06, all remaining 16 RBD-specific Wuhan neutralizers maintained their ability to neutralize Alpha (Table 19, FIG. 30, FIG. 31A, and FIG. 31C). Interestingly, many V_{H}Hs from across different epitope bins showed improved IC₅₀s by as high as 15-fold. Except for NRCoV2-05, which despite showing a reduced potency towards the Alpha variant (˜40-fold) still exhibited the highest potency of all against the variant, the remaining V_{H}Hs demonstrated comparable potencies. Of the 16 Wuhan/Alpha neutralizers, 13 also neutralized the Beta variant (FIG. 31B and FIG. 31D), with the majority (10 of 13) demonstrating comparable potencies and two (NRCoV2-14 and NRCoV2-17) showing reductions (˜10-fold). Although from the most potent bin (2/3/4), NRCoV2-02, NRCoV2-04 and NRCoV2-05, consistent with the cross-reactivity data (FIG. 6B), were completely abrogated presumably by the Beta mutations in the RBD (K417N, E484K, N501Y), several others including NRCoV2-MRed05, NRCoV2-10 and NRCov2-15 did retain their high neutralizing potencies against both Alpha and Beta variants. A similar trend was observed for the NTD-specific neutralizing V_{H}Hs: against the Alpha variant, potencies either remained essentially the same as those for the Wuhan variant or improved, while against the Beta variant, potencies diminished (FIG. 30 and FIGS. 31 A-D). Nonetheless, NRCoV2-SR01 and NRCoV2-SR16 maintained respectable neutralization potencies against Beta. The potencies of S2-specific neutralizers (S2A3, S2G3, S2G4) were also decreased with variants. However, the lead NRCoV2-S2A3 still maintained comparable potencies across all three variants (IC₅₀ of 12.2 nM, 31 nM and 54 nM for Wuhan, Alpha and Beta [Table 19]). Collectively, the neutralization profiles across Wuhan, Alpha and Beta variants were consistent with cross-reactivity profiles (FIG. 6B). Based on the cross-reactivity (FIG. 6B) and surrogate cross-neutralization data (Table 15), it is likely that many V_{H}Hs would also neutralize the Gamma, Kappa, Delta, and Omicron variants in live virus neutralization assays.

TABLE 18
Neutralization capabilities (MN₁₀₀) of SARS-CoV-2-specific V_{H}H-Fcs
obtained by authentic-virus (aka live virus) neutralization assays

| | Domain/subunit | |
|---|---|---|
| V_{H}H | specificity | MN₁₀₀ (nM)² |
| NRCoV2-1d | S1-RBD | 0.25 |
| NRCoV2-02 | S1-RBD | ≤0.01³ |
| NRCoV2-03 | S1-RBD | 6.25 |
| NRCoV2-04 | S1-RBD | 1.25 |
| NRCoV2-07 | S1-RBD | 1.25 |
| NRCoV2-SR01 | S1-NTD | 6.25 |
| NRCoV2-SR02 | S1-NTD | 6.25 |
| NRCoV2-15 × | S1-RBD × null | 1.25 |
| A26.8 | | |
| V_{H}H72 × | S1-RBD × null | — |
| A26.8 | | |
| NRCoV2-S2A4 × | S2 × null | — |
| A26.8 | | |
| NRCoV2-07 × | S1-RBD × null | 31.25 |
| A26.8 | | |
| NRCoV2-1d × | S1-RBD × null | 6.25 |
| A26.8 | | |
| NRCoV2-SR01 × | S1-NTD × null | 31.25 |
| A26.8 | | |
| NRCoV2-02 × | S1-RBD × null | 1.25 |
| A26.8 | | |
| V_{H}H-72¹ | S1-RBD | 1.25³ |
| A20.1¹ | null | — |

¹V_{H}H-72 benchmark is a SARS-CoV S-specific V_{H}H that cross-reacts with SARS-CoV-2 S (Wrapp et al., 2020); A20.1 and A26.8 are C. difficile toxin A-specific negative control V_{H}H (Hussack et al., 2011).
²MN₁₀₀ is the lowest antibody concentration that gave no cytopathic effect (100% neutralization). Dash indicate V_{H}H-Fc does not neutralize SARS-CoV-2 virus at the highest V_{H}H-Fc concentration used. MN₁₀₀ values were used to construct FIG. 14A-B graphs.
³The MN₁₀₀ of monovalent V_{H}H-72 and NRCoV2-02 V_{H}Hs were 156.25 and 1.25 nM, respectively.

TABLE 19
Neutralization capabilities (IC₅₀) of SARS-CoV-2-specific V_{H}H-Fcs
obtained by authentic-virus (aka live virus) neutralization assays

| | Epitope | LVNA IC₅₀ (nM) | | |
|---|---|---|---|---|
| V_{H}H-Fc | bin | Wuhan | Alpha | Beta |

RBD-specific V_{H}H

| | | | | |
|---|---|---|---|---|
| 1d | 1 | 1.94 | 0.37 | 2.14 |
| 07 | | 6.15 | 0.42 | 3.18 |
| 12 | | 2.82 | 1.35 | 2.62 |
| 18 | | 6.4 | 2.82 | 9.48 |
| 20 | | 11.2 | 1.94 | 2.88 |
| MRed04 | | 9.61 | 4.5 | 5.73 |
| 02 | 2, 3 | 0.12 | 0.09 | — |
| 05 | | 0.0008 | 0.03 | — |
| 14 | 2, 4 | 3.1 | 0.88 | 32.8 |
| 17 | 3, 4 | 2.82 | 0.61 | 34.7 |
| 10 | 2, 3, 4 | 1.28 | 0.47 | 2.25 |
| 15 | | 0.73 | 0.16 | 0.43 |
| MRed05 | | 0.17 | 0.13 | 0.11 |
| 04 | 4 | 1.65 | 2.3 | — |
| 06 | 5 | 76 | — | — |
| 03 | 6 | 58 | 16 | 62 |
| 11 | | 9.9 | 2.3 | 18.5 |

NTD-specific V_{H}H

| | | | | |
|---|---|---|---|---|
| SR01 | 7, 9, 10 | 9.42 | 3.77 | 70.3 |
| SR03 | | ~500 | 22.2 | — |
| SR13 | | ~100 | ~100 | — |
| SR16 | | 54.2 | 17.8 | 100 |
| SR04 | 7/9 | ~500 | — | — |
| MRed03 | 8 | — | — | — |
| MRed06 | 8 | — | — | — |
| MRed07 | 9 | — | — | — |
| SR02 | 10 | 14.13 | 9.05 | ~300 |

S2-specific V_{H}H

| | | | | |
|---|---|---|---|---|
| S2A3 | 11 | 12.2 | 31 | 54 |
| S2A4 | 12 | — | — | — |
| S2F3 | | — | — | — |
| MRed18 | | — | ~400 | — |
| MRed19 | 13 | — | — | — |
| MRed20 | | — | — | — |
| MRed22 | | — | — | — |
| S2G3 | 14 | ~200 | — | — |
| S2G4 | 15 | ~200 | — | — |
| MRed11 | 16 | — | — | — |
| MRed25 | 17 | — | — | — |

Reference

| | | | | |
|---|---|---|---|---|
| VHH-72¹ | 1 | 8.46 | 1.86 | 9.34 |
| A20.1¹ | null | — | — | — |

¹V_{H}H-72 benchmark is a SARS-CoV S-specific V_{H}H that cross-reacts with SARS-CoV-2 S (Wrapp et al., 2020); A20.1 is C. difficile toxin A-specific negative control V_{H}H (Hussack et al., 2011. Epitope bin numbers correspond to the bins shown in FIG. 9G.

### Example 10: Stability of V_{H}Hs Against Aerosolization

### Introduction

One effective therapeutic approach against COVID-19 might be the direct delivery of aerosolized antibodies to the nasal and lung epithelia by inhalation. V_{H}Hs in particular, are advantageously fit for such administration approach due to their high stability and robustness. Since aerosolization could compromise the structural integrity and function of antibodies that lack sufficient stability, such as mAbs (Detalle et al., 2016; Respaud et al., 2015), the effect of aerosolization on the stability of V_{H}Hs was tested.

### Materials and Methods

### Aerosolization Studies

Prior to aerosolization, 4 mg of each V_{H}H was purified by size-exclusion chromatography using a Superdex™ 75 GL column (Cytiva) and PBS as running buffer, as described above. Protein fractions corresponding to the chromatogram's monomeric peak were pooled, quantified and the concentration adjusted to 0.5 mg/mL. One mL of each V_{H}H was subsequently aerosolized at room temperature with a portable mesh nebulizer (AeroNeb® Solo, Aerogen, Galway, Ireland), which produces 3.4-μm particles. Aerosolized V_{H}Hs were collected into 15 mL Round-Bottom Polypropylene test tubes (Falcon, Cat #C352059) for 5 min to allow condensation and were subsequently quantified and kept at 4° C. until use. Then 200 μL aliquots of pre- and post-aerosolized V_{H}Hs were subjected to SEC to obtain chromatogram profiles. Additionally, condensed V_{H}Hs were closely monitored for the formation of any visible aggregates, and in cases where aggregate formation was observed, aggregates were removed by centrifugation prior to concentration determination, SEC analysis and ELISA. % soluble aggregate was determined as the proportion of a V_{H}H that gave elution volumes (Vₑs) smaller than that of the monomeric V_{H}H fraction. % recovery was determined as the proportion of a V_{H}H that remained monomerically soluble following aerosolization.

To assess the effect of aerosolization on the functionality of V_{H}Hs, the activities of post-aerosolized V_{H}Hs were determined by ELISA and compared to those for pre-aerosolized V_{H}Hs. To perform ELISA, S1-Fc (ACRO Biosystems, Cat #S1N-05255) was diluted in PBS to 500 ng/mL, and 100 μL/well were coated overnight at 4° C. The next day, plates were washed with PBST and blocked with 200 μL PBSC for 1 h at room temperature. After five washes with PBST, serial dilutions of the pre- and post-aerosolized V_{H}Hs were added to wells and incubated for 1 h at room temperature. Then plates were washed 10 times with PBST and binding of V_{H}Hs to S1-Fc was detected with rabbit anti-6×His Tag antibody HRP Conjugate (Bethyl, Cat #A190-114P), diluted at 10 ng/mL in PBST and added at 100 μL/well. Finally, after 1 h incubation at room temperature, peroxidase activity was detected as described previously.

### Results and Discussion

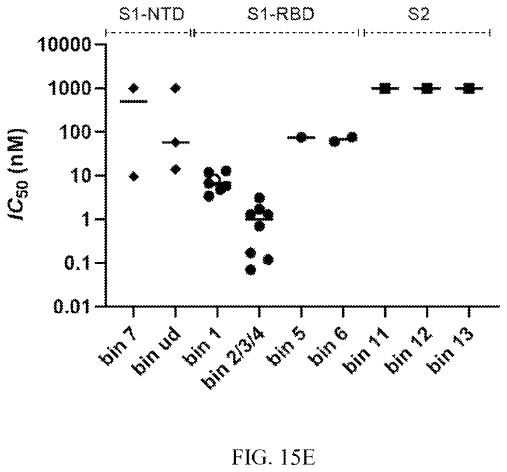

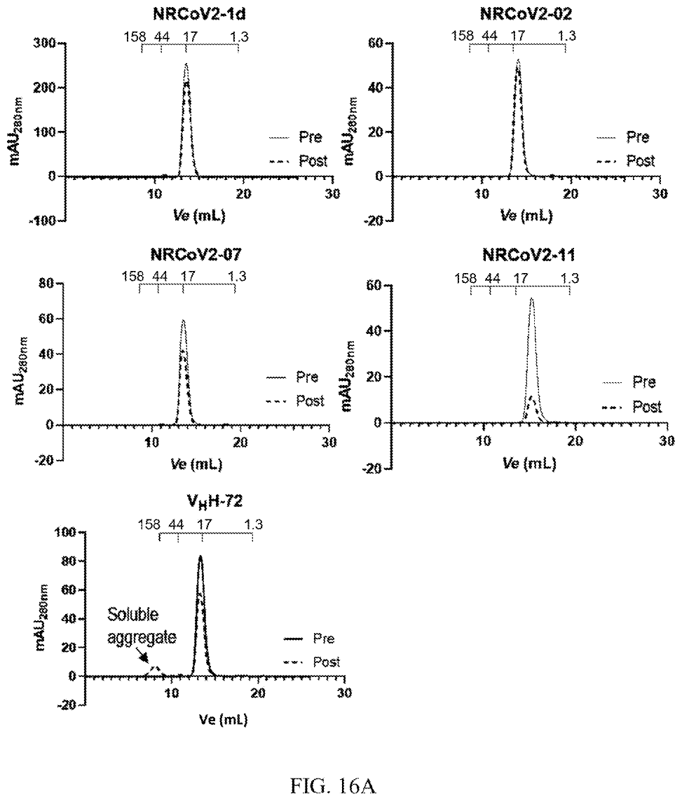

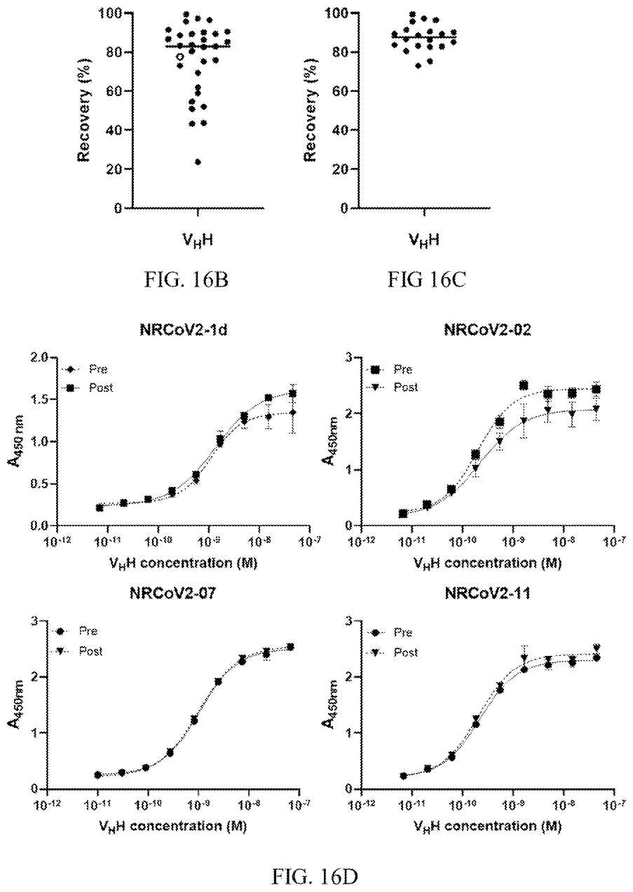

V_{H}Hs including the benchmark V_{H}H-72 were examined for their aggregation resistance/stability against aerosolization. For a few V_{H}Hs, e.g., NRCoV2-MRed20, NRCoV2-S2A4, as well as the V_{H}H-72 benchmark, aerosolization induced some soluble aggregation formation as determined by SEC (FIG. 16A; Table 20). Several V_{H}Hs, e.g., NRCoV2-11, NRCoV2-SR03, formed visible aggregates, which led to their reduced % recovery (FIG. 16A-C; Table 20). However, the majority of V_{H}Hs (20 out of 30 V_{H}Hs tested) were highly stable against aerosolization, that is, they did not form any soluble or visible aggregates and demonstrated high % recovery upon aerosolization treatment. Examples include NRCoV2-1c/1d, NRCoV2-02, NRCoV2-07, NRCoV2-17, NRCoV2-18 and NRCoV2-20. High % recovery indicates these V_{H}Hs advantageously lack non-specific binding to nebulizer surfaces. For therapeutic V_{H}Hs, this is expected to translate to a more effective drug delivery to the site of viral infection. Several V_{H}Hs, i.e., NRCoV2-04, NRCoV2-14, NRCoV2-15, NRCoV2-SR04 and NRCoV2-MRed04, while forming some visible aggregates, still showed a good % recovery upon aerosolization (52-69%). To assess the effect of aerosolization on the functionality of V_{H}Hs, the activities (EC₅₀s) of post-aerosolized V_{H}Hs were determined by ELISA and compared to those for pre-aerosolized V_{H}Hs. ELISAs were performed on a sample of four V_{H}Hs: NRCoV2-1d, NRCoV2-02, NRCoV2-07 and NRCoV2-11 (FIG. 16D). Comparison of EC₅₀s for post-aerosolized V_{H}Hs vs pre-aerosolized V_{H}Hs demonstrated that aerosolization did not compromise the functionality of V_{H}Hs (FIG. 16D; Table 21).

TABLE 20
Stability of V_{H}Hs against aerosolization

| | | Soluble aggregates (%)² | | | |
|---|---|---|---|---|---|
| | Recovery | Pre- | Post- | ΔSoluble | Visible |
| V_{H}H | (%)¹ | aerosolization | aerosolization | agg.³ | aggregates |
| NRCoV2-1c | 91 | 3 | 5 | 2 | No |
| NRCoV2-1d | 83 | 2 | 4 | 2 | No |
| NRCoV2-02 | 89 | 2 | 2 | 0 | No |
| NRCoV2-03 | 81 | 2 | 1 | −1 | No |
| NRCoV2-04 | 62 | 6 | 5 | −1 | Yes |
| NRCoV2-05 | 89 | 2 | 5 | 3 | No |
| NRCoV2-06 | 51 | 7 | 5 | −2 | Yes |
| NRCoV2-07 | 75 | 2 | 3 | 1 | No |
| NRCoV2-10 | 83 | 5 | 5 | 0 | No |
| NRCoV2-11 | 24 | 6 | 5 | −1 | Yes |
| NRCoV2-14 | 55 | 6 | 6 | 0 | Yes |
| NRCoV2-15 | 69 | 4 | 5 | 1 | Yes |
| NRCoV2-17 | 85 | 5 | 6 | 1 | No |
| NRCoV2-18 | 99 | 9 | 5 | −4 | No |
| NRCoV2-20 | 97 | 2 | 1 | −1 | No |
| NRCoV2-SR03 | 43 | 3 | 11 | 8 | Yes |
| NRCoV2-SR04 | 52 | 5 | 3 | −2 | Yes |
| NRCoV2-SR13 | 83 | 4 | 6 | 2 | No |
| NRCoV2-S2A4 | 84 | 7 | 11 | 4 | No |
| NRCoV2-S2G4 | 91 | 3 | 6 | 3 | No |
| NRCoV2-MRed02 | 73 | 1 | 1 | 0 | No |
| NRCoV2-MRed03 | 96 | 3 | 2 | −1 | No |
| NRCoV2-MRed04 | 59 | 4 | 4 | 0 | Yes |
| NRCoV2-MRed07 | 90 | 10 | 3 | −7 | No |
| NRCoV2-MRed11 | 89 | 4 | 5 | 1 | No |
| NRCoV2-MRed18 | 96 | 3 | 10 | 7 | No |
| NRCoV2-MRed19 | 87 | 5 | 9 | 4 | No |
| NRCoV2-MRed20 | 76 | 2 | 18 | 16 | No |
| NRCoV2-MRed22 | 86 | 3 | 9 | 6 | No |
| NRCoV2-MRed25 | 44 | 3 | 3 | 0 | Yes |
| V_{H}H-72 | 78 | 1 | 14 | 13 | No |

¹% recovery were determined as described in Examples 10.
²% soluble aggregate was determined as the proportion of a V_{H}H that gave elution volumes (Vₑs) smaller than that of the monomeric V_{H}H fraction.
³ΔSoluble agg. = “Post-aerosolization” − “Pre-aerosolization”.

TABLE 21
Affinities (EC₅₀s) of pre-aerosolized (“Pre”) vs
post-aerosolized (“Post”) V_{H}Hs

EC₅₀ (nM)

| V_{H}H | Pre | Post |
|---|---|---|
| NRCoV2-1d | 1.1 | 1.3 |
| NRCoV2-02 | 0.2 | 0.2 |
| NRCoV2-07 | 1.1 | 1 |
| NRCoV2-11 | 0.2 | 0.2 |

### Example 11: V_{H}Hs for Diagnosis and Capture of SARS-CoV-2

introduction

V_{H}Hs described herein are promising diagnostic/capture agents against SARS-CoV-2, SARS-CoV and related viruses as well as their spike proteins. To explore the use of these V_{H}Hs as capture agents, four of the V_{H}Hs were tested in sandwich ELISA for their diagnostic/capturing capability against SARS-CoV-2.

### Materials and Methods

### Sandwich ELISA

NUNC® MaxiSorp™ 4 HBX plates (Thermo Fisher) were coated overnight at 4° C. with 4 μg/mL streptavidin (Jackson ImmunoResearch, Cat #016-000-113) in 100 μL PBS, pH 7.4. Wells were blocked with 200 μL PBSC for 1 h at room temperature followed by capturing biotinylated NRCoV2-02 V_{H}H (10 μg/mL in 100 μL PBSCT) for 1 h at room temperature. Wells were washed five times with PBST and incubated with variable concentrations of SARS-CoV-2 S, S1 or S1-RBD diluted in PBSCT for 1 h. Well were washed and incubated with detecting V_{H}H-Fcs at 1 μg/mL. The binding of V_{H}H-Fcs to spike protein fragments was probed using 100 μL 1 μg/mL HRP-conjugated goat anti-human IgG (SIGMA, Cat #A0170). Finally, plates were washed 10 times with PBST and peroxidase activity was determined as described above.

### Results and Discussion

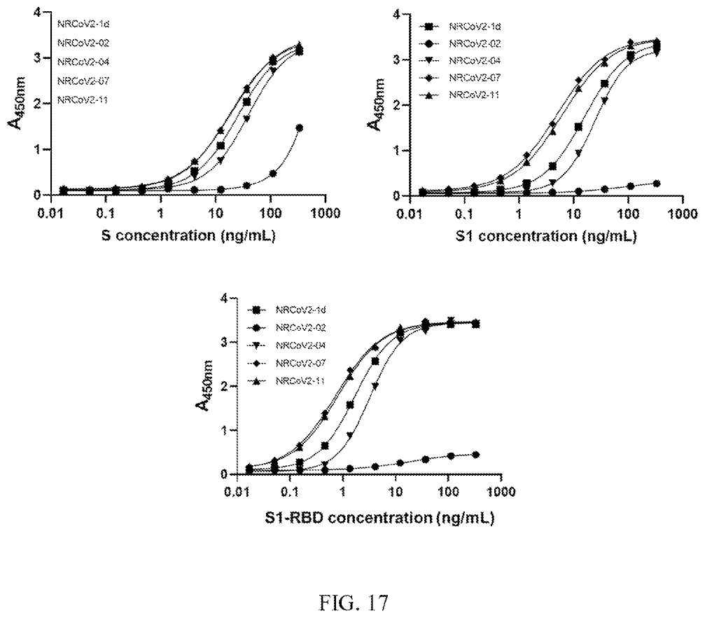

To provide proof of concept for the utility of the V_{H}Hs as detecting/capturing agents against SARS-CoV-2, SARS-CoV and related viruses, sandwich ELISAs were performed with four V_{H}Hs using SARS-CoV-2 spike protein fragments as surrogates for the virus. Wells were coated with NRCoV2-02 V_{H}H as the capturing antibody, followed by the capture of antigens S, S1, or S1-RBD added at variable concentrations. Then a second, V_{H}H-Fc that binds to a non-overlapping epitope in relation to NRCoV2-02 was added as the detecting antibody followed by the addition of a HRP-conjugated probing antibody binding to the detecting antibody. The different V_{H}H-Fcs tested as detecting antibodies were: NRCoV2-1d, NRCoV2-04, NRCoV2-07, and NRCoV2-11. Very low SC₅₀ values were obtained in ELISA assays (FIG. 17, Table 22). In addition, limit of detection values as low as 0.08 ng/mL (8 picogram) spike protein could be detected with confidence (Table 23). These results indicate that the V_{H}Hs are promising virus detecting/capturing agents.

TABLE 22
SC₅₀ values obtained in ELISA assays

| | SC₅₀ (ng/mL) | | |
|---|---|---|---|
| | S | S1 | S1-RBD |
| NRCoV2-1d | 25 | 16 | 1.7 |
| NRCoV2-04 | 39 | 25 | 3.3 |
| NRCoV2-07 | 18 | 5 | 0.7 |
| NRCoV2-11 | 20 | 6 | 0.8 |

TABLE 23
Limit of detection (ng/mL)

| | NRCoV2-1d | NRCoV2-04 | NRCoV2-07 | NRCoV2-11 |
|---|---|---|---|---|
| S | 1.4 | 4.1 | 1.4 | 1.4 |
| S1 | 1.37 | 4.12 | 0.46 | 0.46 |
| S1-RBD | 0.15 | 0.46 | 0.08 | 0.08 |

### Example 12: In Vivo Therapeutic Efficacy of V_{H}H-Fcs

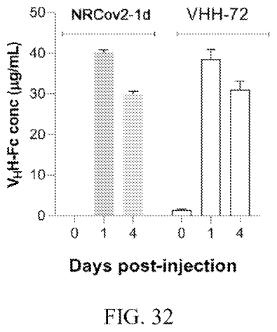

Before testing V_{H}H-Fcs in hamsters for in vivo efficacy, they were assessed for in vivo stability and persistence. NRCov2-1d V_{H}H-Fc was chosen as a representative V_{H}H and V_{H}H-72 V_{H}H-Fc, whose modified/enhanced version is currently in a phase 1 clinical trial, was included as a reference. Hamsters were injected intraperitoneally (IP) with 1 mg of each antibody and serum antibody concentration was monitored for up to four days by ELISA. Significant and comparable V_{H}H-Fc concentrations were present in the hamster sera for both 1 d and V_{H}H-72 V_{H}H-Fcs on days 1 and 4 post injection (FIG. 32), indicating that V_{H}H-Fcs would have the required serum stability and persistence in vivo for the duration of the animal studies.

The in vivo therapeutic efficacy of V_{H}H-Fcs which were neutralizing by live virus neutralization assay was then assessed in a hamster model of SARS-CoV-2 infection. Five V_{H}H-Fcs were selected to cover a wide range of important attributes including in vitro neutralization potencies and breadth, epitope bin, subunit/domain specificity and cross-reactivity pattern. These included three RBD-specific (1d, 05, MRed05), one NTD-specific (SR01) and one S2-specific (S2A3) V_{H}H-Fcs. Cocktails of two V_{H}H-Fcs were also included to explore synergy between the antibody pairs recognizing distinct epitopes within the RBD (1 d/1d/MRed05) or RBD and NTD (1d/SR01).

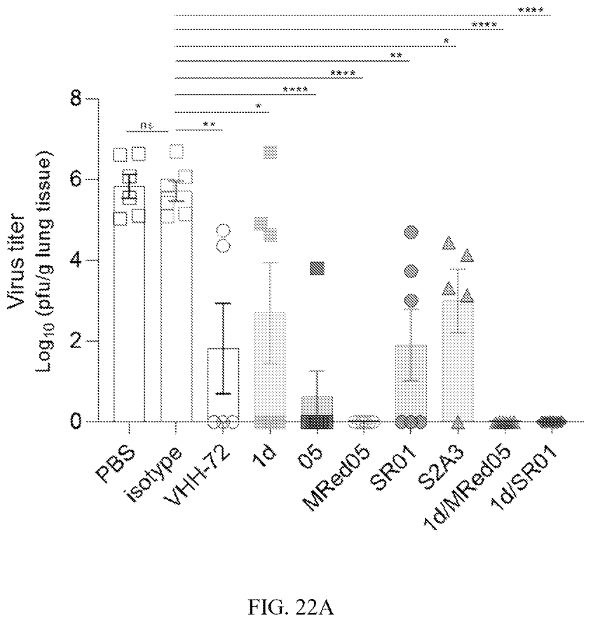

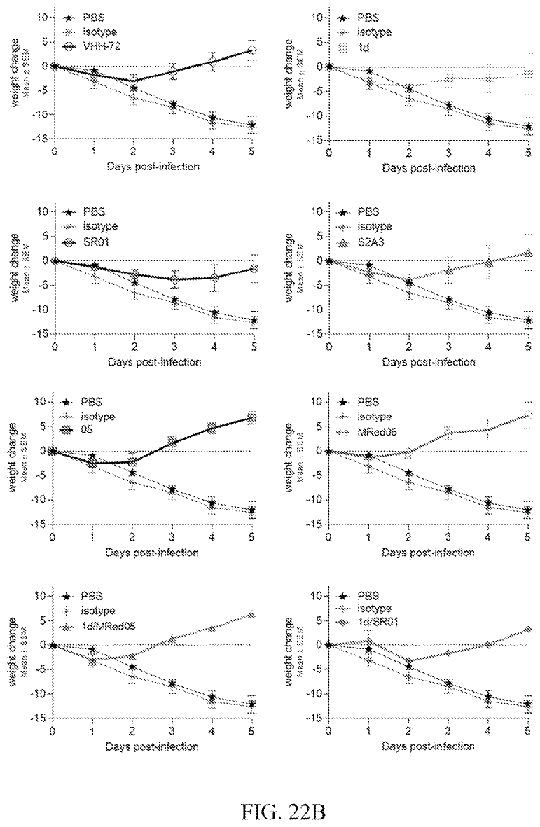

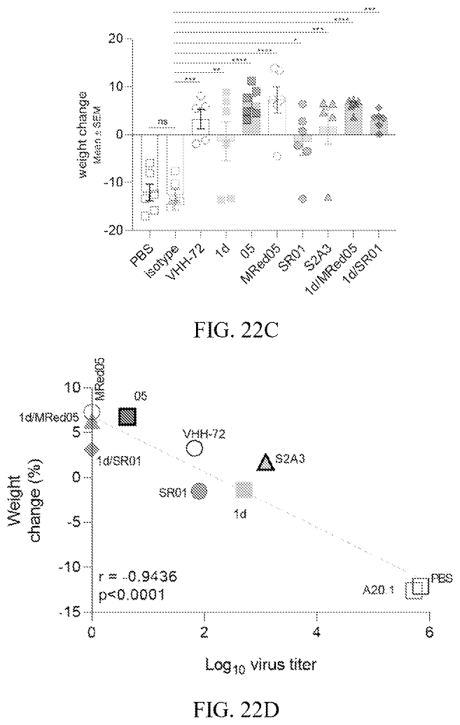

Hamsters were administered IP with 1 mg of V_{H}H-Fcs 24 h prior to intranasal challenge with SARS-CoV-2 Wuhan isolate. Daily weight change and clinical symptoms were monitored. At 5 dpi, lungs were collected to determine viral titers. Viral titer decrease and reversal of weight loss in antibody treated versus control animals were taken as measures of antibody efficacy. Animals treated with RBD binders 1 d, 05, and MRed05 showed reduced lung viral burden by three, five and six orders of magnitude, respectively, relative to PBS or V_{H}H-Fc isotype controls, with 05 and MRed05 reducing viral burden to below detectable levels (FIG. 22A). The RBD-specific V_{H}H-72 benchmark caused a mean viral decrease of four orders of magnitude. The NTD binder SR01, and interestingly, the S2 binder S2A3, were also effective neutralizers, decreasing mean viral titers by four and three orders of magnitude, respectively. Both 1 d/SR01 and 1 d/MRed05 cocktails decreased viral titers by 6 orders of magnitude to undetectable levels of virus infection. While it was not possible to unravel potential synergies for 1d/MRed05, as MRed05 alone displayed essentially the same efficacy as the 1d/MRed05 combination, it was apparent that the 1 d/SR01 combination benefited from synergy, decreasing viral titers by a further 2-3 orders of magnitude to undetectable levels, relative to 1d or SR01 alone. Moreover, in accordance with the viral titer decreases, a gradual reversal of weight loss in infected animals was observed with antibody treatment starting on 2 dpi (FIGS. 22B and 22C). A strong negative correlation (r=−0.9436; p<0.0001) was observed between weight change and viral titer at 5 dpi (FIG. 22D).

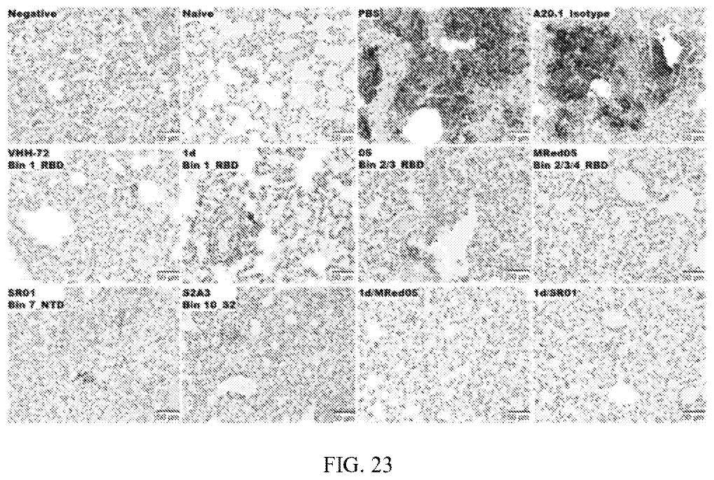

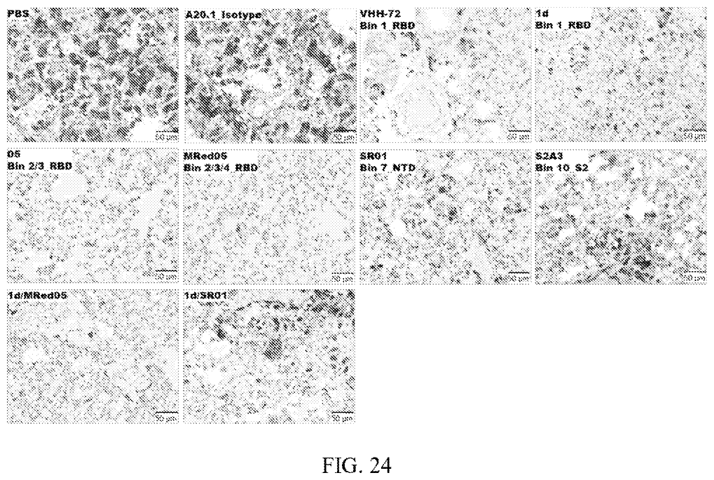

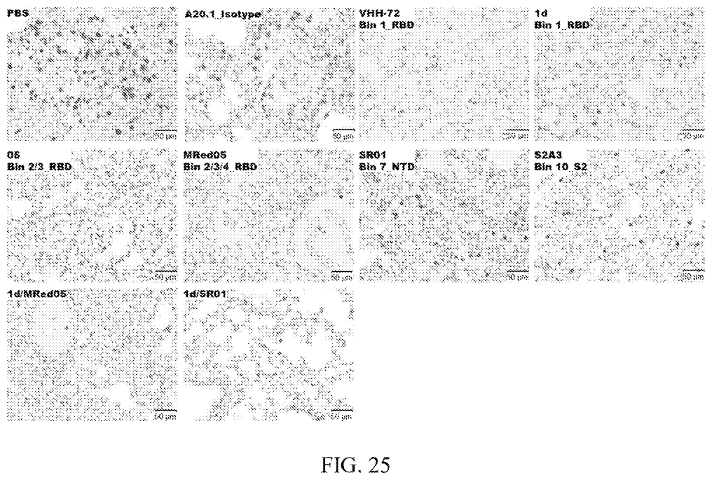

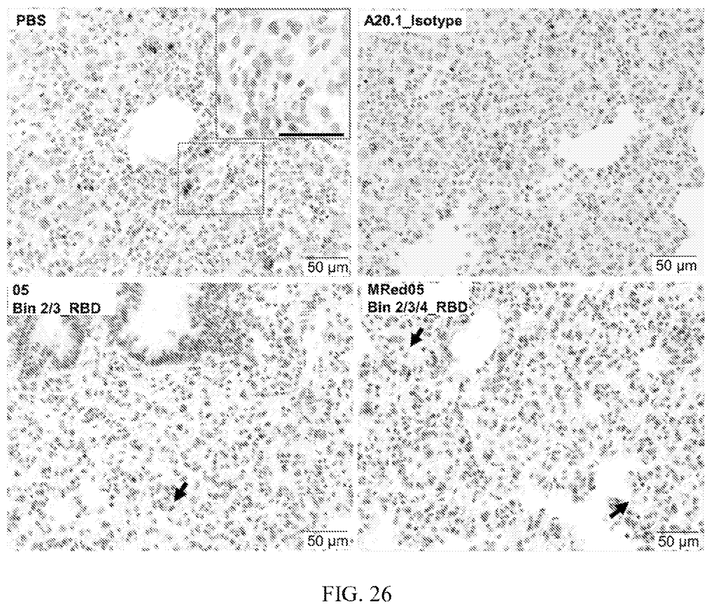

Subsequent immunohistochemistry studies corroborated the viral titer and weight change results. First, in agreement with the viral titer observations, substantial viral antigen (nucleocapsid) reductions in hamster lungs were observed with antibody treatments (FIG. 23; compare non-treated PBS and isotype controls to treated profiles). Although, small foci of viral antigen expression were detected in V_{H}H-72-, 1d-, SR01- and S2A3-treated animals, none were detected in 05-, MRed05-, 1d/SR01- and 1d/MRed05-treated animals. Second, SARS-CoV-2 infection is characterized by an overt inflammatory response in the respiratory tract accompanied by an increased infiltration of inflammatory immune cells, e.g., macrophages and T lymphocytes, in the lung parenchyma 70. As expected, this was the case for the non-treated PBS and isotype control groups. In contrast, we observed a substantial reduction of macrophages and T lymphocytes infiltrate in lung parenchyma with antibody treatment (FIGS. 24, 25). The most dramatic decreases in the number of macrophages and T lymphocytes were seen with 05, MRed05, 1 d/MRed05 and 1d/5R01 treatments. Interestingly, a reduction in inflammatory responses was also associated with a decrease in the number of apoptotic cells in antibody-treated animals (FIG. 26). Altogether, the viral titer, weight change and immunohistochemistry results consistently demonstrate that a single dose of several of the V_{H}H-Fcs reduced viral burden, immune cell infiltration and apoptosis in the lungs of infected hamsters.

The preceding examples have been provided to illustrate various aspects of the disclosure and are non-limiting. The scope of the claims is not limited to specific details provided in the examples; rather the claims are to be given the broadest interpretation consistent with the teachings of the disclosure as a whole.

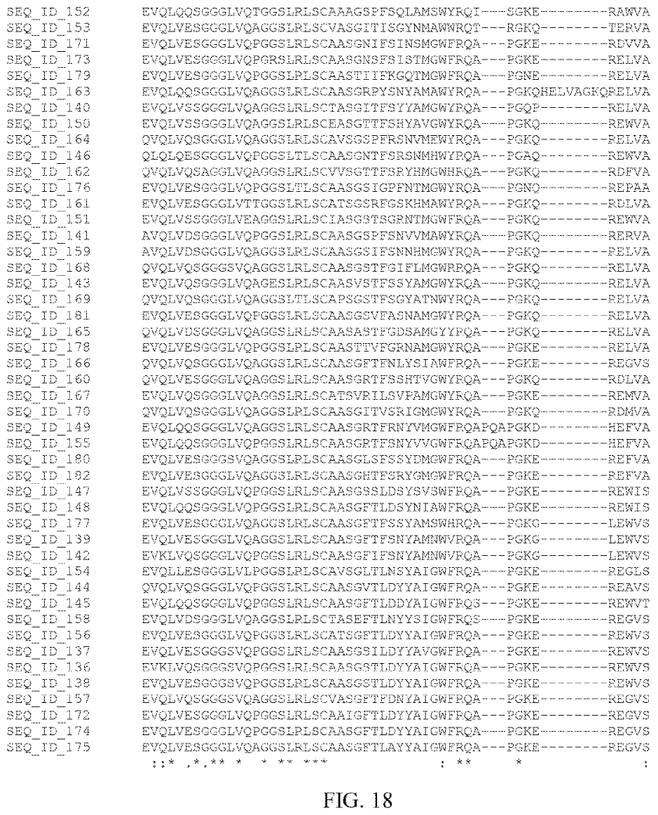

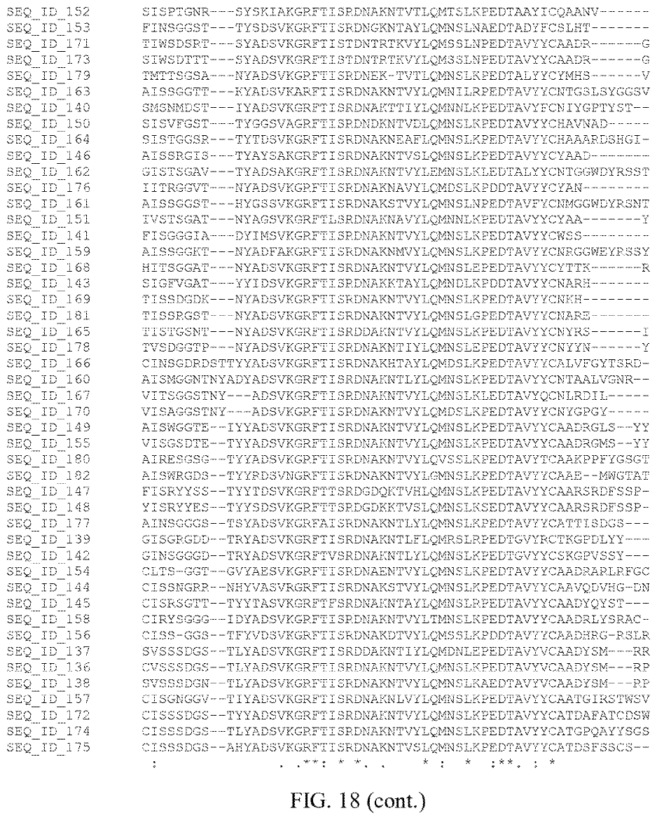

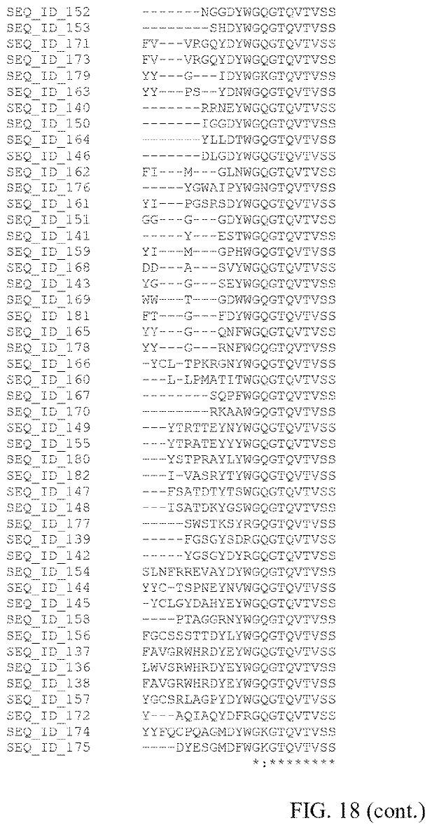

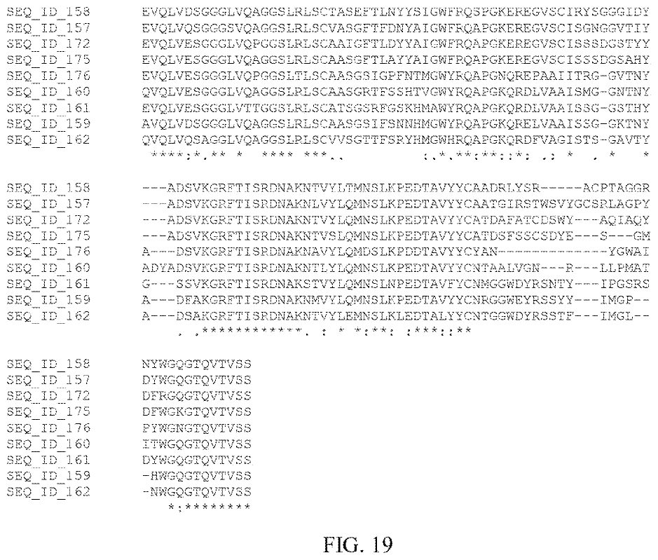

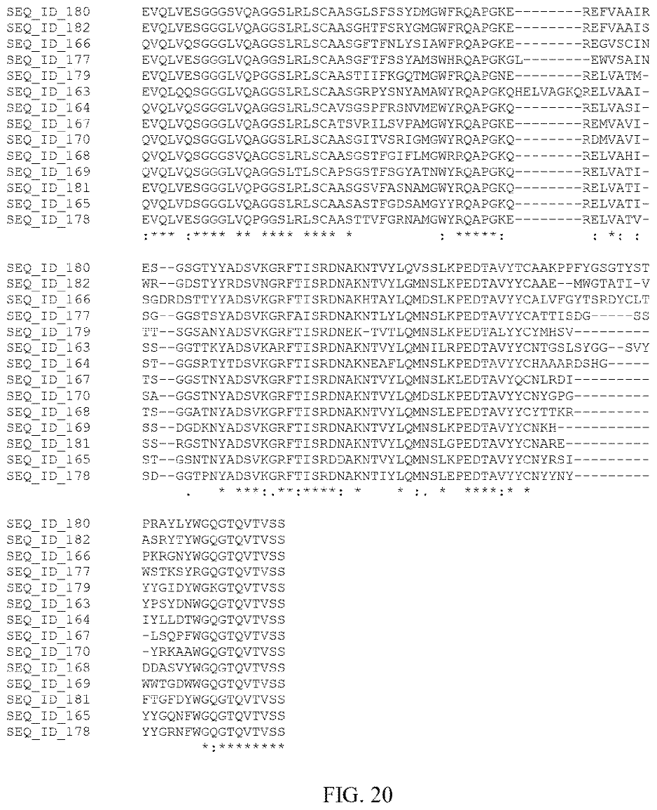

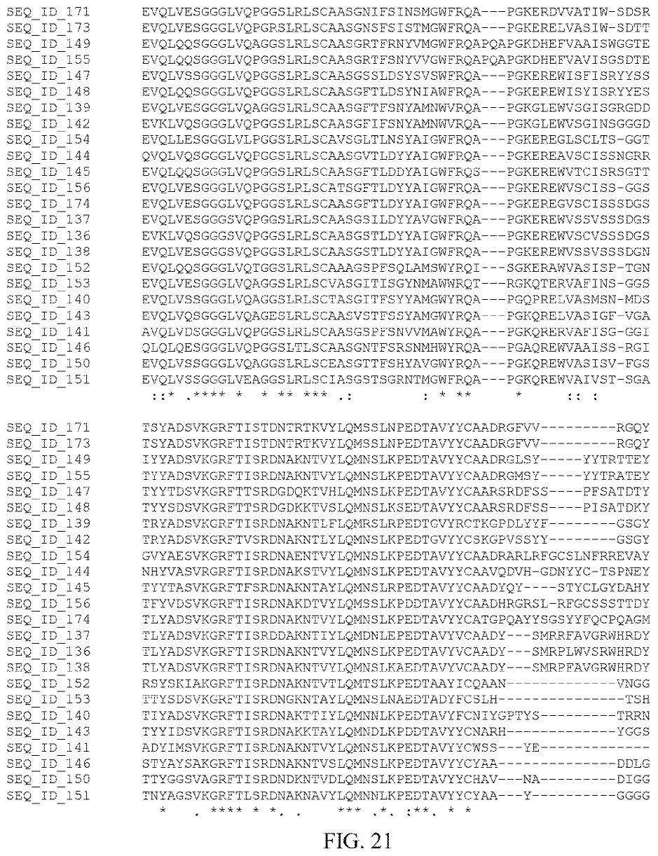

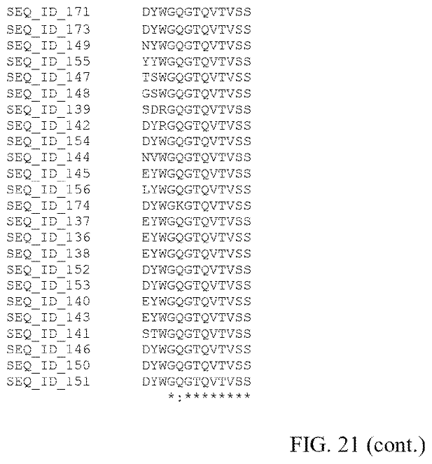

TABLE 24
List of sequences described in the specification

| SEQ | | | Antibody(ies) |
|---|---|---|---|
| ID | | Seq. | including |
| NO: | Sequence | Type | sequence |
| 1 | GSTLDYYA | CDR1 | NRCoV2-1a |
| | | | NRCoV2-1d |
| 2 | GSILDYYA | CDR1 | NRCoV2-1c |
| 3 | GFTFSNYA | CDR1 | NRCoV2-02 |
| 4 | GITFSYYA | CDR1 | NRCoV2-03 |
| 5 | GSPFSNVV | CDR1 | NRCoV2-04 |
| 6 | GFIFSNYA | CDR1 | NRCoV2-05 |
| 7 | VSTFSSYA | CDR1 | NRCoV2-06 |
| 8 | GVTLDYYA | CDR1 | NRCoV2-07 |
| 9 | GFTLDDYA | CDR1 | NRCoV2-08 |
| 10 | GNTFSRSN | CDR1 | NRCoV2-10 |
| 11 | GSSLDSYS | CDR1 | NRCoV2-11 |
| 12 | GFTLDSYN | CDR1 | NRCoV2-11a |
| 13 | GRTFRNYV | CDR1 | NRCoV2-12 |
| 14 | GTTFSHYA | CDR1 | NRCoV2-14 |
| 15 | GSTSGRNT | CDR1 | NRCoV2-15 |
| 16 | GSPFSQLA | CDR1 | NRCoV2-17 |
| 17 | GITISGYN | CDR1 | NRCoV2-18 |
| 18 | GLTLNSYA | CDR1 | NRCoV2-19 |
| 19 | GRTFSNYV | CDR1 | NRCoV2-20 |
| 20 | GFTLDYYA | CDR1 | NRCoV2-21 |
| | | | NRCoV2-MRed03 |
| | | | NRCoV2-MRed05 |
| 21 | GFTFDNYA | CDR1 | NRCoV2-SR01 |
| 22 | EFTLNYYS | CDR1 | NRCoV2-SR02 |
| 23 | GSIFSNNH | CDR1 | NRCoV2-SR03 |
| 24 | GRTFSSHT | CDR1 | NRCoV2-SR04 |
| 25 | GSRFGSKH | CDR1 | NRCoV2-SR13 |
| 26 | GTTFSRYH | CDR1 | NRCoV2-SR16 |
| 27 | GRPYSNYA | CDR1 | NRCoV2-S2A3 |
| 28 | GSPFRSNV | CDR1 | NRCoV2-S2A4 |
| 29 | ASTFGDSA | CDR1 | NRCoV2-S2B3 |
| 30 | GFTFNLYS | CDR1 | NRCoV2-S2H4 |
| 31 | VRILSVPA | CDR1 | NRCoV2-S2F3 |
| 32 | GSTFGIFL | CDR1 | NRCoV2-S2G3 |
| 33 | GSTFSGYA | CDR1 | NRCoV2-S2G4 |
| 34 | GITVSRIG | CDR1 | NRCoV2-S202 |
| 35 | GNIFSINS | CDR1 | NRCoV2-MRed02 |
| 36 | GNSFSINT | CDR1 | NRCoV2-MRed04 |
| 37 | GFTLAYYA | CDR1 | NRCoV2-MRed06 |
| 38 | GSIGPFNT | CDR1 | NRCoV2-MRed07 |
| 39 | GFTFSSYA | CDR1 | NRCoV2-MRed11 |
| 40 | TTVFGRNA | CDR1 | NRCoV2-MRed18 |
| 41 | TIIFKGQT | CDR1 | NRCoV2-MRed19 |
| 42 | GLSFSSYD | CDR1 | NRCoV2-MRed20 |
| 43 | GSVFASNA | CDR1 | NRCoV2-MRed22 |
| 44 | GHTFSRYG | CDR1 | NRCoV2-MRed25 |
| 45 | VSSSDGST | CDR2 | NRCoV2-1a |
| | | | NRCoV2-1c |
| 46 | VSSSDGNT | CDR2 | NRCoV2-1d |
| 47 | ISGRGDDT | CDR2 | NRCoV2-02 |
| 48 | MSNMDST | CDR2 | NRCoV2-03 |
| 49 | ISGGGIA | CDR2 | NRCoV2-04 |
| 50 | INSGGGDT | CDR2 | NRCoV2-05 |
| 51 | IGFVGAT | CDR2 | NRCoV2-06 |
| 52 | ISSNGRRN | CDR2 | NRCoV2-07 |
| 53 | ISRSGTTT | CDR2 | NRCoV2-08 |
| 54 | ISSRGIS | CDR2 | NRCoV2-10 |
| 55 | ISRYYSST | CDR2 | NRCoV2-11 |
| 56 | ISRYYEST | CDR2 | NRCoV2-11a |
| 57 | VAAISWGGTEI | CDR2 | NRCoV2-12 |
| 58 | ISVFGST | CDR2 | NRCoV2-14 |
| 59 | VSTSGAT | CDR2 | NRCoV2-15 |
| 60 | ISPTGNR | CDR2 | NRCoV2-17 |
| 61 | INSGGST | CDR2 | NRCoV2-18 |
| 62 | LTSGGTG | CDR2 | NRCoV2-19 |
| 63 | VAVISGSDTET | CDR2 | NRCoV2-20 |
| 64 | ISSGGST | CDR2 | NRCoV2-21 |
| 65 | ISGNGGVT | CDR2 | NRCoV2-SR01 |
| 66 | IRYSGGGI | CDR2 | NRCoV2-SR02 |
| 67 | ISSGGKT | CDR2 | NRCoV2-SR03 |
| 68 | ISMGGNTNYA | CDR2 | NRCoV2-SR04 |
| 69 | ISSGGST | CDR2 | NRCoV2-SR13 |
| 70 | ISTSGAV | CDR2 | NRCoV2-SR16 |
| 71 | KQRELVAAISSGGTT | CDR2 | NRCoV2-S2A3 |
| 72 | ISTGGSR | CDR2 | NRCoV2-S2A4 |
| 73 | ISTGSNT | CDR2 | NRCoV2-S2B3 |
| 74 | INSGDRDSTT | CDR2 | NRCoV2-S2H4 |
| 75 | ITSGGST | CDR2 | NRCoV2-S2F3 |
| 76 | ITSGGAT | CDR2 | NRCoV2-S2G3 |
| 77 | ISSDGDK | CDR2 | NRCoV2-S2G4 |
| 78 | ISAGGST | CDR2 | NRCoV2-S202 |
| 79 | IWSDSRT | CDR2 | NRCoV2-MRed02 |
| 80 | IWSDTTT | CDR2 | NRCoV2-MRed04 |
| 81 | ISSSDGST | CDR2 | NRCoV2-MRed03 |
| | | | NRCoV2-MRed05 |
| 82 | ISSSDGSA | CDR2 | NRCoV2-MRed06 |
| 83 | ITRGGVT | CDR2 | NRCoV2-MRed07 |
| 84 | INSGGGST | CDR2 | NRCoV2-MRed11 |
| 85 | VSDGGTP | CDR2 | NRCoV2-MRed18 |
| 86 | MTTSGSA | CDR2 | NRCoV2-MRed19 |
| 87 | IRESGSGT | CDR2 | NRCoV2-MRed20 |
| 88 | ISSRGST | CDR2 | NRCoV2-MRed22 |
| 89 | ISWRGDST | CDR2 | NRCoV2-MRed25 |
| 90 | AADYSMRPLWVSRWHRDYEY | CDR3 | NRCoV2-1a |
| 91 | AADYSMRRFAVGRWHRDYEY | CDR3 | NRCoV2-1c |
| 92 | AADYSMRPFAVGRWHRDYEY | CDR3 | NRCoV2-1d |
| 93 | TKGPDLYYFGSGYSD | CDR3 | NRCoV2-02 |
| 94 | NIYGPTYSTRRNEY | CDR3 | NRCoV2-03 |
| 95 | WSSYEST | CDR3 | NRCoV2-04 |
| 96 | SKGPVSSYYGSGYDY | CDR3 | NRCoV2-05 |
| 97 | NARHYGGSEY | CDR3 | NRCoV2-06 |
| 98 | AAVQDVHGDNYYCTSPNEYNV | CDR3 | NRCoV2-07 |
| 99 | AADYQYSTYCLGYDAHYEY | CDR3 | NRCoV2-08 |
| 100 | YAADDLGDY | CDR3 | NRCoV2-10 |
| 101 | AARSRDFSSPFSATDTYTS | CDR3 | NRCoV2-11 |
| 102 | AARSRDFSSPISATDKYGS | CDR3 | NRCoV2-11a |
| 103 | AADRGLSYYYTRTTEYNY | CDR3 | NRCoV2-12 |
| 104 | HAVNADIGGDY | CDR3 | NRCoV2-14 |
| 105 | YAAYGGGGDY | CDR3 | NRCoV2-15 |
| 106 | QAANVNGGDY | CDR3 | NRCoV2-17 |
| 107 | SLHTSHDY | CDR3 | NRCoV2-18 |
| 108 | AADRARLRFGCSLNFRREVAYDY | CDR3 | NRCoV2-19 |
| 109 | AADRGMSYYYTRATEYYY | CDR3 | NRCoV2-20 |
| 110 | AADHRGRSLRFGCSSSTTDYLY | CDR3 | NRCoV2-21 |
| 111 | AATGIRSTWSVYGCSRLAGPYDY | CDR3 | NRCoV2-SR01 |
| 112 | AADRLYSRACPTAGGRNY | CDR3 | NRCoV2-SR02 |
| 113 | NRGGWEYRSSYYIMGPH | CDR3 | NRCoV2-SR03 |
| 114 | NTAALVGNRLLPMATIT | CDR3 | NRCoV2-SR04 |
| 115 | NMGGWDYRSNTYIPGSRSDY | CDR3 | NRCoV2-SR13 |
| 116 | NTGGWDYRSSTFIMGLN | CDR3 | NRCoV2-SR16 |
| 117 | NTGSLSYGGSVYYPSYDN | CDR3 | NRCoV2-S2A3 |
| 118 | HAAARDSHGIYLLDT | CDR3 | NRCoV2-S2A4 |
| 119 | NYRSIYYGQNF | CDR3 | NRCoV2-S2B3 |
| 120 | ALVFGYTSRDYCLTPKRGNY | CDR3 | NRCoV2-S2H4 |
| 121 | NLRDILSQPF | CDR3 | NRCoV2-S2F3 |
| 122 | YTTKRDDASVY | CDR3 | NRCoV2-S2G3 |
| 123 | NKHWWTGDW | CDR3 | NRCoV2-S2G4 |
| 124 | NYGPGYRKAA | CDR3 | NRCoV2-S202 |
| 125 | AADRGFVVRGQYDY | CDR3 | NRCoV2-MRed02 |
| | | | NRCoV2-MRed04 |
| 126 | ATDAFATCDSWYAQIAQYDF | CDR3 | NRCoV2-MRed03 |
| 127 | ATGPQAYYSGSYYFQCPQAGMDY | CDR3 | NRCoV2-MRed05 |
| 128 | ATDSFSSCSDYESGMDF | CDR3 | NRCoV2-MRed06 |
| 129 | YANYGWAIPY | CDR3 | NRCoV2-MRed07 |
| 130 | ATTISDGSSWSTKSY | CDR3 | NRCoV2-MRed11 |
| 131 | NYYNYYYGRNF | CDR3 | NRCoV2-MRed18 |
| 132 | YMHSVYYGIDY | CDR3 | NRCoV2-MRed19 |
| 133 | AAKPPFYGSGTYSTPRAYLY | CDR3 | NRCoV2-MRed20 |
| 134 | NAREFTGFDY | CDR3 | NRCoV2-MRed22 |
| 135 | AAEMWGTATIVASRYTY | CDR3 | NRCoV2-MRed25 |
| 136 | EVKLVQSGGGSVQPGGSLRLSCAASGSTLDYYAIGWF | V_{H}H | NRCoV2-1a |
| | RQAPGKEREWVSCVSSSDGSTLYADSVKGRFTISRDNA | | |
| | KNTVYLQMNSLKPEDTAVYVCAADYSMRPLWVSRWH | | |
| | RDYEYWGQGTQVTVSS | | |
| 137 | EVQLVESGGGSVQPGGSLRLSCAASGSILDYYAVGWF | V_{H}H | NRCoV2-1c |
| | RQAPGKEREWVSSVSSSDGSTLYADSVKGRFTISRDDA | | |
| | KNTIYLQMDNLEPEDTAVYVCAADYSMRRFAVGRWHR | | |
| | DYEYWGQGTQVTVSS | | |
| 138 | EVQLVESGGGSVQPGGSLRLSCAASGSTLDYYAIGWF | V_{H}H | NRCoV2-1d |
| | RQAPGKEREWVSSVSSSDGNTLYADSVKGRFTISRDNA | | |
| | KNTVYLQMNSLKAEDTAVYVCAADYSMRPFAVGRWH | | |
| | RDYEYWGQGTQVTVSS | | |
| 139 | EVQLVESGGGLVQAGGSLRLSCAASGFTFSNYAMNWV | V_{H}H | NRCoV2-02 |
| | RQAPGKGLEWVSGISGRGDDTRYADSVKGRFTISRDN | | |
| | AKNTLFLQMRSLRPEDTGVYRCTKGPDLYYFGSGYSD | | |
| | RGQGTQVTVSS | | |
| 140 | EVQLVSSGGGLVQAGGSLRLSCTASGITFSYYAMGWY | V_{H}H | NRCoV2-03 |
| | RQAPGQPRELVASMSNMDSTIYADSVKGRFTISRDNAK | | |
| | TTIYLQMNNLKPEDTAVYFCNIYGPTYSTRRNEYWGQG | | |
| | TQVTVSS | | |
| 141 | AVQLVDSGGGLVQPGGSLRLSCAASGSPFSNVVMAWY | V_{H}H | NRCoV2-04 |
| | RQAPGKQRERVAFISGGGIADYIMSVKGRFTISRDNAKN | | |
| | TVYLQMNSLKPEDTAVYYCWSSYESTWGQGTQVTVSS | | |
| 142 | EVKLVQSGGGLVQPGGSLRLSCAASGFIFSNYAMNWV | V_{H}H | NRCoV2-05 |
| | RQAPGKGLEWVSGINSGGGDTRYADSVKGRFTVSRDN | | |
| | AKNTLYLQMNSLKPEDTGVYYCSKGPVSSYYGSGYDYR | | |
| | GQGTQVTVSS | | |
| 143 | EVQLVQSGGGLVQAGESLRLSCAASVSTFSSYAMGWY | V_{H}H | NRCoV2-06 |
| | RQAPGKQRELVASIGFVGATYYIDSVKGRFTISRDNAKK | | |
| | TAYLQMNDLKPDDTAVYYCNARHYGGSEYWGQGTQV | | |
| | TVSS | | |
| 144 | QVQLVQSGGGLVQPGGSLRLSCAASGVTLDYYAIGWF | V_{H}H | NRCoV2-07 |
| | RQAPGKEREAVSCISSNGRRNHYVASVRGRFTISRDNA | | |
| | KSTVYLQMNSLKPEDTAVYYCAAVQDVHGDNYYCTSP | | |
| | NEYNVWGQGTQVTVSS | | |
| 145 | EVQLQQSGGGLVQPGGSLRLSCAASGFTLDDYAIGWF | V_{H}H | NRCoV2-08 |
| | RQSPGKEREWVTCISRSGTTTYYTASVKGRFTFSRDNA | | |
| | KNTAYLQMNSLRPEDTAVYYCAADYQYSTYCLGYDAH | | |
| | YEYWGQGTQVTVSS | | |
| 146 | QLQLQESGGGLVQPGGSLTLSCAASGNTFSRSNMHWY | V_{H}H | NRCoV2-10 |
| | RQAPGAQREWVAAISSRGISTYAYSAKGRFTISRDNAKN | | |
| | TVSLQMNSLKPEDTAVYYCYAADDLGDYWGQGTQVTV | | |
| | SS | | |
| 147 | EVQLVSSGGGLVQPGGSLRLSCAASGSSLDSYSVSWFR | V_{H}H | NRCoV2-11 |
| | QAPGKEREWISFISRYYSSTYYTDSVKGRFTTSRDGDQ | | |
| | KTVHLQMNSLKPEDTAVYYCAARSRDFSSPFSATDTYT | | |
| | SWGQGTQVTVSS | | |
| 148 | EVQLQQSGGGLVQPGGSLRLSCAASGFTLDSYNIAWF | V_{H}H | NRCoV2-11a |
| | RQAPGKEREWISYISRYYESTYYSDSVKGRFTTSRDGD | | |
| | KKTVSLQMNSLKSEDTAVYYCAARSRDFSSPISATDKY | | |
| | GSWGQGTQVTVSS | | |
| 149 | EVQLQQSGGGLVQAGGSLRLSCAASGRTFRNYVMGW | V_{H}H | NRCoV2-12 |
| | FRQAPQAPGKDHEFVAAISWGGTEIYYADSVKGRFTIS | | |
| | RDNAKNTVYLQMNSLKPEDTAVYYCAADRGLSYYYTR | | |
| | TTEYNYWGQGTQVTVSS | | |
| 150 | EVQLVSSGGGLVQAGGSLRLSCEASGTTFSHYAVGWY | V_{H}H | NRCoV2-14 |
| | RQAPGKQREWVASISVFGSTTYGGSVAGRFTISRDNDK | | |
| | NTVDLQMNSLKPEDTAVYYCHAVNADIGGDYWGQGTQ | | |
| | VTVSS | | |
| 151 | EVQLVSSGGGLVEAGGSLRLSCIASGSTSGRNTMGWF | V_{H}H | NRCoV2-15 |
| | RQAPGKQREWVAIVSTSGATNYAGSVKGRFTLSRDNA | | |
| | KNAVYLQMNNLKPEDTAVYYCYAAYGGGGDYWGQGT | | |
| | QVTVSS | | |
| 152 | EVQLQQSGGGLVQTGGSLRLSCAAAGSPFSQLAMSWY | V_{H}H | NRCoV2-17 |
| | RQISGKERAWVASISPTGNRSYSKIAKGRFTISRDNAKN | | |
| | TVTLQMTSLKPEDTAAYICQAANVNGGDYWGQGTQVT | | |
| | VSS | | |
| 153 | EVQLVESGGGLVQAGGSLRLSCVASGITISGYNMAWW | V_{H}H | NRCoV2-18 |
| | RQTRGKQTERVAFINSGGSTTYSDSVKGRFTISRDNGK | | |
| | NTAYLQMNSLNAEDTADYFCSLHTSHDYWGQGTQVTV | | |
| | SS | | |
| 154 | EVQLLESGGGLVLPGGSLRLSCAVSGLTLNSYAIGWFR | V_{H}H | NRCoV2-19 |
| | QAPGKEREGLSCLTSGGTGVYAESVKGRFTISRDNAEN | | |
| | TVYLQMNSLKPEDTAVYYCAADRARLRFGCSLNFRRE | | |
| | VAYDYWGQGTQVTVSS | | |
| 155 | EVQLQQSGGGLVQPGGSLRLSCAASGRTFSNYVVGWF | V_{H}H | NRCoV2-20 |
| | RQAPQAPGKDHEFVAVISGSDTETYYADSVKGRFTISR | | |
| | DNAKNTVYLQMNSLKPEDTAVYYCAADRGMSYYYTRA | | |
| | TEYYYWGQGTQVTVSS | | |
| 156 | EVQLVESGGGLVQPGGSLRLSCATSGFTLDYYAIGWFR | V_{H}H | NRCoV2-21 |
| | QAPGKEREWVSCISSGGSTFYVDSVKGRFTISRDNAKD | | |
| | TVYLQMSSLKPDDTAVYYCAADHRGRSLRFGCSSSTT | | |
| | DYLYWGQGTQVTVSS | | |
| 157 | EVQLVQSGGGSVQAGGSLRLSCVASGFTFDNYAIGWF | V_{H}H | NRCoV2-SR01 |
| | RQAPGKEREGVSCISGNGGVTIYADSVKGRFTISRDNA | | |
| | KNLVYLQMNSLKPEDTAVYYCAATGIRSTWSVYGCSRL | | |
| | AGPYDYWGQGTQVTVSS | | |
| 158 | EVQLVDSGGGLVQAGGSLRLSCTASEFTLNYYSIGWFR | V_{H}H | NRCoV2-SR02 |
| | QSPGKEREGVSCIRYSGGGIDYADSVKGRFTISRDNAK | | |
| | NTVYLTMNSLKPEDTAVYYCAADRLYSRACPTAGGRN | | |
| | YWGQGTQVTVSS | | |
| 159 | AVQLVDSGGGLVQAGGSLRLSCAASGSIFSNNHMGWY | V_{H}H | NRCoV2-SR03 |
| | RQAPGKQRELVAAISSGGKTNYADFAKGRFTISRDNAK | | |
| | NMVYLQMNSLKPEDTAVYYCNRGGWEYRSSYYIMGPH | | |
| | WGQGTQVTVSS | | |
| 160 | QVQLVESGGGLVQAGGSLRLSCAASGRTFSSHTVGWY | V_{H}H | NRCoV2-SR04 |
| | RQAPGKQRDLVAAISMGGNTNYADYADSVKGRFTISRD | | |
| | NAKNTLYLQMNSLKPEDTAVYYCNTAALVGNRLLPMAT | | |
| | ITWGQGTQVTVSS | | |
| 161 | EVQLVESGGGLVTTGGSLRLSCATSGSRFGSKHMAWY | V_{H}H | NRCoV2-SR13 |
| | RQAPGKQRDLVAAISSGGSTHYGSSVKGRFTISRDNAK | | |
| | STVYLQMNSLNPEDTAVFYCNMGGWDYRSNTYIPGSR | | |
| | SDYWGQGTQVTVSS | | |
| 162 | QVQLVQSAGGLVQAGGSLRLSCVVSGTTFSRYHMGW | V_{H}H | NRCoV2-SR16 |
| | HRQAPGKQRDFVAGISTSGAVTYADSAKGRFTISRDNA | | |
| | KNTVYLEMNSLKLEDTALYYCNTGGWDYRSSTFIMGL | | |
| | NWGQGTQVTVSS | | |
| 163 | EVQLQQSGGGLVQAGGSLRLSCAASGRPYSNYAMAWY | V_{H}H | NRCoV2-S2A3 |
| | RQAPGKQHELVAGKQRELVAAISSGGTTKYADSVKAR | | |
| | FTISRDNAKNTVYLQMNILRPEDTAVYYCNTGSLSYGGS | | |
| | VYYPSYDNWGQGTQVTVSS | | |
| 164 | QVQLVQSGGGLVQAGGSLRLSCAVSGSPFRSNVMEWY | V_{H}H | NRCoV2-S2A4 |
| | RQAPGKQRELVASISTGGSRTYTDSVKGRFTISRDNAK | | |
| | NEAFLQMNSLKPEDTAVYYCHAAARDSHGIYLLDTWG | | |
| | QGTQVTVSS | | |
| 165 | QVQLVDSGGGLVQAGGSLRLSCAASASTFGDSAMGYY | V_{H}H | NRCoV2-S2B3 |
| | RQAPGKQRELVATISTGSNTNYADSVKGRFTISRDDAK | | |
| | NTVYLQMNSLKPEDTAVYYCNYRSIYYGQNFWGQGTQ | | |
| | VTVSS | | |
| 166 | QVQLVQSGGGLVQAGGSLRLSCAASGFTFNLYSIAWFR | V_{H}H | NRCoV2-S2H4 |
| | QAPGKEREGVSCINSGDRDSTTYYADSVKGRFTISRDN | | |
| | AKHTAYLQMDSLKPEDTAVYYCALVFGYTSRDYCLTPK | | |
| | RGNYWGQGTQVTVSS | | |
| 167 | EVQLVQSGGGLVQAGGSLRLSCATSVRILSVPAMGWY | V_{H}H | NRCoV2-S2F3 |
| | RQAPGKEREMVAVITSGGSTNYADSVKGRFTISRDNAK | | |
| | NTVYLQMNSLKLEDTAVYQCNLRDILSQPFWGQGTQV | | |
| | TVSS | | |
| 168 | QVQLVQSGGGSVQAGGSLRLSCAASGSTFGIFLMGWR | V_{H}H | NRCoV2-S2G3 |
| | RQAPGKQRELVAHITSGGATNYADSVKGRFTISRDNAK | | |
| | NTVYLQMNSLEPEDTAVYYCYTTKRDDASVYWGQGTQ | | |
| | VTVSS | | |
| 169 | QVQLVQSGGGLVQAGGSLTLSCAPSGSTFSGYATNWY | V_{H}H | NRCoV2-S2G4 |
| | RQAPGKQRELVATISSDGDKNYADSVKGRFTISRDNAK | | |
| | NTVYLQMNSLKPEDTAVYYCNKHWWTGDWWGQGTQ | | |
| | VTVSS | | |
| 170 | QVQLVQSGGGLVQAGGSLRLSCAASGITVSRIGMGWY | V_{H}H | NRCoV2-S202 |
| | RQAPGKQRDMVAVISAGGSTNYADSVKGRFTISRDNAK | | |
| | NTVYLQMDSLKPEDTAVYYCNYGPGYRKAAWGQGTQ | | |
| | VTVSS | | |
| 171 | EVQLVESGGGLVQPGGSLRLSCAASGNIFSINSMGWFR | V_{H}H | NRCoV2-MRed02 |
| | QAPGKERDVVATIWSDSRTSYADSVKGRFTISTDNTRT | | |
| | KVYLQMSSLNPEDTAVYYCAADRGFVVRGQYDYWGQ | | |
| | GTQVTVSS | | |
| 172 | EVQLVESGGGLVQPGGSLRLSCAAIGFTLDYYAIGWFR | V_{H}H | NRCoV2-MRed03 |
| | QAPGKEREGVSCISSSDGSTYYADSVKGRFTISRDNAK | | |
| | NTVYLQMNSLKPEDTAVYYCATDAFATCDSWYAQIAQ | | |
| | YDFRGQGTQVTVSS | | |
| 173 | EVQLVESGGGLVQPGRSLRLSCAASGNSFSISTMGWF | V_{H}H | NRCoV2-MRed04 |
| | RQAPGKERELVASIWSDTTTSYADSVKGRFTISTDNTRT | | |
| | KVYLQMSSLNPEDTAVYYCAADRGFVVRGQYDYWGQ | | |
| | GTQVTVSS | | |
| 174 | EVQLVESGGGLVQPGGSLRLSCAASGFTLDYYAIGWFR | V_{H}H | NRCoV2-MRed05 |
| | QAPGKEREGVSCISSSDGSTLYADSVKGRFTISRDNAK | | |
| | NTVYLQMNSLKPEDTAVYYCATGPQAYYSGSYYFQCP | | |
| | QAGMDYWGKGTQVTVSS | | |
| 175 | EVQLVESGGGLVQAGGSLRLSCAASGFTLAYYAIGWFR | V_{H}H | NRCoV2-MRed06 |
| | QAPGKEREGVSCISSSDGSAHYADSVKGRFTISRDNAK | | |
| | NTVSLQMNSLKPEDTAVYYCATDSFSSCSDYESGMDF | | |
| | WGKGTQVTVSS | | |
| 176 | EVQLVESGGGLVQPGGSLTLSCAASGSIGPFNTMGWY | V_{H}H | NRCoV2-MRed07 |
| | RQAPGNQREPAAIITRGGVTNYADSVKGRFTISRDNAK | | |
| | NAVYLQMDSLKPDDTAVYYCYANYGWAIPYWGNGTQV | | |
| | TVSS | | |
| 177 | EVQLVESGGGLVQAGGSLRLSCAASGFTFSSYAMSWH | V_{H}H | NRCoV2-MRed11 |
| | RQAPGKGLEWVSAINSGGGSTSYADSVKGRFAISRDNA | | |
| | KNTLYLQMNSLKPEDTAVYYCATTISDGSSWSTKSYRG | | |
| | QGTQVTVSS | | |
| 178 | EVQLVESGGGLVQPGGSLRLSCAASTTVFGRNAMGWY | V_{H}H | NRCoV2-MRed18 |
| | RQAPGKERELVATVSDGGTPNYADSVKGRFTISRDNAK | | |
| | NTIYLQMNSLEPEDTAVYYCNYYNYYYGRNFWGQGTQ | | |
| | VTVSS | | |
| 179 | EVQLVESGGGLVQPGGSLRLSCAASTIIFKGQTMGWF | V_{H}H | NRCoV2-MRed19 |
| | RQAPGNERELVATMTTSGSANYADSVKGRFTISRDNEK | | |
| | TVTLQMNSLKPEDTALYYCYMHSVYYGIDYWGKGTQV | | |
| | TVSS | | |
| 180 | EVQLVESGGGSVQAGGSLRLSCAASGLSFSSYDMGWF | V_{H}H | NRCoV2-MRed20 |
| | RQAPGKEREFVAAIRESGSGTYYADSVKGRFTISRDNA | | |
| | KNTVYLQVSSLKPEDTAVYTCAAKPPFYGSGTYSTPRA | | |
| | YLYWGQGTQVTVSS | | |
| 181 | EVQLVESGGGLVQPGGSLRLSCAASGSVFASNAMGWY | V_{H}H | NRCoV2-MRed22 |
| | RQAPGKQRELVATISSRGSTNYADSVKGRFTISRDNAK | | |
| | NTVYLQMNSLGPEDTAVYYCNAREFTGFDYWGQGTQV | | |
| | TVSS | | |
| 182 | EVQLVESGGGLVQAGGSLRLSCAASGHTFSRYGMGW | V_{H}H | NRCoV2-MRed25 |
| | FRQAPGKEREFVAAISWRGDSTYYRDSVNGRFTISRDN | | |
| | AKNTVYLGMNSLKPEDTAVYYCAAEMWGTATIVASRY | | |
| | TYWGQGTQVTVSS | | |
| 183 | XXXLXXSXGGXVXXGXSLXLSCXXXXXXXXXXXXXXX | V_{H}H | Consensus sequence |
| | RQXXXXXGXXXXXXXXXXXXXXXXXXXXXXXXXXXXX | cons. | for V_{H}H sequences, |
| | XXXXXRFXXSXDXXXXXXXLXXXXLXXXDTXXXXCXX | seq. | generated based on |
| | XXXXXXXXXXXXXXXXXXXXXXGXGTQVTVSS | | the sequence |
| | X at position 1 is Ala, Glu, or Gln | | alignment shown |
| | X at position 2 is Leu or Val | | in FIG. 18 |
| | X at position 3 is Lys or Gln | | |
| | X at position 5 is Leu, Gln, or Val | | |
| | X at position 6 is Asp, Glu, Gln, or Ser | | |
| | X at position 8 is Ala or Gly | | |
| | X at position 11 is Leu or Ser | | |
| | X at position 13 is Glu, Leu, Gln, or Thr | | |
| | X at position 14 is Ala, Pro, or Thr | | |
| | X at position 16 is Glu, Gly, or Arg | | |
| | X at position 19 is Arg or Thr | | |
| | X at position 23 is Ala, Glu, Ile, Thr, or Val | | |
| | X at position 24 is Ala, Pro, Thr, or Val | | |
| | X at position 25 is Ala, Ile, or Ser | | |
| | X at position 26 is Ala, Glu, Gly, Thr, or Val | | |
| | X at position 27 is Phe, His, Ile, Leu, Asn, | | |
| | Arg, Ser, Thr, or Val | | |
| | X at position 28 is Ile, Pro, Arg, Ser, Thr, | | |
| | or Val | | |
| | X at position 29 is Phe, Gly, Ile, Leu, Ser, | | |
| | Val, or Tyr | | |
| | X at position 30 is Ala, Asp. Gly, Lys, Asn, | | |
| | Pro, Arg, or Ser | | |
| | X at position 31 is Asp, Phe, Gly, His, Ile, | | |
| | Leu, Asn, Gln, Arg, Ser, Val, Tyr | | |
| | X at position 32 is Phe, His, Ile, Lys, Leu, | | |
| | Asn, Pro, Gln, Ser, Val, or Tyr | | |
| | X at position 33 is Ala, Asp, Gly, His, Leu, | | |
| | Asn, Ser, Thr, or Val | | |
| | X at position 34 is Ile, Met, Thr, or Val | | |
| | X at position 35 is Ala, Glu, Gly, His, Asn, | | |
| | or Ser | | |
| | X at position 36 is Trp or Tyr | | |
| | X at position 37 is Phe, His, Arg, Val, Trp, | | |
| | or Tyr | | |
| | X at position 40 is Ala, Ile, Ser, or Thr | | |
| | X at position 41 is Pro or absent | | |
| | X at position 42 is Gln or absent | | |
| | X at position 43 is Ala or absent | | |
| | X at position 44 is Pro, Arg, or Ser | | |
| | X at position 46 is Ala, Lys, Asn, or Gln | | |
| | X at position 47 is Asp, Glu, Gly, Pro, or | | |
| | Gln | | |
| | X at position 48 is His or absent | | |
| | X at position 49 is Glu or absent | | |
| | X at position 50 is Leu or absent | | |
| | X at position 51 is Val or absent | | |
| | X at position 52 is Ala or absent | | |
| | X at position 53 is Gly or absent | | |
| | X at position 54 is Lys or absent | | |
| | X at position 55 is Gln or absent | | |
| | X at position 56 is His, Leu, Arg, or Thr | | |
| | X at position 57 is Ala, Asp, or Glu | | |
| | X at position 58 is Ala, Phe, Gly, Leu, Met, | | |
| | Pro, Arg, Val, or Trp | | |
| | X at position 59 is Ala, Ile, Leu, or Val | | |
| | X at position 60 is Ala, Ser, or Thr | | |
| | X at position 61 is Ala, Cys, Phe, Gly, His, | | |
| | Ile, Ser, Thr, Val, or Tyr | | |
| | X at position 62 is Ile, Leu, Met, or Val | | |
| | X at position 63 is Gly, Asn, Arg, Ser, Thr, | | |
| | or Trp | | |
| | X at position 64 is Ala, Asp, Glu, Phe, Gly, | | |
| | Met, Asn, Pro, Arg, Ser, Thr, Val, Trp, or Tyr | | |
| | X at position 65 is Asp, Phe, Gly, Met, Asn, | | |
| | Arg, Ser, Thr, Val, Tyr, or absent | | |
| | X at position 66 is Asp, Gly, Ser, Thr, or Tyr | | |
| | X at position 67 is Ala, Asp, Glu, Gly, Ile, | | |
| | Lys, Asn, Arg, Ser, Thr, or Val | | |
| | X at position 68 is Ala, Asp, Glu, Gly, Lys, | | |
| | Asn, Pro, Arg, Ser, Thr, or Val | | |
| | X at position 69 is Asn, Ser, or absent | | |
| | X at position 70 is Thr, Tyr, or absent | | |
| | X at position 71 is Ala, Gly, Ile, Asn, Thr, | | |
| | or absent | | |
| | X at position 72 is Asp, Phe, His, Ile, Lys, | | |
| | Leu, Asn, Arg, Ser, Thr, Val, Tyr, or absent | | |
| | X at position 73 is Tyr or absent | | |
| | X at position 74 is Ala, Gly, Ile, Arg, Ser, | | |
| | Thr, or Val | | |
| | X at position 75 is Ala, Asp, Glu, Gly, Lys, | | |
| | Met, Ser, or Tyr | | |
| | X at position 76 is Phe, Ile, or Ser | | |
| | X at position 77 is Ala or Val | | |
| | X at position 78 is Ala, Lys, Asn, or Val | | |
| | X at position 79 is Ala or Gly | | |
| | X at position 82 is Ala or Thr | | |
| | X at position 83 is Phe, Ile, Leu, Thr, or Val | | |
| | X at position 85 is Arg or Thr | | |
| | X at position 87 is Asp, Gly, or Asn | | |
| | X at position 88 is Ala, Asp, Glu, Gly, or Thr | | |
| | X at position 89 is Glu, Lys, Gln, or Arg | | |
| | X at position 90 is Asp, His, Lys, Asn, Ser, | | |
| | Thr, or absent | | |
| | X at position 91 is Ala, Glu, Lys, Leu, Met, | | |
| | or Thr | | |
| | X at position 92 is Ala, Ile, Leu, or Val | | |
| | X at position 93 is Asp, Phe, His, Ser, Thr, | | |
| | or Tyr | | |
| | X at position 95 is Glu, Gly, Gln, or Thr | | |
| | X at position 96 is Met or Val | | |
| | X at position 97 is Asp, Asn, Arg, Ser, or Thr | | |
| | X at position 98 is Asp, Ile, Asn, or Ser | | |
| | X at position 100 is Glu, Gly, Lys, Asn, or Arg | | |
| | X at position 101 is Ala, Leu, Pro, or Ser | | |
| | X at position 102 is Asp or Glu | | |
| | X at position 105 is Ala or Gly | | |
| | X at position 106 is Ala, Asp, Leu, or Val | | |
| | X at position 107 is Phe or Tyr | | |
| | X at position 108 is Phe, Ile, Gln, Arg, Thr, | | |
| | Val, or Tyr | | |
| | X at position 110 is Ala, His, Asn, Gln, Ser, | | |
| | Thr, Trp,or Tyr | | |
| | X at position 111 is Ala, Ile, Lys, Leu, Met, | | |
| | Arg, Ser, Thr, or Tyr | | |
| | X at position 112 is Ala, Asp, Glu, Gly, His, | | |
| | Lys, Asn, Arg, Ser, Thr, Val, or Tyr | | |
| | X at position 113 is Ala, Asp, Glu, Phe, Gly, | | |
| | His, Ile, Lys, Asn, Pro, Gln, Arg, Ser, Thr, | | |
| | Tyr, or absent | | |
| | X at position 114 is Ala, Asp, Phe, Gly, Ile, | | |
| | Leu, Pro, Gln, Arg, Ser, Val, Trp, or absent | | |
| | X at position 115 is Ala, Asp, Glu, Phe, Gly, | | |
| | Leu, Met, Arg, Ser, Thr, Val, Tyr, or absent | | |
| | X at position 116 is Phe, Gly, His, Leu, Ser, | | |
| | Thr, Trp, Tyr, or absent | | |
| | X at position 117 is Cys, Gly, His, Asn, Arg, | | |
| | Ser, Thr, Tyr, or absent | | |
| | X at position 118 is Ala, Asp, Phe, Gly, Arg, | | |
| | Ser, Thr, Trp, or absent | | |
| | X at position 119 is Ala, Cys, Asp, Gly, Ile, | | |
| | Leu, Asn, Pro, Arg, Ser, Tyr, or absent | | |
| | X at position 120 is Cys, Gly, Ile, Asn, Pro, | | |
| | Arg, Ser, Thr, Val, Trp, Tyr, or absent | | |
| | X at position 121 is Asp, Phe, Gly, Leu, Ser, | | |
| | Trp, Tyr, or absent | | |
| | X at position 122 is Ala, Asp, Gly, Ile, Leu, | | |
| | Thr, Val, Trp, Tyr, or absent | | |
| | X at position 123 is Cys, Phe, Asn, Val, or | | |
| | absent | | |
| | X at position 124 is Phe, Gly, Ile, Leu, Gln, | | |
| | Ser, Tyr, or absent | | |
| | X at position 125 is Ala, Cys, Asp, Gly, Pro, | | |
| | Arg, Ser, Thr, or absent | | |
| | X at position 126 is Ala, Phe, Gly, Leu, Met, | | |
| | Pro, Gln, Arg, Ser, Thr, Val, Trp, Tyr, or | | |
| | absent | | |
| | X at position 127 is Ala, Asp, Glu, Gly, His, | | |
| | Ile, Pro, Gln, Arg, Ser, Thr, Trp, or absent | | |
| | X at position 128 is Ala, Asp, Gly, Ile, Lys, | | |
| | Met, Asn, Arg, Ser, Thr, Val, Trp, Tyr, or | | |
| | absent | | |
| | X at position 129 is Ala, Asp, Glu, Gly, His, | | |
| | Lys, Leu, Pro, Gln, Arg, Ser, Thr, or absent | | |
| | X at position 130 is Glu, Phe, Gly, His, Ile, | | |
| | Lys, Leu, Met, Asn, Gln, Arg, Ser, Thr, or Tyr | | |
| | X at position 131 is Ala, Asp, Glu, Gly, Ile, | | |
| | Leu, Asn, Pro, Ser, Thr, Val, or Tyr | | |
| | X at position 132 is Ala, Asp, Phe, His, Asn, | | |
| | Ser, Thr, Val, Trp, or Tyr | | |
| | X at position 133 is Arg or Trp | | |
| | X at position 135 is Lys, Asn, or Gln | | |
| 184 | XVQLVXSXGGXVXXGGSLXLSCXXXXXXXXXXXXXWX | V_{H}H | Consensus sequence |
| | RQXPGXXRXXXXXIXXXXGXXXYXXXXXXXKGRFTISR | cons. | for S1-NTD specific |
| | DNAKXXXXLXMXSLXXXDTAXXYCXXXXXXXXXXXXX | seq. | V_{H}Hs, generated |
| | XXXXXXXXXXXGXGTQVTVSS | | based on the |
| | X at position 1 is Ala, Glu, or Gln | | sequence alignment |
| | X at position 6 is Asp. Glu, or Gln | | shown in FIG. 19 |
| | X at position 8 is Ala or Gly | | |
| | X at position 11 is Leu or Ser | | |
| | X at position 13 is Gln or Thr | | |
| | X at position 14 is Ala, Pro, or Thr | | |
| | X at position 19 is Arg or Thr | | |
| | X at position 23 is Ala, Thr, or Val | | |
| | X at position 24 is Ala, Thr, or Val | | |
| | X at position 25 is Ile or Ser | | |
| | X at position 26 is Glu or Gly | | |
| | X at position 27 is Phe, Arg, Ser, or Thr | | |
| | X at position 28 is Ile, Arg, or Thr | | |
| | X at position 29 is Phe, Gly, or Leu | | |
| | X at position 30 is Asp, Ala, Gly, Asn, Pro, | | |
| | or Ser | | |
| | X at position 31 is Phe, Asn, Arg, Ser, or | | |
| | Tyr | | |
| | X at position 32 is His, Lys, Asn, or Tyr | | |
| | X at position 33 is Ala, His, Ser, or Thr | | |
| | X at position 34 is Ile, Met, or Val | | |
| | X at position 35 is Ala or Gly | | |
| | X at position 37 is Phe, His, or Tyr | | |
| | X at position 40 is Ala or Ser | | |
| | X at position 43 is Lys or Asn | | |
| | X at position 44 is Glu or Gln | | |
| | X at position 46 is Asp or Glu | | |
| | X at position 47 is Phe, Gly, Leu, or Pro | | |
| | X at position 48 is Ala or Val | | |
| | X at position 49 is Ala or Ser | | |
| | X at position 50 is Ala, Cys, Gly, or Ile | | |
| | X at position 52 is Arg, Ser, or Thr | | |
| | X at position 53 is Gly, Met, Arg, Ser, Thr, | | |
| | or Tyr | | |
| | X at position 54 is Gly, Asn, or Ser | | |
| | X at position 55 is Asp, Gly, or absent | | |
| | X at position 57 is Ala, Gly, Lys, Asn, Ser, | | |
| | or Val | | |
| | X at position 58 is Ala, Ile, Thr, or Val | | |
| | X at position 59 is Asp, His, Ile, Asn, Thr, | | |
| | or Tyr | | |
| | X at position 61 is Ala, Gly, or absent | | |
| | X at position 62 is Asp or absent | | |
| | X at position 63 is Tyr or absent | | |
| | X at position 64 is Ala or absent | | |
| | X at position 65 is Asp or Ser | | |
| | X at position 66 is Phe or Ser | | |
| | X at position 67 is Ala or Val | | |
| | X at position 80 is Asn or Ser | | |
| | X at position 81 is Ala, Leu, Met, or Thr | | |
| | X at position 82 is Leu or Val | | |
| | X at position 83 is Ser or Tyr | | |
| | X at position 85 is Glu, Gln, or Thr | | |
| | X at position 87 is Asp or Asn | | |
| | X at position 90 is Lys or Asn | | |
| | X at position 91 is Leu or Pro | | |
| | X at position 92 is Asp or Glu | | |
| | X at position 96 is Leu or Val | | |
| | X at position 97 is Phe or Tyr | | |
| | X at position 100 is Ala, Asn, or Tyr | | |
| | X at position 101 is Ala, Met, Arg, or Thr | | |
| | X at position 102 is Asp, Ala, Gly, Asn, or | | |
| | Thr | | |
| | X at position 103 is Ala, Gly, Arg, Ser, or | | |
| | absent | | |
| | X at position 104 is Phe, Ile, Leu, Trp, or | | |
| | absent | | |
| | X at position 105 is Asp, Glu, Ala, Arg, Ser, | | |
| | Val, Tyr, or absent | | |
| | X at position 106 is Gly, Ser, Thr, Tyr, or | | |
| | absent | | |
| | X at position 107 is Cys, Asn, Arg, Thr, or | | |
| | absent | | |
| | X at position 108 is Asp, Ser, Trp, or absent | | |
| | X at position 109 is Asp, Asn, Ser, or absent | | |
| | X at position 110 is Thr, Val, Trp, Tyr, or | | |
| | absent | | |
| | X at position 111 is Glu, Phe, Arg, Tyr, or | | |
| | absent | | |
| | X at position 112 is Gly or absent | | |
| | X at position 113 is Ala, Cys, or absent | | |
| | X at position 114 is Cys, Ser, or absent | | |
| | X at position 115 is Ala, Ile, Leu, Pro, Arg, | | |
| | Ser, or absent | | |
| | X at position 116 is Gln, Leu, Met, Pro, Thr, | | |
| | Tyr, or absent | | |
| | X at position 117 is Ala, Gly, Ile, Pro, or | | |
| | absent | | |
| | X at position 118 is Ala, Gly, Leu, Met, Pro, | | |
| | Ser, Trp, or absent | | |
| | X at position 119 is Gln, Ala, Gly, Pro, Arg, | | |
| | or absent | | |
| | X at position 120 is Ile, Met, Arg, Ser, Thr, | | |
| | Tyr, or absent | | |
| | X at position 121 is Asp, Ile, Asn, Pro, or | | |
| | absent | | |
| | X at position 122 is Phe, His, Asn, Thr, or | | |
| | Tyr | | |
| | X at position 123 is Arg or Trp | | |
| | X at position 125 is Gln, Lys, or Asn | | |
| 185 | XVQLXXSGGGXVQXGGSLXLSCAXSXXXXXXXXXXXX | V_{H}H | Consensus sequence |
| | RQAPGXXXXXXXXXXXXXVXXXXXXXXXXXXXYXDSV | cons. | for S2 specific V_{H}Hs, |
| | XXRFXISRDXXKXXXXLXXXXLXXEDTAXYXCXXXXXX | seq. | generated based on |
| | XXXXXXXXXXXXXXXGXGTQVTVSS | | the sequence |
| | X at position 1 is Ala or Pro | | alignment shown in |
| | X at position 5 is Pro or Val | | FIG. 20 |
| | X at position 6 is Gln, Ala, or Pro | | |
| | X at position 11 is Lys or Ser | | |
| | X at position 14 is Asp or Asn | | |
| | X at position 19 is Arg or Thr | | |
| | X at position 24 is Asp, Asn, Thr, or Val | | |
| | X at position 26 is Asp, Phe, Thr, or Val | | |
| | X at position 27 is Cys, Gly, His, Lys, Arg, | | |
| | Ser, or Thr | | |
| | X at position 28 is His, Asn, Ser, Thr, or | | |
| | Val | | |
| | X at position 29 is Cys, Lys, Val, or Tyr | | |
| | X at position 30 is Asp, Phe, Ile, Met, Arg, | | |
| | or Ser | | |
| | X at position 31 is Gln, Phe, His, Lys, Met, | | |
| | Arg, Ser, or Val | | |
| | X at position 32 is Cys, His, Met, Asn, Pro, | | |
| | Ser, or Tyr | | |
| | X at position 33 is Asp, Gln, Phe, Lys, Ser, | | |
| | Thr, or Val | | |
| | X at position 34 is His, Leu, or Thr | | |
| | X at position 35 is Asp, Ala, Phe, Met, or | | |
| | Ser | | |
| | X at position 36 is Trp or Tyr | | |
| | X at position 37 is Cys, Gly, Arg, or Tyr | | |
| | X at position 43 is Ile or Met | | |
| | X at position 44 is Ala, Phe, or Pro | | |
| | X at position 45 is Gly, Lys, or absent | | |
| | X at position 46 is Ala or absent | | |
| | X at position 47 is Lys or absent | | |
| | X at position 48 is Val or absent | | |
| | X at position 49 is Asp or absent | | |
| | X at position 50 is Phe or absent | | |
| | X at position 51 is Ile or absent | | |
| | X at position 52 is Pro or absent | | |
| | X at position 53 is Arg or absent | | |
| | X at position 54 is Gln or Ala | | |
| | X at position 55 is Cys, Phe, Lys, Leu, or | | |
| | Trp | | |
| | X at position 57 is Asp or Ser | | |
| | X at position 58 is Asp, Glu, Gly, Ser, Thr, | | |
| | or Val | | |
| | X at position 59 is His, Leu, or Val | | |
| | X at position 60 is Met, Arg, Ser, or absent | | |
| | X at position 61 is Ala, Ser, Thr, or Trp | | |
| | X at position 62 is Asp, Gln, Phe, Arg, Ser, | | |
| | or Thr | | |
| | X at position 63 is Gln or absent | | |
| | X at position 64 is Arg or absent | | |
| | X at position 65 is Gln, Phe, Arg, or Ser | | |
| | X at position 66 is Gln, Phe, or Ser | | |
| | X at position 67 is Asp, Gln, Phe, Met, Ser, | | |
| | or Thr | | |
| | X at position 68 is Asp, Ile, Asn, Arg, or Thr | | |
| | X at position 69 is Ile, Met, Ser, Thr, or Tyr | | |
| | X at position 71 is Asp, Arg, or Thr | | |
| | X at position 75 is Ile or Met | | |
| | X at position 76 is Asp or Phe | | |
| | X at position 79 is Asp or Thr | | |
| | X at position 84 is Gln or Met | | |
| | X at position 85 is Asp or Ala | | |
| | X at position 87 is Gly, Met, or absent | | |
| | X at position 88 is Ala or Thr | | |
| | X at position 89 is Asp, His, Lys, or Val | | |
| | X at position 90 is Cys, Thr, or Tyr | | |
| | X at position 92 is Phe or Pro | | |
| | X at position 93 is Leu or Val | | |
| | X at position 94 is Gln, Met, or Ser | | |
| | X at position 95 is His or Ser | | |
| | X at position 97 is Ala, Phe, Ile, or Arg | | |
| | X at position 98 is Lys or Asn | | |
| | X at position 103 is Lys or Val | | |
| | X at position 105 is Pro, Thr, or Tyr | | |
| | X at position 107 is Asp, Gly, Met, or Tyr | | |
| | X at position 108 is Asp, ile, Lys, Leu, Thr, | | |
| | or Tyr | | |
| | X at position 109 is Asp, Ala, Phe, Gly, Ile, | | |
| | Arg, Thr, Val, or Tyr | | |
| | X at position 110 is Asp, Gln, Ala, Cys, His, | | |
| | Ile, Met, Asn, Ser, or absent | | |
| | X at position 111 is Phe, His, Lys, Asn, Arg, | | |
| | Ser, Val, Tyr, or absent | | |
| | X at position 112 is Gln, Cys, Leu, Ser, Tyr, | | |
| | or absent | | |
| | X at position 113 is Phe, Ser, Thr, Trp, Tyr, | | |
| | or absent | | |
| | X at position 114 is Phe, Gly, Ser, or absent | | |
| | X at position 115 is Phe, Arg, Ser, Thr, or | | |
| | absent | | |
| | X at position 116 is Asp, Gln, Phe, or absent | | |
| | X at position 117 is Thr, Tyr, or absent | | |
| | X at position 118 is Glu, His, Ser, Tyr, or | | |
| | absent | | |
| | X at position 119 is Lys, Ser, Val, or absent | | |
| | X at position 120 is Ser, Thr, Val, Tyr, or | | |
| | absent | | |
| | X at position 121 is Asp, Gln, Cys, His, Asn, | | |
| | Trp, Tyr, or absent | | |
| | X at position 122 is Gln, Ile, Lys, Asn, Arg, | | |
| | Ser, Thr, Trp, or Tyr | | |
| | X at position 123 is Asp, Phe, Lys, Arg, Ser, | | |
| | or Thr | | |
| | X at position 124 is Cys, Phe, His, Ile, Lys, | | |
| | Pro, Arg, Ser, or Tyr | | |
| | X at position 125 is Asp, Gln, Lys, Met, Asn, | | |
| | Ser, Thr, or Val | | |
| | X at position 126 is Asp, Cys, Met, Thr, Trp, | | |
| | or Tyr | | |
| | X at position 127 is Arg or Trp | | |
| | X at position 129 is Ile or Pro | | |
| 186 | XXXLXXSGGGXVXXGXSLXLSCXXXXXXXXXXXXXWX | V_{H}H | Consensus sequence |
| | RQXXXXXGXXXXXXXXXXXXXXXXXYXXXXXGRFTXS | cons. | for S1-RBD specific |
| | XDXXXXXXXLQMXXLXXXDTXXYXCXXXXXXXXXXXX | seq. | V_{H}Hs, generated |
| | XXXXXXXXXXXXGXGTQVTVSS | | based on the |
| | X at position 1 is Asp, Ala, or Pro | | sequence alignment |
| | X at position 2 is Lys or Val | | shown in FIG. 21 |
| | X at position 3 is Ile or Pro | | |
| | X at position 5 is Lys, Pro, or Val | | |
| | X at position 6 is Gln, Ala, Pro, or Ser | | |
| | X at position 11 is Lys or Ser | | |
| | X at position 13 is Ala, Lys, or Pro | | |
| | X at position 14 is Asp, Asn, or Thr | | |
| | X at position 16 is Ala, Phe, or Arg | | |
| | X at position 19 is Arg or Thr | | |
| | X at position 23 is Asp, Ala, His, Thr, or | | |
| | Val | | |
| | X at position 24 is Asp, Thr, or Val | | |
| | X at position 25 is Asp or Ser | | |
| | X at position 26 is Phe or Val | | |
| | X at position 27 is Cys, His, Lys, Met, Arg, | | |
| | Ser, Thr, or Val | | |
| | X at position 28 is His, Asn, Ser, or Thr | | |
| | X at position 29 is Cys, His, Lys, or Ser | | |
| | X at position 30 is Gln, Phe, Met, Arg, or | | |
| | Ser | | |
| | X at position 31 is Gln, Phe, Gly, His, Met, | | |
| | Pro, Arg, Ser, or Tyr | | |
| | X at position 32 is Lys, Met, Ser, Val, or | | |
| | Tyr | | |
| | X at position 33 is Asp, Met, Ser, Thr, or | | |
| | Val | | |
| | X at position 34 is His, Leu, or Val | | |
| | X at position 35 is Asp, Phe, Gly, Met, or | | |
| | Ser | | |
| | X at position 37 is Cys, Val, Trp, or Tyr | | |
| | X at position 40 is Asp, His, Ser, or Thr | | |
| | X at position 41 is Asn or absent | | |
| | X at position 42 is Pro or absent | | |
| | X at position 43 is Asp or absent | | |
| | X at position 44 is Asn, Arg, or Ser | | |
| | X at position 46 is Asp, Ile, or Pro | | |
| | X at position 47 is Gln, Ala, Phe, Asn, or | | |
| | Pro | | |
| | X at position 48 is Gly, Lys, Arg, or Thr | | |
| | X at position 49 is Asp, Gln, or Ala | | |
| | X at position 50 is Asp, Cys, Phe, Lys, | | |
| | Arg, Val, or Trp | | |
| | X at position 51 is His, Lys, or Val | | |
| | X at position 52 is Asp, Ser, or Thr | | |
| | X at position 53 is Asp, Glu, Cys, Phe, | | |
| | His, Ser, Thr, Val, or Tyr | | |
| | X at position 54 is His, Lys, Leu, or Val | | |
| | X at position 55 is Phe, Met, Ser, Thr, or | | |
| | Trp | | |
| | X at position 56 is Cys, Phe, Met, Asn, | | |
| | Arg, Ser, Thr, Val, Trp, or absent | | |
| | X at position 57 is Phe, Met, Arg, Ser, | | |
| | Tyr, or absent | | |
| | X at position 58 is Gln, Cys, Phe, Leu, | | |
| | Arg, Ser, Thr, Val, or Tyr | | |
| | X at position 59 is Gln, Ala, Phe, Arg, | | |
| | Ser, or Thr | | |
| | X at position 60 is Asp, Gln, Ala, His, | | |
| | Met, Arg, Ser, or Thr | | |
| | X at position 61 is Asp, Phe, His, Met, | | |
| | Arg, Ser, or Thr | | |
| | X at position 62 is Gln, Cys, Gly, His, | | |
| | Lys, Met, Arg, Ser, Thr, Val, or Tyr | | |
| | X at position 64 is Asp, Phe, His, Ser, | | |
| | Thr, or Val | | |
| | X at position 65 is Asp, Gln, Ala, Phe, | | |
| | Ile, Leu, or Tyr | | |
| | X at position 66 is His or Ser | | |
| | X at position 67 is Asp or Val | | |
| | X at position 68 is Asp, Ile, or Arg | | |
| | X at position 73 is Cys, His, Lys, Thr, | | |
| | or Val | | |
| | X at position 75 is Arg or Thr | | |
| | X at position 77 is Gln, Phe, or Met | | |
| | X at position 78 is Asp, Gln, Phe, or Thr | | |
| | X at position 79 is Ala, Ile, Pro, or Arg | | |
| | X at position 80 is Gln, Ile, Met, Ser, or | | |
| | Thr | | |
| | X at position 81 is Asp, Ile, or Thr | | |
| | X at position 82 is Asp, His, Lys, or Val | | |
| | X at position 83 is Gln, Cys, Gly, Ser, Thr, | | |
| | or Tyr | | |
| | X at position 87 is Gln, Met, Arg, Ser, or | | |
| | Thr | | |
| | X at position 88 is Gln, Met, or Ser | | |
| | X at position 90 is Ala, Ile, Met, or Arg | | |
| | X at position 91 is Asp, Asn, or Ser | | |
| | X at position 92 is Gln or Ala | | |
| | X at position 95 is Asp or Phe | | |
| | X at position 96 is Asp, Gln, or Val | | |
| | X at position 98 is Cys, His, Arg, Val, | | |
| | or Tyr | | |
| | X at position 100 is Asp, Gly, Met, Pro, | | |
| | Ser, Thr, Trp, or Tyr | | |
| | X at position 101 is Asp, His, Ile, Lys, | | |
| | Ser, or Thr | | |
| | X at position 102 is Asp, Gln, Phe, Gly, | | |
| | Arg, Ser, Val, or Tyr | | |
| | X at position 103 is Phe, Gly, Met, Asn, | | |
| | Pro, Arg, Ser, Tyr, or absent | | |
| | X at position 104 is Asp, Gln, Phe, Asn, | | |
| | Pro, Arg, Val, or absent | | |
| | X at position 105 is Asp, Gln, Cys, Phe, | | |
| | Lys, Leu, Arg, Ser, Thr, Val, Tyr, or | | |
| | absent | | |
| | X at position 106 is Cys, Gly, Lys, Met, | | |
| | Arg, Ser, Val, Tyr, or absent | | |
| | X at position 107 is Asp, Ala, Arg, Ser, | | |
| | Val, Tyr, or absent | | |
| | X at position 108 is Cys, Phe, Lys, Leu, | | |
| | Ser, Tyr, or absent | | |
| | X at position 109 is Gln, Phe, Arg, or | | |
| | absent | | |
| | X at position 110 is Glu, Met, Asn, Arg, | | |
| | Ser, or absent | | |
| | X at position 111 is Cys, Lys, Ser, Thr, | | |
| | Tyr, or absent | | |
| | X at position 112 is Asp, Phe, Lys, Trp, | | |
| | Tyr, or absent | | |
| | X at position 113 is Glu, Cys, Met, Asn, | | |
| | Val, Tyr, or absent | | |
| | X at position 114 is Cys, Phe, His, Lys, | | |
| | Pro, Ser, Tyr, or absent | | |
| | X at position 115 is Glu, Phe, Arg, Ser, | | |
| | Thr, or absent | | |
| | X at position 116 is Asp, Asn, Arg, Ser, | | |
| | Trp, Tyr, or absent | | |
| | X at position 117 is Asp, Gln, Ala, Phe, | | |
| | Gly, Asn, Pro, Arg, Thr, Val, Tyr, or | | |
| | absent | | |
| | X at position 118 is Asp, Gln, Phe, His, | | |
| | Met, Arg, Ser, Thr, Val, or absent | | |
| | X at position 119 is Asp, Gln, Ala, Phe, | | |
| | Gly, Ile, Lys, Pro, Arg, Ser, Thr, or | | |
| | absent | | |
| | X at position 120 is Phe, Gly, Leu, Met, | | |
| | Ser, Tyr, or absent | | |
| | X at position 121 is Gln, Ala, Phe, Lys, | | |
| | Met, Ser, Thr, or Tyr | | |
| | X at position 122 is Gln, Ser, Thr, Val, or Tyr | | |
| | X at position 123 is Arg or Trp | | |
| | X at position 125 is Ile or Pro | | |

Bold highlighting in V_{H}H sequences indicates the locations of CDR sequences, as defined using the IMGT numbering system described in Lefranc et al (2003).

### REFERENCES

All publications identified herein, including each of the references listed below and any published sequences that are identified by name and/or accession number, are hereby incorporated by reference in their entirety.
Adejare, A. (ed.) (2021) “Remington (23^{rd} Edition), The Science and Practice of Pharmacy” Academic Press, ISBN 978-O-12-820007-0.
Akache B, et al. Immunogenic and efficacious SARS-CoV-2 vaccine based on resistin-trimerized spike antigen SmT1 and SLA archaeosome adjuvant. Sci Rep 11, 21849 (2021).
Algaissi, A., and Hashem, A. M. (2020) Evaluation of MERS-CoV Neutralizing Antibodies in Sera Using Live Virus Microneutralization Assay. Methods Mol Biol 2099, 107-116.
Altschul, S. F., Gish, W., Miller, W., Myers, E. W., Lipman, D. J., Basic local alignment search tool, Journal of Molecular Biology, 1990, 215(3): 403-410.
Altschul, S.F., Madden, T. L., Schäffer, A. A., Zhang, J., Zhang, Z., Miller, W., Lipman, D. J., Gapped BLAST and PSI-BLAST: a new generation of protein database search programs, Nucleic Acids Research, 1997, 25(17):3389-402.
Arbabi-Ghahroudi, M. Desmyter A, Wyns L, Hamers R., and Muyldermans S (1997) Selection and identification of single domain antibody fragments from camel heavy-chain antibodies, FEBS Lett 414, 521-526.
Arora, P, Jafferany, M, Lotti, T, Sadoughifar, R, Goldust, M. Learning from history: Coronavirus outbreaks in the past. Dermatologic Therapy. 2020; 33:e13343.
Chi, X., Yan, R., Zhang, J., Zhang, G., Zhang, Y., Hao, M., Zhang, Z., Fan, P., Dong, Y., Yang, Y., Chen, Z., Guo, Y., Zhang, J., Li, Y., Song, X., Chen, Y., Xia, L., Fu, L., Hou, L., Xu, J., Yu, C., Li, J., Zhou, Q., and Chen, W. (2020) A neutralizing human antibody binds to the N-terminal domain of the Spike protein of SARS-CoV-2. Science 369, 650-655.
Colwill K, et al. A “Made-in-Canada” serology solution for profiling humoral immune responses to SARS-CoV-2 infection and vaccination. medRxiv, 2021.2010.2025.21265476 (2021).
Coughlin, M. M., Babcook, J., and Prabhakar, B. S. (2009) Human monoclonal antibodies to SARS-coronavirus inhibit infection by different mechanisms. Virology 394, 39-46.
Davies J., and L. Riechmann, Affinity improvement of single antibody V_{H} domains: residues in all three hypervariable regions affect antigen binding. Immunotechnology 2 (1996) 169-179.
Delfin-Riela, T., Rossotti, M. A., Alvez-Rosado R, Leizagoyen C, and Gonzalez-Sapienza, G. (2020) Highly Sensitive Detection of Zika Virus Nonstructural Protein 1 in Serum Samples by a Two-Site Nanobody ELISAA nanobody-based test for highly sensitive detection of hemoglobin in fecal samples. Biomolecules, 10 (12):1652.
Detalle, L., Stohr, T., Palomo, C., Piedra, P. A., Gilbert, B. E., Mas, V., Millar, A., Power, U. F., Stortelers, C., Allosery, K., Melero, J. A., and Depla, E. (2016) Generation and Characterization of ALX-0171, a Potent Novel Therapeutic Nanobody for the Treatment of Respiratory Syncytial Virus Infection. Antimicrob Agents Chemother 60, 6-13.
Dondelinger, M., Filee, P., Sauvage, E., Quinting, B., Muyldermans, S., Galleni, M., & Vandevenne, M. S. (2018). Understanding the Significance and Implications of Antibody Numbering and Antigen-Binding Surface/Residue Definition. Frontiers in immunology, 9, 2278.
Dumoulin, M., Conrath, K., Van Meirhaighe, A., Meersman, F., Heremans, K., Frenken, L. G., Muyldermans, S., Wyns, L, and Matagne, A. (2002) Protein Sci 11, 500-S15.
Galipeau Y, et al. Relative Ratios of Human Seasonal Coronavirus Antibodies Predict the Efficiency of Cross-Neutralization of SARS-CoV-2 Spike Binding to ACE2. EBioMedicine 74, 103700 (2021).
Hamers-Casterman, C, Atarhouch, T., Muyldermans, S., Robinson, G., Hamers, C, Songa, E. B., Bendahman, N., and Hamers, R. (1993) Nature 363, 446-448 Henry, K. A., Hussack, G., Collins, C., Zwaagstra, J. C., Tanha, J., and MacKenzie, C. R. (2016) Isolation of TGF-beta-neutralizing single-domain antibodies of predetermined epitope specificity using next-generation DNA sequencing. Protein Eng Des Sel 29, 439-443.
Henry, K. A., Kim, D. Y., Kandalaft, H., Lowden, M. J., Yang, Q., Schrag, J. D., Hussack, G., MacKenzie, C. R., and Tanha, J. (2017) Stability-Diversity Tradeoffs Impose Fundamental Constraints on Selection of Synthetic Human V_{H}/V_{L} Single-Domain Antibodies from In Vitro Display Libraries. Front Immunol 8, 1759.
Henry, K. A., Tanha, J., and Hussack, G. (2015) Identification of cross-reactive single-domain antibodies against serum albumin using next-generation DNA sequencing. Protein Eng Des Sel 28, 379-383.
Hussack, G., Arbabi-Ghahroudi, M., van Faassen, H., Songer, J. G., Ng, K. K., MacKenzie, R., and Tanha, J. (2011) Neutralization of Clostridium difficile toxin A with single-domain antibodies targeting the cell receptor binding domain. J Biol Chem 286, 8961-8976.
Jespers, L, Schon, O., Famm, K., and Winter, G. (2004) Nat. Biotechnol. 22, 1161-1165.
Karlin, S., Altschul, S.F., Applications and statistics for multiple high-scoring segments in molecular sequences. PNAS, 1993, 90(12): 5873-5877.
Karlin, S., Altschul, S.F., Methods for assessing the statistical significance of molecular sequence features by using general scoring schemes. PNAS, 1990, 87(6): 2264-2268.
Kim, D. Y., Kandalaft, H., Ding, W., Ryan, S., van Fassen, H., Hirama, T., Foote, S. J., MacKenzie, R., and Tanha, J. (2012) PEDS advance access Aug. 30, 2012, 1-9.
Lan, J., Ge, J., Yu, J., Shan, S., Zhou, H., Fan, S., Zhang, Q., Shi, X., Wang, Q., Zhang, L., Wang, X. (2020), Structure of the SARS-CoV-2 spike receptor-binding domain bound to the ACE2 receptor. Nature 581, 215-220.
Lefranc, M.-P. et al., (2003) Dev. Comp. Immunol., 27, 55-77.
Li, R. (2016) Structure, Function, and Evolution of Coronavirus Spike Proteins, Annual Review of Virology 3, 237-261.
Li S, Zheng W, Kuolee R, Hirama T, Henry M, Makvandi-Nejad S, Fjallman T, Chen W, Zhang J. Pentabody-mediated antigen delivery induces antigen-specific mucosal immune response. Mol Immunol 2009; 46:1718-26.
Myers, E.W., Miller, W., Optimal alignments in linear space, Bioinformatics, 4(1): 11-17. Sliepen K, van Montfort T, Melchers M, Isik G, Sanders R W, 2015. Immunosilencing a highly immunogenic protein trimerization domain. J Biol Chem 290, 7436-7442.
Nuttall, S.D., Krishnan, U. V., Doughty, L., Pearson, K., Ryan, M. T., Hoogenraad, N.J., Hattarki, M., Carmichael, J.A., Irving, R. A., and Hudson, P. J. (2003) Eur. J. Biochem. 270, 3543-3554.
Ravichandran, S., Coyle, E. M., Klenow, L., Tang, J., Grubbs, G., Liu, S., Wang, T., Golding, H., and Khurana, S. (2020) Antibody signature induced by SARS-CoV-2 spike protein immunogens in rabbits. Sci Transl Med 12.
Respaud, R., Vecellio, L., Diot, P., and Heuze-Vourc'h, N. (2015) Nebulization as a delivery method for mAbs in respiratory diseases. Expert Opin Drug Deliv 12, 1027-1039.
Rogers, T. F., Zhao, F., Huang, D., Beutler, N., Burns, A., He, W. T., Limbo, O., Smith, C., Song, G., Woehl, J., Yang, L., Abbott, R. K., Callaghan, S., Garcia, E., Hurtado, J., Parren, M., Peng, L., Ramirez, S., Ricketts, J., Ricciardi, M. J., Rawlings, S. A., Wu, N. C., Yuan, M., Smith, D. M., Nemazee, D., Teijaro, J. R., Voss, J. E., Wilson, I. A., Andrabi, R., Briney, B., Landais, E., Sok, D., Jardine, J. G., and Burton, D. R. (2020) Isolation of potent SARS-CoV-2 neutralizing antibodies and protection from disease in a small animal model. Science 369, 956-963.
Rossotti, M. A., Henry, K. A., van Faassen, H., Tanha, J., Callaghan, D., Hussack, G., Arbabi-Ghahroudi, M., and MacKenzie, C. R. (2019) Camelid single-domain antibodies raised by DNA immunization are potent inhibitors of EGFR signaling. Biochem J 476, 39-50.
Rossotti, M. A., Pirez, M., Gonzalez-Techera, A., Cui, Y., Bever, C. S., Lee, K. S., Morisseau, C., Leizagoyen, C., Gee, S., Hammock, B. D., and Gonzalez-Sapienza, G. (2015a) Method for Sorting and Pairwise Selection of Nanobodies for the Development of Highly Sensitive Sandwich Immunoassays. Anal Chem 87, 11907-11914.
Rossotti, M., Tabares, S., Alfaya, L., Leizagoyen, C., Moron, G., and Gonzalez-Sapienza, G. (2015b) Streamlined method for parallel identification of single domain antibodies to membrane receptors on whole cells. Biochim Biophys Acta 1850, 1397-1404.
Stuible, M. Gervais, C., Lord-Dufour, S., Perret, S., L'Abbe, D., Schrag, J., St-Laurent, G., Durocher, Y. (2021), Rapid, high-yield production of full-length SARS-CoV-2 spike ectodomain by transient gene expression in CHO cells. Journal of Biotechnology 326, 21-27.
Sulea T, Baardsnes J, Stuible M, Rohani N, Tran A, Parat M, Cepero Donates Y, Duchesne M, Plante P, Kour G, Durocher Y. (2022) Structure-based dual affinity optimization of a SARS-CoV-1/2 cross-reactive single-domain antibody. PLoS One. 17(3):e0266250.
Wrapp, D., De Vlieger, D., Corbett, K. S., Torres, G. M., Wang, N., Van Breedam, W., Roose, K., van Schie, L., Team, V.-C. C.-R, Hoffmann, M., Pohlmann, S., Graham, B. S., Callewaert, N., Schepens, B., Saelens, X., and McLellan, J. S. (2020) Structural Basis for Potent Neutralization of Betacoronaviruses by Single-Domain Camelid Antibodies. Cell 181, 1004-1015 e1015.
Wrapp D, et al. Cryo-EM structure of the 2019-nCoV spike in the prefusion conformation. Science 367, 1260-1263 (2020).
Zheng M, Zhao X, Zheng S, et al. Bat SARS-Like WIV1 coronavirus uses the ACE2 of multiple animal species as receptor and evades IFITM3 restriction via TMPRSS2 activation of membrane fusion. Emerg Microbes Infect. 2020; 9(1):1567-1579.
Zhou, P., Yang, XL., Wang, X G. et al. (2020) A pneumonia outbreak associated with a new coronavirus of probable bat origin. Nature 579, 270-273.

1. An isolated or purified antibody that specifically recognizes at least one coronavirus spike polypeptide, wherein the antibody comprises an antigen binding portion of an antibody heavy chain, wherein the antigen binding portion comprises a first complementarity determining region (CDR1), a second complementarity determining region (CDR2), and a third complementarity determining region (CDR3), and wherein CDR1, CDR2, and CDR3, respectively, comprise the amino acid sequence set forth in:
SEQ ID NO: 1, SEQ ID NO: 45, and SEQ ID NO: 90;
SEQ ID NO: 2, SEQ ID NO: 45, and SEQ ID NO: 91;
SEQ ID NO: 1, SEQ ID NO: 46, and SEQ ID NO: 92;
SEQ ID NO: 3, SEQ ID NO: 47, and SEQ ID NO: 93;
SEQ ID NO: 4, SEQ ID NO: 48, and SEQ ID NO: 94;
SEQ ID NO: 5, SEQ ID NO: 49, and SEQ ID NO: 95;
SEQ ID NO: 6, SEQ ID NO: 50, and SEQ ID NO: 96;
SEQ ID NO: 7, SEQ ID NO: 51, and SEQ ID NO: 97;
SEQ ID NO: 8, SEQ ID NO: 52, and SEQ ID NO: 98;
SEQ ID NO: 9, SEQ ID NO: 53, and SEQ ID NO: 99;
SEQ ID NO: 10, SEQ ID NO: 54, and SEQ ID NO: 100;
SEQ ID NO: 11, SEQ ID NO: 55, and SEQ ID NO: 101;
SEQ ID NO: 12, SEQ ID NO: 56, and SEQ ID NO: 102;
SEQ ID NO: 13, SEQ ID NO: 57, and SEQ ID NO: 103;
SEQ ID NO: 14, SEQ ID NO: 58, and SEQ ID NO: 104;
SEQ ID NO: 15, SEQ ID NO: 59, and SEQ ID NO: 105;
SEQ ID NO: 16, SEQ ID NO: 60, and SEQ ID NO: 106;
SEQ ID NO: 17, SEQ ID NO: 61, and SEQ ID NO: 107;
SEQ ID NO: 18, SEQ ID NO: 62, and SEQ ID NO: 108;
SEQ ID NO: 19, SEQ ID NO: 63, and SEQ ID NO: 109;
SEQ ID NO: 20, SEQ ID NO: 64, and SEQ ID NO: 110;
SEQ ID NO: 21, SEQ ID NO: 65, and SEQ ID NO: 111;
SEQ ID NO: 22, SEQ ID NO: 66, and SEQ ID NO: 112;
SEQ ID NO: 23, SEQ ID NO: 67, and SEQ ID NO: 113;
SEQ ID NO: 24, SEQ ID NO: 68, and SEQ ID NO: 114;
SEQ ID NO: 25, SEQ ID NO: 69, and SEQ ID NO: 115;
SEQ ID NO: 26, SEQ ID NO: 70, and SEQ ID NO: 116;
SEQ ID NO: 27, SEQ ID NO: 71, and SEQ ID NO: 117;
SEQ ID NO: 28, SEQ ID NO: 72, and SEQ ID NO: 118;
SEQ ID NO: 29, SEQ ID NO: 73, and SEQ ID NO: 119;
SEQ ID NO: 30, SEQ ID NO: 74, and SEQ ID NO: 120;
SEQ ID NO: 31, SEQ ID NO: 75, and SEQ ID NO: 121;
SEQ ID NO: 32, SEQ ID NO: 76, and SEQ ID NO: 122;
SEQ ID NO: 33, SEQ ID NO: 77, and SEQ ID NO: 123;
SEQ ID NO: 34, SEQ ID NO: 78, and SEQ ID NO: 124;
SEQ ID NO: 35, SEQ ID NO: 79, and SEQ ID NO: 125;
SEQ ID NO: 36, SEQ ID NO: 80, and SEQ ID NO: 125;
SEQ ID NO: 20, SEQ ID NO: 81, and SEQ ID NO: 126;
SEQ ID NO: 20, SEQ ID NO: 81, and SEQ ID NO: 127;
SEQ ID NO: 37, SEQ ID NO: 82, and SEQ ID NO: 128;
SEQ ID NO: 38, SEQ ID NO: 83, and SEQ ID NO: 129;
SEQ ID NO: 39, SEQ ID NO: 84, and SEQ ID NO: 130;
SEQ ID NO: 40, SEQ ID NO: 85, and SEQ ID NO: 131;
SEQ ID NO: 41, SEQ ID NO: 86, and SEQ ID NO: 132;
SEQ ID NO: 42, SEQ ID NO: 87, and SEQ ID NO: 133;
SEQ ID NO: 43, SEQ ID NO: 88, and SEQ ID NO: 134; or
SEQ ID NO: 44, SEQ ID NO: 89, and SEQ ID NO: 135.

2. The antibody of claim 1, wherein the antibody is a neutralizing antibody and wherein CDR1, CDR2, and CDR3, respectively, comprise the amino acid sequence set forth in:
SEQ ID NO: 11, SEQ ID NO: 55, and SEQ ID NO: 101;
SEQ ID NO: 13, SEQ ID NO: 57, and SEQ ID NO: 103;
SEQ ID NO: 19, SEQ ID NO: 63, and SEQ ID NO: 109; or
SEQ ID NO: 21, SEQ ID NO: 65, and SEQ ID NO: 111.

3. The antibody of claim 1, wherein the antibody comprises the amino acid sequence set forth in SEQ ID NO: 183, SEQ ID NO: 184, SEQ ID NO: 185, or SEQ ID NO: 186.

4. The antibody of claim 1, wherein the antibody comprises the amino acid sequence set forth in SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, or SEQ ID NO: 182, or an amino acid sequence having at least 75% sequence identity to the full length of the amino acid sequence set forth in SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, or SEQ ID NO: 182.

5. The antibody of claim 1, wherein the antibody is a single domain antibody.

6-7. (canceled)

8. The antibody of claim 1, wherein the antibody is in a multivalent display format.

9. The antibody of claim 8, wherein the antibody is linked to an Fc fragment.

10. (canceled)

11. A nucleic acid molecule encoding the antibody of claim 1.

12. A vector comprising the nucleic acid molecule of claim 11.

13. (canceled)

14. A host cell comprising the vector of claim 12.

15. A pharmaceutical composition comprising at least one antibody as defined in claim 1 and a pharmaceutically acceptable carrier and/or diluent.

16. (canceled)

17. A composition comprising at least one antibody as defined in claim 1 linked to another molecule.

18. (canceled)

19. The composition of claim 17, wherein the other molecule is an ACE2 polypeptide or a fragment thereof.

20. A composition comprising at least one antibody as defined in claim 1 immobilized on a substrate.

21-26. (canceled)

27. A method for treating or preventing a coronavirus infection, the method comprising administering at least one antibody as defined claim 1 to a subject in need thereof.

28-30. (canceled)

31. A method for detecting the presence of a coronavirus or a coronavirus spike polypeptide or fragment thereof in a sample, the method comprising exposing the sample to at least one antibody as defined in claim 1 and assaying for specific binding between the at least one antibody and the sample, wherein specific binding indicates a presence of the at least one coronavirus or coronavirus spike polypeptide or fragment thereof in the sample.

32. A method for capturing a coronavirus or a coronavirus spike polypeptide or fragment thereof from a sample, the method comprising exposing the sample to the composition as defined in claim 20.

33. The method of claim 31, wherein the coronavirus is a coronavirus that specifically binds an ACE2 receptor or the coronavirus spike polypeptide is a coronavirus spike polypeptide that specifically binds an ACE2 receptor.

34. The method of claim 31, wherein the coronavirus is SARS-CoV-2 or SARS-CoV, or the coronavirus spike polypeptide or fragment thereof is a SARS-CoV-2 or SARS-CoV coronavirus spike polypeptide or fragment thereof.

35-44. (canceled)

45. An antibody cocktail composition comprising two or more of the antibodies of claim 1.
